SEQUENCE LISTING (412 sequences)

SEQ ID NO: 1 - DNA, 852 bp, synthetic construct
  source 1..852: mol_type = other DNA
caggctgtgctgaatcagccggcctctgtgtctgcagccctggggcagagggtcaccatctcctgtaacaccaacatcggcagtccttatgatgtacaatggtaccagcagctcccaggaaagtcccctaaaactatcatttatggtaatagcaatcgaccctcgggggtcccggttcgattctctggctccaagtcaggcagcacagccaccctgaccatcgctgggatccaggctgaggatgaggctgattattactgccagtcctatgatgacagcctcgatggtcgtgttgtgttcggcggagggacccaactgaccgtcctcggtggtggttcctctagatcttcctcctctggtggcggtggctcgggcggtggtggggaggtgcagctggtggagtctgggggagacctggtgaagcctggggggtccttgagactctcctgtgtggcctctggattcaccttcagtaaccacgaaatgtactgggtccgccaggctccagggaaagggctggagtgggtcgcaaggatttatgatagtggaagtagaacaaggtatgcagacgctgtgaagggccgattcaccatctccagagacaacgccaagaacacgctgtatcttcagatgaacagcctgacagccgaggacacggccgtttatttctgtgcgggggggaattctcgtggttggaccgagtttggtatggactactggggcccgggaaccctggtcaccgtctcctcagcctccaccacggccccctcggtcactagtggccaggccggccagcaccatcaccatcaccatggcgcatacccgtacgacgttccggactacgcttcttag

SEQ ID NO: 2 - DNA, 852 bp, synthetic construct
  source 1..852: mol_type = other DNA
tcctatgtgctgactcagtcaccctcactgtctgggtccctgggccagaaggtcaccatctcctgcactggaagtcgttccaatctcggtggaaattatgtgggctggtaccaacaagtcccaggaatggcccctagaaatgtcatatatgctaatgattttcgaccttcacggattcccgctcgattctccgcctccaagtcaggcacttcagccaccctgaccatctctgggctccaggctgaggacgaggctgactatttttgttcatcatgggatgatgatctcgggagtcacgtgttcggctcagggacccagctgaccatcctcggtggtggttcctctagatcttcctcctctggtggcggtggctcgggcggtggtggggaggagcaactggtggagtttgggggagacttggtgaagcctggggggtccctgagactctcctgtgtagcctctggattcagtttcagtagctacgacatgagctgggtccgccaggctcctgggaaggggctgcaatgggtcgcatctactaactttgatggaagtcgcacatactacactgacgctgtggagggccgattcaccatctccagagacaacgccaggaacacagtgtatctgcagatgaacagcctgagagccgaggacacggctgtgtattactgtgcgagggggatgggtagttggggcgcttcgacctttgggtactggggccagggaaccctggtcaccgtctcctcagcctccaccacggccccctcggtcactagtggccaggccggccagcaccatcaccatcaccatggcgcatacccgtacgacgttccggactacgcttcttag

SEQ ID NO: 3 - DNA, 843 bp, synthetic construct
  source 1..843: mol_type = other DNA
cagtctgtgctgaatcagccaccttctgtgtctggagccctggggcagaccgtcaccatctcctgctctggaagtcacaccaacatcggcagtggatttgatgtacagtggtaccaacagctcccaggaaagtctcctcaaactctcgtctatgctaatatcaatcgaccctcgggggtcccggctcgattctctggctccaagtcaggcagcacagccaccctgaccatcactggggtccaggctgaagatgaggctgattattactgccagtcgtatgatgacaacttcgatgggcacgtgttcggctcaggaacccaactgaccgtcctcggcggtggttcctctagatcttcctcctctggtggcggtggctcgggcggtggtggggaggtccagctggtgcagtctgggggagacctggtgaaacctgggggatccctgagactgtcctgtgtggcctctggattcaccttcagtaactacaccatgagctgggtccgccaggctccagggcaggggctgcagtgggtcgcatacattaactatgatgggagtatcacatactacgcagaggctgtgaagggccggttcaccgtctccagagacaacgccaagaacacgctgtatctgcagatgaacagtctgagagccgaggacacggccgtgtatttctgtgtgaaggaaactttcatgataccaactgagtggggccagggaacccttatcaccgtctcctcagcctccaccacggccccctcggtcactagtggccaggccggccagcaccatcaccatcaccatggcgcatacccgtacgacgttccggactacgcttcttag

SEQ ID NO: 4 - DNA, 846 bp, synthetic construct
  source 1..846: mol_type = other DNA
cagtctgtgctgaatcagccgccttctgtgtctggagccctggggcagaccgtcaccatctcctgttctggaagtcacaccaacatcggcagtggatttgatgtacagtggtaccaacagctcccaggaaagtcccctcaaactatcatctatgctaatatcaatcgaccctcgggggtcccggctcgattctctggctccaagtcaggcagcacagccaccctgaccatcactggggtccaggctgaagatgaggctgattattactgccagtcgtatgatgacaatttcgattacgtgttcggctcagggacccaactgaccgtccttggtggtggttcctctagatcttcctcctctggtggcggtggctcgggcggtggtggggaggagcaactggtggagtttgggggagacctggtgaagcctggagggtccttgagactgtcctgtgtggtctctggattcgatttcagtcgctacggcatgtcctgggtccgtcagtctccagggaagggtctccagtgggtcgcagatatcagttacaggggaactacattttacgcagacgatgtgaagggccgatttaccgtctccagagacagtgccaagaacacactctacctcgaaatgaccagcctgacagtcgaggacacggccgtatattattgtgcgaatagtgccaactggtatgtctccccttttgaccactggggccagggaaccctggtcaccgtctcctcagcctccaccacggccccctcggtcactagtggccaggccggccagcaccatcaccatcaccatggcgcatacccgtacgacgttccggactacgcttcttag

SEQ ID NO: 5 - DNA, 840 bp, synthetic construct
  source 1..840: mol_type = other DNA
cagcctgtgctgacccagccaccctcagtgtcggggtcccttggccagagggtcaccatctcctgttctggaagcacgaacaccatcggtgttgttggtgcgaactggtaccaacagctcccaggaaagtcccctaaactcctcgtgtacagtgatggggttcgaccgtcaggggtccctgaccggttttccggctccaggtctgccaactcagacaccctgaccatcactgggcttcaggctgaggacgaggctgattattactgccagtcctttgattccacacataatgccattgtattcggcggaggcacccatctgaccgtcctcggtggtggttcctctagatcttcctcctctggtggcggtggctcgggcggtggtggggaggtgcagctggtggagactgggggagacctggtgaagcctgcggggtccctgagactgtcgtgtgcggcctctggattccccttcagtggctacagtatgacctgggtccgccaggcccctgaaaagggtctgcaattggtcgcaggtattaacagcgatggaagtcacacatactacacagacgatgtgaagggccgattcaccatctccagagacaacaccaaatacatactgtatctgcaaatgaacagtctgagagccgaggacacggccatgtattattgtggctctcatagttggggcaattttcactactggggccagggaaccctggtcaccgtctcctcagagagtccatcccctccaaacctcactagtggccaggccggccagcaccatcaccatcaccatggcgcatacccgtacgacgttccggactacgcttcttag

SEQ ID NO: 6 - DNA, 849 bp, synthetic construct
  source 1..849: mol_type = other DNA
cagcctgtgctcactcagccgccctcagtgtctgggtccctgggccagagggtcaccatctcctgcaccggaactaataccaacatcggcagtggttatagtgtacaatggtaccagcagctcccaggagagtccccaaaacctatcatttatggtagtagtaatcgaccctcgggggtcccggctcgattctctggctccaagtcaggcagcacaggcaccctgaccatcactgggatccaggctgaggatgaggctgattaccactgccagtcctatgatgacagcctcgatggtcatgctgtattcggcggagggacccagctgaccatcctcggtggtggttcctctagatcttcctcctctggtggcggtggctcgggcggtggtggggaggtccagctggtgcagtctgggggagacttggtgaagcctggggggtccttgagactgtcctgtgtggcctctggattcaccttcagtgacaattacatgagctggatccgtcaggttccagggaagggcctgcagtgggtctcacgaattagcactgatggaagaggcacaagctacgcagacgctgtgaagggccgattcaccatctccagagacaatgccaagaacacgctgtatctgcagatgaacagcctgagagacgaggacacggccgtgtattactgtgcaaggcggtctggtatttgggggtttgactactggggccagggcgccctggtcaccgtctcctcagagagtccatcccctccaaacctcactagtggccaggccggccagcaccatcaccatcaccatggcgcatacccgtacgacgttccggactacgcttcttag

SEQ ID NO: 7 - DNA, 855 bp, synthetic construct
  source 1..855: mol_type = other DNA
tcctatgagctgactcagccaccatctatgtctgcagccctggggcagagggtcaccatctcctgcactggaagtaacaccaacatcggcagtggttatgatgtacaatggtaccaacaggtcccaggaaagtcccctacacctctgatttatggtaataccaatcgaccctcgggggtcccggtgcgattctctggctccaagtcaggcaacacagccaccctgaccatcactgggatccaggctgaggatgaggctgattattactgccagtcctatgatgacaatttcgatggtcatcacgtgttcggctcagggacccagctgaccgtcctcggtggtggttcctctagatcttcctcctctggtggcggtggctcgggcggtggtggggaggtacagctggtggaatctgggggagacctggtgaagcctgcggggtccctgagactgtcctgtgtggcctctggattcaccttcagtaactatggcatgaattgggtccgccaggctcccgggaaggggctgcagtgggtcgcaggtattaacagtggtggaagtaccacaacctacgcagacgttgtgaagggtcgattcaccatctccagagacaacgccaagaacacactgtatctgcagatgaacagcctgggagccgaggacacggccgtgtattactgtgcgagggtggattatagtagcagctggttggttgactactggggccagggaaccctggtcaccgtctcctcagcctccaccacggccccctcggtcactagtggccaggccggccagcaccatcaccatcaccatggcgcatacccgtacgacgttccggactacgcttcttag

SEQ ID NO: 8 - DNA, 843 bp, synthetic construct
  source 1..843: mol_type = other DNA
cagtctgtgttgactcaaccggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgaatattattgttcatcctgggatgatagtctcagaggtaccgtgttcggctcagggacccaactgaccgtccttggcggtggttcctctagatcttcctcctctggtgacggtggctcgggcggtggtggggaggtacagctggtggaatctgggggagacctggtgaagcctggggaatccctgagactgtcctgtgtggcctctggattcaccttcagtagctatggcatgagctgggtccgacagtctccagggaaggggttgcagtgggtcgcagatattagttatggaggaggtacatactacgcagacgctgtgaagggccgattcaccatctccagagacaacgccaggaacacagtatatctgcagatgaacagcctgagagccgaggacacggccgtgtattactgtgcgtcccaatggggtgattggggccactttgagtactggggccagggcaccctggtcaccgtctcctcagcctccaccacggccccctcggtcactagtggccaggccggccagcaccatcaccatcaccatggcgcatacccgtacgacgttccggactacgcttcttag

SEQ ID NO: 9 - DNA, 852 bp, synthetic construct
  source 1..852: mol_type = other DNA
cagcctgtgctgacccagccaccctctgtgtctgcagccctgggacagagggtcaccctctcctgcactggaagtaagaccaacatcggcagtggttatgatgtacaatggtaccagcagttcccaggaaagtcccctaaaaccatcatctatggtaatagcaatcgattttcgggggtcccggctcgattctctggctccaagtcaggcagcatcgccaccctgaccatcactgggatccaggctgaggatgaggctgattattactgccagtcctttgatgacaatgtcgatggttacgtgttcggctcagggacccagctgaccatcctcggtggtggttcctctagatcttcctcctctggtggcggtggctcgggcggtggtggggaggtacagctggtggagtctgggggagacctggtgaagcctggggggtccctgagactctcctgtgtggcctctggattcaccttcagtagcgacagcatacattgggtccgtcaggctccaggaaaggggctgcagtgggtcgcatacattaacagtggtggaagtaccacatactacgcagacgctgtgaagggccgattcaccatctccagagacaacgccaggaacacagtgtatctgaagatgaacagcctgagagccgaggacacggctgtgtattactgtgcgaaagattcaggctttggcagctggtaccttgaatcctggggccagggaaccctggtcaccgtctccccagcctccaccacggccccctcggtcactagtggccaggccggccagcaccatcaccatcaccatggcgcatacccgtacgacgttccggactacgcttcttag

SEQ ID NO: 10 - DNA, 843 bp, synthetic construct
  source 1..843: mol_type = other DNA
caggctgtgctgaatcagccggcctctgtgtctggagccctggggcagaccgtcaccatctcctgttctggaagtcacaccaacatcggcagtggatttgatgtacagtggtaccaacagctcccaggaaagtcccctcaaactatcatctatgctaatatcaatcgaccctcgggggtcccggctcgattctctggctccaagtcaggcagcacagccaccctgaccatcactggggtccaggctgaagatgaggctgattatcactgccagtcctttgatgacgacctcgatggtcctgtgttcggcggaggcacccatctgaccgtcctcggtggtggttcctctagatcttcctcctctggtggcggtggctcgggcggtggtggggaggtccagctggtgcagtctggaggagacctggtgaagcctggggggtccctgaaaatttcctgtgtggcctctggactcaccttcaatagaaactacatgacctggatccgccaggctccagggaaggggctgcagtgggtctcagaaattaaccctgatggaagtaccacgagttacacagacgctgtgaggggccgattcaccatctccagagacaatgccaagaacacgttgtatctgcagatgaatagtatgagagatgaggacacggcagtgtattactgcgcaaggagtagttggggttcttttgactattggggccagggaaccctggtcaccgtctcctcagcctccaccacggccccctcggtcactagtggccaggccggccagcaccatcaccatcaccatggcgcatacccgtacgacgttccggactacgcttcttag

SEQ ID NO: 11 - DNA, 831 bp, synthetic construct
  source 1..831: mol_type = other DNA
cagcctgtgctgacccagccaccctctgtgtctggagccctggggcagaccgtcaccatctcctgctctggaagtcacaccaacatcggcagtggatttgatgtacagtggtaccaacagctcccaggaaagtcccctcaaactctcgtctatgctaatatcaatcgaccctcgggggtcccggctcgattctccggctccaagtcaggcagcacagccaccctgaccatcactggggtccaggctgaagatgaggctgattattactgccagtcgtatgatgacaatttcgattacgtgttcggctcagggacccaactgaccgtccttggtggtggttcctctagatcttcctcctctggtggcggtggctcgggcggtggtggggaggtacagctggtggaatctgggggagacctggtgaagcctggggggtccttgagactgtcctgtgtggcctctggattcaccttcagtgactatagcatgagctgggtccgtcagtctccagggaaggggctgcagtgggtcgcagctattatgactagtggaagcacatactacgcagacgctgtgaagggccgattcaccatctccagagacaacgccaagaatacgctgtatctgcagatggacagcctgagagccgaggacacggccgtttattactgtgcgcacgactactggtttgacttctggggccagggcaccctggtcaccgtctcctcagcctccaccacggccccctcggtcactagtggccaggccggccagcaccatcaccatcaccatggcgcatacccgtacgacgttccggactacgcttcttag

SEQ ID NO: 12 - DNA, 843 bp, synthetic construct
  source 1..843: mol_type = other DNA
tcctatgagctgactcagccaccatcagcgactgtgaccctgaggcagacggcccgcctcacctgtgggggagagaacattggaagaaaaagtgttcaatggtaccaacagaagccgggccagtcccccctactcattatctatgctgattccagcaggccgtcagggatccctgagcgattctctggcgccaattcggggaacacggccagcctgaccatcagcggggccctggccgaggacgaggctgactattactgccagatttgggaccgcaatattaatgtgttcggctccgggacccagctgaccgtccttggcggtggttcctctagatcttcctcctctggtggcggtggctcgggcggtggtggggaggagcaactggtggagtttgggggagacctggtgaagcctgcggggtccctgagactgtcctgtgtggcctcgggattcaccttcagtagctacgacatgagctgggtccgccaggctcctgggaaggggctgcagtgggtcgcgactattagttatgatggaagtagtacatactacactgacgctgtgaagggccgattcaccgtctccagagacaacgccaagaacacgttgtatctgcagatgaacagcctgagagccgaggacacggctctgtattactgtgcaaaggattctggcagttggtcccatttctatggtttggacacctggggccatggcacctcagtcttcgtgtcttccgcctccaccacggccccctcggtcactagtggccaggccggccagcaccatcaccatcaccatggcgcatacccgtacgacgttccggactacgcttcttag

SEQ ID NO: 13 - DNA, 840 bp, synthetic construct
  source 1..840: mol_type = other DNA
cagcctgtgctgacccagccaccctctgtgtctggagccctggggcagaccgtcaccatctcctgctctggaagtcacaccaacatcggcagtggatttgatgtacagtggtaccaacagctcccaggaaagtcccctcaaactctcgtctatgctaatatcaatcgaccctcgggggtcccggctcgattctctggctccaagtcaggcagcacagccaccctgaccatcactggggtccaggctgaagatgaggctgattattactgccagtcgtatgatgacaatttcgattacgtgttcggctcagggacccagctgaccgtcctcggcggtggttcctctagatcttcctcctctggtgacggtggctcgggcggtggtggggaggtacagctggtggagtctgggggaggcctgatgaagcctggggggtccctgaaactctcctgtttggcctctggtttcaccttcagtcgctactatatgtattgggtccgccaggctccagggaagggacttcagtgggtcgcacaaattagcagtgatggcagaagaacatactacgcagacgctgtgcagggccgattcaccatctccagagacaatgtcaagaacacggtatatctgcagatgaacagcctgagcgacgaggacacggccgtgtattattgtgtccgagaggactacttcgactttgactactggggccagggcaccctggtcaccgtctcctcagcctccaccacggccccctcggtcactagtggccaggccggccagcaccatcaccatcaccatggcgcatacccgtacgacgttccggactacgcttcttag

SEQ ID NO: 14 - DNA, 846 bp, synthetic construct
  source 1..846: mol_type = other DNA
cagtctgtgctgaatcagccaccttctgtgtctgcggccctggggcagaggatcaccatctcctgcactggaagtcacaccaacatcggcagtggttatgatgtacaatggtaccagcagctccccggaaagtcccctagaactatcatttatggtaatagcgatcggccctctggggtcccggttcgattctctggctccaagtcaggcaacacagccaccctgaccatcactgggatccaggctgaggatgaggctgattattattgccagtcctatgatgacaacctcaatgatccttacgtgttcggctcagggacccaactgaccgtccttggtggtggttcctctagatcttcctcctctggtggcggtggctcgggcggtggtggggaggtccagctggtgcagtctgggggagacctggtgaagcctggggggtccctgagactctcctgtgtggcctctggattcgccttcagtggcaaaacaatgcaatgggtccgtcaggctccagggaaggggctacagtgggtcgcatacattcaacatgatgggagtagaacatcctacgcagacgctgtgaagggccgattcaccatctccagagacaacgccaagaacacgctctatctccagatggacagcctgagagccgaggacacggccgtgtattactgtgcggatcatccctggtacgggcctgagtattggggccagggaaccccggtcaccgtctcctcagagagtccatcccctccaaacctcactagtggccaggccggccagcaccatcaccatcaccatggcgcatacccgtacgacgttccggactacgcttcttag

SEQ ID NO: 15 - DNA, 858 bp, synthetic construct
  source 1..858: mol_type = other DNA
tcctatgagctgactcagccaccatcggtgtctgcagccctggggcagggggtcaccatctcctgcatcggcagtgacaccaacatcggcaatcattatgatgtacaatggtaccagcagctcccaggaaagtcccctagaactattatttatggtaatagcgatcgaccctcgggggtcccgggtcgattctctggctccaagtcaggcaacacagccaccctgaccatcactgggatccaggctgaggatgaggctgattatcactgccagtcctatgatgacgacctcgatggccatattgtgttcggcggaggcacccatctgaccgtcctcggtggtggttcctctagatcttcctcctctggtggcggtggctcgggcggtggtggggaggtccagctggtgcagtctgggggagacctggtgaagcctgggggttccctgagactgtcctgtgtggcatctggattcaccttcaataagtaccacatgagttgggtccgccaggctccagggaaggggctgcagtgggtcggaaagataaccagtgatggaagtgccacagggtatgcagacgctgtaaagggccgattcaccatctccagagacaatgccaagaacacgttctatctacagatggacagcctgagagccgaggacacggctatgtattactgtgcaagggatcagagtagttggggaacgggtaattttgactactggggccagggaaccctggtcaccgtctcctcagcctccaccacggccccctcggtcactagtggccaggccggccagcaccatcaccatcaccatggcgcatacccgtacgacgttccggactacgcttcttag

SEQ ID NO: 16 - DNA, 843 bp, synthetic construct
  source 1..843: mol_type = other DNA
cagtctgtgctgactcagccgacctcagtgtctgggtccctgggccagagggtcaccatctcctgcactggaagcagctccaatgttggttatggcaattatgtgggctggtaccagcagctcccaggaacaggccccagaaccctcatctatggtagtcattaccgaccctcgggggtccctgatcgattctctggctccagttcaggcagctcagccacactgaccatctctgggctccaggctgaggatgaggctgattattactgctcatcctacgacagcactctcagtggttacgtgtttggctcagggacccagctgaccatcctcggcggtggttcctctagatcttcctcctctggtggcggtggctcgggcggtggtggggaggtacagctggtggaatctgggggagacctggtgaagcctggggggtccctgagactctcctgtgtgggctctggattcaccttgagtagttattggatgagctgggtccgccaggctccagggaaggggctacagtggattgcagaaatcagcggcagtggaaataccatatattatggggacgctgtgaagggccgattcaccatctccagagacaatgccaagaacacgctgtatctgcagatgaacagcctgagagatgaggacacggcagtgtattactgtgcaacggaatactactggaatattgacaactggggccagggaaccctggtcaccgtctcctcagagagtccattccctccaaacctcactagtggccaggccggccagcaccatcaccatcaccatggcgcatacccgtacgacgttccggactacgcttcttag

SEQ ID NO: 17 - DNA, 849 bp, synthetic construct
  source 1..849: mol_type = other DNA
cagcctgtgctgacccagccaccctctgtgtctgcagccctggggcagagggtcaccatctcctgcactggaagtaagaccaacatcggcagtggttatgatgtacaatggtaccagcagctcccaggaaagtcccctaaaaccatcatttatggtaatagcaatcgaccctcgggggtcccggctcgattctctggctccaagtcaggcagcatcgccaccctgaccatcactgggatccaggctgaggatgaggctgattattactgccagtcctttgatgacaatgtcgatggttacgtgttcggctcagggacccagctgaccatcctcggtggtggttcctctagatcttcctcctctggtggcggtggctcgggcggtggtggggaggtgccactggtggaatctgggggagacctggtgaagcctgggggatccctgagactgtcctgtgtggcctctggattcaccttcagtagctacggcatgagctgggtccgccaggctccagggaagggactacagtgggtcgcatacattaatggtggtggaagtagtacatcctacgcagacgctgtgaggggccgattcaccatctccagagacaacgccgagaacacgctgtatctgcagatgaacagcctgagagccgacgatacggctgtatattactgttcgaattactacggtatctactgggattctgactactggggccagggaaccctggtcaccgtctcctcagagagtccatcccctccaaacctcactagtggccaggccggccagcaccatcaccatcaccatggcgcatacccgtacgacgttccggactacgcttcttag

SEQ ID NO: 18 - DNA, 843 bp, synthetic construct
  source 1..843: mol_type = other DNA
cagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagcctccaccacggccccctcggtcactagtggccaggccggccagcaccatcaccatcaccatggcgcatacccgtacgacgttccggactacgcttcttag

SEQ ID NO: 19 - DNA, 840 bp, synthetic construct
  source 1..840: mol_type = other DNA
caggctgtgctgactcagccggcctcagtgtcggggccccttggccagagggtcaccatttcctgctctggaagcacgaacaacatcggtattgttggtgcggcctggtaccaacagtttccaggaaaggcccctaaactcctcgtatacagtgatgggagtcgaccgtcaggggtccctgaccggttttccggctccaactctggcaactcagccaccctgaccatcagtggacttcaggctgaggacgaggctgattattactgtcagtctgttgatgccacgcatggtactattctgttcggcggagggacccagctgaccgtcctcggtggtggttcctctagatcttcctcctctggtggcggtggctcgggcggtggtggggaggtgcagctggtggagactgggggagacctggtgaagcctgcggggtccctgagactgtcatgtgcggcctctggattccccttcagtggctacagtatgacctgggtccgccaggcccctgaaaagggtctgcaattggtcgcaggtattaacagcgatggaagtcacacatactacacagacgatgtgaagggccgattcaccgtctccagagacaacaccaaatacatactgtatctgcagatgaacagtctgagagtcgaggacacggccatatattattgtggctctcatagttggggcaattttcactactggggccagggaaccctggtcaccgtctcctcagagagtccatcccctccaaacctcactagtggccaggccggccagcaccatcaccatcaccatggcgcatacccgtacgacgttccggactacgcttcttag

SEQ ID NO: 20 - DNA, 828 bp, synthetic construct
  source 1..828: mol_type = other DNA
cagtctgtgctgaatcagccaccttcagtatcagtctctctgggacagacagcaaccatctcctgctctggagagagtctgagtaaatattatgcacaatggttccagcagaaggcaggccaagtccctgtgttggtcatatataaggacactgagcggccctctgggatccctgaccgattctccggctccagttcagggaacacacacaccctgaccatcagcggggctcgggccgaggacgaggctgactattactgcgagtcagaagtcagtcctgatactattgtgttcggcggaggcacccacctgaccgtcctcggtggtggttcctctagatcttcctcctctggtggcggtggctcgggcggtggtggggaggtacagctggtggaatctgggggagacctggtgaagcctgggggatccctgagactgtcctgtgtggcctctggattcaccttcagtagctatgccatgagctgggtccgccaggctccaggggaggggctgcagtgggtcgcatacattaacagtgctggaagtaccacatactacgcagacgctgtgaagggccgattcaccatctccagagacaacgccaggaacacagtgtatctgcagatgaacagcctgagagccgaggacacagccgtgtattactgtggccgatattattatggtctggactactggggtcctggcacctcagtcttcgtgtcctcagagagtccatcccctccaaacctcactagtggccaggccggccagcaccatcaccatcaccatggcgcatacccgtacgacgttccggactacgcttcttag

SEQ ID NO: 21 - DNA, 834 bp, synthetic construct
  source 1..834: mol_type = other DNA
cagcctgtgctgacccagccaccctcggtgtcagtgtctctgggacagacagcaaccatctcctgctctggagagagtctgagtaaatattatgcacaatggttccagcagaaggcaggccaagcccctgtgttggtcatatataaggacactgagcggccctctgggatccctgaccgattctctggctccagttcagggaacacacacaccctgaccatcagcggggctcgggccgaggacgaggctgactattactgcgagtcagcagtcagtagtgatactgcagtgttcggctcagggacccaactgaccgtcctcggcggtggttcctctagatcttcctcctctggtggcggtggctcgggcggtggtggggaggtacagctggtggagtctgggggagatatggtgaagcctggggggtccttgagactgtcctgtgtggcctctggattcaccttcagtagtttctggatgtactgggtccgccaggctcctgggaaggggctgcagtgggtcggaggtattaatagtggtggaagtattacaagttacgcagacgctgtgaagggccgattcaccatctccagagacaacgccaggaacacactgtatctgcagatggacagtctgagagatgaagacacagccgtctactactgtgcgaagccggatatatatgggagttttgaccactggggccagggcaccctggtcaccgtctcctcagagagtccatcccctccaaacctcactagtggccaggccggccagcaccatcaccatcaccatggcgcatacccgtacgacgttccggactacgcttcttag

SEQ ID NO: 22 - DNA, 843 bp, synthetic construct
  source 1..843: mol_type = other DNA
cagcctgtgctcactcagccgccctcagtgtctgggtccctgggccagagggtcaccatctcctgcactggaagcagctccaatgttggttatggcgattatgtgggctggtaccagcagctcccaggaacaagccccagaaccctcatttattctagtagtagccgaccctcgggagtccccgatcgattctctggctccaggtcaggcagcacagcaaccctgaccatctctgggctccaggctgaagatgaagccgattatcactgctcatcctatgacaccagtctcaatggtttcgtgttcggctcagggacccagctgaccatcctcggtggtggttcctctagatcttcctcctctggtggcggtggctcgggcggtggtggggaactcacactgcaggagtcagggccaggactggtgaagcccgcacagaccctctctctcacctgtactgtgtccggagactccgtcaccagcagtcacttctggcactggatccgccagcgccctgggaggggactggaatggatgggatactggtcagacattacgatttacagtccaacattccaaggacgcatctccatcactgttgacacggccaggaaccagttctccctgcatttgaactccgtgacgaccgaggactcggccgtttatttctgtgcaagatataatggatacgtcgattatttagacgtctggggccagggcaccctgatcaccgtctcctcagcctccaccacggccccctcggtcactagtggccaggccggccagcaccatcaccatcaccatggcgcatacccgtacgacgttccggactacgcttcttag

SEQ ID NO: 23 - DNA, 837 bp, synthetic construct
  source 1..837: mol_type = other DNA
gatattgtcatgacacagacgccactgtccctgtccgtcagtcctggagagccggcctccatctcctgcaaggccagtcagagcctcctgcacagtaacgggaacacctatttgtattggttccgacagaagccaggccagtctccacagcgcctgatctacttggtttccaacagagacgctggggtcccagacaggttcagtggcagcgggtcagggacagatttcaccctgagaatcagcagagtagaggctgatgattctggaatttattactgcggccaaggtaaacaagatcctatcacctttggcgaagggacacatctggagattaaaggtggttcctctagatcttcctcctctggtggcggtggctcgggcggtggtggggaactcacactgcaggagtcagggccaggactggtgaagccctcacagaccctctctctcacctgtgttgtgtccggaggctccgtcaccaccagtgactactggaactggctccgccagcgccctgggaggggactggaatacatgggatactggaaaggtcggactgagtacaacccggcattccagggacgcatctccatcactgctgacacggccaagaaccagttctccctgcagttaaactccgtgaccgccgacgacacggccatttattattgtgcacattgggggtcaggcaatcttgactattggggcccgggaaccctggtcaccgtctcctcagagagtccatcccctccaaacctcactagtggccaggccggccagcaccatcaccatcaccatggcgcatacccgtacgacgttccggactacgcttcttag

SEQ ID NO: 24 - DNA, 843 bp, synthetic construct
  source 1..843: mol_type = other DNA
cagtctgtgctgaatcagccaccttctgtgtctggagccctggggcagaccgtcaccatctcctgctctggaagtcacaccaacatcggcagtggatttgatgtacagtggtaccaacagctcccaggaaagtcccctcaaactctcgtctatgctaatatcaatcgaccctcgggggtcccggttcgattctctggctccaagtcaggcaccacagccaccctgaccatcactgggatccaggctgaggatgaggctgattattactgccagtcctatgatgacaacgtcgatggttacgtgttcggctcagggacccagctgaccgtccttggtggtggttcctctagatcttcctcctctggtggcggtggctcgggcggtggtggggaggtccagctggtgcagtctgggggaracctggtgaagcctggggggtccctgagactctcctgtgtggcctctggattcaccctcagtagtaactatatggactgggtccgccaggctccagggaaggggcctcagtgggtcgcacaaattagcactgatggaagtaggacatattacgcagacgctgtgaagggccgattcaccatttccagagacaccgccaagaacacactgtatctgcagatgaacagtctgagagccgaggacacggctatgtattactgtgcaaagaacgaatacttggcccctacgtactggggccagggcaccctggtcaccgtctcctcagagagtccatcccctccaaacctcactagtggccaggccggccagcaccatcaccatcaccatggcgcatacccgtacgacgttccggactacgcttcttag

SEQ ID NO: 25 - DNA, 852 bp, synthetic construct
  source 1..852: mol_type = other DNA
gatatcgtcatgacacagactccgctgtccctgtccgtcagccctggagagacggcctccatctcctgcaaggccagtcagagcctcctgcacagtaacgggaacacctatttgaattggttccgacagaagccaggccagtctccacaggtcctgatctatcaggtctccaacagagacactggggtcccagacaggttcagtggcagcgggtcagggacagatttcaccctgagaatcagtagagtggaggctgacgatgctggagtttattattgcgggcaaggtatacaatatcctccgacgttcggagcaggaaccaaggtggagctcaaaggtggttcctctagatcttcctcctctggtggcggtggctcgggcggtggtggggaggagcaactggtggagtttgggggagacctggtgaagcctggggggtccttgagactctcctgtgtggcctctggattcaccttcagtagtctttccatgaactggatccgccaggctccagggaaggggctgcagtgggtctcacaaattagccctgatggaactatcacaggctacacagacgctgtgaagggccgattcaccatctccagagacaatgccaagaacacgctgtatcttcagatgaacagcctgagagatgaagacacggcaatatattactgtgcgaagagtcgatatacggatacgtacccctttgactactggggcccgggaaccctggtcaccgtctcctcagagagtccatcccctccaaacctcactagtggccaggccggccagcaccatcaccatcaccatggcgcatacccgtacgacgttccggactacgcttcttag

SEQ ID NO: 26 - DNA, 825 bp, synthetic construct
  source 1..825: mol_type = other DNA
tccagtgtgctgactcagcctccctcggtatcagtgtctctgggacagacagcaaccatctcctgctctggagagagtctgagtaaatattatactcaatggttccagcagaaggcaggccaagcccctatcttggtcatttataaggacactgagcggccctctgggatccctgaccggttctctggctccagttcaggcaacacacacaccctgaccatcagcggggctcgggccgaggacgaggctgactattattgcgagtcattagtcgattctgatgcttacatgttcggctctggaacccagctgaccgtccttggcggtggttcctctagatcttcctcctctggtggcggtggctcgggcggtggtggggaggtgcagctggtggagactgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacrgccccctcggtcactagtggccaggccggccagcaccatcaccatcaccatggcgcatacccgtacgacgttccggactacgcttcttag

SEQ ID NO: 27 - DNA, 828 bp, synthetic construct
  source 1..828: mol_type = other DNA
cagcctgtgctgacccagccaccctcggtatcagtgtctctgggacagacagcaaccatctcctgctctggagagagtctgagtaaatattatgcacaatggttccagcagaaggcaggccaagcccctgtgttggtcatatataaggacactgagcggccctctgggatccctgaccgattctctggctccagttcagggaacacacacaccctgaccatcactgggatccaggctgaggatgagggtgattatcactgccactcctatgatgagaacctcgatggttacgtgttcggctcaggaacccaactgaccgtccttggcggtggttcctctagatcttcctcctctggtggcggtggctcgggcggtggtggggaggtgcagctggtggagtctgggggagacctggtgaagcctggggggtctctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacggccccctcggtcactagtggccaggccggccagcaccatcaccatcaccatggcgcatacccgtacgacgttccggactacgcttcttag

SEQ ID NO: 28 - DNA, 825 bp, synthetic construct
  source 1..825: mol_type = other DNA
cagtctgccctgactcaaccttcctcggtatcagtgtctctgggacagacagcaaccatctcctgctctggagagagtctgaataaatattatgtacagtggttccagcagaaggcaggccaagtccctatattggtcatatatagggacactgagcggccctctgggatccctgaccgattctctggctccagttcagggaacacacacaccctgaccatcaccgggactcgggccgaggacgaggctgactattactgcgagtcactagtcaatgataatacttacgtgttcggctcagggacccaactgaccgtccttggcggtggttcctctagatcttcctcctctggtggcggtggctcgggcggtggtggggaggtgcagctggtggagactgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacggccccctcggtcactagtggccaggccggccagcaccatcaccatcaccatggcgcatacccgtacgacgttccggactacgcttcttag

SEQ ID NO: 29 - DNA, 825 bp, synthetic construct
  source 1..825: mol_type = other DNA
cagtctgtgctgaatcagccgccttcagtatcagtgtctctgggacagacagcaaccatctcctgctctggagagagtctgaataaatattatacacagtggttccagcagaaggcaggccaagcccctgtgttggtcatatataaggacactgagcggccctctgggatccctgaccgattctccggatccagttcagggaacacacacaccctgaccatcagcggggctcgggccgaggacgaggctgactattactgcgagtcagtagacagtactgatacttacgtgttcggctcagggacccagctgaccatcctcggtggtggttcctctagatcttcctcctctggtggcggtggctcgggcggtggtggggaggtacatctggtggaatctgggggagacttggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacggccccctcggtcactagtggccaggccggccagcaccatcaccatcaccatggcgcatacccgtacgacgttccggactacgcttcttag

SEQ ID NO: 30 - DNA, 828 bp, synthetic construct
  source 1..828: mol_type = other DNA
cagtctgtcttgactcagccagcctcggtatcagtgtctctgggacagacagcaaccatctcctgctctggagagagtctgagtaaatattatacacagtggttccagcagaaggcaggccaggcccctgtgttggtcatctataaggacactgagcggccctctgggatccctgaccgattctctggctccagctcagggaacacgcacaccctgaccatcagcggggctcaggccgaggatgaggctgattattactgccagtcctatgatgacaacgtcgatggttacgtgttcggctctgggacccaactgaccgtcctcggtggtggttcctctagatcttcctcctctggtggcggtggctcgggcggtggtggggaggtacagctggtggaatctgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacggccccctcggtcactagtggccaggccggccagcaccatcaccatcaccatggcgcatacccgtacgacgttccggactacgcttcttag

SEQ ID NO: 31 - DNA, 825 bp, synthetic construct
  source 1..825: mol_type = other DNA
cagtctgccctgactcaaccttcctcggtatcagtgtctctgggacagacagcaaccatctcctgctctggagagagtctgactaaatattatgtacaatggttccaacagaaggcaggccaagcccctgtattggtcatatatagggacactgagcggccctctgggatccctgaccgattctctggctccagttcaggtaatacacacaccctgaccatcagtggggctcgggccgaggacgaggctgactattattgcgagtcaatggacggtcgtgaggcttacgtgttcggctcagggacccaactgaccgtcctcggcggtggttcctctagatcttcctcctctggtggcggtggctcgggcggtggtggggaggtacagctggtggagtctgggggagacctggtgaagccgggggggtccctgagactgtcctgtgtggcctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacggccccctcggtcactagtggccaggccggccagcaccatcaccatcaccatggcgcatacccgtacgacgttccggactacgcttcttag

SEQ ID NO: 32 - DNA, 828 bp, synthetic construct
  source 1..828: mol_type = other DNA
cagtctgtgttgactcaaccagcctcggtatcagtgtctctgggacagacagcaaccatctcctgctctggagagagtctgagtaaatattatgcacaatggttccagcagaaggcaggccaagcccctgtgttggtcatatataaggacactgagcggccctctgggatccctgaccgattctctggctccagttcagggaacacacacaccctgaccatcactgggatccaggctgcggatgaggctgattattactgccagtcctatgatgacaacctcgatggttacgtgttcggctcaggaacccagctgaccgtccttggtggtggttcctctagatcttcctcctctggtggcggtggctcgggcggtggtggggaggtacagctggtggagtctgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctcagcctccaccacggccccctcggtcactagtggccaggccggccagcaccatcaccatcaccatggcgcatacccgtacgacgttccggactacgcttcttag

SEQ ID NO: 33 - DNA, 831 bp, synthetic construct
  source 1..831: mol_type = other DNA
gatattgtcatgacacagacgccactgtccctgtccgtcagccctggagagccggcctccatctcctgcaaggccagtcagagcctcctgcacagtgacgggaacacctatttgcattggtacctgcaaaagccaggccagtctccacagcttctgatctactcggtttccaaccgcttcactggcgtgtcagacaggttcagtggcagcgggtcagggacagatttcaccctgagaatcagcagagtggaggctgacgatactggagtttattactgcgggcaaggtacacacctcccgttcacttttggccaagggaccaaactggagatcaaaggtggttcctctagatcttcctcctctggtggcggtggctcgggcggtggtggggaggtacagctggtggagtctgggggagacctggtgaagcctggggggtccctgagactctcctgtgcagcctctggattcaccttcagtcactacgacatgaactgggtccgccaggctcctgggaaggggctgcagtgggtcgcatctattgcctatgatggatctaacacatactacactgacgctgtgaagggccgattcaccatctccagagacaacgccaagaatatactgtttttgctggtgaatagcctgagggccgaggacacggccatatattattgtgcgcgggggctggtctactggggccagggaactttggtcaccgtctcctcagcctccaccacggccccctcggtcactagtggccaggccggccagcaccatcaccatcaccatggcgcatacccgtacgacgttccggactacgcttcttag

SEQ ID NO: 34 - DNA, 849 bp, synthetic construct
  source 1..849: mol_type = other DNA
tcctatgagctgactcagccaccatctgtgtctgcggccctgggacagagggtcaccatctcctgcactggaactaataccaacatcggcagtggttatagtgtacaatggtaccagcagctcccaggagagtccccaaaacctatcatttatggtagtagtaatcgaccctcgggggtcccggctcgattctctggctccaagtcaggcagcacaggcaccctgaccatcactgggatccaggctgaggatgaggctgattaccactgccagtcctatgatgacagcctcgatggccatgctgtattcggcggagggacccagctgaccatcctcggcggtggttcctctagatcttcctcctctggtggcggtggctcgggcggtggtggggaggtacagctggtggagtctgggggagacctggtgaagcctggggggtccctgagactctcctgtgtggcctctggattaaccttcagtgacaattacatgtcctggatccgtcaggttccagggaagggcctgcagtgggtctcacgaattagcactgatggaagaggcacaagttacgcagacgctgtgaagggccgattcaccatctccagagacaatgccaagaacacgctgtatctgcagatgaacagcctgagagacgaggacacggccgtgtattactgtgcaaggcggtctggtatttgggggtttgactactggggccagggcaccctggtcaccgtctcctctgagagtccatcccctccaaacctcactagtggccaggccggccagcaccatcaccatcaccatggcgcatacccgtacgacgttccggactacgcttcttag

SEQ ID NO: 35 - DNA, 825 bp, synthetic construct
  source 1..825: mol_type = other DNA
tccagtgtgctgactcagcctccttcggtatcagtgtctctgggacagacagcaaccatctcctgctctggagagagtctgagtgaatattatgcacaatggttccagcagaaggcaggccaagcccctgtattggtcatatataaggacactgagcgggcctctggaatccctgaccgattctctggctccagttcggggaacacacacaccctgaccatcagcggggctcgggccgaggacgaggctgactattactgcgagtcagcagtcaatagtgatggtattgtgttcggcggaggcacccatctgaccgtcctcggcggtggttcctctagatcttcctcctctggtggcggtggctcgggcggtggtggggaggtacagctggtggagtctgggggagacctggtgaagcctgcggggtccctgagactgtcctgtgtgacctctggattcaccttcagcagctatgccatgagctgggtccgccaggctcctgggaaggggctgcagtgggtcggaagtattaacagtggtggaagtaccacaagctacgcagacgctgtgaagggccgattcaccatctccagagacaacgccaagaacacagtgtatctgcagatgaacagcctgagagccgaggacacggccgtgtattactgtgcccggtggtgggactttgagtactggggccagggcaccctggtcaccgtctcctcagagagtccatcccctccaaacctcactagtggccaggccggccagcaccatcaccatcaccatggcgcatacccgtacgacgttccggactacgcttcttag

SEQ ID NO: 36 - DNA, 840 bp, synthetic construct
  source 1..840: mol_type = other DNA
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtgtacagtgatggggatcgaccgtcaggggtccctgaccggttttccggctccaactctggcaactcagacaccctgaccatcactgggcttcaggctgaggacgaggctgattattactgccagtcctttgataccacgcttgatgcttacgtgttcggctcaggaacccagctgaccgtccttggtggtggttcctctagatcttcctcctctggtggcggtggctcgggcggtggtggggaggagcaactggtggagtttgggggaggcctggtgaggcctgggggatccctgagactgtcctgtgtggcctctggattcaccttcagtagctatggcatgacctgggtccgtcagtctccagggaaggggctgcagtgggtcgctactattagcgataccggaagcacatactacgcagacactgtgaagggccgattcagcatctccagagacaacgccaagaacacgctgtatctgcagatgaacagtctgagagccgacgacacggccctgtattattgtacgaaggacctcggggtgtggggtatggacaactggggtcctggcacctcactcttcgtgtcctcagcctccaccacggccccctcggtcactagtggccaggccggccagcaccatcaccatcaccatggcgcatacccgtacgacgttccggactacgcttcttag

SEQ ID NO: 37 - DNA, 822 bp, synthetic construct
  source 1..822: mol_type = other DNA
tccagtgtgctgactcagcctccctcagtatcagtgtctctgggacagacagcaaccatctcctgctctggagagagtctgaatgactattatgcacaatggttccagcagaaggcaggccaagtccctgtgttggtcatatatcgggacactgagcggccctctgggatccctgaccgattctccggctccagttcagggaacacacacaccctgaccatcagcggggctcgggccgaggacgaggctgactattactgcgagtcagaagtcagtgctggtggtgctgtattcggcgaaggcacccacctgaccgtcctcggtggtggttcctctagatcttcctcctctggtggcggtggctcgggcggtggtggggaggtgcagctggtggagactgggggagacctggtgaagccgggggggtccctgagactgtcctgtgtggcctctggattcaccctcagagactacagcatgacctgggtccgccagactcctgagaaggggctgcagttggtcgcaggtattagcaccggtggaagtaccacatactacatagacgctgtgaagggccgattcaccatctccagagacaacgccaagaacacagtgtatctgcagatgaacagcctgagagccgaggacacggccatgtatcactgtacaaccacggccttaggttactggggccagggcaccctggtcactgtctcctcagcctccaccacgaccccctcggtcactagtggccaggccggccagcaccatcaccatcaccatggcgcatacccgtacgacgttccggactacgcttcttag

SEQ ID NO: 38 - DNA, 825 bp, synthetic construct
  source 1..825: mol_type = other DNA
tccagtgtgctgactcagcctccctcagtatcagtctctctgggacagacagcaaccatctcctgctctggagagagtctgagtgagtattatgcacaatggttccagcagaaggcaggccaagcccctgtgttggtcatatataaggacactgagcggccctctgggatccctgaccgattctccggctccagttcagggaacacacacaccctgaccatcagcggggctcgggccgaggacgaggctgactattactgcgcgtcagcagacagtagtgctattcctgtgttcggcggaggcacccatctgaccgtcctcggcggtggttcctctagatcttcctcctctggtggcggtggctcgggcggtggtggggaggtgcagctggtggagtctgggggagacctggtgaagcctggggggtccctgagactctcctgtgtggcctcaggattcaatttcggaaactatgccgtgagctgggtccgccaggctcctgggaagggactgcagtgggtcgcaggtatcagtaacgatggaactataacaggctacacagatgttgtggagggccgtttcatcatctccagagacaatgccaagaacacgctgtatctacagatgaacagcctgagagccgaggacatggctatatattattgtgtggctgggggctttcttgactactggggccagggaaccctggtcaccgtctcctcagcctccaccacggccccctcggtcactagtggccaggccggccagcaccatcaccatcaccatggcgcatacccgtacgacgttccggactacgcttcttag

SEQ ID NO: 39 - DNA, 849 bp, synthetic construct
  source 1..849: mol_type = other DNA
cagtctgtgctgactcagccgccctcagtgtcggggtcccctggccagagggtcaccatctcctgctctggaaccacgaagaacatcggtatagtcggtgcgagttggtaccaacaactcccaggaaaggcccctaaactcctcgttcacagtgatgggtttcgaccgtcaggggtccctgaccggttttccgcctccaactctggcgactcagacattctgaccatcactgggcttcaggctgaggacgaggctgattattactgtcagtcctttgataccacgcttgatgctcacgtcttcggctcagggacccagctgaccgtccttggcggtggttcctctagatcttcctcctctggtggcggtggctcgggcggtggtggggaggtgccactggtggaatctgggggagacctggtgaagcctggggggtccttgagactgtcctgtgtggcctctggattcacctttagtagttatggcatgagctgggtccgtcagtctccagggaaggggctgcagtgggtcgcaggtattacctatgatggaagtaatacatactacactgacactgtgaggggccgattcaccatctccagagacaacgccaggaacacagtgtatctgcagatgaacagcctgagagccgaggacacagctgtgtattactgtgtggccagccccctctacggaacttatggtttcgactactggggccatggcacctcagtcttcgtgtcctcagcctccaccacggccccctcggtcactagtggccaggccggccagcaccatcaccatcaccatggcgcatacccgtacgacgttccggactacgcttcttag

SEQ ID NO: 40 - DNA, 840 bp, synthetic construct
  source 1..840: mol_type = other DNA
cagtctgtgctaactcagccgccctcagtgtctgggtccctgggccagaaggtcaccatctcctgcactggaagcagttccagcgtcggtactcattatgtgggctggtaccaaatacccccaggaataggccctagaaccgtcatctataataatgattaccgaccttcaggggtccccgatcgattctctggctccaagtcaggcaattcagccaccctgaccatctctgggctccaggctgaggacgaggctgattattactgctcatcatgggatgacagtctcggagcttacgtgctcggctcagggacccagctgaccatcctcggtggtggttcctctagatcttcctcctctggtggcggtggctcgggcggtggtggggaactcacactgcaggagtcagggccaggactggtgaagccctcacagaccctctctctcacctgcgctgtgtccggaggctccgtcatcagaggttactactggacctggatccgccagcgccctgggaggggactggaatggatgggttactgggcagggagtacagattataacccggcattccagggacgcatcttcatcactgctggcacggccaagaatcagttctccctggagatgacttccatgaccacggacgacacggccatatattactgtgtcggatcccttcggggcggttggctctttgacaactggggccagggaaccctagtcaccgtctcctccgcctccaccacggccccctcggtcactagtggccaggccggccagcaccatcaccatcaccatggcgcatacccgtacgacgttccggactacgcttcttag

SEQ ID NO: 41 - DNA, 825 bp, synthetic construct
  source 1..825: mol_type = other DNA
cagcctgtgctcactcagccgccctcggtctcagtgtctctgggacagacagcaaccatctcctgctctggagagagtctgagtaaatattatgcacagtggttccagcagaaggcaggccaagcccctgtcttggtcatatataaggacactgagcggccctctgggatccctgaccgattctctggctccagttcagggaacacacacaccctgaccatcagcggggctcgggccgaggacgaggctgactattactgcgagtcagcagtcagtactgagactgttgtgttcggcggaggcacccatctgaccgtcctcggcggtggttcctctagatcttcctcctctggtggcggtggctcgggcggtggtggggaggtgcagctggtggagactgggggagacctggtgatgcctgcagggtccctgagactgtcttgtgtggcctctggattcaccttcagtagttctgtcatgagctgggtccgtcaggctcctgggaaggggctggagtgggtcgcaggtattaatggtggcggaagttttgcggcctacggagacgctgtgaagggccgattcaccatatccagagacaacgccaagaacacgctctatctgcagatgaacagcctgagagccgaggacacggccatgtattactgtgtggggggcggttatatggacttctggggtcctggcacctcactcttcgtgtcctcagagagtccatcccctccaaacctcactagtggccaggccggccagcaccatcaccatcaccatggcgcatacccgtacgacgttccggactacgcttcttag

SEQ ID NO: 42 - DNA, 834 bp, synthetic construct
  source 1..834: mol_type = other DNA
cagtctgtgctgactcagccgacctcagtgtcggggtcccttggccagagggtcaccatttcctgctctggaagcacgaacgacatcggtattattggtgcggcctggtaccaacagctcccagggaaggcccctaaactcctcctgtacagtaataagaatcgaccgtcaggagtccctgaccgattttccggctccaactctggcatctcagccaccctgaccatcactgggcttcaggctgaggacgaggctgattattactgtcagtctgttgatcccgcgcttcatacttacgtgtttggctcagggacccagctgaccatcctcggcggtggttcctctagatcttcctcctctggtggcggtggctcgggcggtggtggggaggtgcagctggtggagattgggggagaccttgtgaaacctgaggggtccctgagactctcctgtgtggtctctggcttcaccttcagtggctacgacatgaactgggtccgccaggctccagggaaggggcttcagtgggtcgcatacattagcagtgatgggaggagaacagtttacacagacgctgtgaagggccgattcaccatctccagagacaatgccaagaacacgctgtatctgcagatgaacagcctgaaatctgaggacacagccgtatattactgtgtgaaggggggttggttggacttctggggccagggaaccctggtcaccgtctcctcagagagtccatcccctccaaacctcactagtggccaggccggccagcaccatcaccatcaccatggcgcatacccgtacgacgttccggactacgcttcttag

SEQ ID NO: 43 - DNA, 834 bp, synthetic construct
  source 1..834: mol_type = other DNA
gatattgtcatgacacagaacccactgtccctggccgtcacccctggagagctggccactatctcctgcagggccagtcagagtctcctgcacagtgatggaaaatcctatttgaattggtacctgcagaagccaggccagactcctcggccgctgatttatgaggcttccaggcgtttctctggggtctcagacaggttcaatggcagcgggtcagggacagatttcacccttaaaatcagtagggtggaggctgaggatgttggagtttattactgtcagcaaggtctacattttcctccgacgttcggagcaggaaccaaggtggagctcaaaggtggttcctctagatcttcctcctctggtggcggtggctcgggcggtggtggggaggtacagctggtggaatctgggggagacctggtgaagcctggggggtccctgagactctcctgtgcagcctctggattcaccttcactgactacgacatgagttgggtccgccaggctcctgggaaggggctgcagtgggtcgcagctattagctatgatggaagtagcacatactacactgacgctgtgaagggccgattcaccatctccagagacaacgccaggaacacactgtatctgcagatgagcagcctgagagccgaggacacggccatgtattactgtgtagtctcgaatatgaacctctggggccatggcacctcagtcttcgtgtcctctgcctccaccacggccccctcggtcactagtggccaggccggccagcaccatcaccatcaccatggcgcatacccgtacgacgttccggactacgcttcttag

SEQ ID NO: 44 - protein, 283 aa, synthetic construct
QAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVLGGGSSRSSSSGGGGSGGGGEVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSSASTTAPSVTSGQAGQHHHHHHGAYPYDVPDYAS

SEQ ID NO: 45 - protein, 283 aa, synthetic construct
SYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTILGGGSSRSSSSGGGGSGGGGEEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSSASTTAPSVTSGQAGQHHHHHHGAYPYDVPDYAS

SEQ ID NO: 46 - protein, 280 aa, synthetic construct
QSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVLGGGSSRSSSSGGGGSGGGGEVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSSASTTAPSVTSGQAGQHHHHHHGAYPYDVPDYAS

SEQ ID NO: 47 - protein, 281 aa, synthetic construct
QSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVLGGGSSRSSSSGGGGSGGGGEEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSSASTTAPSVTSGQAGQHHHHHHGAYPYDVPDYAS

SEQ ID NO: 48 - protein, 279 aa, synthetic construct
QPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVLGGGSSRSSSSGGGGSGGGGEVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSSESPSPPNLTSGQAGQHHHHHHGAYPYDVPDYAS

SEQ ID NO: 49 - protein, 282 aa, synthetic construct
QPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSSESPSPPNLTSGQAGQHHHHHHGAYPYDVPDYAS

SEQ ID NO: 50 - protein, 284 aa, synthetic construct
SYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVLGGGSSRSSSSGGGGSGGGGEVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSSASTTAPSVTSGQAGQHHHHHHGAYPYDVPDYAS

SEQ ID NO: 51 - protein, 280 aa, synthetic construct
QSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVLGGGSSRSSSSGDGGSGGGGEVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSSASTTAPSVTSGQAGQHHHHHHGAYPYDVPDYAS

SEQ ID NO: 52 - protein, 283 aa, synthetic construct
QPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTILGGGSSRSSSSGGGGSGGGGEVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSPASTTAPSVTSGQAGQHHHHHHGAYPYDVPDYAS

SEQ ID NO: 53 - protein, 280 aa, synthetic construct
QAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVLGGGSSRSSSSGGGGSGGGGEVQLVQSGGDLVKPGGSLKISCVASGLTFNRNYMTWIRQAPGKGLQWVSEINPDGSTTSYTDAVRGRFTISRDNAKNTLYLQMNSMRDEDTAVYYCARSSWGSFDYWGQGTLVTVSSASTTAPSVTSGQAGQHHHHHHGAYPYDVPDYAS

SEQ ID NO: 54 - protein, 276 aa, synthetic construct
QPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGGGGSGGGGEVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSSASTTAPSVTSGQAGQHHHHHHGAYPYDVPDYAS

SEQ ID NO: 55 - protein, 280 aa, synthetic construct
SYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVLGGGSSRSSSSGGGGSGGGGEEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSSASTTAPSVTSGQAGQHHHHHHGAYPYDVPDYAS

SEQ ID NO: 56 - protein, 279 aa, synthetic construct
QPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGDGGSGGGGEVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSSASTTAPSVTSGQAGQHHHHHHGAYPYDVPDYAS

SEQ ID NO: 57 - protein, 281 aa, synthetic construct
QSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVLGGGSSRSSSSGGGGSGGGGEVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSSESPSPPNLTSGQAGQHHHHHHGAYPYDVPDYAS

SEQ ID NO: 58 - protein, 285 aa, synthetic construct
SYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVLGGGSSRSSSSGGGGSGGGGEVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSSASTTAPSVTSGQAGQHHHHHHGAYPYDVPDYAS

SEQ ID NO: 59 - protein, 280 aa, synthetic construct
QSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTILGGGSSRSSSSGGGGSGGGGEVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSSESPFPPNLTSGQAGQHHHHHHGAYPYDVPDYAS

SEQ ID NO: 60 - protein, 282 aa, synthetic construct
QPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTILGGGSSRSSSSGGGGSGGGGEVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSSESPSPPNLTSGQAGQHHHHHHGAYPYDVPDYAS

SEQ ID NO: 61 - protein, 280 aa, synthetic construct
QSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASTTAPSVTSGQAGQHHHHHHGAYPYDVPDYAS

SEQ ID NO: 62 - protein, 279 aa, synthetic construct
QAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVLGGGSSRSSSSGGGGSGGGGEVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSSESPSPPNLTSGQAGQHHHHHHGAYPYDVPDYAS

SEQ ID NO: 63 - protein, 275 aa, synthetic construct
QSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVLGGGSSRSSSSGGGGSGGGGEVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSSESPSPPNLTSGQAGQHHHHHHGAYPYDVPDYAS

SEQ ID NO: 64 - protein, 277 aa, synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVLGGGSSRSSSSGGGGSGGGGEVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSSESPSPPNLTSGQAGQHHHHHHGAYPYDVPDYAS

SEQ ID NO: 65 - protein, 280 aa, synthetic construct
QPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTILGGGSSRSSSSGGGGSGGGGELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSSASTTAPSVTSGQAGQHHHHHHGAYPYDVPDYAS

SEQ ID NO: 66 - protein, 278 aa, synthetic construct
DIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIKGGSSRSSSSGGGGSGGGGELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSSESPSPPNLTSGQAGQHHHHHHGAYPYDVPDYAS

SEQ ID NO: 67 - protein, 280 aa, synthetic construct
QSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVLGGGSSRSSSSGGGGSGGGGEVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSSESPSPPNLTSGQAGQHHHHHHGAYPYDVPDYAS

SEQ ID NO: 68 - protein, 283 aa, synthetic construct
DIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELKGGSSRSSSSGGGGSGGGGEEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSSESPSPPNLTSGQAGQHHHHHHGAYPYDVPDYAS

SEQ ID NO: 69 - protein, 274 aa, synthetic construct
SSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 70 - protein, 275 aa, synthetic construct
QPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 71 - protein, 274 aa, synthetic construct
QSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 72 - protein, 274 aa, synthetic construct
QSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTILGGGSSRSSSSGGGGSGGGGEVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 73 - protein, 275 aa, synthetic construct
QSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 74 - protein, 274 aa, synthetic construct
QSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVLGGGSSRSSSSGGGGSGGGGEVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 75 - protein, 275 aa, synthetic construct
QSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 76 - protein, 276 aa, synthetic construct
DIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIKGGSSRSSSSGGGGSGGGGEVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSSASTTAPSVTSGQAGQHHHHHHGAYPYDVPDYAS

SEQ ID NO: 77 - protein, 282 aa, synthetic construct
SYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSSESPSPPNLTSGQAGQHHHHHHGAYPYDVPDYAS

SEQ ID NO: 78 - protein, 274 aa, synthetic construct
SSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVLGGGSSRSSSSGGGGSGGGGEVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSSESPSPPNLTSGQAGQHHHHHHGAYPYDVPDYAS

SEQ ID NO: 79 - protein, 279 aa, synthetic construct
QSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVLGGGSSRSSSSGGGGSGGGGEEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSSASTTAPSVTSGQAGQHHHHHHGAYPYDVPDYAS

SEQ ID NO: 80 - protein, 273 aa, synthetic construct
SSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVLGGGSSRSSSSGGGGSGGGGEVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSSASTTTPSVTSGQAGQHHHHHHGAYPYDVPDYAS

SEQ ID NO: 81 - protein, 274 aa, synthetic construct
SSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVLGGGSSRSSSSGGGGSGGGGEVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSSASTTAPSVTSGQAGQHHHHHHGAYPYDVPDYAS

SEQ ID NO: 82 - protein, 282 aa, synthetic construct
QSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVLGGGSSRSSSSGGGGSGGGGEVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSSASTTAPSVTSGQAGQHHHHHHGAYPYDVPDYAS

SEQ ID NO: 83 - protein, 279 aa, synthetic construct
QSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTILGGGSSRSSSSGGGGSGGGGELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSSASTTAPSVTSGQAGQHHHHHHGAYPYDVPDYAS

SEQ ID NO: 84 - protein, 274 aa, synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVLGGGSSRSSSSGGGGSGGGGEVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSSESPSPPNLTSGQAGQHHHHHHGAYPYDVPDYAS

SEQ ID NO: 85 - protein, 277 aa, synthetic construct
QSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTILGGGSSRSSSSGGGGSGGGGEVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSSESPSPPNLTSGQAGQHHHHHHGAYPYDVPDYAS

SEQ ID NO: 86 - protein, 277 aa, synthetic construct
DIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELKGGSSRSSSSGGGGSGGGGEVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSSASTTAPSVTSGQAGQHHHHHHGAYPYDVPDYAS

SEQ ID NO: 87 - protein, 8 aa, synthetic construct
GFTFSNHE

SEQ ID NO: 88 - protein, 8 aa, synthetic construct
IYDSGSRT

SEQ ID NO: 89 - protein, 15 aa, synthetic construct
AGGNSRGWTEFGMDY

SEQ ID NO: 90 - protein, 9 aa, synthetic construct
NTNIGSPYD

SEQ ID NO: 91 - protein, 12 aa, synthetic construct
QSYDDSLDGRVV

SEQ ID NO: 92 - protein, 8 aa, synthetic construct
GFSFSSYD

SEQ ID NO: 93 - protein, 8 aa, synthetic construct
TNFDGSRT

SEQ ID NO: 94 - protein, 14 aa, synthetic construct
ARGMGSWGASTFGY

SEQ ID NO: 95 - protein, 8 aa, synthetic construct
RSNLGGNY

SEQ ID NO: 96 - protein, 11 aa, synthetic construct
SSWDDDLGSHV

SEQ ID NO: 97 - protein, 8 aa, synthetic construct
GFTFSNYT

SEQ ID NO: 98 - protein, 8 aa, synthetic construct
INYDGSIT

SEQ ID NO: 99 - protein, 10 aa, synthetic construct
VKETFMIPTE

SEQ ID NO: 100 - protein, 9 aa, synthetic construct
HTNIGSGFD

SEQ ID NO: 101 - protein, 11 aa, synthetic construct
QSYDDNFDGHV

SEQ ID NO: 102 - protein, 8 aa, synthetic construct
GFDFSRYG

SEQ ID NO: 103 - protein, 8 aa, synthetic construct
ISYRGTTF

SEQ ID NO: 104 - protein, 12 aa, synthetic construct
NSANWYVSPFDH

SEQ ID NO: 105 - protein, 10 aa, synthetic construct
QSYDDNFDYV

SEQ ID NO: 106 - protein, 8 aa, synthetic construct
GFPFSGYS

SEQ ID NO: 107 - protein, 8 aa, synthetic construct
INSDGSHT

SEQ ID NO: 108 - protein, 10 aa, synthetic construct
GSHSWGNFHY

SEQ ID NO: 109 - protein, 8 aa, synthetic construct
TNTIGVVG

SEQ ID NO: 110 - protein, 11 aa, synthetic construct
QSFDSTHNAIV

SEQ ID NO: 111 - protein, 8 aa, synthetic construct
GFTFSDNY

SEQ ID NO: 112 - protein, 8 aa, synthetic construct
ISTDGRGT

SEQ ID NO: 113 - protein, 11 aa, synthetic construct
ARRSGIWGFDY

SEQ ID NO: 114 - protein, 9 aa, synthetic construct
NTNIGSGYS

SEQ ID NO: 115 - protein, 12 aa, synthetic construct
QSYDDSLDGHAV

SEQ ID NO: 116 - protein, 8 aa, synthetic construct
GFTFSNYG

SEQ ID NO: 117 - protein, 8 aa, synthetic construct
INSGGSTT

SEQ ID NO: 118 - protein, 13 aa, synthetic construct
ARVDYSSSWLVDY

SEQ ID NO: 119 - protein, 9 aa, synthetic construct
NTNIGSGYD

SEQ ID NO: 120 - protein, 12 aa, synthetic construct
QSYDDNFDGHHV

SEQ ID NO: 121 - protein, 8 aa, synthetic construct
GFTFSSYG

SEQ ID NO: 122 - protein, 8 aa, synthetic construct
ISYGGGTY

SEQ ID NO: 123 - protein, 11 aa, synthetic construct
SQWGDWGHFEY

SEQ ID NO: 124 - protein, 8 aa, synthetic construct
RSNIGANY

SEQ ID NO: 125 - protein, 11 aa, synthetic construct
SSWDDSLRGTV

SEQ ID NO: 126 - protein, 8 aa, synthetic construct
GFTFSSDS

SEQ ID NO: 127 - protein, 13 aa, synthetic construct
AKDSGFGSWYLES

SEQ ID NO: 128 - protein, 9 aa, synthetic construct
KTNIGSGYD

SEQ ID NO: 129 - protein, 11 aa, synthetic construct
QSFDDNVDGYV

SEQ ID NO: 130 - protein, 8 aa, synthetic construct
GLTFNRNY

SEQ ID NO: 131 - protein, 8 aa, synthetic construct
INPDGSTT

SEQ ID NO: 132 - protein, 10 aa, synthetic construct
ARSSWGSFDY

SEQ ID NO: 133 - protein, 11 aa, synthetic construct
QSFDDDLDGPV

SEQ ID NO: 134 - protein, 8 aa, synthetic construct
GFTFSDYS

SEQ ID NO: 135 - protein, 8 aa, synthetic construct
IMTSGSTY

SEQ ID NO: 136 - protein, 7 aa, synthetic construct
HDYWFDF

SEQ ID NO: 137 - protein, 8 aa, synthetic construct
GFTFSSYD

SEQ ID NO: 138 - protein, 8 aa, synthetic construct
ISYDGSST

SEQ ID NO: 139 - protein, 15 aa, synthetic construct
AKDSGSWSHFYGLDT

SEQ ID NO: 140 - protein, 6 aa, synthetic construct
NIGRKS

SEQ ID NO: 141 - protein, 9 aa, synthetic construct
QIWDRNINV

SEQ ID NO: 142 - protein, 8 aa, synthetic construct
GFTFSRYY

SEQ ID NO: 143 - protein, 8 aa, synthetic construct
ISSDGRRT

SEQ ID NO: 144 - protein, 10 aa, synthetic construct
VREDYFDFDY

SEQ ID NO: 145 - protein, 8 aa, synthetic construct
GFAFSGKT

SEQ ID NO: 146 - protein, 8 aa, synthetic construct
IQHDGSRT

SEQ ID NO: 147 - protein, 10 aa, synthetic construct
ADHPWYGPEY

SEQ ID NO: 148 - protein, 9 aa, synthetic construct
HTNIGSGYD

SEQ ID NO: 149 - protein, 12 aa, synthetic construct
QSYDDNLNDPYV

SEQ ID NO: 150 - protein, 8 aa, synthetic construct
GFTFNKYH

SEQ ID NO: 151 - protein, 8 aa, synthetic construct
ITSDGSAT

SEQ ID NO: 152 - protein, 14 aa, synthetic construct
ARDQSSWGTGNFDY

SEQ ID NO: 153 - protein, 9 aa, synthetic construct
DTNIGNHYD

SEQ ID NO: 154 - protein, 12 aa, synthetic construct
QSYDDDLDGHIV

SEQ ID NO: 155 - protein, 8 aa, synthetic construct
GFTLSSYW

SEQ ID NO: 156 - protein, 8 aa, synthetic construct
ISGSGNTI

SEQ ID NO: 157 - protein, 10 aa, synthetic construct
ATEYYWNIDN

SEQ ID NO: 158 - protein, 9 aa, synthetic construct
SSNVGYGNY

SEQ ID NO: 159 - protein, 11 aa, synthetic construct
SSYDSTLSGYV

SEQ ID NO: 160 - protein, 8 aa, synthetic construct
INGGGSST

SEQ ID NO: 161 - protein, 12 aa, synthetic construct
SNYYGIYWDSDY

SEQ ID NO: 162 - protein, 11 aa, synthetic construct
QSFDDNVDGYV

SEQ ID NO: 163 - protein, 8 aa, synthetic construct
GFTLSNYG

SEQ ID NO: 164 - protein, 8 aa, synthetic construct
IRSGGNTY

SEQ ID NO: 165 - protein, 11 aa, synthetic construct
STWDSSLNAYV

SEQ ID NO: 166 - protein, 8 aa, synthetic construct
TNNIGIVG

SEQ ID NO: 167 - protein, 11 aa, synthetic construct
QSVDATHGTIL

SEQ ID NO: 168 - protein, 8 aa, synthetic construct
GFTFSSYA

SEQ ID NO: 169 - protein, 8 aa, synthetic construct
INSAGSTT

SEQ ID NO: 170 - protein, 9 aa, synthetic construct
GRYYYGLDY

SEQ ID NO: 171 - protein, 6 aa, synthetic construct
SLSKYY

SEQ ID NO: 172 - protein, 10 aa, synthetic construct
ESEVSPDTIV

SEQ ID NO: 173 - protein, 8 aa, synthetic construct
GFTFSSFW

SEQ ID NO: 174 - protein, 8 aa, synthetic construct
INSGGSIT

SEQ ID NO: 175 - protein, 11 aa, synthetic construct
AKPDIYGSFDH

SEQ ID NO: 176 - protein, 10 aa, synthetic construct
ESAVSSDTAV

SEQ ID NO: 177 - protein, 9 aa, synthetic construct
GDSVTSSHF

SEQ ID NO: 178 - protein, 5 aa, synthetic construct
WSDIT

SEQ ID NO: 179 - protein, 12 aa, synthetic construct
ARYNGYVDYLDV

SEQ ID NO: 180 - protein, 9 aa, synthetic construct
SSNVGYGDY

SEQ ID NO: 181 - protein, 11 aa, synthetic construct
SSYDTSLNGFV

SEQ ID NO: 182 - protein, 9 aa, synthetic construct
GGSVTTSDY

SEQ ID NO: 183 - protein, 5 aa, synthetic construct
WKGRT

SEQ ID NO: 184 - protein, 10 aa, synthetic construct
AHWGSGNLDY

SEQ ID NO: 185 - protein, 11 aa, synthetic construct
QSLLHSNGNTY

SEQ ID NO: 186 - protein, 9 aa, synthetic construct
GQGKQDPIT

SEQ ID NO: 187 - protein, 8 aa, synthetic construct
GFTLSSNY

SEQ ID NO: 188 - protein, 8 aa, synthetic construct
ISTDGSRT

SEQ ID NO: 189 - protein, 10 aa, synthetic construct
AKNEYLAPTY

SEQ ID NO: 190 - protein, 11 aa, synthetic construct
QSYDDNVDGYV

SEQ ID NO: 191 - protein, 8 aa, synthetic construct
GFTFSSLS

SEQ ID NO: 192 - protein, 8 aa, synthetic construct
ISPDGTIT

SEQ ID NO: 193 - protein, 13 aa, synthetic construct
AKSRYTDTYPFDY

SEQ ID NO: 194 - protein, 9 aa, synthetic construct
GQGIQYPPT

SEQ ID NO: 195 - protein, 8 aa, synthetic construct
GFTFSSYW

SEQ ID NO: 196 - protein, 8 aa, synthetic construct
ISDSGSST

SEQ ID NO: 197 - protein, 8 aa, synthetic construct
ARGLGMDY

SEQ ID NO: 198 - protein, 10 aa, synthetic construct
ESLVDSDAYM

SEQ ID NO: 199 - protein, 11 aa, synthetic construct
HSYDENLDGYV

SEQ ID NO: 200 - protein, 6 aa, synthetic construct
SLNKYY

SEQ ID NO: 201 - protein, 10 aa, synthetic construct
ESLVNDNTYV

SEQ ID NO: 202 - protein, 10 aa, synthetic construct
ESVDSTDTYV

SEQ ID NO: 203 - protein, 6 aa, synthetic construct
SLTKYY

SEQ ID NO: 204 - protein, 10 aa, synthetic construct
ESMDGREAYV

SEQ ID NO: 205 - protein, 11 aa, synthetic construct
QSYDDNLDGYV

SEQ ID NO: 206 - protein, 8 aa, synthetic construct
GFTFSHYD

SEQ ID NO: 207 - protein, 8 aa, synthetic construct
IAYDGSNT

SEQ ID NO: 208 - protein, 6 aa, synthetic construct
ARGLVY

SEQ ID NO: 209 - protein, 11 aa, synthetic construct
QSLLHSDGNTY

SEQ ID NO: 210 - protein, 9 aa, synthetic construct
GQGTHLPFT

SEQ ID NO: 211 - protein, 8 aa, synthetic construct
GLTFSDNY

SEQ ID NO: 212 - protein, 8 aa, synthetic construct
ARWWDFEY

SEQ ID NO: 213 - protein, 6 aa, synthetic construct
SLSEYY

SEQ ID NO: 214 - protein, 10 aa, synthetic construct
ESAVNSDGIV

SEQ ID NO: 215 - protein, 8 aa, synthetic construct
ISDTGSTY

SEQ ID NO: 216 - protein, 10 aa, synthetic construct
KDLGVWGMDN

SEQ ID NO: 217 - protein, 11 aa, synthetic construct
QSFDTTLDAYV

SEQ ID NO: 218 - protein, 8 aa, synthetic construct
GFTLRDYS

SEQ ID NO: 219 - protein, 8 aa, synthetic construct
ISTGGSTT

SEQ ID NO: 220 - protein, 7 aa, synthetic construct
TTTALGY

SEQ ID NO: 221 - protein, 6 aa, synthetic construct
SLNDYY

SEQ ID NO: 222 - protein, 10 aa, synthetic construct
ESEVSAGGAV

SEQ ID NO: 223 - protein, 8 aa, synthetic construct
GFNFGNYA

SEQ ID NO: 224 - protein, 8 aa, synthetic construct
ISNDGTIT

SEQ ID NO: 225 - protein, 8 aa, synthetic construct
VAGGFLDY

SEQ ID NO: 226 - protein, 10 aa, synthetic construct
ASADSSAIPV

SEQ ID NO: 227 - protein, 8 aa, synthetic construct
ITYDGSNT

SEQ ID NO: 228 - protein, 13 aa, synthetic construct
VASPLYGTYGFDY

SEQ ID NO: 229 - protein, 8 aa, synthetic construct
TKNIGIVG

SEQ ID NO: 230 - protein, 11 aa, synthetic construct
QSFDTTLDAHV

SEQ ID NO: 231 - protein, 9 aa, synthetic construct
GGSVIRGYY

SEQ ID NO: 232 - protein, 5 aa, synthetic construct
WAGST

SEQ ID NO: 233 - protein, 12 aa, synthetic construct
VGSLRGGWLFDN

SEQ ID NO: 234 - protein, 8 aa, synthetic construct
SSSVGTHY

SEQ ID NO: 235 - protein, 11 aa, synthetic construct
SSWDDSLGAYV

SEQ ID NO: 236 - protein, 8 aa, synthetic construct
GFTFSSSV

SEQ ID NO: 237 - protein, 8 aa, synthetic construct
INGGGSFA

SEQ ID NO: 238 - protein, 8 aa, synthetic construct
VGGGYMDF

SEQ ID NO: 239 - protein, 10 aa, synthetic construct
ESAVSTETVV

SEQ ID NO: 240 - protein, 8 aa, synthetic construct
GFTFSGYD

SEQ ID NO: 241 - protein, 8 aa, synthetic construct
VKGGWLDF

SEQ ID NO: 242 - protein, 8 aa, synthetic construct
TNDIGIIG

SEQ ID NO: 243 - protein, 11 aa, synthetic construct
QSVDPALHTYV

SEQ ID NO: 244 - protein, 8 aa, synthetic construct
GFTFTDYD

SEQ ID NO: 245 - protein, 7 aa, synthetic construct
VVSNMNL

SEQ ID NO: 246 - protein, 11 aa, synthetic construct
QSLLHSDGKSY

SEQ ID NO: 247 - protein, 9 aa, synthetic construct
QQGLHFPPT

SEQ ID NO: 248 - protein, 5 aa, synthetic construct
  REPEAT 1..5: note = Repeat n time where n represents an integer of at least 1
GSGGS

SEQ ID NO: 249 - protein, 4 aa, synthetic construct
  REGION 1..4: note = Repeat n time where n represents an integer of at least 1
GGGS

SEQ ID NO: 250 - protein, 5 aa, synthetic construct
  REPEAT 1..5: note = Repeat n time where n represents an integer of at least 1
GGGGS

SEQ ID NO: 251 - protein, 4 aa, synthetic construct
GGSG

SEQ ID NO: 252 - protein, 5 aa, synthetic construct
GGSGG

SEQ ID NO: 253 - protein, 5 aa, synthetic construct
GSGSG

SEQ ID NO: 254 - protein, 5 aa, synthetic construct
GSGGG

SEQ ID NO: 255 - protein, 5 aa, synthetic construct
GGGSG

SEQ ID NO: 256 - protein, 5 aa, synthetic construct
GSSSG

SEQ ID NO: 257 - protein, 5 aa, synthetic construct
GGGGS

SEQ ID NO: 258 - protein, 15 aa, synthetic construct
GGGGSGGGGSGGGGS

SEQ ID NO: 259 - protein, 19 aa, synthetic construct
GGGSSRSSSSGGGGSGGGG

SEQ ID NO: 260 - protein, 11 aa, synthetic construct
SGGGGSGGGGS

SEQ ID NO: 261 - DNA, 57 bp, synthetic construct
  source 1..57: mol_type = other DNA
ggcggtggttcctctagatcttcctcctctggtggcggtggctcgggcggtggtggg

SEQ ID NO: 262 - DNA, 369 bp, synthetic construct
  source 1..369: mol_type = other DNA
  misc_feature 69: note = scFv insertion site
atggccttaccagtgaccgccttgctcctgccgctggccttgctgctccacgccgccaggccgggatccgctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 263 - protein, 122 aa, synthetic construct
  VARIANT 23: note = scFv insertion site
MALPVTALLLPLALLLHAARPGSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 264 - protein, 30 aa, synthetic construct
GAGCAGAAATTAATCTCAGAGGAGGACCTG

SEQ ID NO: 265 - DNA, 228 bp, synthetic construct
  source 1..228: mol_type = other DNA
tcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 266 - protein, 74 aa, synthetic construct
PTEPSSKTGNPRHLHVLIGTSVVKIPFTILLFFLLHRWCSNKKNAAVMDQEPAGNRTVNSEDSDEQDHQEVSYA

SEQ ID NO: 267 - protein, 7 aa, synthetic construct
AAEPKSS

SEQ ID NO: 268 - protein, 17 aa, synthetic construct
AAEPKSSDKTHTCPPCP

SEQ ID NO: 269 - protein, 4 aa, synthetic construct
GGGG

SEQ ID NO: 270 - protein, 14 aa, synthetic construct
GGGGDKTHTCPPCP

SEQ ID NO: 271 - protein, 109 aa, synthetic construct
QAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVL

SEQ ID NO: 272 - protein, 110 aa, synthetic construct
SYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTIL

SEQ ID NO: 273 - protein, 111 aa, synthetic construct
QSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVL

SEQ ID NO: 274 - protein, 110 aa, synthetic construct
QSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVL

SEQ ID NO: 275 - protein, 110 aa, synthetic construct
QPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVL

SEQ ID NO: 276 - protein, 112 aa, synthetic construct
QPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTIL

SEQ ID NO: 277 - protein, 112 aa, synthetic construct
SYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVL

SEQ ID NO: 278 - protein, 110 aa, synthetic construct
QSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVL

SEQ ID NO: 279 - protein, 111 aa, synthetic construct
QPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTIL

SEQ ID NO: 280 - protein, 111 aa, synthetic construct
QAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVL

SEQ ID NO: 281 - protein, 110 aa, synthetic construct
QPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVL

SEQ ID NO: 282 - protein, 106 aa, synthetic construct
SYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVL

SEQ ID NO: 283 - protein, 110 aa, synthetic construct; identical to SEQ ID NO: 281

SEQ ID NO: 284 - protein, 112 aa, synthetic construct
QSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVL

SEQ ID NO: 285 - protein, 112 aa, synthetic construct
SYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVL

SEQ ID NO: 286 - protein, 111 aa, synthetic construct
QSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTIL

SEQ ID NO: 287 - protein, 111 aa, synthetic construct
QPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTIL

SEQ ID NO: 288 - protein, 110 aa, synthetic construct
QSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVL

SEQ ID NO: 289 - protein, 110 aa, synthetic construct
QAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVL

SEQ ID NO: 290 - protein, 107 aa, synthetic construct
QSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVL

SEQ ID NO: 291 - protein, 107 aa, synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVL

SEQ ID NO: 292 - protein, 111 aa, synthetic construct
QPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTIL

SEQ ID NO: 293 - protein, 112 aa, synthetic construct
DIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIK

SEQ ID NO: 294 - protein, 111 aa, synthetic construct
QSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVL

SEQ ID NO: 295 - protein, 112 aa, synthetic construct
DIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELK

SEQ ID NO: 296 - protein, 107 aa, synthetic construct
SSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVL

SEQ ID NO: 297 - protein, 108 aa, synthetic construct
QPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVL

SEQ ID NO: 298 - protein, 107 aa, synthetic construct
QSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVL

SEQ ID NO: 299 - protein, 107 aa, synthetic construct
QSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTIL

SEQ ID NO: 300 - protein, 108 aa, synthetic construct
QSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVL

SEQ ID NO: 301 - protein, 107 aa, synthetic construct
QSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVL

SEQ ID NO: 302 - protein, 108 aa, synthetic construct
QSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVL

SEQ ID NO: 303 - protein, 112 aa, synthetic construct
DIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIK

SEQ ID NO: 304 - protein, 112 aa, synthetic construct
SYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTIL

SEQ ID NO: 305 - protein, 107 aa, synthetic construct
SSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVL

SEQ ID NO: 306 - protein, 110 aa, synthetic construct
QSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVL

SEQ ID NO: 307 - protein, 107 aa, synthetic construct
SSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVL

SEQ ID NO: 308 - protein, 107 aa, synthetic construct
SSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVL

SEQ ID NO: 309 - protein, 110 aa, synthetic construct
QSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVL

SEQ ID NO: 310 - protein, 110 aa, synthetic construct
QSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTIL

SEQ ID NO: 311 - protein, 107 aa, synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVL

SEQ ID NO: 312 - protein, 110 aa, synthetic construct
QSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTIL

SEQ ID NO: 313 - protein, 112 aa, synthetic construct
DIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELK

SEQ ID NO: 314 - protein, 155 aa, synthetic construct
EVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSSASTTAPSVTSGQAGQHHHHHHGAYPYDVPDYAS

SEQ ID NO: 315 - protein, 154 aa, synthetic construct
EEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSSASTTAPSVTSGQAGQHHHHHHGAYPYDVPDYAS

SEQ ID NO: 316 - protein, 150 aa, synthetic construct
EVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSSASTTAPSVTSGQAGQHHHHHHGAYPYDVPDYAS

SEQ ID NO: 317 - protein, 152 aa, synthetic construct
EEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSSASTTAPSVTSGQAGQHHHHHHGAYPYDVPDYAS

SEQ ID NO: 318 - protein, 150 aa, synthetic construct
EVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSSESPSPPNLTSGQAGQHHHHHHGAYPYDVPDYAS

SEQ ID NO: 319 - protein, 151 aa, synthetic construct
EVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSSESPSPPNLTSGQAGQHHHHHHGAYPYDVPDYAS

SEQ ID NO: 320 - protein, 153 aa, synthetic construct
EVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSSASTTAPSVTSGQAGQHHHHHHGAYPYDVPDYAS

SEQ ID NO: 321 - protein, 151 aa, synthetic construct
EVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSSASTTAPSVTSGQAGQHHHHHHGAYPYDVPDYAS

SEQ ID NO: 322 - protein, 153 aa, synthetic construct
EVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSPASTTAPSVTSGQAGQHHHHHHGAYPYDVPDYAS

SEQ ID NO: 323 - protein, 150 aa, synthetic construct
EVQLVQSGGDLVKPGGSLKISCVASGLTFNRNYMTWIRQAPGKGLQWVSEINPDGSTTSYTDAVRGRFTISRDNAKNTLYLQMNSMRDEDTAVYYCARSSWGSFDYWGQGTLVTVSSASTTAPSVTSGQAGQHHHHHHGAYPYDVPDYAS

SEQ ID NO: 324 - protein, 147 aa, synthetic construct
EVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSSASTTAPSVTSGQAGQHHHHHHGAYPYDVPDYAS

SEQ ID NO: 325 - protein, 155 aa, synthetic construct
EEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSSASTTAPSVTSGQAGQHHHHHHGAYPYDVPDYAS

SEQ ID NO: 326 - protein, 150 aa, synthetic construct
EVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSSASTTAPSVTSGQAGQHHHHHHGAYPYDVPDYAS

SEQ ID NO: 327 - protein, 150 aa, synthetic construct
EVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSSESPSPPNLTSGQAGQHHHHHHGAYPYDVPDYAS

SEQ ID NO: 328 - protein, 154 aa, synthetic construct
EVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSSASTTAPSVTSGQAGQHHHHHHGAYPYDVPDYAS

SEQ ID NO: 329 - protein, 150 aa, synthetic construct
EVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSSESPFPPNLTSGQAGQHHHHHHGAYPYDVPDYAS

SEQ ID NO: 330 - protein, 152 aa, synthetic construct
EVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSSESPSPPNLTSGQAGQHHHHHHGAYPYDVPDYAS

SEQ ID NO: 331 - protein, 151 aa, synthetic construct
EVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASTTAPSVTSGQAGQHHHHHHGAYPYDVPDYAS

SEQ ID NO: 332 - protein, 150 aa, synthetic construct
EVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSSESPSPPNLTSGQAGQHHHHHHGAYPYDVPDYAS

SEQ ID NO: 333 - protein, 149 aa, synthetic construct
EVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSSESPSPPNLTSGQAGQHHHHHHGAYPYDVPDYAS

SEQ ID NO: 334 - protein, 151 aa, synthetic construct
EVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSSESPSPPNLTSGQAGQHHHHHHGAYPYDVPDYAS

SEQ ID NO: 335 - protein, 150 aa, synthetic construct
ELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSSASTTAPSVTSGQAGQHHHHHHGAYPYDVPDYAS

SEQ ID NO: 336 - protein, 148 aa, synthetic construct
ELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSSESPSPPNLTSGQAGQHHHHHHGAYPYDVPDYAS

SEQ ID NO: 337 - protein, 150 aa, synthetic construct
EVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSSESPSPPNLTSGQAGQHHHHHHGAYPYDVPDYAS

SEQ ID NO: 338 - protein, 153 aa, synthetic construct
EEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSSESPSPPNLTSGQAGQHHHHHHGAYPYDVPDYAS

SEQ ID NO: 339 - protein, 148 aa, synthetic construct
EVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 340 - protein, 148 aa, synthetic construct
EVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 341 - protein, 148 aa, synthetic construct; identical to SEQ ID NO: 339

SEQ ID NO: 342 - protein, 148 aa, synthetic construct
EVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 343 - protein, 148 aa, synthetic construct; identical to SEQ ID NO: 340

SEQ ID NO: 344 - protein, 148 aa, synthetic construct
EVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYAS

SEQ ID NO: 345 - protein, 148 aa, synthetic construct; identical to SEQ ID NO: 340

SEQ ID NO: 346 - protein, 146 aa, synthetic construct
EVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSSASTTAPSVTSGQAGQHHHHHHGAYPYDVPDYAS

SEQ ID NO: 347 - protein, 151 aa, synthetic construct
EVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSSESPSPPNLTSGQAGQHHHHHHGAYPYDVPDYAS

SEQ ID NO: 348 - protein, 148 aa, synthetic construct
EVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSSESPSPPNLTSGQAGQHHHHHHGAYPYDVPDYAS

SEQ ID NO: 349 - protein, 150 aa, synthetic construct
EEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSSASTTAPSVTSGQAGQHHHHHHGAYPYDVPDYAS

SEQ ID NO: 350 - protein, 147 aa, synthetic construct
EVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSSASTTTPSVTSGQAGQHHHHHHGAYPYDVPDYAS

SEQ ID NO: 351 - protein, 148 aa, synthetic construct
EVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSSASTTAPSVTSGQAGQHHHHHHGAYPYDVPDYAS

SEQ ID NO: 352 - protein, 153 aa, synthetic construct
EVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSSASTTAPSVTSGQAGQHHHHHHGAYPYDVPDYAS

SEQ ID NO: 353 - protein, 150 aa, synthetic construct
ELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSSASTTAPSVTSGQAGQHHHHHHGAYPYDVPDYAS

SEQ ID NO: 354 - protein, 148 aa, synthetic construct
EVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSSESPSPPNLTSGQAGQHHHHHHGAYPYDVPDYAS

SEQ ID NO: 355 - protein, 148 aa, synthetic construct
EVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSSESPSPPNLTSGQAGQHHHHHHGAYPYDVPDYAS

SEQ ID NO: 356 - protein, 147 aa, synthetic construct
EVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSSASTTAPSVTSGQAGQHHHHHHGAYPYDVPDYAS

SEQ ID NO: 357 - protein, 21 aa, synthetic construct
MALPVTALLLPLALLLHAARP

SEQ ID NO: 358 - protein, 5 aa, synthetic construct
DKTHT

SEQ ID NO: 359 - protein, 4 aa, synthetic construct
CPPC

SEQ ID NO: 360 - protein, 15 aa, synthetic construct
CPEPKSCDTPPPCPR

SEQ ID NO: 361 - protein, 12 aa, synthetic construct
ELKTPLGDTTHT

SEQ ID NO: 362 - protein, 10 aa, synthetic construct
KSCDKTHTCP

SEQ ID NO: 363 - protein, 7 aa, synthetic construct
KCCVDCP

SEQ ID NO: 364 - protein, 7 aa, synthetic construct
KYGPPCP

SEQ ID NO: 365 - protein, 15 aa, synthetic construct
EPKSCDKTHTCPPCP

SEQ ID NO: 366 - protein, 12 aa, synthetic construct
ERKCCVECPPCP

SEQ ID NO: 367 - protein, 17 aa, synthetic construct
ELKTPLGDTTHTCPRCP

SEQ ID NO: 368 - protein, 12 aa, synthetic construct
SPNMVPHAHHAQ

SEQ ID NO: 369 - protein, 15 aa, synthetic construct
EPKSCDKTYTCPPCP

SEQ ID NO: 370 - protein, 405 aa, synthetic construct
MALPVTALLLPLALLLHAARPGSQAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVLGGGSSRSSSSGGGGSGGGGEVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 371 - protein, 405 aa, synthetic construct
MALPVTALLLPLALLLHAARPGSSYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTILGGGSSRSSSSGGGGSGGGGEEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 372 - protein, 402 aa, synthetic construct
MALPVTALLLPLALLLHAARPGSQSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVLGGGSSRSSSSGGGGSGGGGEVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 373 - protein, 403 aa, synthetic construct
MALPVTALLLPLALLLHAARPGSQSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVLGGGSSRSSSSGGGGSGGGGEEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 374 - protein, 401 aa, synthetic construct
MALPVTALLLPLALLLHAARPGSQPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVLGGGSSRSSSSGGGGSGGGGEVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 375 - protein, 404 aa, synthetic construct
MALPVTALLLPLALLLHAARPGSQPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 376 - protein, 406 aa, synthetic construct
MALPVTALLLPLALLLHAARPGSSYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVLGGGSSRSSSSGGGGSGGGGEVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 377 - protein, 402 aa, synthetic construct
MALPVTALLLPLALLLHAARPGSQSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVLGGGSSRSSSSGDGGSGGGGEVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 378 - protein, 405 aa, synthetic construct
MALPVTALLLPLALLLHAARPGSQPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTILGGGSSRSSSSGGGGSGGGGEVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSPASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 379 - protein, 402 aa, synthetic construct
MALPVTALLLPLALLLHAARPGSQAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVLGGGSSRSSSSGGGGSGGGGEVQLVQSGGDLVKPGGSLKISCVASGLTFNRNYMTWIRQAPGKGLQWVSEINPDGSTTSYTDAVRGRFTISRDNAKNTLYLQMNSMRDEDTAVYYCARSSWGSFDYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 380 - protein, 398 aa, synthetic construct
MALPVTALLLPLALLLHAARPGSQPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGGGGSGGGGEVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 381 - protein, 402 aa, synthetic construct
MALPVTALLLPLALLLHAARPGSSYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVLGGGSSRSSSSGGGGSGGGGEEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 382 - protein, 401 aa, synthetic construct
MALPVTALLLPLALLLHAARPGSQPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGDGGSGGGGEVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 383 - protein, 403 aa, synthetic construct
MALPVTALLLPLALLLHAARPGSQSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVLGGGSSRSSSSGGGGSGGGGEVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 384 - protein, 407 aa, synthetic construct
MALPVTALLLPLALLLHAARPGSSYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVLGGGSSRSSSSGGGGSGGGGEVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 385 - protein, 402 aa, synthetic construct
MALPVTALLLPLALLLHAARPGSQSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTILGGGSSRSSSSGGGGSGGGGEVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSSESPFPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 386 - protein, 404 aa, synthetic construct
MALPVTALLLPLALLLHAARPGSQPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTILGGGSSRSSSSGGGGSGGGGEVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 387 - protein, 402 aa, synthetic construct
MALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 388 - protein, 401 aa, synthetic construct
MALPVTALLLPLALLLHAARPGSQAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVLGGGSSRSSSSGGGGSGGGGEVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 389 - protein, 397 aa, synthetic construct
MALPVTALLLPLALLLHAARPGSQSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVLGGGSSRSSSSGGGGSGGGGEVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 390 - protein, 399 aa, synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVLGGGSSRSSSSGGGGSGGGGEVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 391 - protein, 402 aa, synthetic construct
MALPVTALLLPLALLLHAARPGSQPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTILGGGSSRSSSSGGGGSGGGGELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 392 - protein, 400 aa, synthetic construct
MALPVTALLLPLALLLHAARPGSDIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIKGGSSRSSSSGGGGSGGGGELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 393 - protein, 402 aa, synthetic construct
MALPVTALLLPLALLLHAARPGSQSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVLGGGSSRSSSSGGGGSGGGGEVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 394 - protein, 405 aa, synthetic construct
MALPVTALLLPLALLLHAARPGSDIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELKGGSSRSSSSGGGGSGGGGEEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 395 - protein, 396 aa, synthetic construct
MALPVTALLLPLALLLHAARPGSSSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 396 - protein, 397 aa, synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 397 - protein, 396 aa, synthetic construct
MALPVTALLLPLALLLHAARPGSQSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 398 - protein, 396 aa, synthetic construct
MALPVTALLLPLALLLHAARPGSQSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTILGGGSSRSSSSGGGGSGGGGEVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 399 - protein, 397 aa, synthetic construct
MALPVTALLLPLALLLHAARPGSQSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 400 - protein, 396 aa, synthetic construct
MALPVTALLLPLALLLHAARPGSQSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVLGGGSSRSSSSGGGGSGGGGEVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 401 - protein, 397 aa, synthetic construct
MALPVTALLLPLALLLHAARPGSQSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 402 - protein, 398 aa, synthetic construct
MALPVTALLLPLALLLHAARPGSDIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIKGGSSRSSSSGGGGSGGGGEVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 403 - protein, 404 aa, synthetic construct
MALPVTALLLPLALLLHAARPGSSYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 404 - protein, 396 aa, synthetic construct
MALPVTALLLPLALLLHAARPGSSSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVLGGGSSRSSSSGGGGSGGGGEVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 405 - protein, 401 aa, synthetic construct
MALPVTALLLPLALLLHAARPGSQSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVLGGGSSRSSSSGGGGSGGGGEEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 406 - protein, 395 aa, synthetic construct
MALPVTALLLPLALLLHAARPGSSSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVLGGGSSRSSSSGGGGSGGGGEVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSSASTTTPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 407 - protein, 396 aa, synthetic construct
MALPVTALLLPLALLLHAARPGSSSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVLGGGSSRSSSSGGGGSGGGGEVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 408 - protein, 404 aa, synthetic construct
MALPVTALLLPLALLLHAARPGSQSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVLGGGSSRSSSSGGGGSGGGGEVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 409 - protein, 401 aa, synthetic construct
MALPVTALLLPLALLLHAARPGSQSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTILGGGSSRSSSSGGGGSGGGGELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 410 - protein, 396 aa, synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVLGGGSSRSSSSGGGGSGGGGEVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 411 - protein, 399 aa, synthetic construct
MALPVTALLLPLALLLHAARPGSQSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTILGGGSSRSSSSGGGGSGGGGEVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSSESPSPPNLTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 412 - protein, 399 aa, synthetic construct
MALPVTALLLPLALLLHAARPGSDIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELKGGSSRSSSSGGGGSGGGGEVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSSASTTAPSVTSGQAGQHHHHHHGAYPYDVPDYASASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

(19) United States
(12) Patent Application Publication
(10) Pub. No.: US 2024/0358834 A1
(43) Pub. Date: Oct. 31, 2024

(54) NOVEL CD19-TARGETING POLYPEPTIDES AND CD19-DIRECTED IMMUNOTHERAPIES

(71) Applicant: THE TRUSTEES OF THE UNIVERSITY OF PENNSYLVANIA, Philadelphia, PA (US)

(72) Inventors: Donald L. Siegel, Lansdale, PA (US); Michael C. Milone, Cherry Hill, NJ (US)

(21) Appl. No.: 18/648,206

(22) Filed: Apr. 26, 2024

Related U.S. Application Data
(60) Provisional application No. 63/499,055, filed on Apr. 28, 2023.

(51) Int. Cl. A61K 39/00; A61P 35/02; C07K 14/725; C07K 16/28

(52) U.S. Cl. CPC A61K 39/464412; A61K 39/4611; A61K 39/4631; A61K 39/4632; A61P 35/02; C07K 14/7051; C07K 16/2803; A61K 2239/13; A61K 2239/28; C07K 2317/622

(57) ABSTRACT

The present disclosure relates to antibodies, binding polypeptides, scFvs, and chimeric antigen receptors (CARs) specific for CD19, nucleic acids encoding the same, methods of treatments, and other methods or uses thereof.

### CROSS-REFERENCE TO RELATED APPLICATIONS

The present application is entitled to priority under 35 U.S.C. § 119 (e) to U.S. Provisional Patent Application No. 63/499,055, filed Apr. 28, 2023, which is hereby incorporated by reference in its entirety.

### SEQUENCE LISTING

The instant application contains a Sequence Listing which has been submitted in XML format via Patent Center and is hereby incorporated by reference in its entirety. Said XML copy, created on Apr. 26, 2024, is named “046483-7422US1 Sequence Listing.txt” and is 426 kilobytes in size.

### BACKGROUND

CD19, a cell-surface molecule on B lymphocytes, has been a clinically significant target for the treatment of acute leukemias and lymphomas due to its robust and reliable expression on most B cell cancer cells. The success of CD19-direct therapy is exemplified by the number of FDA-approved CD19-directed antibody and chimeric antigen receptor (CAR) T cell drugs, although several challenges limit the efficacy of existing therapies. CD19-directed CAR-T cells are also showing promise outside of cancer for counteracting “unwanted immunity” in cases of autoantibody-mediated autoimmune disease, such as for the treatment of Lupus erythematosus as well as other autoimmune diseases and for prevention of alloantibody-mediated solid organ transplant immune incompatibility due to the presence of donor-specific HLA antibodies. In both cases, CD19-directed CAR T cells act to deplete B cells producing pathogenic antibody proteins.

There is a need in the art for the development of novel CD19-directed immunotherapies.

### SUMMARY

This disclosure addresses the above need in various aspects. In one aspect, the invention includes an antibody or antigen-binding fragment thereof that specifically binds to human CD19, wherein the antibody or antigen-binding fragment thereof comprises:
(a) a heavy chain complementarity determining region 1 (HCDR1), comprising the amino acid sequence set forth in SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, or 244;
(b) a heavy chain complementarity determining region 2 (HCDR2), comprising the amino acid sequence set forth in SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, or 237;
(c) a heavy chain complementarity determining region 3 (HCDR3), comprising the amino acid sequence set forth in SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, or 245;
(d) a light chain complementarity determining region 1 (LCDR1), comprising the amino acid sequence set forth in SEQ ID NOs: 90, 95, 100, 109, 114, 119, 124, 128, 140, 148, 153, 158, 166, 171, 180, 185, 200, 203, 209, 213, 221, 229, 234, 242, or 246;
(e) a light chain complementarity determining region 2 (LCDR2), comprising the amino acid sequence set forth in SEQ ID NOs: ADS, AND, ANI, EAS, GNS, GNT, GSH, GSS, GYN, KDT, LVS, NND, QVS, RDT, SDG, SNK, SSS, or SVS, or
(f) a light chain complementarity determining region 3 (LCDR3), comprising the amino acid sequence set forth in SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, or 247.

In certain embodiments, HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

In certain embodiments, the antibody or antigen-binding fragment thereof comprises:
(a) a heavy chain variable region comprising an amino acid sequence having at least 80%, 85%, 90%, 95%, 96%, 96%, 97%, 98%, 99% identity to an amino acid sequence set forth in SEQ ID NOs: 314-356; and/or
(b) a light chain variable region comprising an amino acid sequence having at least 80%, 85%, 90%, 95%, 96%, 97%, 98%, 99% identity to an amino acid sequence set forth in SEQ ID NOs: 271-313.

In certain embodiments, the antibody or antigen-binding fragment thereof comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In another aspect, the invention includes an antibody or antigen-binding fragment thereof that specifically binds to human CD19, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to an amino acid sequence set forth in SEQ ID NOs: 314-356; and
(b) a light chain variable region comprising an amino acid sequence having at least 80%, 85%, 90%, 95%, 96%, 97%, 98%, 99% identity to the amino acid sequence set forth in SEQ ID NOs: 271-313.

In certain embodiments, the antibody or antigen-binding fragment thereof comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In another aspect, the invention includes an antibody or antigen-binding fragment thereof that specifically binds to CD19, wherein the antibody or antigen-binding fragment thereof comprises:
(a) a heavy chain variable region comprising an amino acid sequence set forth in SEQ ID NOs: 314-356; and
(b) a light chain variable region comprising an amino acid sequence set forth in SEQ ID NOs: 271-313.

In certain embodiments, the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

In certain embodiments, the antigen-binding fragment comprises an scFv.

In another aspect, the invention includes a single-chain variable fragment (scFv) that specifically binds to human CD19, comprising:
(a) a heavy chain variable region that comprises an HCDR1, HCDR2, and HCDR3, wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises an amino acid sequence selected from the group consisting of SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises an, LCDR1, LCDR2, and LCDR3, wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises an amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247,
wherein the heavy chain variable region and the light chain variable region are connected by a linker.

In certain embodiments, HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(i) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

In another aspect, the invention includes a single-chain variable fragment (scFv) specifically binds to CD19, comprising:
(a) a heavy chain variable region comprising an amino acid sequence set forth in SEQ ID NOs: 314-356; and
(b) a light chain variable region comprising an amino acid sequence set forth in SEQ ID NOs: 271-313,
wherein the heavy chain variable region and the light chain variable region are connected by a linker.

In certain embodiments, the scFv comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In another aspect, the invention includes a single chain variable fragment (scFv) specifically binds to CD19, comprising an amino acid sequence set forth in SEQ ID NOs: 44-86.

In another aspect, the invention includes a chimeric antigen receptor (CAR) comprising the scFv of any one of the embodiments or aspects disclosed herein.

In another aspect, the invention includes a chimeric antigen receptor (CAR) comprising an antigen-binding domain specific for human CD19 (hCD19), a transmembrane domain, and an intracellular signaling domain, wherein the CAR comprises:
(a) a heavy chain variable region that comprises heavy chain complementarity determining region 1 (HCDR1), HCDR2, and HCDR3, wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises an amino acid sequence selected from the group consisting of SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises light chain complementarity determining region 1 (LCDR1), LCDR2, and LCDR3, wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises an amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

In certain embodiments, the HCDR1, HCDR2, HCDR3, LCDR1, LCDR2, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

In certain embodiments, the antigen-binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303; (hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In certain embodiments, the antigen binding domain comprises a heavy chain variable region comprising an amino acid sequence having at least 95%-99% identity to an amino acid sequence of any of the heavy chain variable regions set forth in SEQ ID NOs: 314-356.

In certain embodiments, the antigen binding domain comprises a heavy chain variable region comprising an amino acid sequence of any of the heavy chain variable regions set forth in SEQ ID NOs: 314-356.

In certain embodiments, the antigen binding domain comprises a heavy chain variable region consisting of an amino acid sequence of any of the heavy chain variable regions set forth in SEQ ID NOs: 314-356.

In certain embodiments, the antigen binding domain comprises a light chain variable region comprising an amino acid sequence having at least 95-99% identity to an amino acid sequence of any of the light chain variable regions set forth in SEQ ID NOs: 271-313.

In certain embodiments, the antigen binding domain comprises a light chain variable region comprising an amino acid sequence of any of the light chain variable regions set forth in SEQ ID NOs: 271-313.

In certain embodiments, the antigen binding domain consists of a light chain variable region consisting of an amino acid sequence of any of the light chain variable regions set forth in SEQ ID NOs: 271-313.

In another aspect, the invention includes a chimeric antigen receptor (CAR) comprising an antigen binding domain, a transmembrane domain, and an intracellular domain, wherein the antigen binding domain comprises a heavy chain variable region comprising any of the amino acid sequences set forth in SEQ ID NO: 314-356; and a light chain variable region comprising any of the amino acid sequences set forth in SEQ ID NO: 271-313.

In certain embodiments, the antigen-binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In another aspect, the invention includes a chimeric antigen receptor (CAR) comprising an antigen binding domain, a transmembrane domain, and an intracellular signaling domain, wherein the antigen binding domain comprises any one of the amino acid sequences set forth in SEQ ID NOs: 44-86.

In certain embodiments, the intracellular signaling domain is derived from a human NK cell receptor (NKR), and wherein the CAR is an NKR-CAR.

In certain embodiments, the NKR domain is selected from the group consisting of an actKIR domain, an NCR domain, a SLAMF domain, an FcR domain, a CD16 domain, a CD64 domain, an actLy49 domain, and an inhLy49 domain.

In certain embodiments, the intracellular signaling domain is a KIR signaling domain selected from the group consisting of a KIR2DS2 signaling domain, a KIR2DS2 signaling domain, a KIR2DL3 signaling domain, a KIR2DL1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain and a KIR3DP1 signaling domain.

In certain embodiments, the transmembrane domain is a KIR transmembrane domain selected from the group consisting of a KIR2DS2 transmembrane domain, a KIR2DS2 transmembrane domain, a KIR2DL3 transmembrane domain, a KIR2DL1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL 1 transmembrane domain, a KIR3DS1 transmembrane domain, a KIR3DL2 transmembrane domain, a KIR3DL3 transmembrane domain, a KIR2DP1 transmembrane domain and a KIR3DP1 transmembrane domain.

In certain embodiments, the transmembrane domain can bind the transmembrane domain of DAP12

In certain embodiments, the KIR signaling domain comprises the amino acid sequence set forth in SEQ ID NO: 266.

In certain embodiments, the CAR comprises an amino acid sequence set forth in SEQ ID NOs: 370-412.

In another aspect, the invention includes an NK cell receptor (NKR)-CAR complex, comprising an NKR-CAR comprising the amino acid sequence set forth in any one of SEQ ID NOs: 370-412 and an adaptor molecule.

In certain embodiments, the adaptor molecule is DAP12 or FcεRγ.

In certain embodiments, the NKR-CAR interacts with the adaptor molecule upon binding of the antigen binding domain of the NKR-CAR to human CD19.

In another aspect, the invention includes an NKR-CAR comprising:
(a) an antigen-binding domain that binds specifically to human CD19 (hCD19), and one or both of;
(b) an NKR transmembrane domain; or
(c) an NKR intracellular signaling domain.

In certain embodiments, the antigen binding domain comprises:
(a) an antigen binding domain comprising: a heavy chain variable region that comprises three heavy chain complementarity determining regions (HCDRs), wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

In certain embodiments, HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

In certain embodiments, the antigen binding domain comprises a heavy chain variable region comprising any of the amino acid sequences set forth in SEQ ID NO: 314-356; and a light chain variable region comprising any of the amino acid sequences set forth in SEQ ID NO: 271-313.

In certain embodiments, the antigen-binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In certain embodiments, the NKR intracellular signaling domain is selected from the group consisting of an actKIR domain, an NCR domain, a SLAMF domain, an FcR domain, a CD16 domain, a CD64 domain, an actLy49 domain, and an inhLy49 domain.

In certain embodiments, the NKR-CAR is a KIR-CAR.

In certain embodiments, the intracellular signaling domain is a KIR signaling domain selected from the group consisting of a KIR2DS2 signaling domain, a KIR2DS2 signaling domain, a KIR2DL3 signaling domain, a KIR2DL1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain and a KIR3 DP1 signaling domain.

In certain embodiments, the transmembrane domain is a KIR transmembrane domain selected from the group consisting of a KIR2DS2 transmembrane domain, a KIR2DS2 transmembrane domain, a KIR2DL3 transmembrane domain, a KIR2DL1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL 1 transmembrane domain, a KIR3DS1 transmembrane domain, a KIR3DL2 transmembrane domain, a KIR3DL3 transmembrane domain, a KIR2DP1 transmembrane domain and a KIR3DP1 transmembrane domain.

In certain embodiments, the transmembrane domain is capable of binding and/or activating DAP12 through the transmembrane domain of DAP12.

In certain embodiments, the KIR signaling domain comprises the amino acid sequence set forth in SEQ ID NO: 266.

In certain embodiments, the CAR comprising an amino acid sequence set forth in SEQ ID NOs: 370-412.

In another aspect, the invention includes a chimeric antigen receptor (CAR) comprising:
(a) an antigen binding domain comprising: a heavy chain variable region that comprises three heavy chain complementarity determining regions (HCDRs), wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247;
(b) a transmembrane domain; and
(c) a KIR2DS2 intracellular signaling domain.

In certain embodiments, HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

In another aspect, the invention includes a chimeric antigen receptor (CAR) comprising:
(a) an antigen binding domain comprising: a heavy chain variable region comprising any of the amino acid sequences set forth in SEQ ID NOs: 314-356; and a light chain variable region comprising any of the amino acid sequences set forth in SEQ ID NOs: 271-313;
(b) a transmembrane domain; and
(c) a KIR2DS2 intracellular signaling domain.

In certain embodiments, the antigen binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In another aspect, the invention includes a chimeric antigen receptor (CAR) comprising:
(a) the CD8 leader amino acid sequence set forth in SEQ ID NO: 357;
(b) an scFv amino acid sequence set forth in SEQ ID NOs: 44-86;
(c) the myc tag amino acid sequence set forth in SEQ ID NO: 264; and
(d) the transmembrane/KIR2DS2 signaling domain amino acid sequence set forth in SEQ ID NO: 266.

In certain embodiments, the CAR comprises an amino acid sequence set forth in SEQ ID NOs: 370-412.

In another aspect, the invention includes an isolated nucleic acid encoding the antibody or antigen-binding fragment thereof of any one of the embodiments disclosed herein.

In certain embodiments, the invention includes an isolated nucleic acid encoding the CAR of any one of the aspects or embodiments disclosed herein, or the NKR-CAR of any one of the aspects or embodiments disclosed herein.

In another aspect, the invention includes an isolated nucleic acid encoding an antibody or antigen-binding fragment thereof, wherein the nucleic acid antibody or antigen-binding fragment thereof comprises a nucleic acid having at least 80%, 85%, 90%, 95%, 96%, 96%, 97%, 98%, 99% identity to the polynucleotide sequence set forth in SEQ ID NOs: 1-43.

In another aspect, the invention includes an isolated nucleic acid encoding an antibody or antigen-binding fragment thereof, wherein the nucleic acid antibody or antigen-binding fragment thereof comprises a polynucleotide sequence set forth in SEQ ID NOs: 1-43.

In certain embodiments, the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

In another aspect, the invention includes an isolated nucleic acid encoding a single-chain variable fragment (scFv) comprising a polynucleotide sequence set forth in SEQ ID NOs: 1-43.

In certain embodiments, the antibody or antigen-binding fragment thereof or the scFv or the antigen-binding domain of the CAR or NKR-CAR specifically binds to human CD19.

In another aspect, the invention includes an isolated nucleic acid encoding a chimeric antigen receptor (CAR) comprising an antigen binding domain, a transmembrane domain, and an intracellular signaling domain, wherein the antigen binding domain comprises:
(a) a heavy chain variable region comprising a nucleic acid encoding any of the amino acid sequences set forth in SEQ ID NOs: 314-356; and
(b) a light chain variable region comprising a nucleic acid encoding any of the amino acid sequences set forth in SEQ ID NOs: 271-313.

In certain embodiments, the antigen binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

In another aspect, the invention includes an isolated nucleic acid encoding a chimeric antigen receptor (CAR) comprising an antigen binding domain, a transmembrane domain, and an intracellular signaling domain, wherein the antigen binding domain is encoded by any one of the nucleotide polynucleotide sequences set forth in SEQ ID NOs: 1-43.

In certain embodiments, the intracellular signaling domain comprises an intracellular domain comprising one or more cytoplasmic signaling domains of a human NK cell KIR receptor.

In certain embodiments, the KIR signaling domain is comprises a KIR2DS2 signaling domain.

In certain embodiments, the KIR signaling domain is encoded by a polynucleotide sequence comprising the sequence set forth in SEQ ID NO: 265.

In another aspect, the invention includes a vector comprising the isolated nucleic acid of any one of the aspects or embodiments disclosed herein.

In certain embodiments, the vector is an expression vector.

In certain embodiments, the vector is selected from the group consisting of a DNA vector, an RNA vector, a plasmid, a lentiviral vector, an adenoviral vector, an adeno-associated viral vector, and a retroviral vector.

In another aspect, the invention includes a host cell comprising the isolated nucleic acid of any one of the aspects or embodiments disclosed herein or the vector of any one of the aspects or embodiments disclosed herein.

In certain embodiments, the host cell is of eukaryotic or prokaryotic origin.

In certain embodiments, the host cell is of mammalian origin.

In certain embodiments, the host cell is of bacterial origin.

In certain embodiments, the host cell is a Chinese Hamster Ovary cell.

In another aspect, the invention includes a modified immune cell or precursor cell thereof, comprising a chimeric antigen receptor (CAR) comprising an antigen binding domain, a transmembrane domain, and an intracellular domain, wherein the antigen binding domain comprises:
(a) a heavy chain variable region that comprises three heavy chain complementarity determining regions (HCDRs), wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of SEQ ID NOs: ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

In certain embodiments, HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
SEQ ID NOs: 206, 207, 208, 209, and 210, and wherein LCDR2 comprises the (gg) amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

In certain embodiments, the CAR binds human CD19.

In certain embodiments, the CAR comprises an antigen binding domain selected from the group consisting of an antibody, an scFv, and a Fab.

In certain embodiments, the CAR comprises an intracellular domain comprising cytoplasmic signaling domains of a human NK cell KIR receptor.

In certain embodiments, the KIR signaling domain comprises a KIR2DS2 signaling domain.

In certain embodiments, the modified cell is an autologous cell.

In certain embodiments, the modified cell is an allogeneic cell.

In certain embodiments, the modified cell is a cell isolated from a human subject.

In certain embodiments, the modified cell is a modified T cell.

In another aspect, the invention includes a pharmaceutical composition comprising the antibody or antigen-binding fragment thereof, the scFv, the CAR, the NKR-CAR, the isolated nucleic acid, the vector, or the modified immune cell of any one of the aspects or embodiments disclosed herein, or any combination thereof, and a pharmaceutically acceptable excipient, carrier, or diluent.

In another aspect, the invention includes a method for generating a modified immune cell or precursor cell thereof, comprising introducing into the immune or precursor cell an isolated nucleic acid encoding the chimeric antigen receptor (CAR) of any one of the aspects or embodiments disclosed herein or the NKR-CAR of any one of the aspects or embodiments disclosed herein.

In certain embodiments, the modified immune cell is a T cell.

In another aspect, the invention includes a method of treating cancer in a subject in need thereof, comprising administering to the subject an effective amount of the modified immune cell disclosed herein, thereby treating the cancer.

In certain embodiments, the cancer is a hematologic cancer.

In certain embodiments, the cancer is associated with the expression of CD19.

In certain embodiments, the CD19 is expressed on tumor cells.

In certain embodiments, the cancer is selected from the group consisting of Burkitt lymphoma, chronic lymphocytic leukemia (CLL), acute lymphoblastic leukemia (ALL), B-cell lymphoma, and B-cell leukemia.

In certain embodiments, the subject is a human.

In another aspect, the invention includes a method of treating an autoimmune disorder or disease in a subject in need thereof, comprising administering to the subject an effective amount of the modified immune cell disclosed herein, thereby treating the autoimmune disorder.

In certain embodiments, the autoimmune disorder is antibody mediated.

In certain embodiments, administration of the modified immune cell depletes autoimmune B cells.

In certain embodiments, the autoimmune disorder is selected from the group comprised of rheumatoid arthritis, systemic lupus erythematosus (SLE), lupus nephritis (LN), idiopathic/autoimmune thrombocytopenia purpura (ITP), idiopathic thrombotic thrombocytopenia purpura (TTP), pemphigus-related disorders, diabetes, scleroderma, myasthenia gravis, multiple sclerosis, vasculitis, and autoimmune hemolytic anemia.

In another aspect, the invention includes a method of treating an alloantibody-mediated disorder or disease in a subject in need thereof, comprising administering to the subject an effective amount of the modified immune cell disclosed herein, thereby treating the alloantibody-mediated disorder or disease.

In certain embodiments, the administration of the modified immune cell depletes B cells that produce alloantibodies.

In certain embodiments, the alloantibody-mediated disorder is selected from the group consisting of solid organ transplant immune incompatibility, resistance to enzyme replacement therapy or acute or delayed hemolytic reactions to transfusion, and chronic alloantibody mediated rejection (CAMR), organ transplant immune incompatibility hemophilia (hemophilia A or B), lysosomal storage diseases (LSD), urea cycle disorder, adenosine deaminase deficiency, neuronal ceroid lipofuscinoses (NCL), hyperammonemia, and chronic graft-versus-host disease (cGvHD).

In certain embodiments, the lysosomal storage disease is selected from the group consisting of glycogen storage disease, Gaucher's disease, Niemann-Pick disease, Fabry's disease, and mucopolysaccharidosis (MPS) I, MPS II, and MPS VI.

### BRIEF DESCRIPTION OF THE DRAWINGS

The following detailed description of embodiments of the present disclosure will be better understood when read in conjunction with the appended drawings. For the purpose of illustrating the present disclosure, there are shown in the drawings embodiments which are presently preferred. It should be understood, however, that the present disclosure is not limited to the precise arrangements and instrumentalities of the embodiments shown in the drawings.

FIG. 1 illustrates an overview of the anti-CD19 discovery campaign.

FIG. 2 illustrates an assessment of enrichment of anti-CD19 scFvs. FIG. 2 (top) illustrates enrichment in the capture of phage beginning in panning round 3. FIG. 2 (bottom) is an ELISA where microplate wells were coated with human CD19 or controls (streptavidin alone or streptavidin loaded with biotinylated-AviTag™-human BCMA or a biotinylated anti-idiotypic antibody to FMC63, a murine anti-human CD19 antibody used in a number of FDA-approved CD19-directed CAR-T therapies including tisagenlecleucel).

FIG. 3 illustrates typical ELISA results for a cohort of mostly positive scFvs (38 of the 84 randomly selected clones are shown in this example).

FIG. 4 is a graph depicting results for scFv clones that were blocked by FMC63 (e.g., scFv 1, 4, 7, etc.), those that do not appear to be blocked at all (e.g., scFv 20, 21, 22, etc.), and those that appear partially blocked (e.g., scFv 31, 33, etc.).

FIG. 5 is a graph showing that CD19 scFv-based CARs are activated in a target specific manner as measured by the Jurkat-NFAT-GFP (JNG) reporter assay.

FIG. 6 illustrates dot plots that show CD19 scFv-based CARs are activated in a target specific manner according to a Jurkat-NFAT-GFP (JNG) reporter assay as assessed by flow cytometry.

FIG. 7 illustrates expression of CD19 scFv-based CAR constructs as measured by flow cytometry staining for the myc tag domain.

FIG. 8 illustrates that two exemplary CD19-CARs are efficiently expressed in human T cells.

FIG. 9 illustrates that CD19-CAR expressing T cells show similar proliferation and expansion rates to each other and other CAR constructs.

FIG. 10 illustrate that in the presence of cognate target antigen, CD19, scFv 18 and 19 CAR-transduced T cells produce cytokines.

FIG. 11 illustrates antigen-specific killing of a CD19 expressing cell line by CD19 scFv CAR-T cells.

FIG. 12 illustrates the treatment of mice bearing NALM6 tumors with CD19 scFv 18 and scFv 19 CAR-T cells leads to temporary tumor regression.

FIG. 13 illustrates the engraftment of human CAR-T cells in mice of the study in FIG. 12 10 days after injection.

FIG. 14 illustrates the rate of growth of NALM6-CBG tumors implanted into NSG mice which then received varying doses of FMC63, scFv 18, and scFv 19-based CAR-T cells. Tumor growth was assessed by in vivo imaging.

### DETAILED DESCRIPTION

This disclosure provides novel polypeptides (such as antigen-binding fragments, scFvs) targeting CD19 (such as human CD19). The disclosed polypeptides exhibit high binding affinity and high specificity against human CD19 and can be used in various immunotherapies for treating CD19-related diseases.

Binding CD19-Targeting Polypeptides, Antibodies, and scFvs

In aspects disclosed herein are antibodies or antigen-binding fragments thereof (or “binding polypeptides,” wherein these terms are used interchangeably) which are characterized by particular functional features or properties of the antibodies or antigen-binding fragments thereof. For example, the binding polypeptides and antibodies specifically bind to CD19, e.g., human CD19. The binding polypeptides and antibodies of the present disclosure can bind to human CD19 with high affinity.

The binding polypeptides and antibodies of the present disclosure can specifically recognize human CD19 protein expressed on a cell. In some cases, the binding polypeptides and antibodies of the present disclosure do not cross-react to other surface molecules on such cell. In some cases, the cell is a tumor cell (e.g., chronic lymphocytic leukemia (CLL), acute lymphoblastic leukemia (ALL), B-cell lymphoma, and B-cell leukemia). In some cases, the cell is an immune cell. In some cases, the immune cell comprises a T cell (e.g., an activated T cell), a B cell, a Natural Killer (NK) cell, a regulatory T cell, a macrophage, a monocyte, or a Dendritic cell (DC). In some cases, the immune cell comprises an activated T cell. In some cases, the immune cell comprises a tumor-infiltrating lymphocyte. In some cases, the CD19 is expressed or overexpressed on a tumor cell.

In certain aspects, the present disclosure provides an antibody or antigen-binding fragment thereof that specifically binds to human CD19. In some cases, the antibody or antigen-binding fragment thereof comprises a heavy chain complementarity determining region 1 (HCDR1). In some cases, the antibody or antigen-binding fragment thereof comprises a heavy chain complementarity determining region 2 (HCDR2). In some cases, the antibody or antigen-binding fragment thereof comprises a heavy chain complementarity determining region 3 (HCDR3). In some cases, the antibody or antigen-binding fragment thereof comprises a light chain complementarity determining region 1 (LCDR1). In some cases, the antibody or antigen-binding fragment thereof comprises a light chain complementarity determining region 2 (LCDR2). In some cases, the antibody or antigen-binding fragment thereof comprises a light chain complementarity determining region 3 (LCDR3). In some cases, the antibody or antigen-binding fragment thereof comprises an antigen-binding domain that specifically binds to human CD19. In certain embodiments, the antigen-binding domain comprises a heavy chain variable region that comprises three heavy chain complementarity determining regions (HCDRs) and a light chain variable region that comprises three light chain complementarity determining regions (LCDRs).

In certain aspects, the present disclosure provides an antibody or antigen-binding fragment thereof that specifically binds to human CD19, comprising (a) a HCDR1, comprising the amino acid sequence set forth in SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, or 244; (b) a HCDR2, comprising the amino acid sequence set forth in SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, or 237; (c) a HCDR3, comprising the amino acid sequence set forth in SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, or 245; (d) a LCDR1, comprising the amino acid sequence set forth in SEQ ID NOs: 90, 95, 100, 109, 114, 119, 124, 128, 140, 148, 153, 158, 166, 171, 180, 185, 200, 203, 209, 213, 221, 229, 234, 242, or 246; (e) a LCDR2, comprising the amino acid sequence ADS, AND, ANI, EAS, GNS, GNT, GSH, GSS, GYN, KDT, LVS, NND, QVS, RDT, SDG, SNK, SSS, or SVS; or (f) a LCDR3, comprising the amino acid sequence set forth in SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, or 247.

In some embodiments, provided herein is an antibody or antigen-binding fragment thereof which binds to human CD19 and comprises an HCDR1, HCDR2, HCDR3, LCDR1, and LCDR3, which comprise the respective amino acid sequences set forth in:
SEQ ID NOs: 87, 88, 89, 90, and 91 and wherein LCDR2 comprises the amino acid sequence GNS;
SEQ ID NOs: 92, 93, 94, 95, and 96, and wherein LCDR2 comprises the amino acid sequence AND;
SEQ ID NOs: 97, 98, 99, 100, and 101, and wherein LCDR2 comprises the amino acid sequence ANI;
SEQ ID NOs: 102, 103, 104, 100, and 105, and wherein LCDR2 comprises the amino acid sequence ANI;
SEQ ID NOs: 106, 107, 108, 109, and 110, and wherein LCDR2 comprises the amino acid sequence SDG;
SEQ ID NOs: 111, 112, 113, 114, and 115, and wherein LCDR2 comprises the amino acid sequence GSS;
SEQ ID NOs: 116, 117, 118, 119, and 120, and wherein LCDR2 comprises the amino acid sequence GNT;
SEQ ID NOs: 121, 122, 123, 124, and 125, and wherein LCDR2 comprises the amino acid sequence GYN;
SEQ ID NOs: 126, 117, 127, 128, and 129, and wherein LCDR2 comprises the amino acid sequence GNS;
SEQ ID NOs: 130, 131, 132, 100, and 133, and wherein LCDR2 comprises the amino acid sequence ANI;
SEQ ID NOs: 134, 135, 136, 100, and 105, and wherein LCDR2 comprises the amino acid sequence ANI;
SEQ ID NOs: 137, 138, 139, 140, and 141, and wherein LCDR2 comprises the amino acid sequence ADS;
SEQ ID NOs: 142, 143, 144, 100, and 105, and wherein LCDR2 comprises the amino acid sequence ANI;
SEQ ID NOs: 145, 146, 147, 148, and 149, and wherein LCDR2 comprises the amino acid sequence GNS;
SEQ ID NOs: 150, 151, 152, 153, and 154, and wherein LCDR2 comprises the amino acid sequence GNS;
SEQ ID NOs: 155, 156, 157, 158, and 159, and wherein LCDR2 comprises the amino acid sequence GSH;
SEQ ID NOs: 121, 160, 161, 128, and 162, and wherein LCDR2 comprises the amino acid sequence GNS;
SEQ ID NOs: 163, 164, 123, 124, and 165, and wherein LCDR2 comprises the amino acid sequence GYN;
SEQ ID NOs: 106, 107, 108, 166, and 167, and wherein LCDR2 comprises the amino acid sequence SDG;
SEQ ID NOs: 168, 169, 170, 171, and 172, and wherein LCDR2 comprises the amino acid sequence KDT;
SEQ ID NOs: 173, 174, 175, 171, and 176, and wherein LCDR2 comprises the amino acid sequence KDT;
SEQ ID NOs: 177, 178, 179, 180, and 181, and wherein LCDR2 comprises the amino acid sequence SSS;
SEQ ID NOs: 182, 183, 184, 185, and 186, and wherein LCDR2 comprises the amino acid sequence LVS;
SEQ ID NOs: 187, 188, 189, 100, and 190, and wherein LCDR2 comprises the amino acid sequence ANI;
SEQ ID NOs: 191, 192, 193, 185, and 194, and wherein LCDR2 comprises the amino acid sequence QVS;
SEQ ID NOs: 195, 196, 197, 171, and 198, and wherein LCDR2 comprises the amino acid sequence KDT;
SEQ ID NOs: 195, 196, 197, 171, and 199, and wherein LCDR2 comprises the amino acid sequence KDT;
SEQ ID NOs: 195, 196, 197, 200, and 201, and wherein LCDR2 comprises the amino acid sequence RDT;
SEQ ID NOs: 195, 196, 197, 200, and 202, and wherein LCDR2 comprises the amino acid sequence KDT;
SEQ ID NOs: 195, 196, 197, 171, and 190, and wherein LCDR2 comprises the amino acid sequence KDT;
SEQ ID NOs: 195, 196, 197, 203, and 204, and wherein LCDR2 comprises the amino acid sequence RDT;
SEQ ID NOs: 195, 196, 197, 171, and 205, and wherein LCDR2 comprises the amino acid sequence KDT;
SEQ ID NOs: 206, 207, 208, 209, and 210, and wherein LCDR2 comprises the amino acid sequence SVS;
SEQ ID NOs: 211, 112, 113, 114, and 115, and wherein LCDR2 comprises the amino acid sequence GSS;
SEQ ID NOs: 168, 117, 212, 213, and 214, and wherein LCDR2 comprises the amino acid sequence KDT;
SEQ ID NOs: 121, 215, 216, 166, and 217, and wherein LCDR2 comprises the amino acid sequence SDG;
SEQ ID NOs: 218, 219, 220, 221, and 222, and wherein LCDR2 comprises the amino acid sequence RDT;
SEQ ID NOs: 223, 224, 225, 213, and 226, and wherein LCDR2 comprises the amino acid sequence KDT;
SEQ ID NOs: 121, 227, 228, 229, and 230, and wherein LCDR2 comprises the amino acid sequence SDG;
SEQ ID NOs: 231, 232, 233, 234, and 235, and wherein LCDR2 comprises the amino acid sequence NND;
SEQ ID NOs: 236, 237, 238, 171, and 239, and wherein LCDR2 comprises the amino acid sequence KDT;
SEQ ID NOs: 240, 143, 241, 242, and 243, and wherein LCDR2 comprises the amino acid sequence SNK; or
SEQ ID NOs: 244, 138, 245, 246, and 247, and wherein LCDR2 comprises the amino acid sequence EAS.

Disclosed herein, in some aspects, is an antibody or antigen-binding fragment thereof that specifically binds to human CD19, comprising: (a) a heavy chain variable region comprising an amino acid sequence having at least 80%, 85%, 90%, 95%, 96%, 96%, 97%, 98%, 99% identity to the amino acid sequence set forth in SEQ ID NOs: 314-356; and (b) a light chain variable region comprising an amino acid sequence having at least 80%, 85%, 90%, 95%, 96%, 97%, 98%, 99% identity to the amino acid sequence set forth in SEQ ID NOs: 271-313.

In some aspects, provided herein is a single-chain variable fragment (scFv) that specifically binds human CD19 comprising (a) a heavy chain variable region that comprises heavy chain complementarity determining region 1 (HCDR1), HCDR2, and HCDR3, wherein

HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises an amino acid sequence selected from the group consisting of SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and (b) a light chain variable region that comprises light chain complementarity determining region 1 (LCDR1), LCDR2, and LCDR3, wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises an amino acid sequence selected from the group consisting of SEQ ID NOs: ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247, wherein the heavy chain variable region and the light chain variable region are connected by a linker.

Also provided is an scFv which binds to human CD19, which comprises an HCDR1, HCDR2, HCDR3, LCDR1, and LCDR3 which comprise SEQ ID NOs: 87, 88, 89, 90, and 91 and wherein LCDR2 comprises the amino acid sequence GNS.

Also provided is an scFv which binds to human CD19, which comprises an HCDR1, HCDR2, HCDR3, LCDR1, and LCDR3 which comprise SEQ ID NOs: 92, 93, 94, 95, and 96, and wherein LCDR2 comprises the amino acid sequence AND.

Also provided is an scFv which binds to human CD19, which comprises an HCDR1, HCDR2, HCDR3, LCDR1, and LCDR3 which comprise SEQ ID NOs: 97, 98, 99, 100, and 101, and wherein LCDR2 comprises the amino acid sequence ANI.

Also provided is an scFv which binds to human CD19, which comprises an HCDR1, HCDR2, HCDR3, LCDR1, and LCDR3 which comprise SEQ ID NOs: 102, 103, 104, 100, and 105, and wherein LCDR2 comprises the amino acid sequence ANI.

Also provided is an scFv which binds to human CD19, which comprises an HCDR1, HCDR2, HCDR3, LCDR1, and LCDR3 which comprise SEQ ID NOs: 106, 107, 108, 109, and 110, and wherein LCDR2 comprises the amino acid sequence SDG.

Also provided is an scFv which binds to human CD19, which comprises an HCDR1, HCDR2, HCDR3, LCDR1, and LCDR3 which comprise SEQ ID NOs: 111, 112, 113, 114, and 115, and wherein LCDR2 comprises the amino acid sequence GSS.

Also provided is an scFv which binds to human CD19, which comprises an HCDR1, HCDR2, HCDR3, LCDR1, and LCDR3 which comprise SEQ ID NOs: 116, 117, 118, 119, and 120, and wherein LCDR2 comprises the amino acid sequence GNT.

Also provided is an scFv which binds to human CD19, which comprises an HCDR1, HCDR2, HCDR3, LCDR1, and LCDR3 which comprise SEQ ID NOs: 121, 122, 123, 124, and 125, and wherein LCDR2 comprises the amino acid sequence GYN.

Also provided is an scFv which binds to human CD19, which comprises an HCDR1, HCDR2, HCDR3, LCDR1, and LCDR3 which comprise SEQ ID NOs: 126, 117, 127, 128, and 129, and wherein LCDR2 comprises the amino acid sequence GNS.

Also provided is an scFv which binds to human CD19, which comprises an HCDR1, HCDR2, HCDR3, LCDR1, and LCDR3 which comprise SEQ ID NOs: 130, 131, 132, 100, and 133, and wherein LCDR2 comprises the amino acid sequence ANI.

Also provided is an scFv which binds to human CD19, which comprises an HCDR1, HCDR2, HCDR3, LCDR1, and LCDR3 which comprise SEQ ID NOs: 134, 135, 136, 100, and 105, and wherein LCDR2 comprises the amino acid sequence ANI.

Also provided is an scFv which binds to human CD19, which comprises an HCDR1, HCDR2, HCDR3, LCDR1, and LCDR3 which comprise SEQ ID NOs: 137, 138, 139, 140, and 141, and wherein LCDR2 comprises the amino acid sequence ADS.

Also provided is an scFv which binds to human CD19, which comprises an HCDR1, HCDR2, HCDR3, LCDR1, and LCDR3 which comprise SEQ ID NOs: 142, 143, 144, 100, and 105, and wherein LCDR2 comprises the amino acid sequence ANI.

Also provided is an scFv which binds to human CD19, which comprises an HCDR1, HCDR2, HCDR3, LCDR1, and LCDR3 which comprise SEQ ID NOs: 145, 146, 147, 148, and 149, and wherein LCDR2 comprises the amino acid sequence GNS.

Also provided is an scFv which binds to human CD19, which comprises an HCDR1, HCDR2, HCDR3, LCDR1, and LCDR3 which comprise SEQ ID NOs: 150, 151, 152, 153, and 154, and wherein LCDR2 comprises the amino acid sequence GNS.

Also provided is an scFv which binds to human CD19, which comprises an HCDR1, HCDR2, HCDR3, LCDR1, and LCDR3 which comprise SEQ ID NOs: 155, 156, 157, 158, and 159, and wherein LCDR2 comprises the amino acid sequence GSH.

Also provided is an scFv which binds to human CD19, which comprises an HCDR1, HCDR2, HCDR3, LCDR1, and LCDR3 which comprise SEQ ID NOs: 121, 160, 161, 128, and 162, and wherein LCDR2 comprises the amino acid sequence GNS.

Also provided is an scFv which binds to human CD19, which comprises an HCDR1, HCDR2, HCDR3, LCDR1, and LCDR3 which comprise SEQ ID NOs: 163, 164, 123, 124, and 165, and wherein LCDR2 comprises the amino acid sequence GYN.

Also provided is an scFv which binds to human CD19, which comprises an HCDR1, HCDR2, HCDR3, LCDR1, and LCDR3 which comprise SEQ ID NOs: 106, 107, 108, 166, and 167, and wherein LCDR2 comprises the amino acid sequence SDG.

Also provided is an scFv which binds to human CD19, which comprises an HCDR1, HCDR2, HCDR3, LCDR1, and LCDR3 which comprise SEQ ID NOs: 168, 169, 170, 171, and 172, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 173, 174, 175, 171, and 176, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 177, 178, 179, 180, and 181, and wherein LCDR2 comprises the amino acid sequence SSS.

Also provided is an scFv which binds to human CD19, which comprises an HCDR1, HCDR2, HCDR3, LCDR1, and LCDR3 which comprise SEQ ID NOs: 182, 183, 184, 185, and 186, and wherein LCDR2 comprises the amino acid sequence LVS.

Also provided is an scFv which binds to human CD19, which comprises an HCDR1, HCDR2, HCDR3, LCDR1, and LCDR3 which comprise SEQ ID NOs: 187, 188, 189, 100, and 190, and wherein LCDR2 comprises the amino acid sequence ANI.

Also provided is an scFv which binds to human CD19, which comprises an HCDR1, HCDR2, HCDR3, LCDR1, and LCDR3 which comprise SEQ ID NOs: 191, 192, 193, 185, and 194, and wherein LCDR2 comprises the amino acid sequence QVS.

Also provided is an scFv which binds to human CD19, which comprises an HCDR1, HCDR2, HCDR3, LCDR1, and LCDR3 which comprise SEQ ID NOs: 195, 196, 197, 171, and 198, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 195, 196, 197, 171, and 199, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 195, 196, 197, 200, and 201, and wherein LCDR2 comprises the amino acid sequence RDT.

Also provided is an scFv which binds to human CD19, which comprises an HCDR1, HCDR2, HCDR3, LCDR1, and LCDR3 which comprise SEQ ID NOs: 195, 196, 197, 200, and 202, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 195, 196, 197, 171, and 190, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 195, 196, 197, 203, and 204, and wherein LCDR2 comprises the amino acid sequence RDT.

Also provided is an scFv which binds to human CD19, which comprises an HCDR1, HCDR2, HCDR3, LCDR1, and LCDR3 which comprise SEQ ID NOs: 195, 196, 197, 171, and 205, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 206, 207, 208, 209, and 210, and wherein LCDR2 comprises the amino acid sequence SVS.

Also provided is an scFv which binds to human CD19, which comprises an HCDR1, HCDR2, HCDR3, LCDR1, and LCDR3 which comprise SEQ ID NOs: 211, 112, 113, 114, and 115, and wherein LCDR2 comprises the amino acid sequence GSS.

Also provided is an scFv which binds to human CD19, which comprises an HCDR1, HCDR2, HCDR3, LCDR1, and LCDR3 which comprise SEQ ID NOs: 168, 117, 212, 213, and 214, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 121, 215, 216, 166, and 217, and wherein LCDR2 comprises the amino acid sequence SDG.

Also provided is an scFv which binds to human CD19, which comprises an HCDR1, HCDR2, HCDR3, LCDR1, and LCDR3 which comprise SEQ ID NOs: 218, 219, 220, 221, and 222, and wherein LCDR2 comprises the amino acid sequence RDT.

Also provided is an scFv which binds to human CD19, which comprises an HCDR1, HCDR2, HCDR3, LCDR1, and LCDR3 which comprise SEQ ID NOs: 223, 224, 225, 213, and 226, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 121, 227, 228, 229, and 230, and wherein LCDR2 comprises the amino acid sequence SDG.

Also provided is an scFv which binds to human CD19, which comprises an HCDR1, HCDR2, HCDR3, LCDR1, and LCDR3 which comprise SEQ ID NOs: 231, 232, 233, 234, and 235, and wherein LCDR2 comprises the amino acid sequence NND.

Also provided is an scFv which binds to human CD19, which comprises an HCDR1, HCDR2, HCDR3, LCDR1, and LCDR3 which comprise SEQ ID NOs: 236, 237, 238, 171, and 239, and wherein LCDR2 comprises the amino acid sequence KDT.

Also provided is an scFv which binds to human CD19, which comprises an HCDR1, HCDR2, HCDR3, LCDR1, and LCDR3 which comprise SEQ ID NOs: 240, 143, 241, 242, and 243, and wherein LCDR2 comprises the amino acid sequence SNK.

Also provided is an scFv which binds to human CD19, which comprises an HCDR1, HCDR2, HCDR3, LCDR1, and LCDR3 which comprise SEQ ID NOs: 244, 138, 245, 246, and 247, and wherein LCDR2 comprises the amino acid sequence EAS.

In some aspects, provided herein is a single-chain variable fragment (scFv) comprising (a) a heavy chain variable region comprising the amino acid sequence set forth in SEQ ID NOs: 314-356; and (b) a light chain variable region comprising the amino acid sequence set forth in SEQ ID NOs: 271-313, wherein the heavy chain variable region and the light chain variable region are connected by a linker.

Also provided is an scFv comprising a heavy chain variable region comprising the amino acid sequence set forth in SEQ ID NO: 314 and a light chain variable region comprising the amino acid sequence set forth in SEQ ID NO: 271.

Also provided is an scFv comprising a heavy chain variable region comprising the amino acid sequence set forth in SEQ ID NO: 315 and a light chain variable region comprising the amino acid sequence set forth in SEQ ID NO: 272.

Also provided is an scFv comprising a heavy chain variable region comprising the amino acid sequence set forth in SEQ ID NO: 316 and a light chain variable region comprising the amino acid sequence set forth in SEQ ID NO: 273.

Also provided is an scFv comprising a heavy chain variable region comprising the amino acid sequence set forth in SEQ ID NO: 317 and a light chain variable region comprising the amino acid sequence set forth in SEQ ID NO: 274.

Also provided is an scFv comprising a heavy chain variable region comprising the amino acid sequence set forth in SEQ ID NO: 318 and a light chain variable region comprising the amino acid sequence set forth in SEQ ID NO: 275.

Also provided is an scFv comprising a heavy chain variable region comprising the amino acid sequence set forth in SEQ ID NO: 319 and a light chain variable region comprising the amino acid sequence set forth in SEQ ID NO: 276.

Also provided is an scFv comprising a heavy chain variable region comprising the amino acid sequence set forth in SEQ ID NO: 320 and a light chain variable region comprising the amino acid sequence set forth in SEQ ID NO: 277.

Also provided is an scFv comprising a heavy chain variable region comprising the amino acid sequence set forth in SEQ ID NO: 321 and a light chain variable region comprising the amino acid sequence set forth in SEQ ID NO: 278.

Also provided is an scFv comprising a heavy chain variable region comprising the amino acid sequence set forth in SEQ ID NO: 322 and a light chain variable region comprising the amino acid sequence set forth in SEQ ID NO: 279.

Also provided is an scFv comprising a heavy chain variable region comprising the amino acid sequence set forth in SEQ ID NO: 323 and a light chain variable region comprising the amino acid sequence set forth in SEQ ID NO: 280.

Also provided is an scFv comprising a heavy chain variable region comprising the amino acid sequence set forth in SEQ ID NO: 324 and a light chain variable region comprising the amino acid sequence set forth in SEQ ID NO: 281.

Also provided is an scFv comprising a heavy chain variable region comprising the amino acid sequence set forth in SEQ ID NO: 325 and a light chain variable region comprising the amino acid sequence set forth in SEQ ID NO: 282.

Also provided is an scFv comprising a heavy chain variable region comprising the amino acid sequence set forth in SEQ ID NO: 326 and a light chain variable region comprising the amino acid sequence set forth in SEQ ID NO: 283.

Also provided is an scFv comprising a heavy chain variable region comprising the amino acid sequence set forth in SEQ ID NO: 327 and a light chain variable region comprising the amino acid sequence set forth in SEQ ID NO: 284.

Also provided is an scFv comprising a heavy chain variable region comprising the amino acid sequence set forth in SEQ ID NO: 328 and a light chain variable region comprising the amino acid sequence set forth in SEQ ID NO: 285.

Also provided is an scFv comprising a heavy chain variable region comprising the amino acid sequence set forth in SEQ ID NO: 329 and a light chain variable region comprising the amino acid sequence set forth in SEQ ID NO: 286.

Also provided is an scFv comprising a heavy chain variable region comprising the amino acid sequence set forth in SEQ ID NO: 330 and a light chain variable region comprising the amino acid sequence set forth in SEQ ID NO: 287.

Also provided is an scFv comprising a heavy chain variable region comprising the amino acid sequence set forth in SEQ ID NO: 331 and a light chain variable region comprising the amino acid sequence set forth in SEQ ID NO: 288.

Also provided is an scFv comprising a heavy chain variable region comprising the amino acid sequence set forth in SEQ ID NO: 332 and a light chain variable region comprising the amino acid sequence set forth in SEQ ID NO: 289.

Also provided is an scFv comprising a heavy chain variable region comprising the amino acid sequence set forth in SEQ ID NO: 333 and a light chain variable region comprising the amino acid sequence set forth in SEQ ID NO: 290.

Also provided is an scFv comprising a heavy chain variable region comprising the amino acid sequence set forth in SEQ ID NO: 334 and a light chain variable region comprising the amino acid sequence set forth in SEQ ID NO: 291.

Also provided is an scFv comprising a heavy chain variable region comprising the amino acid sequence set forth in SEQ ID NO: 335 and a light chain variable region comprising the amino acid sequence set forth in SEQ ID NO: 292.

Also provided is an scFv comprising a heavy chain variable region comprising the amino acid sequence set forth in SEQ ID NO: 336 and a light chain variable region comprising the amino acid sequence set forth in SEQ ID NO: 293.

Also provided is an scFv comprising a heavy chain variable region comprising the amino acid sequence set forth in SEQ ID NO: 337 and a light chain variable region comprising the amino acid sequence set forth in SEQ ID NO: 294.

Also provided is an scFv comprising a heavy chain variable region comprising the amino acid sequence set forth in SEQ ID NO: 338 and a light chain variable region comprising the amino acid sequence set forth in SEQ ID NO: 295.

Also provided is an scFv comprising a heavy chain variable region comprising the amino acid sequence set forth in SEQ ID NO: 339 and a light chain variable region comprising the amino acid sequence set forth in SEQ ID NO: 296.

Also provided is an scFv comprising a heavy chain variable region comprising the amino acid sequence set forth in SEQ ID NO: 340 and a light chain variable region comprising the amino acid sequence set forth in SEQ ID NO: 297.

Also provided is an scFv comprising a heavy chain variable region comprising the amino acid sequence set forth in SEQ ID NO: 341 and a light chain variable region comprising the amino acid sequence set forth in SEQ ID NO: 298.

Also provided is an scFv comprising a heavy chain variable region comprising the amino acid sequence set forth in SEQ ID NO: 342 and a light chain variable region comprising the amino acid sequence set forth in SEQ ID NO: 299.

Also provided is an scFv comprising a heavy chain variable region comprising the amino acid sequence set forth in SEQ ID NO: 343 and a light chain variable region comprising the amino acid sequence set forth in SEQ ID NO: 300.

Also provided is an scFv comprising a heavy chain variable region comprising the amino acid sequence set forth in SEQ ID NO: 344 and a light chain variable region comprising the amino acid sequence set forth in SEQ ID NO: 301.

Also provided is an scFv comprising a heavy chain variable region comprising the amino acid sequence set forth in SEQ ID NO: 345 and a light chain variable region comprising the amino acid sequence set forth in SEQ ID NO: 302.

Also provided is an scFv comprising a heavy chain variable region comprising the amino acid sequence set forth in SEQ ID NO: 346 and a light chain variable region comprising the amino acid sequence set forth in SEQ ID NO: 303.

Also provided is an scFv comprising a heavy chain variable region comprising the amino acid sequence set forth in SEQ ID NO: 347 and a light chain variable region comprising the amino acid sequence set forth in SEQ ID NO: 304.

Also provided is an scFv comprising a heavy chain variable region comprising the amino acid sequence set forth in SEQ ID NO: 348 and a light chain variable region comprising the amino acid sequence set forth in SEQ ID NO: 305.

Also provided is an scFv comprising a heavy chain variable region comprising the amino acid sequence set forth in SEQ ID NO: 349 and a light chain variable region comprising the amino acid sequence set forth in SEQ ID NO: 306.

Also provided is an scFv comprising a heavy chain variable region comprising the amino acid sequence set forth in SEQ ID NO: 350 and a light chain variable region comprising the amino acid sequence set forth in SEQ ID NO: 307.

Also provided is an scFv comprising a heavy chain variable region comprising the amino acid sequence set forth in SEQ ID NO: 351 and a light chain variable region comprising the amino acid sequence set forth in SEQ ID NO: 308.

Also provided is an scFv comprising a heavy chain variable region comprising the amino acid sequence set forth in SEQ ID NO: 352 and a light chain variable region comprising the amino acid sequence set forth in SEQ ID NO: 309.

Also provided is an scFv comprising a heavy chain variable region comprising the amino acid sequence set forth in SEQ ID NO: 353 and a light chain variable region comprising the amino acid sequence set forth in SEQ ID NO: 310.

Also provided is an scFv comprising a heavy chain variable region comprising the amino acid sequence set forth in SEQ ID NO: 354 and a light chain variable region comprising the amino acid sequence set forth in SEQ ID NO: 311.

Also provided is an scFv comprising a heavy chain variable region comprising the amino acid sequence set forth in SEQ ID NO: 355 and a light chain variable region comprising the amino acid sequence set forth in SEQ ID NO: 312.

Also provided is an scFv comprising a heavy chain variable region comprising the amino acid sequence set forth in SEQ ID NO: 356 and a light chain variable region comprising the amino acid sequence set forth in SEQ ID NO: 313.

In some aspects, provided herein is a scFv which specifically binds human CD19 and comprises the amino acid sequence set forth in SEQ ID NOs: 44-86.

Also provided are CARs, bispecific molecules, immunoconjugates, nucleic acids, vectors, host cells, kits, compositions for the human CD19-specific antibodies and antigen-binding fragments or the scFv described herein, and methods of producing and using the same for the treatment of cancers in subjects.

Tolerable variations of the CDR sequences will be known to those of skill in the art. For example, in some embodiments, the isolated binding polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences ADS, AND, ANI, EAS, GNS, GNT, GSH, GSS, GYN, KDT, LVS, NND, QVS, RDT, SDG, SNK, SSS, or SVS; or the amino acid sequences set forth in SEQ ID NOs: 87-247. In some embodiments, the isolated binding polypeptide is an antibody or antigen-binding fragment thereof that specifically binds to human CD19.

In some embodiments, the isolated binding polypeptide binds a CD19 protein, for example, human CD19. In some embodiments, the binding polypeptide comprises an antibody or an antigen-binding fragment thereof. In some embodiments, the antigen-binding fragment is selected from the group consisting of a full-length antibody, a Fab, a single-chain variable fragment (scFv), a single-domain antibody, sc (Fv) 2, dsFv, Fab, Fab′, (Fab′) 2 and a diabody. In some embodiments, the antibody or antigen-binding fragment is an scFv. In some embodiments, the antibody or antigen-binding fragment is a canine scFv.

In certain embodiments, the binding polypeptide comprises a heavy chain variable region comprising an amino acid sequence having at least 80%, 85%, 90%, 95%, 96%, 96%, 97%, 98%, or 99% identity to the amino acid sequence of the heavy chain variable region set forth in SEQ ID NOs: 314-356. In certain embodiments, the binding polypeptide comprises a heavy chain variable region comprising the amino acid sequence set forth in SEQ ID NOs: 314-356. In certain embodiments, the binding polypeptide comprises a heavy chain variable region consisting of the amino acid sequences set forth in SEQ ID NOs: 314-356.

In certain embodiments, the binding polypeptide comprises a light chain variable region comprising an amino acid sequence having at least 80%, 85%, 90%, 95%, 96%, 97%, 98%, or 99% identity to the amino acid sequence set forth in SEQ ID NOs: 271-313. In certain embodiments, the binding polypeptide comprises a light chain variable region comprising an amino acid sequence set forth in SEQ ID NOs: 271-313. In certain embodiments, the binding polypeptide consists of a light chain variable region comprising an amino acid sequence set forth in SEQ ID NOs: 271-313.

Also provided is an isolated binding polypeptide comprising a heavy chain variable region comprising the amino acid sequence set forth in SEQ ID NO: 314 and a light chain variable region comprising the amino acid sequence set forth in SEQ ID NO: 271.

Also provided is an isolated binding polypeptide comprising a heavy chain variable region comprising the amino acid sequence set forth in SEQ ID NO: 315 and a light chain variable region comprising the amino acid sequence set forth in SEQ ID NO: 272.

Also provided is an isolated binding polypeptide comprising a heavy chain variable region comprising the amino acid sequence set forth in SEQ ID NO: 316 and a light chain variable region comprising the amino acid sequence set forth in SEQ ID NO: 273.

Also provided is an isolated binding polypeptide comprising a heavy chain variable region comprising the amino acid sequence set forth in SEQ ID NO: 317 and a light chain variable region comprising the amino acid sequence set forth in SEQ ID NO: 274.

Also provided is an isolated binding polypeptide comprising a heavy chain variable region comprising the amino acid sequence set forth in SEQ ID NO: 318 and a light chain variable region comprising the amino acid sequence set forth in SEQ ID NO: 275.

Also provided is an isolated binding polypeptide comprising a heavy chain variable region comprising the amino acid sequence set forth in SEQ ID NO: 319 and a light chain variable region comprising the amino acid sequence set forth in SEQ ID NO: 276.

Also provided is an isolated binding polypeptide comprising a heavy chain variable region comprising the amino acid sequence set forth in SEQ ID NO: 320 and a light chain variable region comprising the amino acid sequence set forth in SEQ ID NO: 277.

Also provided is an isolated binding polypeptide comprising a heavy chain variable region comprising the amino acid sequence set forth in SEQ ID NO: 321 and a light chain variable region comprising the amino acid sequence set forth in SEQ ID NO: 278.

Also provided is an isolated binding polypeptide comprising a heavy chain variable region comprising the amino acid sequence set forth in SEQ ID NO: 322 and a light chain variable region comprising the amino acid sequence set forth in SEQ ID NO: 279.

Also provided is an isolated binding polypeptide comprising a heavy chain variable region comprising the amino acid sequence set forth in SEQ ID NO: 323 and a light chain variable region comprising the amino acid sequence set forth in SEQ ID NO: 280.

Also provided is an isolated binding polypeptide comprising a heavy chain variable region comprising the amino acid sequence set forth in SEQ ID NO: 324 and a light chain variable region comprising the amino acid sequence set forth in SEQ ID NO: 281.

Also provided is an isolated binding polypeptide comprising a heavy chain variable region comprising the amino acid sequence set forth in SEQ ID NO: 325 and a light chain variable region comprising the amino acid sequence set forth in SEQ ID NO: 282.

Also provided is an isolated binding polypeptide comprising a heavy chain variable region comprising the amino acid sequence set forth in SEQ ID NO: 326 and a light chain variable region comprising the amino acid sequence set forth in SEQ ID NO: 283.

Also provided is an isolated binding polypeptide comprising a heavy chain variable region comprising the amino acid sequence set forth in SEQ ID NO: 327 and a light chain variable region comprising the amino acid sequence set forth in SEQ ID NO: 284.

Also provided is an isolated binding polypeptide comprising a heavy chain variable region comprising the amino acid sequence set forth in SEQ ID NO: 328 and a light chain variable region comprising the amino acid sequence set forth in SEQ ID NO: 285.

Also provided is an isolated binding polypeptide comprising a heavy chain variable region comprising the amino acid sequence set forth in SEQ ID NO: 329 and a light chain variable region comprising the amino acid sequence set forth in SEQ ID NO: 286.

Also provided is an isolated binding polypeptide comprising a heavy chain variable region comprising the amino acid sequence set forth in SEQ ID NO: 330 and a light chain variable region comprising the amino acid sequence set forth in SEQ ID NO: 287.

Also provided is an isolated binding polypeptide comprising a heavy chain variable region comprising the amino acid sequence set forth in SEQ ID NO: 331 and a light chain variable region comprising the amino acid sequence set forth in SEQ ID NO: 288.

Also provided is an isolated binding polypeptide comprising a heavy chain variable region comprising the amino acid sequence set forth in SEQ ID NO: 332 and a light chain variable region comprising the amino acid sequence set forth in SEQ ID NO: 289.

Also provided is an isolated binding polypeptide comprising a heavy chain variable region comprising the amino acid sequence set forth in SEQ ID NO: 333 and a light chain variable region comprising the amino acid sequence set forth in SEQ ID NO: 290

Also provided is an isolated binding polypeptide comprising a heavy chain variable region comprising the amino acid sequence set forth in SEQ ID NO: 334 and a light chain variable region comprising the amino acid sequence set forth in SEQ ID NO: 291.

Also provided is an isolated binding polypeptide comprising a heavy chain variable region comprising the amino acid sequence set forth in SEQ ID NO: 335 and a light chain variable region comprising the amino acid sequence set forth in SEQ ID NO: 292.

Also provided is an isolated binding polypeptide comprising a heavy chain variable region comprising the amino acid sequence set forth in SEQ ID NO: 336 and a light chain variable region comprising the amino acid sequence set forth in SEQ ID NO: 293.

Also provided is an isolated binding polypeptide comprising a heavy chain variable region comprising the amino acid sequence set forth in SEQ ID NO: 337 and a light chain variable region comprising the amino acid sequence set forth in SEQ ID NO: 294.

Also provided is an isolated binding polypeptide comprising a heavy chain variable region comprising the amino acid sequence set forth in SEQ ID NO: 338 and a light chain variable region comprising the amino acid sequence set forth in SEQ ID NO: 295.

Also provided is an isolated binding polypeptide comprising a heavy chain variable region comprising the amino acid sequence set forth in SEQ ID NO: 339 and a light chain variable region comprising the amino acid sequence set forth in SEQ ID NO: 296.

Also provided is an isolated binding polypeptide comprising a heavy chain variable region comprising the amino acid sequence set forth in SEQ ID NO: 340 and a light chain variable region comprising the amino acid sequence set forth in SEQ ID NO: 297.

Also provided is an isolated binding polypeptide comprising a heavy chain variable region comprising the amino acid sequence set forth in SEQ ID NO: 341 and a light chain variable region comprising the amino acid sequence set forth in SEQ ID NO: 298.

Also provided is an isolated binding polypeptide comprising a heavy chain variable region comprising the amino acid sequence set forth in SEQ ID NO: 342 and a light chain variable region comprising the amino acid sequence set forth in SEQ ID NO: 299.

Also provided is an isolated binding polypeptide comprising a heavy chain variable region comprising the amino acid sequence set forth in SEQ ID NO: 343 and a light chain variable region comprising the amino acid sequence set forth in SEQ ID NO: 300.

Also provided is an isolated binding polypeptide comprising a heavy chain variable region comprising the amino acid sequence set forth in SEQ ID NO: 344 and a light chain variable region comprising the amino acid sequence set forth in SEQ ID NO: 301.

Also provided is an isolated binding polypeptide comprising a heavy chain variable region comprising the amino acid sequence set forth in SEQ ID NO: 345 and a light chain variable region comprising the amino acid sequence set forth in SEQ ID NO: 302.

Also provided is an isolated binding polypeptide comprising a heavy chain variable region comprising the amino acid sequence set forth in SEQ ID NO: 346 and a light chain variable region comprising the amino acid sequence set forth in SEQ ID NO: 303.

Also provided is an isolated binding polypeptide comprising a heavy chain variable region comprising the amino acid sequence set forth in SEQ ID NO: 347 and a light chain variable region comprising the amino acid sequence set forth in SEQ ID NO: 304.

Also provided is an isolated binding polypeptide comprising a heavy chain variable region comprising the amino acid sequence set forth in SEQ ID NO: 348 and a light chain variable region comprising the amino acid sequence set forth in SEQ ID NO: 305.

Also provided is an isolated binding polypeptide comprising a heavy chain variable region comprising the amino acid sequence set forth in SEQ ID NO: 349 and a light chain variable region comprising the amino acid sequence set forth in SEQ ID NO: 306.

Also provided is an isolated binding polypeptide comprising a heavy chain variable region comprising the amino acid sequence set forth in SEQ ID NO: 350 and a light chain variable region comprising the amino acid sequence set forth in SEQ ID NO: 307.

Also provided is an isolated binding polypeptide comprising a heavy chain variable region comprising the amino acid sequence set forth in SEQ ID NO: 351 and a light chain variable region comprising the amino acid sequence set forth in SEQ ID NO: 308.

Also provided is an isolated binding polypeptide comprising a heavy chain variable region comprising the amino acid sequence set forth in SEQ ID NO: 352 and a light chain variable region comprising the amino acid sequence set forth in SEQ ID NO: 309.

Also provided is an isolated binding polypeptide comprising a heavy chain variable region comprising the amino acid sequence set forth in SEQ ID NO: 353 and a light chain variable region comprising the amino acid sequence set forth in SEQ ID NO: 310.

Also provided is an isolated binding polypeptide comprising a heavy chain variable region comprising the amino acid sequence set forth in SEQ ID NO: 354 and a light chain variable region comprising the amino acid sequence set forth in SEQ ID NO: 311.

Also provided is an isolated binding polypeptide comprising a heavy chain variable region comprising the amino acid sequence set forth in SEQ ID NO: 355 and a light chain variable region comprising the amino acid sequence set forth in SEQ ID NO: 312.

Also provided is an isolated binding polypeptide comprising a heavy chain variable region comprising the amino acid sequence set forth in SEQ ID NO: 356 and a light chain variable region comprising the amino acid sequence set forth in SEQ ID NO: 313.

An antibody of the present disclosure can be prepared using an antibody having one or more of the VH and/or VL sequences or any fragments thereof disclosed herein as a starting material to engineer a modified antibody, which modified antibody can have altered properties as compared with the starting antibody. An antibody can be engineered by modifying one or more amino acids within one or both variable regions (i.e., VH and/or VL), for example within one or more CDR regions and/or within one or more framework regions. Additionally, or alternatively, an antibody can be engineered by modifying residues within the constant region(s), for example to alter the effector function(s) of the antibody.

Also provided is a canine antibody or antigen-binding fragment thereof that specifically binds to human CD19. The canine antibody or antigen-binding fragment thereof can be generated from a canine antibody phage display library. The process for generating canine antibodies or antigen-binding fragments thereof that specifically bind hCD19 is illustrated in Example 1. In some cases, such canine antibody comprises any antigen-binding fragments or binding peptide disclosed herein.

Also provided is a single-chain variable fragment (scFv) specifically binds to human CD19. In some cases, the scFv comprises an antigen-binding domain. In some cases, the specific binding of antibody or antigen-binding fragment thereof to hCD19 disrupts the interaction of hCD19.

As used herein, the term “single-chain variable fragment” or “scFv” is a fusion protein of the variable regions of the heavy (VH) and light chains (VL) of an immunoglobulin (e.g., mouse, canine, or human) covalently linked to form a VH: VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., CD19 binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present disclosure.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80 (6): 1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 248), (GGGS)ₙ (SEQ ID NO: 249), and (GGGGS)ₙ (SEQ ID NO: 250), where n represents an integer of at least 1. Exemplary linker sequences can comprise amino acid sequences including, without limitation, GGSG (SEQ ID NO: 251), GGSGG (SEQ ID NO: 252), GSGSG (SEQ ID NO: 253), GSGGG (SEQ ID NO: 254), GGGSG (SEQ ID NO: 255), GSSSG (SEQ ID NO: 256), GGGGS (SEQ ID NO: 257), GGGGSGGGGSGGGGS (SEQ ID NO: 258), GGGSSRSSSSGGGGSGGGG (SEQ ID NO: 259), SGGGGSGGGGS (SEQ ID NO: 260) and the like. Those of skill in the art would be able to select the appropriate linker sequence for use in the present disclosure. In one embodiment, an scFv of the present disclosure comprises a heavy chain variable region (VH) and a light chain variable region (VL), wherein the VH and VL are connected by the linker sequence having the amino acid sequence GGGSSRSSSSGGGGSGGGG (SEQ ID NO: 259), which can be encoded by the nucleic acid sequence GGCGGTGGTTCCTCTAGATCTTCCTCCTCTGGTGG CGGTGGCTCGGGCGGTGGTGGG (SEQ ID NO: 261) in which an arginine residue, R, is present as a result of including a nucleotide sequence for the restriction endonuclease Xba I. The presence of restriction sites in the linker along with those flanking an scFv construct will be recognized by those skilled in the art to be useful for performing heavy chain/light chain “swapping” experiments for antibody optimization, if desired.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston et al. (Proc. Nat. Acad. Sci. USA, 85:5879-5883, 1988). See also U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27 (6): 455-51; Peter et al., J Cachexia Sarcopenia Muscle 2012 Aug. 12; Shieh et al., J Imunol 2009 183 (4): 2277-85; Giomarelli et al., Thromb Haemost 2007 97 (6): 955-63; Fife eta., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Biol Chem 2003 25278 (38): 36740-7; Xie et al., Nat Biotech 1997 15 (8): 768-71; Ledbetter et al., Crit Rev Immunol 1997 17 (5-6): 427-55; Ho et al., BioChim Biophys Acta 2003 1638 (3): 257-66).

In certain embodiments, the antigen-binding domain of the scFv comprises a heavy chain variable region that comprises three heavy chain complementarity determining regions (HCDRs) and a light chain variable region that comprises three light chain complementarity determining regions (LCDRs). HCDR1 comprises the amino acid sequence (SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, or 244), and/or HCDR2 comprises the amino acid sequence (SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, or 237), and/or HCDR3 comprises the amino acid sequence (SEQ ID NO: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, or 245) and/or LCDR1 comprises the amino acid sequence (SEQ ID NOs: 90, 95, 100, 109, 114, 119, 124, 128, 140, 148, 153, 158, 166, 171, 180, 185, 200, 203, 209, 213, 221, 229, 234, 242, or 246), and/or LCDR2 comprises the amino acid sequence (ADS, AND, ANI, EAS, GNS, GNT, GSH, GSS, GYN, KDT, LVS, NND, QVS, RDT, SDG, SNK, SSS, or SVS), and/or LCDR3 comprises the amino acid sequence (SEQ ID NO: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, or 247). The heavy chain variable region and the light chain variable region are connected by a linker.

Also provided is a single-chain variable fragment (scFv) comprising a heavy chain variable region comprising the amino acid sequence set forth in SEQ ID NOs: 314-356 and a light chain variable region comprising the amino acid sequence set forth in SEQ ID NOs: 271-313. The heavy chain variable region and the light chain variable region are connected by a linker.

In another aspect, a single chain variable fragment (scFv) comprising an amino acid sequence set forth in SEQ ID NOs: 44-86, is provided. In another aspect, a single chain variable fragment (scFv) consisting of an amino acid sequence set forth in SEQ ID NOs: 44-86, is provided.

Tolerable variations of the scFv sequences will be known to those of skill in the art. For example, in some embodiments, the scFv comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 44-86.

Also provided is a chimeric antigen receptor (CAR) construct comprising an antigen binding domain, a transmembrane domain, and an intracellular signaling domain. In certain embodiments, the antigen-binding domain comprises a single chain variable fragment (scFv) comprising an amino acid sequence set forth in SEQ ID NOs: 44-86. In another aspect, the CAR comprises an antigen-binding domain comprising a single chain variable fragment (scFv) consisting of an amino acid sequence set forth in SEQ ID NOs: 44-86.

TABLE 1
Example anti-CD19 scFv nucleotide
and amino acid sequences (VL-linker-VH)

| SEQ | | | |
|---|---|---|---|
| ID | | | |
| NO: | Name | Type | Sequence |
| 1 | scFv 1 | DNA | CAGGCTGTGCTGAATCAGCCGGCCTCTGTGTCTGCAGCCCTGG |
| | | | GGCAGAGGGTCACCATCTCCTGTAACACCAACATCGGCAGTCC |
| | | | TTATGATGTACAATGGTACCAGCAGCTCCCAGGAAAGTCCCCT |
| | | | AAAACTATCATTTATGGTAATAGCAATCGACCCTCGGGGGTCC |
| | | | CGGTTCGATTCTCTGGCTCCAAGTCAGGCAGCACAGCCACCCT |
| | | | GACCATCGCTGGGATCCAGGCTGAGGATGAGGCTGATTATTAC |
| | | | TGCCAGTCCTATGATGACAGCCTCGATGGTCGTGTTGTGTTCGG |
| | | | CGGAGGGACCCAACTGACCGTCCTCGGTGGTGGTTCCTCTAGA |
| | | | TCTTCCTCCTCTGGTGGCGGTGGCTCGGGCGGTGGTGGGGAGG |
| | | | TGCAGCTGGTGGAGTCTGGGGGAGACCTGGTGAAGCCTGGGGG |
| | | | GTCCTTGAGACTCTCCTGTGTGGCCTCTGGATTCACCTTCAGTA |
| | | | ACCACGAAATGTACTGGGTCCGCCAGGCTCCAGGGAAAGGGCT |
| | | | GGAGTGGGTCGCAAGGATTTATGATAGTGGAAGTAGAACAAG |
| | | | GTATGCAGACGCTGTGAAGGGCCGATTCACCATCTCCAGAGAC |
| | | | AACGCCAAGAACACGCTGTATCTTCAGATGAACAGCCTGACAG |
| | | | CCGAGGACACGGCCGTTTATTTCTGTGCGGGGGGGAATTCTCG |
| | | | TGGTTGGACCGAGTTTGGTATGGACTACTGGGGCCCGGGAACC |
| | | | CTGGTCACCGTCTCCTCA |
| 44 | scFv 1 | AA | QAVLNQPASVSAALGQRVTISCNTNIGSPYDVQWYQQLPGKSPKT |
| | | | IIYGNSNRPSGVPVRFSGSKSGSTATLTIAGIQAEDEADYYCQSYD |
| | | | DSLDGRVVFGGGTQLTVLGGGSSRSSSSGGGGSGGGGEVQLVES |
| | | | GGDLVKPGGSLRLSCVASGFTFSNHEMYWVRQAPGKGLEWVARI |
| | | | YDSGSRTRYADAVKGRFTISRDNAKNTLYLQMNSLTAEDTAVYF |
| | | | CAGGNSRGWTEFGMDYWGPGTLVTVSS |
| 2 | scFv 2 | DNA | TCCTATGTGCTGACTCAGTCACCCTCACTGTCTGGGTCCCTGGG |
| | | | CCAGAAGGTCACCATCTCCTGCACTGGAAGTCGTTCCAATCTC |
| | | | GGTGGAAATTATGTGGGCTGGTACCAACAAGTCCCAGGAATGG |
| | | | CCCCTAGAAATGTCATATATGCTAATGATTTTCGACCTTCACGG |
| | | | ATTCCCGCTCGATTCTCCGCCTCCAAGTCAGGCACTTCAGCCAC |
| | | | CCTGACCATCTCTGGGCTCCAGGCTGAGGACGAGGCTGACTAT |
| | | | TTTTGTTCATCATGGGATGATGATCTCGGGAGTCACGTGTTCGG |
| | | | CTCAGGGACCCAGCTGACCATCCTCGGTGGTGGTTCCTCTAGAT |
| | | | CTTCCTCCTCTGGTGGCGGTGGCTCGGGCGGTGGTGGGGAGGA |
| | | | GCAACTGGTGGAGTTTGGGGGAGACTTGGTGAAGCCTGGGGGG |
| | | | TCCCTGAGACTCTCCTGTGTAGCCTCTGGATTCAGTTTCAGTAG |
| | | | CTACGACATGAGCTGGGTCCGCCAGGCTCCTGGGAAGGGGCTG |
| | | | CAATGGGTCGCATCTACTAACTTTGATGGAAGTCGCACATACT |
| | | | ACACTGACGCTGTGGAGGGCCGATTCACCATCTCCAGAGACAA |
| | | | CGCCAGGAACACAGTGTATCTGCAGATGAACAGCCTGAGAGCC |
| | | | GAGGACACGGCTGTGTATTACTGTGCGAGGGGGATGGGTAGTT |
| | | | GGGGCGCTTCGACCTTTGGGTACTGGGGCCAGGGAACCCTGGT |
| | | | CACCGTCTCCTCA |
| 45 | scFv 2 | AA | SYVLTQSPSLSGSLGQKVTISCTGSRSNLGGNYVGWYQQVPGMAP |
| | | | RNVIYANDFRPSRIPARFSASKSGTSATLTISGLQAEDEADYFCSS |
| | | | WDDDLGSHVFGSGTQLTILGGGSSRSSSSGGGGSGGGGEEQLVEF |
| | | | GGDLVKPGGSLRLSCVASGFSFSSYDMSWVRQAPGKGLQWVAST |
| | | | NFDGSRTYYTDAVEGRFTISRDNARNTVYLQMNSLRAEDTAVYY |
| | | | CARGMGSWGASTFGYWGQGTLVTVSS |
| 3 | scFv 3 | DNA | CAGTCTGTGCTGAATCAGCCACCTTCTGTGTCTGGAGCCCTGGG |
| | | | GCAGACCGTCACCATCTCCTGCTCTGGAAGTCACACCAACATC |
| | | | GGCAGTGGATTTGATGTACAGTGGTACCAACAGCTCCCAGGAA |
| | | | AGTCTCCTCAAACTCTCGTCTATGCTAATATCAATCGACCCTCG |
| | | | GGGGTCCCGGCTCGATTCTCTGGCTCCAAGTCAGGCAGCACAG |
| | | | CCACCCTGACCATCACTGGGGTCCAGGCTGAAGATGAGGCTGA |
| | | | TTATTACTGCCAGTCGTATGATGACAACTTCGATGGGCACGTGT |
| | | | TCGGCTCAGGAACCCAACTGACCGTCCTCGGCGGTGGTTCCTCT |
| | | | AGATCTTCCTCCTCTGGTGGCGGTGGCTCGGGCGGTGGTGGGG |
| | | | AGGTCCAGCTGGTGCAGTCTGGGGGAGACCTGGTGAAACCTGG |
| | | | GGGATCCCTGAGACTGTCCTGTGTGGCCTCTGGATTCACCTTCA |
| | | | GTAACTACACCATGAGCTGGGTCCGCCAGGCTCCAGGGCAGGG |
| | | | GCTGCAGTGGGTCGCATACATTAACTATGATGGGAGTATCACA |
| | | | TACTACGCAGAGGCTGTGAAGGGCCGGTTCACCGTCTCCAGAG |
| | | | ACAACGCCAAGAACACGCTGTATCTGCAGATGAACAGTCTGAG |
| | | | AGCCGAGGACACGGCCGTGTATTTCTGTGTGAAGGAAACTTTC |
| | | | ATGATACCAACTGAGTGGGGCCAGGGAACCCTTATCACCGTCT |
| | | | CCTCA |
| 46 | scFv 3 | AA | QSVLNQPPSVSGALGQTVTISCSGSHTNIGSGFDVQWYQQLPGKSP |
| | | | QTLVYANINRPSGVPARFSGSKSGSTATLTITGVQAEDEADYYCQS |
| | | | YDDNFDGHVFGSGTQLTVLGGGSSRSSSSGGGGSGGGGEVQLVQ |
| | | | SGGDLVKPGGSLRLSCVASGFTFSNYTMSWVRQAPGQGLQWVA |
| | | | YINYDGSITYYAEAVKGRFTVSRDNAKNTLYLQMNSLRAEDTAV |
| | | | YFCVKETFMIPTEWGQGTLITVSS |
| 4 | scFv 4 | DNA | CAGTCTGTGCTGAATCAGCCGCCTTCTGTGTCTGGAGCCCTGGG |
| | | | GCAGACCGTCACCATCTCCTGTTCTGGAAGTCACACCAACATC |
| | | | GGCAGTGGATTTGATGTACAGTGGTACCAACAGCTCCCAGGAA |
| | | | AGTCCCCTCAAACTATCATCTATGCTAATATCAATCGACCCTCG |
| | | | GGGGTCCCGGCTCGATTCTCTGGCTCCAAGTCAGGCAGCACAG |
| | | | CCACCCTGACCATCACTGGGGTCCAGGCTGAAGATGAGGCTGA |
| | | | TTATTACTGCCAGTCGTATGATGACAATTTCGATTACGTGTTCG |
| | | | GCTCAGGGACCCAACTGACCGTCCTTGGTGGTGGTTCCTCTAG |
| | | | ATCTTCCTCCTCTGGTGGCGGTGGCTCGGGCGGTGGTGGGGAG |
| | | | GAGCAACTGGTGGAGTTTGGGGGAGACCTGGTGAAGCCTGGA |
| | | | GGGTCCTTGAGACTGTCCTGTGTGGTCTCTGGATTCGATTTCAG |
| | | | TCGCTACGGCATGTCCTGGGTCCGTCAGTCTCCAGGGAAGGGT |
| | | | CTCCAGTGGGTCGCAGATATCAGTTACAGGGGAACTACATTTT |
| | | | ACGCAGACGATGTGAAGGGCCGATTTACCGTCTCCAGAGACAG |
| | | | TGCCAAGAACACACTCTACCTCGAAATGACCAGCCTGACAGTC |
| | | | GAGGACACGGCCGTATATTATTGTGCGAATAGTGCCAACTGGT |
| | | | ATGTCTCCCCTTTTGACCACTGGGGCCAGGGAACCCTGGTCACC |
| | | | GTCTCCTCA |
| 47 | scFv 4 | AA | QSVLNQPPSVSGALGQTVTISCSGSHTNIGSGFDVQWYQQLPGKSP |
| | | | QTIIYANINRPSGVPARFSGSKSGSTATLTITGVQAEDEADYYCQS |
| | | | YDDNFDYVFGSGTQLTVLGGGSSRSSSSGGGGSGGGGEEQLVEFG |
| | | | GDLVKPGGSLRLSCVVSGFDFSRYGMSWVRQSPGKGLQWVADIS |
| | | | YRGTTFYADDVKGRFTVSRDSAKNTLYLEMTSLTVEDTAVYYCA |
| | | | NSANWYVSPFDHWGQGTLVTVSS |
| 5 | scFv 5 | DNA | CAGCCTGTGCTGACCCAGCCACCCTCAGTGTCGGGGTCCCTTG |
| | | | GCCAGAGGGTCACCATCTCCTGTTCTGGAAGCACGAACACCAT |
| | | | CGGTGTTGTTGGTGCGAACTGGTACCAACAGCTCCCAGGAAAG |
| | | | TCCCCTAAACTCCTCGTGTACAGTGATGGGGTTCGACCGTCAG |
| | | | GGGTCCCTGACCGGTTTTCCGGCTCCAGGTCTGCCAACTCAGAC |
| | | | ACCCTGACCATCACTGGGCTTCAGGCTGAGGACGAGGCTGATT |
| | | | ATTACTGCCAGTCCTTTGATTCCACACATAATGCCATTGTATTC |
| | | | GGCGGAGGCACCCATCTGACCGTCCTCGGTGGTGGTTCCTCTA |
| | | | GATCTTCCTCCTCTGGTGGCGGTGGCTCGGGCGGTGGTGGGGA |
| | | | GGTGCAGCTGGTGGAGACTGGGGGAGACCTGGTGAAGCCTGC |
| | | | GGGGTCCCTGAGACTGTCGTGTGCGGCCTCTGGATTCCCCTTCA |
| | | | GTGGCTACAGTATGACCTGGGTCCGCCAGGCCCCTGAAAAGGG |
| | | | TCTGCAATTGGTCGCAGGTATTAACAGCGATGGAAGTCACACA |
| | | | TACTACACAGACGATGTGAAGGGCCGATTCACCATCTCCAGAG |
| | | | ACAACACCAAATACATACTGTATCTGCAAATGAACAGTCTGAG |
| | | | AGCCGAGGACACGGCCATGTATTATTGTGGCTCTCATAGTTGG |
| | | | GGCAATTTTCACTACTGGGGCCAGGGAACCCTGGTCACCGTCT |
| | | | CCTCA |
| 48 | scFv 5 | AA | QPVLTQPPSVSGSLGQRVTISCSGSTNTIGVVGANWYQQLPGKSPK |
| | | | LLVYSDGVRPSGVPDRFSGSRSANSDTLTITGLQAEDEADYYCQSF |
| | | | DSTHNAIVFGGGTHLTVLGGGSSRSSSSGGGGSGGGGEVQLVETG |
| | | | GDLVKPAGSLRLSCAASGFPFSGYSMTWVRQAPEKGLQLVAGINS |
| | | | DGSHTYYTDDVKGRFTISRDNTKYILYLQMNSLRAEDTAMYYCG |
| | | | SHSWGNFHYWGQGTLVTVSS |
| 6 | scFv 6 | DNA | CAGCCTGTGCTCACTCAGCCGCCCTCAGTGTCTGGGTCCCTGGG |
| | | | CCAGAGGGTCACCATCTCCTGCACCGGAACTAATACCAACATC |
| | | | GGCAGTGGTTATAGTGTACAATGGTACCAGCAGCTCCCAGGAG |
| | | | AGTCCCCAAAACCTATCATTTATGGTAGTAGTAATCGACCCTCG |
| | | | GGGGTCCCGGCTCGATTCTCTGGCTCCAAGTCAGGCAGCACAG |
| | | | GCACCCTGACCATCACTGGGATCCAGGCTGAGGATGAGGCTGA |
| | | | TTACCACTGCCAGTCCTATGATGACAGCCTCGATGGTCATGCTG |
| | | | TATTCGGCGGAGGGACCCAGCTGACCATCCTCGGTGGTGGTTC |
| | | | CTCTAGATCTTCCTCCTCTGGTGGCGGTGGCTCGGGCGGTGGTG |
| | | | GGGAGGTCCAGCTGGTGCAGTCTGGGGGAGACTTGGTGAAGCC |
| | | | TGGGGGGTCCTTGAGACTGTCCTGTGTGGCCTCTGGATTCACCT |
| | | | TCAGTGACAATTACATGAGCTGGATCCGTCAGGTTCCAGGGAA |
| | | | GGGCCTGCAGTGGGTCTCACGAATTAGCACTGATGGAAGAGGC |
| | | | ACAAGCTACGCAGACGCTGTGAAGGGCCGATTCACCATCTCCA |
| | | | GAGACAATGCCAAGAACACGCTGTATCTGCAGATGAACAGCCT |
| | | | GAGAGACGAGGACACGGCCGTGTATTACTGTGCAAGGCGGTCT |
| | | | GGTATTTGGGGGTTTGACTACTGGGGCCAGGGCGCCCTGGTCA |
| | | | CCGTCTCCTCA |
| 49 | scFv 6 | AA | QPVLTQPPSVSGSLGQRVTISCTGTNTNIGSGYSVQWYQQLPGESP |
| | | | KPIIYGSSNRPSGVPARFSGSKSGSTGTLTITGIQAEDEADYHCQSY |
| | | | DDSLDGHAVFGGGTQLTILGGGSSRSSSSGGGGSGGGGEVQLVQS |
| | | | GGDLVKPGGSLRLSCVASGFTFSDNYMSWIRQVPGKGLQWVSRIS |
| | | | TDGRGTSYADAVKGRFTISRDNAKNTLYLQMNSLRDEDTAVYYC |
| | | | ARRSGIWGFDYWGQGALVTVSS |
| 7 | scFv 7 | DNA | TCCTATGAGCTGACTCAGCCACCATCTATGTCTGCAGCCCTGGG |
| | | | GCAGAGGGTCACCATCTCCTGCACTGGAAGTAACACCAACATC |
| | | | GGCAGTGGTTATGATGTACAATGGTACCAACAGGTCCCAGGAA |
| | | | AGTCCCCTACACCTCTGATTTATGGTAATACCAATCGACCCTCG |
| | | | GGGGTCCCGGTGCGATTCTCTGGCTCCAAGTCAGGCAACACAG |
| | | | CCACCCTGACCATCACTGGGATCCAGGCTGAGGATGAGGCTGA |
| | | | TTATTACTGCCAGTCCTATGATGACAATTTCGATGGTCATCACG |
| | | | TGTTCGGCTCAGGGACCCAGCTGACCGTCCTCGGTGGTGGTTCC |
| | | | TCTAGATCTTCCTCCTCTGGTGGCGGTGGCTCGGGCGGTGGTGG |
| | | | GGAGGTACAGCTGGTGGAATCTGGGGGAGACCTGGTGAAGCCT |
| | | | GCGGGGTCCCTGAGACTGTCCTGTGTGGCCTCTGGATTCACCTT |
| | | | CAGTAACTATGGCATGAATTGGGTCCGCCAGGCTCCCGGGAAG |
| | | | GGGCTGCAGTGGGTCGCAGGTATTAACAGTGGTGGAAGTACCA |
| | | | CAACCTACGCAGACGTTGTGAAGGGTCGATTCACCATCTCCAG |
| | | | AGACAACGCCAAGAACACACTGTATCTGCAGATGAACAGCCTG |
| | | | GGAGCCGAGGACACGGCCGTGTATTACTGTGCGAGGGTGGATT |
| | | | ATAGTAGCAGCTGGTTGGTTGACTACTGGGGCCAGGGAACCCT |
| | | | GGTCACCGTCTCCTCA |
| 50 | scFv 7 | AA | SYELTQPPSMSAALGQRVTISCTGSNTNIGSGYDVQWYQQVPGKS |
| | | | PTPLIYGNTNRPSGVPVRFSGSKSGNTATLTITGIQAEDEADYYCQ |
| | | | SYDDNFDGHHVFGSGTQLTVLGGGSSRSSSSGGGGSGGGGEVQL |
| | | | VESGGDLVKPAGSLRLSCVASGFTFSNYGMNWVRQAPGKGLQW |
| | | | VAGINSGGSTTTYADVVKGRFTISRDNAKNTLYLQMNSLGAEDT |
| | | | AVYYCARVDYSSSWLVDYWGQGTLVTVSS |
| 8 | scFv 8 | DNA | CAGTCTGTGTTGACTCAACCGGCCTCAGTGTCTGGGTCCCTGGG |
| | | | CCAGAAGGTCACCATCTCCTGCACTGGAAGCAGGTCCAACATC |
| | | | GGTGCAAATTATGTGGGCTGGTATCAACAGGTCCCAGGAATAG |
| | | | GCCCTAGAACCGTCATTTTTGGTTATAATTATCGACCTTCAGGG |
| | | | GTCCCCGATCGATTCTCTAGCTCCAAGTCAGGCAATTCAGCCAC |
| | | | CCTGACCATCTCTGGGCTCCAGGCTGAGGACGAGGCTGAATAT |
| | | | TATTGTTCATCCTGGGATGATAGTCTCAGAGGTACCGTGTTCGG |
| | | | CTCAGGGACCCAACTGACCGTCCTTGGCGGTGGTTCCTCTAGAT |
| | | | CTTCCTCCTCTGGTGACGGTGGCTCGGGCGGTGGTGGGGAGGT |
| | | | ACAGCTGGTGGAATCTGGGGGAGACCTGGTGAAGCCTGGGGA |
| | | | ATCCCTGAGACTGTCCTGTGTGGCCTCTGGATTCACCTTCAGTA |
| | | | GCTATGGCATGAGCTGGGTCCGACAGTCTCCAGGGAAGGGGTT |
| | | | GCAGTGGGTCGCAGATATTAGTTATGGAGGAGGTACATACTAC |
| | | | GCAGACGCTGTGAAGGGCCGATTCACCATCTCCAGAGACAACG |
| | | | CCAGGAACACAGTATATCTGCAGATGAACAGCCTGAGAGCCGA |
| | | | GGACACGGCCGTGTATTACTGTGCGTCCCAATGGGGTGATTGG |
| | | | GGCCACTTTGAGTACTGGGGCCAGGGCACCCTGGTCACCGTCT |
| | | | CCTCA |
| 51 | scFv 8 | AA | QSVLTQPASVSGSLGQKVTISCTGSRSNIGANYVGWYQQVPGIGP |
| | | | RTVIFGYNYRPSGVPDRFSSSKSGNSATLTISGLQAEDEAEYYCSS |
| | | | WDDSLRGTVFGSGTQLTVLGGGSSRSSSSGDGGSGGGGEVQLVES |
| | | | GGDLVKPGESLRLSCVASGFTFSSYGMSWVRQSPGKGLQWVADI |
| | | | SYGGGTYYADAVKGRFTISRDNARNTVYLQMNSLRAEDTAVYY |
| | | | CASQWGDWGHFEYWGQGTLVTVSS |
| 9 | scFv 9 | DNA | CAGCCTGTGCTGACCCAGCCACCCTCTGTGTCTGCAGCCCTGGG |
| | | | ACAGAGGGTCACCCTCTCCTGCACTGGAAGTAAGACCAACATC |
| | | | GGCAGTGGTTATGATGTACAATGGTACCAGCAGTTCCCAGGAA |
| | | | AGTCCCCTAAAACCATCATCTATGGTAATAGCAATCGATTTTCG |
| | | | GGGGTCCCGGCTCGATTCTCTGGCTCCAAGTCAGGCAGCATCG |
| | | | CCACCCTGACCATCACTGGGATCCAGGCTGAGGATGAGGCTGA |
| | | | TTATTACTGCCAGTCCTTTGATGACAATGTCGATGGTTACGTGT |
| | | | TCGGCTCAGGGACCCAGCTGACCATCCTCGGTGGTGGTTCCTCT |
| | | | AGATCTTCCTCCTCTGGTGGCGGTGGCTCGGGCGGTGGTGGGG |
| | | | AGGTACAGCTGGTGGAGTCTGGGGGAGACCTGGTGAAGCCTGG |
| | | | GGGGTCCCTGAGACTCTCCTGTGTGGCCTCTGGATTCACCTTCA |
| | | | GTAGCGACAGCATACATTGGGTCCGTCAGGCTCCAGGAAAGGG |
| | | | GCTGCAGTGGGTCGCATACATTAACAGTGGTGGAAGTACCACA |
| | | | TACTACGCAGACGCTGTGAAGGGCCGATTCACCATCTCCAGAG |
| | | | ACAACGCCAGGAACACAGTGTATCTGAAGATGAACAGCCTGA |
| | | | GAGCCGAGGACACGGCTGTGTATTACTGTGCGAAAGATTCAGG |
| | | | CTTTGGCAGCTGGTACCTTGAATCCTGGGGCCAGGGAACCCTG |
| | | | GTCACCGTCTCCCCA |
| 52 | scFv 9 | AA | QPVLTQPPSVSAALGQRVTLSCTGSKTNIGSGYDVQWYQQFPGKS |
| | | | PKTIIYGNSNRFSGVPARFSGSKSGSIATLTITGIQAEDEADYYCQSF |
| | | | DDNVDGYVFGSGTQLTILGGGSSRSSSSGGGGSGGGGEVQLVESG |
| | | | GDLVKPGGSLRLSCVASGFTFSSDSIHWVRQAPGKGLQWVAYINS |
| | | | GGSTTYYADAVKGRFTISRDNARNTVYLKMNSLRAEDTAVYYCA |
| | | | KDSGFGSWYLESWGQGTLVTVSP |
| 10 | scFv 10 | DNA | CAGGCTGTGCTGAATCAGCCGGCCTCTGTGTCTGGAGCCCTGG |
| | | | GGCAGACCGTCACCATCTCCTGTTCTGGAAGTCACACCAACAT |
| | | | CGGCAGTGGATTTGATGTACAGTGGTACCAACAGCTCCCAGGA |
| | | | AAGTCCCCTCAAACTATCATCTATGCTAATATCAATCGACCCTC |
| | | | GGGGGTCCCGGCTCGATTCTCTGGCTCCAAGTCAGGCAGCACA |
| | | | GCCACCCTGACCATCACTGGGGTCCAGGCTGAAGATGAGGCTG |
| | | | ATTATCACTGCCAGTCCTTTGATGACGACCTCGATGGTCCTGTG |
| | | | TTCGGCGGAGGCACCCATCTGACCGTCCTCGGTGGTGGTTCCTC |
| | | | TAGATCTTCCTCCTCTGGTGGCGGTGGCTCGGGCGGTGGTGGG |
| | | | GAGGTCCAGCTGGTGCAGTCTGGAGGAGACCTGGTGAAGCCTG |
| | | | GGGGGTCCCTGAAAATTTCCTGTGTGGCCTCTGGACTCACCTTC |
| | | | AATAGAAACTACATGACCTGGATCCGCCAGGCTCCAGGGAAGG |
| | | | GGCTGCAGTGGGTCTCAGAAATTAACCCTGATGGAAGTACCAC |
| | | | GAGTTACACAGACGCTGTGAGGGGCCGATTCACCATCTCCAGA |
| | | | GACAATGCCAAGAACACGTTGTATCTGCAGATGAATAGTATGA |
| | | | GAGATGAGGACACGGCAGTGTATTACTGCGCAAGGAGTAGTTG |
| | | | GGGTTCTTTTGACTATTGGGGCCAGGGAACCCTGGTCACCGTCT |
| | | | CCTCA |
| 53 | scFv 10 | AA | QAVLNQPASVSGALGQTVTISCSGSHTNIGSGFDVQWYQQLPGKS |
| | | | PQTIIYANINRPSGVPARFSGSKSGSTATLTITGVQAEDEADYHCQS |
| | | | FDDDLDGPVFGGGTHLTVLGGGSSRSSSSGGGGSGGGGEVQLVQ |
| | | | SGGDLVKPGGSLKISCVASGLTFNRNYMTWIRQAPGKGLQWVSEI |
| | | | NPDGSTTSYTDAVRGRFTISRDNAKNTLYLQMNSMRDEDTAVYY |
| | | | CARSSWGSFDYWGQGTLVTVSS |
| 11 | scFv 11 | DNA | CAGCCTGTGCTGACCCAGCCACCCTCTGTGTCTGGAGCCCTGG |
| | | | GGCAGACCGTCACCATCTCCTGCTCTGGAAGTCACACCAACAT |
| | | | CGGCAGTGGATTTGATGTACAGTGGTACCAACAGCTCCCAGGA |
| | | | AAGTCCCCTCAAACTCTCGTCTATGCTAATATCAATCGACCCTC |
| | | | GGGGGTCCCGGCTCGATTCTCCGGCTCCAAGTCAGGCAGCACA |
| | | | GCCACCCTGACCATCACTGGGGTCCAGGCTGAAGATGAGGCTG |
| | | | ATTATTACTGCCAGTCGTATGATGACAATTTCGATTACGTGTTC |
| | | | GGCTCAGGGACCCAACTGACCGTCCTTGGTGGTGGTTCCTCTA |
| | | | GATCTTCCTCCTCTGGTGGCGGTGGCTCGGGCGGTGGTGGGGA |
| | | | GGTACAGCTGGTGGAATCTGGGGGAGACCTGGTGAAGCCTGGG |
| | | | GGGTCCTTGAGACTGTCCTGTGTGGCCTCTGGATTCACCTTCAG |
| | | | TGACTATAGCATGAGCTGGGTCCGTCAGTCTCCAGGGAAGGGG |
| | | | CTGCAGTGGGTCGCAGCTATTATGACTAGTGGAAGCACATACT |
| | | | ACGCAGACGCTGTGAAGGGCCGATTCACCATCTCCAGAGACAA |
| | | | CGCCAAGAATACGCTGTATCTGCAGATGGACAGCCTGAGAGCC |
| | | | GAGGACACGGCCGTTTATTACTGTGCGCACGACTACTGGTTTG |
| | | | ACTTCTGGGGCCAGGGCACCCTGGTCACCGTCTCCTCA |
| 54 | scFv 11 | AA | QPVLTQPPSVSGALGQTVTISCSGSHTNIGSGFDVQWYQQLPGKSP |
| | | | QTLVYANINRPSGVPARFSGSKSGSTATLTITGVQAEDEADYYCQS |
| | | | YDDNFDYVFGSGTQLTVLGGGSSRSSSSGGGGSGGGGEVQLVES |
| | | | GGDLVKPGGSLRLSCVASGFTFSDYSMSWVRQSPGKGLQWVAAI |
| | | | MTSGSTYYADAVKGRFTISRDNAKNTLYLQMDSLRAEDTAVYYC |
| | | | AHDYWFDFWGQGTLVTVSS |
| 12 | scFv 12 | DNA | TCCTATGAGCTGACTCAGCCACCATCAGCGACTGTGACCCTGA |
| | | | GGCAGACGGCCCGCCTCACCTGTGGGGGAGAGAACATTGGAA |
| | | | GAAAAAGTGTTCAATGGTACCAACAGAAGCCGGGCCAGTCCCC |
| | | | CCTACTCATTATCTATGCTGATTCCAGCAGGCCGTCAGGGATCC |
| | | | CTGAGCGATTCTCTGGCGCCAATTCGGGGAACACGGCCAGCCT |
| | | | GACCATCAGCGGGGCCCTGGCCGAGGACGAGGCTGACTATTAC |
| | | | TGCCAGATTTGGGACCGCAATATTAATGTGTTCGGCTCCGGGA |
| | | | CCCAGCTGACCGTCCTTGGCGGTGGTTCCTCTAGATCTTCCTCC |
| | | | TCTGGTGGCGGTGGCTCGGGCGGTGGTGGGGAGGAGCAACTGG |
| | | | TGGAGTTTGGGGGAGACCTGGTGAAGCCTGCGGGGTCCCTGAG |
| | | | ACTGTCCTGTGTGGCCTCGGGATTCACCTTCAGTAGCTACGACA |
| | | | TGAGCTGGGTCCGCCAGGCTCCTGGGAAGGGGCTGCAGTGGGT |
| | | | CGCGACTATTAGTTATGATGGAAGTAGTACATACTACACTGAC |
| | | | GCTGTGAAGGGCCGATTCACCGTCTCCAGAGACAACGCCAAGA |
| | | | ACACGTTGTATCTGCAGATGAACAGCCTGAGAGCCGAGGACAC |
| | | | GGCTCTGTATTACTGTGCAAAGGATTCTGGCAGTTGGTCCCATT |
| | | | TCTATGGTTTGGACACCTGGGGCCATGGCACCTCAGTCTTCGTG |
| | | | TCTTCC |
| 55 | scFv 12 | AA | SYELTQPPSATVTLRQTARLTCGGENIGRKSVQWYQQKPGQSPLLI |
| | | | IYADSSRPSGIPERFSGANSGNTASLTISGALAEDEADYYCQIWDR |
| | | | NINVFGSGTQLTVLGGGSSRSSSSGGGGSGGGGEEQLVEFGGDLV |
| | | | KPAGSLRLSCVASGFTFSSYDMSWVRQAPGKGLQWVATISYDGS |
| | | | STYYTDAVKGRFTVSRDNAKNTLYLQMNSLRAEDTALYYCAKDS |
| | | | GSWSHFYGLDTWGHGTSVFVSS |
| 13 | scFv 13 | DNA | CAGCCTGTGCTGACCCAGCCACCCTCTGTGTCTGGAGCCCTGG |
| | | | GGCAGACCGTCACCATCTCCTGCTCTGGAAGTCACACCAACAT |
| | | | CGGCAGTGGATTTGATGTACAGTGGTACCAACAGCTCCCAGGA |
| | | | AAGTCCCCTCAAACTCTCGTCTATGCTAATATCAATCGACCCTC |
| | | | GGGGGTCCCGGCTCGATTCTCTGGCTCCAAGTCAGGCAGCACA |
| | | | GCCACCCTGACCATCACTGGGGTCCAGGCTGAAGATGAGGCTG |
| | | | ATTATTACTGCCAGTCGTATGATGACAATTTCGATTACGTGTTC |
| | | | GGCTCAGGGACCCAGCTGACCGTCCTCGGCGGTGGTTCCTCTA |
| | | | GATCTTCCTCCTCTGGTGACGGTGGCTCGGGCGGTGGTGGGGA |
| | | | GGTACAGCTGGTGGAGTCTGGGGGAGGCCTGATGAAGCCTGGG |
| | | | GGGTCCCTGAAACTCTCCTGTTTGGCCTCTGGTTTCACCTTCAG |
| | | | TCGCTACTATATGTATTGGGTCCGCCAGGCTCCAGGGAAGGGA |
| | | | CTTCAGTGGGTCGCACAAATTAGCAGTGATGGCAGAAGAACAT |
| | | | ACTACGCAGACGCTGTGCAGGGCCGATTCACCATCTCCAGAGA |
| | | | CAATGTCAAGAACACGGTATATCTGCAGATGAACAGCCTGAGC |
| | | | GACGAGGACACGGCCGTGTATTATTGTGTCCGAGAGGACTACT |
| | | | TCGACTTTGACTACTGGGGCCAGGGCACCCTGGTCACCGTCTCC |
| | | | TCA |
| 56 | scFv 13 | AA | QPVLTQPPSVSGALGQTVTISCSGSHTNIGSGFDVQWYQQLPGKSP |
| | | | QTLVYANINRPSGVPARFSGSKSGSTATLTITGVQAEDEADYYCQS |
| | | | YDDNFDYVFGSGTQLTVLGGGSSRSSSSGDGGSGGGGEVQLVES |
| | | | GGGLMKPGGSLKLSCLASGFTFSRYYMYWVRQAPGKGLQWVAQ |
| | | | ISSDGRRTYYADAVQGRFTISRDNVKNTVYLQMNSLSDEDTAVY |
| | | | YCVREDYFDFDYWGQGTLVTVSS |
| 14 | scFv 14 | DNA | CAGTCTGTGCTGAATCAGCCACCTTCTGTGTCTGCGGCCCTGGG |
| | | | GCAGAGGATCACCATCTCCTGCACTGGAAGTCACACCAACATC |
| | | | GGCAGTGGTTATGATGTACAATGGTACCAGCAGCTCCCCGGAA |
| | | | AGTCCCCTAGAACTATCATTTATGGTAATAGCGATCGGCCCTCT |
| | | | GGGGTCCCGGTTCGATTCTCTGGCTCCAAGTCAGGCAACACAG |
| | | | CCACCCTGACCATCACTGGGATCCAGGCTGAGGATGAGGCTGA |
| | | | TTATTATTGCCAGTCCTATGATGACAACCTCAATGATCCTTACG |
| | | | TGTTCGGCTCAGGGACCCAACTGACCGTCCTTGGTGGTGGTTCC |
| | | | TCTAGATCTTCCTCCTCTGGTGGCGGTGGCTCGGGCGGTGGTGG |
| | | | GGAGGTCCAGCTGGTGCAGTCTGGGGGAGACCTGGTGAAGCCT |
| | | | GGGGGGTCCCTGAGACTCTCCTGTGTGGCCTCTGGATTCGCCTT |
| | | | CAGTGGCAAAACAATGCAATGGGTCCGTCAGGCTCCAGGGAA |
| | | | GGGGCTACAGTGGGTCGCATACATTCAACATGATGGGAGTAGA |
| | | | ACATCCTACGCAGACGCTGTGAAGGGCCGATTCACCATCTCCA |
| | | | GAGACAACGCCAAGAACACGCTCTATCTCCAGATGGACAGCCT |
| | | | GAGAGCCGAGGACACGGCCGTGTATTACTGTGCGGATCATCCC |
| | | | TGGTACGGGCCTGAGTATTGGGGCCAGGGAACCCCGGTCACCG |
| | | | TCTCCTCA |
| 57 | scFv 14 | AA | QSVLNQPPSVSAALGQRITISCTGSHTNIGSGYDVQWYQQLPGKSP |
| | | | RTIIYGNSDRPSGVPVRFSGSKSGNTATLTITGIQAEDEADYYCQSY |
| | | | DDNLNDPYVFGSGTQLTVLGGGSSRSSSSGGGGSGGGGEVQLVQ |
| | | | SGGDLVKPGGSLRLSCVASGFAFSGKTMQWVRQAPGKGLQWVA |
| | | | YIQHDGSRTSYADAVKGRFTISRDNAKNTLYLQMDSLRAEDTAV |
| | | | YYCADHPWYGPEYWGQGTPVTVSS |
| 15 | scFv 15 | DNA | TCCTATGAGCTGACTCAGCCACCATCGGTGTCTGCAGCCCTGG |
| | | | GGCAGGGGGTCACCATCTCCTGCATCGGCAGTGACACCAACAT |
| | | | CGGCAATCATTATGATGTACAATGGTACCAGCAGCTCCCAGGA |
| | | | AAGTCCCCTAGAACTATTATTTATGGTAATAGCGATCGACCCTC |
| | | | GGGGGTCCCGGGTCGATTCTCTGGCTCCAAGTCAGGCAACACA |
| | | | GCCACCCTGACCATCACTGGGATCCAGGCTGAGGATGAGGCTG |
| | | | ATTATCACTGCCAGTCCTATGATGACGACCTCGATGGCCATATT |
| | | | GTGTTCGGCGGAGGCACCCATCTGACCGTCCTCGGTGGTGGTT |
| | | | CCTCTAGATCTTCCTCCTCTGGTGGCGGTGGCTCGGGCGGTGGT |
| | | | GGGGAGGTCCAGCTGGTGCAGTCTGGGGGAGACCTGGTGAAG |
| | | | CCTGGGGGTTCCCTGAGACTGTCCTGTGTGGCATCTGGATTCAC |
| | | | CTTCAATAAGTACCACATGAGTTGGGTCCGCCAGGCTCCAGGG |
| | | | AAGGGGCTGCAGTGGGTCGGAAAGATAACCAGTGATGGAAGT |
| | | | GCCACAGGGTATGCAGACGCTGTAAAGGGCCGATTCACCATCT |
| | | | CCAGAGACAATGCCAAGAACACGTTCTATCTACAGATGGACAG |
| | | | CCTGAGAGCCGAGGACACGGCTATGTATTACTGTGCAAGGGAT |
| | | | CAGAGTAGTTGGGGAACGGGTAATTTTGACTACTGGGGCCAGG |
| | | | GAACCCTGGTCACCGTCTCCTCA |
| 58 | scFv 15 | AA | SYELTQPPSVSAALGQGVTISCIGSDTNIGNHYDVQWYQQLPGKSP |
| | | | RTIIYGNSDRPSGVPGRFSGSKSGNTATLTITGIQAEDEADYHCQSY |
| | | | DDDLDGHIVFGGGTHLTVLGGGSSRSSSSGGGGSGGGGEVQLVQS |
| | | | GGDLVKPGGSLRLSCVASGFTFNKYHMSWVRQAPGKGLQWVGK |
| | | | ITSDGSATGYADAVKGRFTISRDNAKNTFYLQMDSLRAEDTAMY |
| | | | YCARDQSSWGTGNFDYWGQGTLVTVSS |
| 16 | scFv 16 | DNA | CAGTCTGTGCTGACTCAGCCGACCTCAGTGTCTGGGTCCCTGGG |
| | | | CCAGAGGGTCACCATCTCCTGCACTGGAAGCAGCTCCAATGTT |
| | | | GGTTATGGCAATTATGTGGGCTGGTACCAGCAGCTCCCAGGAA |
| | | | CAGGCCCCAGAACCCTCATCTATGGTAGTCATTACCGACCCTC |
| | | | GGGGGTCCCTGATCGATTCTCTGGCTCCAGTTCAGGCAGCTCA |
| | | | GCCACACTGACCATCTCTGGGCTCCAGGCTGAGGATGAGGCTG |
| | | | ATTATTACTGCTCATCCTACGACAGCACTCTCAGTGGTTACGTG |
| | | | TTTGGCTCAGGGACCCAGCTGACCATCCTCGGCGGTGGTTCCTC |
| | | | TAGATCTTCCTCCTCTGGTGGCGGTGGCTCGGGCGGTGGTGGG |
| | | | GAGGTACAGCTGGTGGAATCTGGGGGAGACCTGGTGAAGCCTG |
| | | | GGGGGTCCCTGAGACTCTCCTGTGTGGGCTCTGGATTCACCTTG |
| | | | AGTAGTTATTGGATGAGCTGGGTCCGCCAGGCTCCAGGGAAGG |
| | | | GGCTACAGTGGATTGCAGAAATCAGCGGCAGTGGAAATACCAT |
| | | | ATATTATGGGGACGCTGTGAAGGGCCGATTCACCATCTCCAGA |
| | | | GACAATGCCAAGAACACGCTGTATCTGCAGATGAACAGCCTGA |
| | | | GAGATGAGGACACGGCAGTGTATTACTGTGCAACGGAATACTA |
| | | | CTGGAATATTGACAACTGGGGCCAGGGAACCCTGGTCACCGTC |
| | | | TCCTCA |
| 59 | scFv 16 | AA | QSVLTQPTSVSGSLGQRVTISCTGSSSNVGYGNYVGWYQQLPGTG |
| | | | PRTLIYGSHYRPSGVPDRFSGSSSGSSATLTISGLQAEDEADYYCSS |
| | | | YDSTLSGYVFGSGTQLTILGGGSSRSSSSGGGGSGGGGEVQLVESG |
| | | | GDLVKPGGSLRLSCVGSGFTLSSYWMSWVRQAPGKGLQWIAEIS |
| | | | GSGNTIYYGDAVKGRFTISRDNAKNTLYLQMNSLRDEDTAVYYC |
| | | | ATEYYWNIDNWGQGTLVTVSS |
| 17 | scFv 17 | DNA | CAGCCTGTGCTGACCCAGCCACCCTCTGTGTCTGCAGCCCTGGG |
| | | | GCAGAGGGTCACCATCTCCTGCACTGGAAGTAAGACCAACATC |
| | | | GGCAGTGGTTATGATGTACAATGGTACCAGCAGCTCCCAGGAA |
| | | | AGTCCCCTAAAACCATCATTTATGGTAATAGCAATCGACCCTC |
| | | | GGGGGTCCCGGCTCGATTCTCTGGCTCCAAGTCAGGCAGCATC |
| | | | GCCACCCTGACCATCACTGGGATCCAGGCTGAGGATGAGGCTG |
| | | | ATTATTACTGCCAGTCCTTTGATGACAATGTCGATGGTTACGTG |
| | | | TTCGGCTCAGGGACCCAGCTGACCATCCTCGGTGGTGGTTCCTC |
| | | | TAGATCTTCCTCCTCTGGTGGCGGTGGCTCGGGCGGTGGTGGG |
| | | | GAGGTGCCACTGGTGGAATCTGGGGGAGACCTGGTGAAGCCTG |
| | | | GGGGATCCCTGAGACTGTCCTGTGTGGCCTCTGGATTCACCTTC |
| | | | AGTAGCTACGGCATGAGCTGGGTCCGCCAGGCTCCAGGGAAGG |
| | | | GACTACAGTGGGTCGCATACATTAATGGTGGTGGAAGTAGTAC |
| | | | ATCCTACGCAGACGCTGTGAGGGGCCGATTCACCATCTCCAGA |
| | | | GACAACGCCGAGAACACGCTGTATCTGCAGATGAACAGCCTGA |
| | | | GAGCCGACGATACGGCTGTATATTACTGTTCGAATTACTACGG |
| | | | TATCTACTGGGATTCTGACTACTGGGGCCAGGGAACCCTGGTC |
| | | | ACCGTCTCCTCA |
| 60 | scFv 17 | AA | QPVLTQPPSVSAALGQRVTISCTGSKTNIGSGYDVQWYQQLPGKS |
| | | | PKTIIYGNSNRPSGVPARFSGSKSGSIATLTITGIQAEDEADYYCQSF |
| | | | DDNVDGYVFGSGTQLTILGGGSSRSSSSGGGGSGGGGEVPLVESG |
| | | | GDLVKPGGSLRLSCVASGFTFSSYGMSWVRQAPGKGLQWVAYIN |
| | | | GGGSSTSYADAVRGRFTISRDNAENTLYLQMNSLRADDTAVYYC |
| | | | SNYYGIYWDSDYWGQGTLVTVSS |
| 18 | scFv 18 | DNA | CAGTCTGCGCTGACTCAAACGGCCTCAGTGTCTGGGTCCCTGG |
| | | | GCCAGAAGGTCACCATCTCCTGCACTGGAAGCAGGTCCAACAT |
| | | | CGGTGCAAATTATGTGGGCTGGTATCAACAGGTCCCAGGAATA |
| | | | GGCCCTAGAACCGTCATTTTTGGTTATAATTATCGACCTTCAGG |
| | | | GGTCCCCGATCGATTCTCTAGCTCCAAGTCAGGCAATTCAGCC |
| | | | ACCCTGACCATCTCTGGGCTCCAGGCTGAGGACGAGGCTGAGT |
| | | | ATTATTGTTCAACATGGGACAGCAGTCTCAACGCTTACGTGTTC |
| | | | GGCTCAGGGACCCAGCTGACCGTCCTCGGCGGTGGTTCCTCTA |
| | | | GATCTTCCTCCTCTGGTGGCGGTGGCTCGGGCGGTGGTGGGGA |
| | | | GGTGCCACTGGTGGAATCTGGGGGAGACCTGGTGAAGCCTGGG |
| | | | GGGTCCTTGAGACTGTCCTGTGTGGCCTCTGGATTCACCCTCAG |
| | | | TAACTATGGCATGAGCTGGGTCCGTCAGTCTCCAGGGAAGGGG |
| | | | CTGCAGTGGGTCGCAGCTATTAGGAGTGGTGGAAACACATACT |
| | | | ACGCAGACGCTGTGAAGGGCCGATTCACCATCTCCAGAGACAA |
| | | | CGCCAAGAACACGCTGTATCTGCAGATGAACAGCCTGAGAGCC |
| | | | GAGGACACGGCCGTGTATTACTGTGCGTCCCAATGGGGTGATT |
| | | | GGGGCCACTTTGAGTACTGGGGCCAGGGCACCCTGGTCACCGT |
| | | | CTCCTCA |
| 61 | scFv 18 | AA | QSALTQTASVSGSLGQKVTISCTGSRSNIGANYVGWYQQVPGIGP |
| | | | RTVIFGYNYRPSGVPDRESSSKSGNSATLTISGLQAEDEAEYYCST |
| | | | WDSSLNAYVFGSGTQLTVLGGGSSRSSSSGGGGSGGGGEVPLVES |
| | | | GGDLVKPGGSLRLSCVASGFTLSNYGMSWVRQSPGKGLQWVAAI |
| | | | RSGGNTYYADAVKGRFTISRDNAKNTLYLQMNSLRAEDTAVYYC |
| | | | ASQWGDWGHFEYWGQGTLVTVSS |
| 19 | scFv 19 | DNA | CAGGCTGTGCTGACTCAGCCGGCCTCAGTGTCGGGGCCCCTTG |
| | | | GCCAGAGGGTCACCATTTCCTGCTCTGGAAGCACGAACAACAT |
| | | | CGGTATTGTTGGTGCGGCCTGGTACCAACAGTTTCCAGGAAAG |
| | | | GCCCCTAAACTCCTCGTATACAGTGATGGGAGTCGACCGTCAG |
| | | | GGGTCCCTGACCGGTTTTCCGGCTCCAACTCTGGCAACTCAGCC |
| | | | ACCCTGACCATCAGTGGACTTCAGGCTGAGGACGAGGCTGATT |
| | | | ATTACTGTCAGTCTGTTGATGCCACGCATGGTACTATTCTGTTC |
| | | | GGCGGAGGGACCCAGCTGACCGTCCTCGGTGGTGGTTCCTCTA |
| | | | GATCTTCCTCCTCTGGTGGCGGTGGCTCGGGCGGTGGTGGGGA |
| | | | GGTGCAGCTGGTGGAGACTGGGGGAGACCTGGTGAAGCCTGC |
| | | | GGGGTCCCTGAGACTGTCATGTGCGGCCTCTGGATTCCCCTTCA |
| | | | GTGGCTACAGTATGACCTGGGTCCGCCAGGCCCCTGAAAAGGG |
| | | | TCTGCAATTGGTCGCAGGTATTAACAGCGATGGAAGTCACACA |
| | | | TACTACACAGACGATGTGAAGGGCCGATTCACCGTCTCCAGAG |
| | | | ACAACACCAAATACATACTGTATCTGCAGATGAACAGTCTGAG |
| | | | AGTCGAGGACACGGCCATATATTATTGTGGCTCTCATAGTTGG |
| | | | GGCAATTTTCACTACTGGGGCCAGGGAACCCTGGTCACCGTCT |
| | | | CCTCA |
| 62 | scFv 19 | AA | QAVLTQPASVSGPLGQRVTISCSGSTNNIGIVGAAWYQQFPGKAP |
| | | | KLLVYSDGSRPSGVPDRFSGSNSGNSATLTISGLQAEDEADYYCQ |
| | | | SVDATHGTILFGGGTQLTVLGGGSSRSSSSGGGGSGGGGEVQLVE |
| | | | TGGDLVKPAGSLRLSCAASGFPFSGYSMTWVRQAPEKGLQLVAGI |
| | | | NSDGSHTYYTDDVKGRFTVSRDNTKYILYLQMNSLRVEDTAIYY |
| | | | CGSHSWGNFHYWGQGTLVTVSS |
| 20 | scFv 20 | DNA | CAGTCTGTGCTGAATCAGCCACCTTCAGTATCAGTCTCTCTGGG |
| | | | ACAGACAGCAACCATCTCCTGCTCTGGAGAGAGTCTGAGTAAA |
| | | | TATTATGCACAATGGTTCCAGCAGAAGGCAGGCCAAGTCCCTG |
| | | | TGTTGGTCATATATAAGGACACTGAGCGGCCCTCTGGGATCCC |
| | | | TGACCGATTCTCCGGCTCCAGTTCAGGGAACACACACACCCTG |
| | | | ACCATCAGCGGGGCTCGGGCCGAGGACGAGGCTGACTATTACT |
| | | | GCGAGTCAGAAGTCAGTCCTGATACTATTGTGTTCGGCGGAGG |
| | | | CACCCACCTGACCGTCCTCGGTGGTGGTTCCTCTAGATCTTCCT |
| | | | CCTCTGGTGGCGGTGGCTCGGGCGGTGGTGGGGAGGTACAGCT |
| | | | GGTGGAATCTGGGGGAGACCTGGTGAAGCCTGGGGGATCCCTG |
| | | | AGACTGTCCTGTGTGGCCTCTGGATTCACCTTCAGTAGCTATGC |
| | | | CATGAGCTGGGTCCGCCAGGCTCCAGGGGAGGGGCTGCAGTGG |
| | | | GTCGCATACATTAACAGTGCTGGAAGTACCACATACTACGCAG |
| | | | ACGCTGTGAAGGGCCGATTCACCATCTCCAGAGACAACGCCAG |
| | | | GAACACAGTGTATCTGCAGATGAACAGCCTGAGAGCCGAGGA |
| | | | CACAGCCGTGTATTACTGTGGCCGATATTATTATGGTCTGGACT |
| | | | ACTGGGGTCCTGGCACCTCAGTCTTCGTGTCCTCA |
| 63 | scFv 20 | AA | QSVLNQPPSVSVSLGQTATISCSGESLSKYYAQWFQQKAGQVPVL |
| | | | VIYKDTERPSGIPDRFSGSSSGNTHTLTISGARAEDEADYYCESEVS |
| | | | PDTIVFGGGTHLTVLGGGSSRSSSSGGGGSGGGGEVQLVESGGDL |
| | | | VKPGGSLRLSCVASGFTFSSYAMSWVRQAPGEGLQWVAYINSAG |
| | | | STTYYADAVKGRFTISRDNARNTVYLQMNSLRAEDTAVYYCGRY |
| | | | YYGLDYWGPGTSVFVSS |
| 21 | scFv 21 | DNA | CAGCCTGTGCTGACCCAGCCACCCTCGGTGTCAGTGTCTCTGGG |
| | | | ACAGACAGCAACCATCTCCTGCTCTGGAGAGAGTCTGAGTAAA |
| | | | TATTATGCACAATGGTTCCAGCAGAAGGCAGGCCAAGCCCCTG |
| | | | TGTTGGTCATATATAAGGACACTGAGCGGCCCTCTGGGATCCC |
| | | | TGACCGATTCTCTGGCTCCAGTTCAGGGAACACACACACCCTG |
| | | | ACCATCAGCGGGGCTCGGGCCGAGGACGAGGCTGACTATTACT |
| | | | GCGAGTCAGCAGTCAGTAGTGATACTGCAGTGTTCGGCTCAGG |
| | | | GACCCAACTGACCGTCCTCGGCGGTGGTTCCTCTAGATCTTCCT |
| | | | CCTCTGGTGGCGGTGGCTCGGGCGGTGGTGGGGAGGTACAGCT |
| | | | GGTGGAGTCTGGGGGAGATATGGTGAAGCCTGGGGGGTCCTTG |
| | | | AGACTGTCCTGTGTGGCCTCTGGATTCACCTTCAGTAGTTTCTG |
| | | | GATGTACTGGGTCCGCCAGGCTCCTGGGAAGGGGCTGCAGTGG |
| | | | GTCGGAGGTATTAATAGTGGTGGAAGTATTACAAGTTACGCAG |
| | | | ACGCTGTGAAGGGCCGATTCACCATCTCCAGAGACAACGCCAG |
| | | | GAACACACTGTATCTGCAGATGGACAGTCTGAGAGATGAAGAC |
| | | | ACAGCCGTCTACTACTGTGCGAAGCCGGATATATATGGGAGTT |
| | | | TTGACCACTGGGGCCAGGGCACCCTGGTCACCGTCTCCTCA |
| 64 | scFv 21 | AA | QPVLTQPPSVSVSLGQTATISCSGESLSKYYAQWFQQKAGQAPVL |
| | | | VIYKDTERPSGIPDRFSGSSSGNTHTLTISGARAEDEADYYCESAVS |
| | | | SDTAVFGSGTQLTVLGGGSSRSSSSGGGGSGGGGEVQLVESGGD |
| | | | MVKPGGSLRLSCVASGFTFSSFWMYWVRQAPGKGLQWVGGINS |
| | | | GGSITSYADAVKGRFTISRDNARNTLYLQMDSLRDEDTAVYYCA |
| | | | KPDIYGSFDHWGQGTLVTVSS |
| 22 | scFv 22 | DNA | CAGCCTGTGCTCACTCAGCCGCCCTCAGTGTCTGGGTCCCTGGG |
| | | | CCAGAGGGTCACCATCTCCTGCACTGGAAGCAGCTCCAATGTT |
| | | | GGTTATGGCGATTATGTGGGCTGGTACCAGCAGCTCCCAGGAA |
| | | | CAAGCCCCAGAACCCTCATTTATTCTAGTAGTAGCCGACCCTCG |
| | | | GGAGTCCCCGATCGATTCTCTGGCTCCAGGTCAGGCAGCACAG |
| | | | CAACCCTGACCATCTCTGGGCTCCAGGCTGAAGATGAAGCCGA |
| | | | TTATCACTGCTCATCCTATGACACCAGTCTCAATGGTTTCGTGT |
| | | | TCGGCTCAGGGACCCAGCTGACCATCCTCGGTGGTGGTTCCTCT |
| | | | AGATCTTCCTCCTCTGGTGGCGGTGGCTCGGGCGGTGGTGGGG |
| | | | AACTCACACTGCAGGAGTCAGGGCCAGGACTGGTGAAGCCCGC |
| | | | ACAGACCCTCTCTCTCACCTGTACTGTGTCCGGAGACTCCGTCA |
| | | | CCAGCAGTCACTTCTGGCACTGGATCCGCCAGCGCCCTGGGAG |
| | | | GGGACTGGAATGGATGGGATACTGGTCAGACATTACGATTTAC |
| | | | AGTCCAACATTCCAAGGACGCATCTCCATCACTGTTGACACGG |
| | | | CCAGGAACCAGTTCTCCCTGCATTTGAACTCCGTGACGACCGA |
| | | | GGACTCGGCCGTTTATTTCTGTGCAAGATATAATGGATACGTCG |
| | | | ATTATTTAGACGTCTGGGGCCAGGGCACCCTGATCACCGTCTCC |
| | | | TCA |
| 65 | scFv 22 | AA | QPVLTQPPSVSGSLGQRVTISCTGSSSNVGYGDYVGWYQQLPGTS |
| | | | PRTLIYSSSSRPSGVPDRFSGSRSGSTATLTISGLQAEDEADYHCSS |
| | | | YDTSLNGFVFGSGTQLTILGGGSSRSSSSGGGGSGGGGELTLQESG |
| | | | PGLVKPAQTLSLTCTVSGDSVTSSHFWHWIRQRPGRGLEWMGYW |
| | | | SDITIYSPTFQGRISITVDTARNQFSLHLNSVTTEDSAVYFCARYNG |
| | | | YVDYLDVWGQGTLITVSS |
| 23 | scFv 23 | DNA | GATATTGTCATGACACAGACGCCACTGTCCCTGTCCGTCAGTCC |
| | | | TGGAGAGCCGGCCTCCATCTCCTGCAAGGCCAGTCAGAGCCTC |
| | | | CTGCACAGTAACGGGAACACCTATTTGTATTGGTTCCGACAGA |
| | | | AGCCAGGCCAGTCTCCACAGCGCCTGATCTACTTGGTTTCCAAC |
| | | | AGAGACGCTGGGGTCCCAGACAGGTTCAGTGGCAGCGGGTCA |
| | | | GGGACAGATTTCACCCTGAGAATCAGCAGAGTAGAGGCTGATG |
| | | | ATTCTGGAATTTATTACTGCGGCCAAGGTAAACAAGATCCTAT |
| | | | CACCTTTGGCGAAGGGACACATCTGGAGATTAAAGGTGGTTCC |
| | | | TCTAGATCTTCCTCCTCTGGTGGCGGTGGCTCGGGCGGTGGTGG |
| | | | GGAACTCACACTGCAGGAGTCAGGGCCAGGACTGGTGAAGCC |
| | | | CTCACAGACCCTCTCTCTCACCTGTGTTGTGTCCGGAGGCTCCG |
| | | | TCACCACCAGTGACTACTGGAACTGGCTCCGCCAGCGCCCTGG |
| | | | GAGGGGACTGGAATACATGGGATACTGGAAAGGTCGGACTGA |
| | | | GTACAACCCGGCATTCCAGGGACGCATCTCCATCACTGCTGAC |
| | | | ACGGCCAAGAACCAGTTCTCCCTGCAGTTAAACTCCGTGACCG |
| | | | CCGACGACACGGCCATTTATTATTGTGCACATTGGGGGTCAGG |
| | | | CAATCTTGACTATTGGGGCCCGGGAACCCTGGTCACCGTCTCCT |
| | | | CA |
| 66 | scFv 23 | AA | DIVMTQTPLSLSVSPGEPASISCKASQSLLHSNGNTYLYWFRQKPG |
| | | | QSPQRLIYLVSNRDAGVPDRFSGSGSGTDFTLRISRVEADDSGIYY |
| | | | CGQGKQDPITFGEGTHLEIKGGSSRSSSSGGGGSGGGGELTLQESG |
| | | | PGLVKPSQTLSLTCVVSGGSVTTSDYWNWLRQRPGRGLEYMGY |
| | | | WKGRTEYNPAFQGRISITADTAKNQFSLQLNSVTADDTAIYYCAH |
| | | | WGSGNLDYWGPGTLVTVSS |
| 24 | scFv 24 | DNA | CAGTCTGTGCTGAATCAGCCACCTTCTGTGTCTGGAGCCCTGGG |
| | | | GCAGACCGTCACCATCTCCTGCTCTGGAAGTCACACCAACATC |
| | | | GGCAGTGGATTTGATGTACAGTGGTACCAACAGCTCCCAGGAA |
| | | | AGTCCCCTCAAACTCTCGTCTATGCTAATATCAATCGACCCTCG |
| | | | GGGGTCCCGGTTCGATTCTCTGGCTCCAAGTCAGGCACCACAG |
| | | | CCACCCTGACCATCACTGGGATCCAGGCTGAGGATGAGGCTGA |
| | | | TTATTACTGCCAGTCCTATGATGACAACGTCGATGGTTACGTGT |
| | | | TCGGCTCAGGGACCCAGCTGACCGTCCTTGGTGGTGGTTCCTCT |
| | | | AGATCTTCCTCCTCTGGTGGCGGTGGCTCGGGCGGTGGTGGGG |
| | | | AGGTCCAGCTGGTGCAGTCTGGGGGARACCTGGTGAAGCCTGG |
| | | | GGGGTCCCTGAGACTCTCCTGTGTGGCCTCTGGATTCACCCTCA |
| | | | GTAGTAACTATATGGACTGGGTCCGCCAGGCTCCAGGGAAGGG |
| | | | GCCTCAGTGGGTCGCACAAATTAGCACTGATGGAAGTAGGACA |
| | | | TATTACGCAGACGCTGTGAAGGGCCGATTCACCATTTCCAGAG |
| | | | ACACCGCCAAGAACACACTGTATCTGCAGATGAACAGTCTGAG |
| | | | AGCCGAGGACACGGCTATGTATTACTGTGCAAAGAACGAATAC |
| | | | TTGGCCCCTACGTACTGGGGCCAGGGCACCCTGGTCACCGTCT |
| | | | CCTCA |
| 67 | scFv 24 | AA | QSVLNQPPSVSGALGQTVTISCSGSHTNIGSGFDVQWYQQLPGKSP |
| | | | QTLVYANINRPSGVPVRFSGSKSGTTATLTITGIQAEDEADYYCQS |
| | | | YDDNVDGYVFGSGTQLTVLGGGSSRSSSSGGGGSGGGGEVQLVQ |
| | | | SGGXLVKPGGSLRLSCVASGFTLSSNYMDWVRQAPGKGPQWVA |
| | | | QISTDGSRTYYADAVKGRFTISRDTAKNTLYLQMNSLRAEDTAM |
| | | | YYCAKNEYLAPTYWGQGTLVTVSS |
| 25 | scFv 25 | DNA | GATATCGTCATGACACAGACTCCGCTGTCCCTGTCCGTCAGCCC |
| | | | TGGAGAGACGGCCTCCATCTCCTGCAAGGCCAGTCAGAGCCTC |
| | | | CTGCACAGTAACGGGAACACCTATTTGAATTGGTTCCGACAGA |
| | | | AGCCAGGCCAGTCTCCACAGGTCCTGATCTATCAGGTCTCCAA |
| | | | CAGAGACACTGGGGTCCCAGACAGGTTCAGTGGCAGCGGGTCA |
| | | | GGGACAGATTTCACCCTGAGAATCAGTAGAGTGGAGGCTGACG |
| | | | ATGCTGGAGTTTATTATTGCGGGCAAGGTATACAATATCCTCCG |
| | | | ACGTTCGGAGCAGGAACCAAGGTGGAGCTCAAAGGTGGTTCCT |
| | | | CTAGATCTTCCTCCTCTGGTGGCGGTGGCTCGGGCGGTGGTGG |
| | | | GGAGGAGCAACTGGTGGAGTTTGGGGGAGACCTGGTGAAGCC |
| | | | TGGGGGGTCCTTGAGACTCTCCTGTGTGGCCTCTGGATTCACCT |
| | | | TCAGTAGTCTTTCCATGAACTGGATCCGCCAGGCTCCAGGGAA |
| | | | GGGGCTGCAGTGGGTCTCACAAATTAGCCCTGATGGAACTATC |
| | | | ACAGGCTACACAGACGCTGTGAAGGGCCGATTCACCATCTCCA |
| | | | GAGACAATGCCAAGAACACGCTGTATCTTCAGATGAACAGCCT |
| | | | GAGAGATGAAGACACGGCAATATATTACTGTGCGAAGAGTCG |
| | | | ATATACGGATACGTACCCCTTTGACTACTGGGGCCCGGGAACC |
| | | | CTGGTCACCGTCTCCTCA |
| 68 | scFv 25 | AA | DIVMTQTPLSLSVSPGETASISCKASQSLLHSNGNTYLNWFRQKPG |
| | | | QSPQVLIYQVSNRDTGVPDRFSGSGSGTDFTLRISRVEADDAGVY |
| | | | YCGQGIQYPPTFGAGTKVELKGGSSRSSSSGGGGSGGGGEEQLVE |
| | | | FGGDLVKPGGSLRLSCVASGFTFSSLSMNWIRQAPGKGLQWVSQI |
| | | | SPDGTITGYTDAVKGRFTISRDNAKNTLYLQMNSLRDEDTAIYYC |
| | | | AKSRYTDTYPFDYWGPGTLVTVSS |
| 26 | scFv 26 | DNA | TCCAGTGTGCTGACTCAGCCTCCCTCGGTATCAGTGTCTCTGGG |
| | | | ACAGACAGCAACCATCTCCTGCTCTGGAGAGAGTCTGAGTAAA |
| | | | TATTATACTCAATGGTTCCAGCAGAAGGCAGGCCAAGCCCCTA |
| | | | TCTTGGTCATTTATAAGGACACTGAGCGGCCCTCTGGGATCCCT |
| | | | GACCGGTTCTCTGGCTCCAGTTCAGGCAACACACACACCCTGA |
| | | | CCATCAGCGGGGCTCGGGCCGAGGACGAGGCTGACTATTATTG |
| | | | CGAGTCATTAGTCGATTCTGATGCTTACATGTTCGGCTCTGGAA |
| | | | CCCAGCTGACCGTCCTTGGCGGTGGTTCCTCTAGATCTTCCTCC |
| | | | TCTGGTGGCGGTGGCTCGGGCGGTGGTGGGGAGGTGCAGCTGG |
| | | | TGGAGACTGGGGGAGACCTGGTGAAGCCTGGGGGGTCCCTGA |
| | | | GACTCTCCTGTGTGGGCTCTGGATTCACCTTCAGTAGCTACTGG |
| | | | ATGAGCTGGGTCCGCCAGGCTCCAGGGAAGGGGCTACAGTGG |
| | | | ATTGCAGATATTAGCGATAGTGGAAGTAGCACAGACTATGCAG |
| | | | ACGCTGTGAAGGGCCGATTCACCATCTCCAGAGACAATGCCAA |
| | | | GAACACACTGTATCTGCAGATGAACAGCCTGAGAGCCGAGGAC |
| | | | ACGGCCATGTATTACTGTGCAAGGGGTCTGGGTATGGACTACT |
| | | | GGGGCCATGGCACCTCACTCTTCGTGTCCTCA |
| 69 | scFv 26 | AA | SSVLTQPPSVSVSLGQTATISCSGESLSKYYTQWFQQKAGQAPILVI |
| | | | YKDTERPSGIPDRFSGSSSGNTHTLTISGARAEDEADYYCESLVDS |
| | | | DAYMFGSGTQLTVLGGGSSRSSSSGGGGSGGGGEVQLVETGGDL |
| | | | VKPGGSLRLSCVGSGFTFSSYWMSWVRQAPGKGLQWIADISDSG |
| | | | SSTDYADAVKGRFTISRDNAKNTLYLQMNSLRAEDTAMYYCARG |
| | | | LGMDYWGHGTSLFVSS |
| 27 | scFv 27 | DNA | CAGCCTGTGCTGACCCAGCCACCCTCGGTATCAGTGTCTCTGGG |
| | | | ACAGACAGCAACCATCTCCTGCTCTGGAGAGAGTCTGAGTAAA |
| | | | TATTATGCACAATGGTTCCAGCAGAAGGCAGGCCAAGCCCCTG |
| | | | TGTTGGTCATATATAAGGACACTGAGCGGCCCTCTGGGATCCC |
| | | | TGACCGATTCTCTGGCTCCAGTTCAGGGAACACACACACCCTG |
| | | | ACCATCACTGGGATCCAGGCTGAGGATGAGGGTGATTATCACT |
| | | | GCCACTCCTATGATGAGAACCTCGATGGTTACGTGTTCGGCTCA |
| | | | GGAACCCAACTGACCGTCCTTGGCGGTGGTTCCTCTAGATCTTC |
| | | | CTCCTCTGGTGGCGGTGGCTCGGGCGGTGGTGGGGAGGTGCAG |
| | | | CTGGTGGAGTCTGGGGGAGACCTGGTGAAGCCTGGGGGGTCTC |
| | | | TGAGACTCTCCTGTGTGGGCTCTGGATTCACCTTCAGTAGCTAC |
| | | | TGGATGAGCTGGGTCCGCCAGGCTCCAGGGAAGGGGCTACAGT |
| | | | GGATTGCAGATATTAGCGATAGTGGAAGTAGCACAGACTATGC |
| | | | AGACGCTGTGAAGGGCCGATTCACCATCTCCAGAGACAATGCC |
| | | | AAGAACACACTGTATCTGCAGATGAACAGCCTGAGAGCCGAG |
| | | | GACACGGCCATGTATTACTGTGCAAGGGGTCTGGGTATGGACT |
| | | | ACTGGGGCCATGGCACCTCACTCTTCGTGTCCTCA |
| 70 | scFv 27 | AA | QPVLTQPPSVSVSLGQTATISCSGESLSKYYAQWFQQKAGQAPVL |
| | | | VIYKDTERPSGIPDRFSGSSSGNTHTLTITGIQAEDEGDYHCHSYDE |
| | | | NLDGYVFGSGTQLTVLGGGSSRSSSSGGGGSGGGGEVQLVESGG |
| | | | DLVKPGGSLRLSCVGSGFTFSSYWMSWVRQAPGKGLQWIADISD |
| | | | SGSSTDYADAVKGRFTISRDNAKNTLYLQMNSLRAEDTAMYYCA |
| | | | RGLGMDYWGHGTSLFVSS |
| 28 | scFv 28 | DNA | CAGTCTGCCCTGACTCAACCTTCCTCGGTATCAGTGTCTCTGGG |
| | | | ACAGACAGCAACCATCTCCTGCTCTGGAGAGAGTCTGAATAAA |
| | | | TATTATGTACAGTGGTTCCAGCAGAAGGCAGGCCAAGTCCCTA |
| | | | TATTGGTCATATATAGGGACACTGAGCGGCCCTCTGGGATCCC |
| | | | TGACCGATTCTCTGGCTCCAGTTCAGGGAACACACACACCCTG |
| | | | ACCATCACCGGGACTCGGGCCGAGGACGAGGCTGACTATTACT |
| | | | GCGAGTCACTAGTCAATGATAATACTTACGTGTTCGGCTCAGG |
| | | | GACCCAACTGACCGTCCTTGGCGGTGGTTCCTCTAGATCTTCCT |
| | | | CCTCTGGTGGCGGTGGCTCGGGCGGTGGTGGGGAGGTGCAGCT |
| | | | GGTGGAGACTGGGGGAGACCTGGTGAAGCCTGGGGGGTCCCT |
| | | | GAGACTCTCCTGTGTGGGCTCTGGATTCACCTTCAGTAGCTACT |
| | | | GGATGAGCTGGGTCCGCCAGGCTCCAGGGAAGGGGCTACAGT |
| | | | GGATTGCAGATATTAGCGATAGTGGAAGTAGCACAGACTATGC |
| | | | AGACGCTGTGAAGGGCCGATTCACCATCTCCAGAGACAATGCC |
| | | | AAGAACACACTGTATCTGCAGATGAACAGCCTGAGAGCCGAG |
| | | | GACACGGCCATGTATTACTGTGCAAGGGGTCTGGGTATGGACT |
| | | | ACTGGGGCCATGGCACCTCACTCTTCGTGTCCTCA |
| 71 | scFv 28 | AA | QSALTQPSSVSVSLGQTATISCSGESLNKYYVQWFQQKAGQVPIL |
| | | | VIYRDTERPSGIPDRFSGSSSGNTHTLTITGTRAEDEADYYCESLVN |
| | | | DNTYVFGSGTQLTVLGGGSSRSSSSGGGGSGGGGEVQLVETGGD |
| | | | LVKPGGSLRLSCVGSGFTFSSYWMSWVRQAPGKGLQWIADISDS |
| | | | GSSTDYADAVKGRFTISRDNAKNTLYLQMNSLRAEDTAMYYCAR |
| | | | GLGMDYWGHGTSLFVSS |
| 29 | scFv 29 | DNA | CAGTCTGTGCTGAATCAGCCGCCTTCAGTATCAGTGTCTCTGGG |
| | | | ACAGACAGCAACCATCTCCTGCTCTGGAGAGAGTCTGAATAAA |
| | | | TATTATACACAGTGGTTCCAGCAGAAGGCAGGCCAAGCCCCTG |
| | | | TGTTGGTCATATATAAGGACACTGAGCGGCCCTCTGGGATCCC |
| | | | TGACCGATTCTCCGGATCCAGTTCAGGGAACACACACACCCTG |
| | | | ACCATCAGCGGGGCTCGGGCCGAGGACGAGGCTGACTATTACT |
| | | | GCGAGTCAGTAGACAGTACTGATACTTACGTGTTCGGCTCAGG |
| | | | GACCCAGCTGACCATCCTCGGTGGTGGTTCCTCTAGATCTTCCT |
| | | | CCTCTGGTGGCGGTGGCTCGGGCGGTGGTGGGGAGGTACATCT |
| | | | GGTGGAATCTGGGGGAGACTTGGTGAAGCCTGGGGGGTCCCTG |
| | | | AGACTCTCCTGTGTGGGCTCTGGATTCACCTTCAGTAGCTACTG |
| | | | GATGAGCTGGGTCCGCCAGGCTCCAGGGAAGGGGCTACAGTG |
| | | | GATTGCAGATATTAGCGATAGTGGAAGTAGCACAGACTATGCA |
| | | | GACGCTGTGAAGGGCCGATTCACCATCTCCAGAGACAATGCCA |
| | | | AGAACACACTGTATCTGCAGATGAACAGCCTGAGAGCCGAGG |
| | | | ACACGGCCATGTATTACTGTGCAAGGGGTCTGGGTATGGACTA |
| | | | CTGGGGCCATGGCACCTCACTCTTCGTGTCCTCA |
| 72 | scFv 29 | AA | QSVLNQPPSVSVSLGQTATISCSGESLNKYYTQWFQQKAGQAPVL |
| | | | VIYKDTERPSGIPDRFSGSSSGNTHTLTISGARAEDEADYYCESVDS |
| | | | TDTYVFGSGTQLTILGGGSSRSSSSGGGGSGGGGEVHLVESGGDL |
| | | | VKPGGSLRLSCVGSGFTFSSYWMSWVRQAPGKGLQWIADISDSG |
| | | | SSTDYADAVKGRFTISRDNAKNTLYLQMNSLRAEDTAMYYCARG |
| | | | LGMDYWGHGTSLFVSS |
| 30 | scFv 30 | DNA | CAGTCTGTCTTGACTCAGCCAGCCTCGGTATCAGTGTCTCTGGG |
| | | | ACAGACAGCAACCATCTCCTGCTCTGGAGAGAGTCTGAGTAAA |
| | | | TATTATACACAGTGGTTCCAGCAGAAGGCAGGCCAGGCCCCTG |
| | | | TGTTGGTCATCTATAAGGACACTGAGCGGCCCTCTGGGATCCCT |
| | | | GACCGATTCTCTGGCTCCAGCTCAGGGAACACGCACACCCTGA |
| | | | CCATCAGCGGGGCTCAGGCCGAGGATGAGGCTGATTATTACTG |
| | | | CCAGTCCTATGATGACAACGTCGATGGTTACGTGTTCGGCTCTG |
| | | | GGACCCAACTGACCGTCCTCGGTGGTGGTTCCTCTAGATCTTCC |
| | | | TCCTCTGGTGGCGGTGGCTCGGGCGGTGGTGGGGAGGTACAGC |
| | | | TGGTGGAATCTGGGGGAGACCTGGTGAAGCCTGGGGGGTCCCT |
| | | | GAGACTCTCCTGTGTGGGCTCTGGATTCACCTTCAGTAGCTACT |
| | | | GGATGAGCTGGGTCCGCCAGGCTCCAGGGAAGGGGCTACAGT |
| | | | GGATTGCAGATATTAGCGATAGTGGAAGTAGCACAGACTATGC |
| | | | AGACGCTGTGAAGGGCCGATTCACCATCTCCAGAGACAATGCC |
| | | | AAGAACACACTGTATCTGCAGATGAACAGCCTGAGAGCCGAG |
| | | | GACACGGCCATGTATTACTGTGCAAGGGGTCTGGGTATGGACT |
| | | | ACTGGGGCCATGGCACCTCACTCTTCGTGTCCTCA |
| 73 | scFv 30 | AA | QSVLTQPASVSVSLGQTATISCSGESLSKYYTQWFQQKAGQAPVL |
| | | | VIYKDTERPSGIPDRFSGSSSGNTHTLTISGAQAEDEADYYCQSYD |
| | | | DNVDGYVFGSGTQLTVLGGGSSRSSSSGGGGSGGGGEVQLVESG |
| | | | GDLVKPGGSLRLSCVGSGFTFSSYWMSWVRQAPGKGLQWIADIS |
| | | | DSGSSTDYADAVKGRFTISRDNAKNTLYLQMNSLRAEDTAMYYC |
| | | | ARGLGMDYWGHGTSLFVSS |
| 31 | scFv 31 | DNA | CAGTCTGCCCTGACTCAACCTTCCTCGGTATCAGTGTCTCTGGG |
| | | | ACAGACAGCAACCATCTCCTGCTCTGGAGAGAGTCTGACTAAA |
| | | | TATTATGTACAATGGTTCCAACAGAAGGCAGGCCAAGCCCCTG |
| | | | TATTGGTCATATATAGGGACACTGAGCGGCCCTCTGGGATCCC |
| | | | TGACCGATTCTCTGGCTCCAGTTCAGGTAATACACACACCCTGA |
| | | | CCATCAGTGGGGCTCGGGCCGAGGACGAGGCTGACTATTATTG |
| | | | CGAGTCAATGGACGGTCGTGAGGCTTACGTGTTCGGCTCAGGG |
| | | | ACCCAACTGACCGTCCTCGGCGGTGGTTCCTCTAGATCTTCCTC |
| | | | CTCTGGTGGCGGTGGCTCGGGCGGTGGTGGGGAGGTACAGCTG |
| | | | GTGGAGTCTGGGGGAGACCTGGTGAAGCCGGGGGGGTCCCTG |
| | | | AGACTGTCCTGTGTGGCCTCTGGATTCACCTTCAGTAGCTACTG |
| | | | GATGAGCTGGGTCCGCCAGGCTCCAGGGAAGGGGCTACAGTG |
| | | | GATTGCAGATATTAGCGATAGTGGAAGTAGCACAGACTATGCA |
| | | | GACGCTGTGAAGGGCCGATTCACCATCTCCAGAGACAATGCCA |
| | | | AGAACACACTGTATCTGCAGATGAACAGCCTGAGAGCCGAGG |
| | | | ACACGGCCATGTATTACTGTGCAAGGGGTCTGGGTATGGACTA |
| | | | CTGGGGCCATGGCACCTCACTCTTCGTGTCCTCA |
| 74 | scFv 31 | AA | QSALTQPSSVSVSLGQTATISCSGESLTKYYVQWFQQKAGQAPVL |
| | | | VIYRDTERPSGIPDRFSGSSSGNTHTLTISGARAEDEADYYCESMD |
| | | | GREAYVFGSGTQLTVLGGGSSRSSSSGGGGSGGGGEVQLVESGG |
| | | | DLVKPGGSLRLSCVASGFTFSSYWMSWVRQAPGKGLQWIADISD |
| | | | SGSSTDYADAVKGRFTISRDNAKNTLYLQMNSLRAEDTAMYYCA |
| | | | RGLGMDYWGHGTSLFVSS |
| 32 | scFv 32 | DNA | CAGTCTGTGTTGACTCAACCAGCCTCGGTATCAGTGTCTCTGGG |
| | | | ACAGACAGCAACCATCTCCTGCTCTGGAGAGAGTCTGAGTAAA |
| | | | TATTATGCACAATGGTTCCAGCAGAAGGCAGGCCAAGCCCCTG |
| | | | TGTTGGTCATATATAAGGACACTGAGCGGCCCTCTGGGATCCC |
| | | | TGACCGATTCTCTGGCTCCAGTTCAGGGAACACACACACCCTG |
| | | | ACCATCACTGGGATCCAGGCTGCGGATGAGGCTGATTATTACT |
| | | | GCCAGTCCTATGATGACAACCTCGATGGTTACGTGTTCGGCTCA |
| | | | GGAACCCAGCTGACCGTCCTTGGTGGTGGTTCCTCTAGATCTTC |
| | | | CTCCTCTGGTGGCGGTGGCTCGGGCGGTGGTGGGGAGGTACAG |
| | | | CTGGTGGAGTCTGGGGGAGACCTGGTGAAGCCTGGGGGGTCCC |
| | | | TGAGACTCTCCTGTGTGGGCTCTGGATTCACCTTCAGTAGCTAC |
| | | | TGGATGAGCTGGGTCCGCCAGGCTCCAGGGAAGGGGCTACAGT |
| | | | GGATTGCAGATATTAGCGATAGTGGAAGTAGCACAGACTATGC |
| | | | AGACGCTGTGAAGGGCCGATTCACCATCTCCAGAGACAATGCC |
| | | | AAGAACACACTGTATCTGCAGATGAACAGCCTGAGAGCCGAG |
| | | | GACACGGCCATGTATTACTGTGCAAGGGGTCTGGGTATGGACT |
| | | | ACTGGGGCCATGGCACCTCACTCTTCGTGTCCTCA |
| 75 | scFv 32 | AA | QSVLTQPASVSVSLGQTATISCSGESLSKYYAQWFQQKAGQAPVL |
| | | | VIYKDTERPSGIPDRFSGSSSGNTHTLTITGIQAADEADYYCQSYD |
| | | | DNLDGYVFGSGTQLTVLGGGSSRSSSSGGGGSGGGGEVQLVESG |
| | | | GDLVKPGGSLRLSCVGSGFTFSSYWMSWVRQAPGKGLQWIADIS |
| | | | DSGSSTDYADAVKGRFTISRDNAKNTLYLQMNSLRAEDTAMYYC |
| | | | ARGLGMDYWGHGTSLFVSS |
| 33 | scFv 33 | DNA | GATATTGTCATGACACAGACGCCACTGTCCCTGTCCGTCAGCCC |
| | | | TGGAGAGCCGGCCTCCATCTCCTGCAAGGCCAGTCAGAGCCTC |
| | | | CTGCACAGTGACGGGAACACCTATTTGCATTGGTACCTGCAAA |
| | | | AGCCAGGCCAGTCTCCACAGCTTCTGATCTACTCGGTTTCCAAC |
| | | | CGCTTCACTGGCGTGTCAGACAGGTTCAGTGGCAGCGGGTCAG |
| | | | GGACAGATTTCACCCTGAGAATCAGCAGAGTGGAGGCTGACGA |
| | | | TACTGGAGTTTATTACTGCGGGCAAGGTACACACCTCCCGTTCA |
| | | | CTTTTGGCCAAGGGACCAAACTGGAGATCAAAGGTGGTTCCTC |
| | | | TAGATCTTCCTCCTCTGGTGGCGGTGGCTCGGGCGGTGGTGGG |
| | | | GAGGTACAGCTGGTGGAGTCTGGGGGAGACCTGGTGAAGCCTG |
| | | | GGGGGTCCCTGAGACTCTCCTGTGCAGCCTCTGGATTCACCTTC |
| | | | AGTCACTACGACATGAACTGGGTCCGCCAGGCTCCTGGGAAGG |
| | | | GGCTGCAGTGGGTCGCATCTATTGCCTATGATGGATCTAACAC |
| | | | ATACTACACTGACGCTGTGAAGGGCCGATTCACCATCTCCAGA |
| | | | GACAACGCCAAGAATATACTGTTTTTGCTGGTGAATAGCCTGA |
| | | | GGGCCGAGGACACGGCCATATATTATTGTGCGCGGGGGCTGGT |
| | | | CTACTGGGGCCAGGGAACTTTGGTCACCGTCTCCTCA |
| 76 | scFv 33 | AA | DIVMTQTPLSLSVSPGEPASISCKASQSLLHSDGNTYLHWYLQKPG |
| | | | QSPQLLIYSVSNRFTGVSDRFSGSGSGTDFTLRISRVEADDTGVYY |
| | | | CGQGTHLPFTFGQGTKLEIKGGSSRSSSSGGGGSGGGGEVQLVES |
| | | | GGDLVKPGGSLRLSCAASGFTFSHYDMNWVRQAPGKGLQWVASI |
| | | | AYDGSNTYYTDAVKGRFTISRDNAKNILFLLVNSLRAEDTAIYYC |
| | | | ARGLVYWGQGTLVTVSS |
| 34 | scFv 34 | DNA | TCCTATGAGCTGACTCAGCCACCATCTGTGTCTGCGGCCCTGGG |
| | | | ACAGAGGGTCACCATCTCCTGCACTGGAACTAATACCAACATC |
| | | | GGCAGTGGTTATAGTGTACAATGGTACCAGCAGCTCCCAGGAG |
| | | | AGTCCCCAAAACCTATCATTTATGGTAGTAGTAATCGACCCTCG |
| | | | GGGGTCCCGGCTCGATTCTCTGGCTCCAAGTCAGGCAGCACAG |
| | | | GCACCCTGACCATCACTGGGATCCAGGCTGAGGATGAGGCTGA |
| | | | TTACCACTGCCAGTCCTATGATGACAGCCTCGATGGCCATGCTG |
| | | | TATTCGGCGGAGGGACCCAGCTGACCATCCTCGGCGGTGGTTC |
| | | | CTCTAGATCTTCCTCCTCTGGTGGCGGTGGCTCGGGCGGTGGTG |
| | | | GGGAGGTACAGCTGGTGGAGTCTGGGGGAGACCTGGTGAAGC |
| | | | CTGGGGGGTCCCTGAGACTCTCCTGTGTGGCCTCTGGATTAACC |
| | | | TTCAGTGACAATTACATGTCCTGGATCCGTCAGGTTCCAGGGA |
| | | | AGGGCCTGCAGTGGGTCTCACGAATTAGCACTGATGGAAGAGG |
| | | | CACAAGTTACGCAGACGCTGTGAAGGGCCGATTCACCATCTCC |
| | | | AGAGACAATGCCAAGAACACGCTGTATCTGCAGATGAACAGCC |
| | | | TGAGAGACGAGGACACGGCCGTGTATTACTGTGCAAGGCGGTC |
| | | | TGGTATTTGGGGGTTTGACTACTGGGGCCAGGGCACCCTGGTC |
| | | | ACCGTCTCCTCT |
| 77 | scFv 34 | AA | SYELTQPPSVSAALGQRVTISCTGTNTNIGSGYSVQWYQQLPGESP |
| | | | KPIIYGSSNRPSGVPARFSGSKSGSTGTLTITGIQAEDEADYHCQSY |
| | | | DDSLDGHAVFGGGTQLTILGGGSSRSSSSGGGGSGGGGEVQLVES |
| | | | GGDLVKPGGSLRLSCVASGLTFSDNYMSWIRQVPGKGLQWVSRIS |
| | | | TDGRGTSYADAVKGRFTISRDNAKNTLYLQMNSLRDEDTAVYYC |
| | | | ARRSGIWGFDYWGQGTLVTVSS |
| 35 | scFv 35 | DNA | TCCAGTGTGCTGACTCAGCCTCCTTCGGTATCAGTGTCTCTGGG |
| | | | ACAGACAGCAACCATCTCCTGCTCTGGAGAGAGTCTGAGTGAA |
| | | | TATTATGCACAATGGTTCCAGCAGAAGGCAGGCCAAGCCCCTG |
| | | | TATTGGTCATATATAAGGACACTGAGCGGGCCTCTGGAATCCC |
| | | | TGACCGATTCTCTGGCTCCAGTTCGGGGAACACACACACCCTG |
| | | | ACCATCAGCGGGGCTCGGGCCGAGGACGAGGCTGACTATTACT |
| | | | GCGAGTCAGCAGTCAATAGTGATGGTATTGTGTTCGGCGGAGG |
| | | | CACCCATCTGACCGTCCTCGGCGGTGGTTCCTCTAGATCTTCCT |
| | | | CCTCTGGTGGCGGTGGCTCGGGCGGTGGTGGGGAGGTACAGCT |
| | | | GGTGGAGTCTGGGGGAGACCTGGTGAAGCCTGCGGGGTCCCTG |
| | | | AGACTGTCCTGTGTGACCTCTGGATTCACCTTCAGCAGCTATGC |
| | | | CATGAGCTGGGTCCGCCAGGCTCCTGGGAAGGGGCTGCAGTGG |
| | | | GTCGGAAGTATTAACAGTGGTGGAAGTACCACAAGCTACGCAG |
| | | | ACGCTGTGAAGGGCCGATTCACCATCTCCAGAGACAACGCCAA |
| | | | GAACACAGTGTATCTGCAGATGAACAGCCTGAGAGCCGAGGA |
| | | | CACGGCCGTGTATTACTGTGCCCGGTGGTGGGACTTTGAGTACT |
| | | | GGGGCCAGGGCACCCTGGTCACCGTCTCCTCA |
| 78 | scFv 35 | AA | SSVLTQPPSVSVSLGQTATISCSGESLSEYYAQWFQQKAGQAPVL |
| | | | VIYKDTERASGIPDRFSGSSSGNTHTLTISGARAEDEADYYCESAV |
| | | | NSDGIVFGGGTHLTVLGGGSSRSSSSGGGGSGGGGEVQLVESGGD |
| | | | LVKPAGSLRLSCVTSGFTFSSYAMSWVRQAPGKGLQWVGSINSG |
| | | | GSTTSYADAVKGRFTISRDNAKNTVYLQMNSLRAEDTAVYYCAR |
| | | | WWDFEYWGQGTLVTVSS |
| 36 | scFv 36 | DNA | CAGTCTGTGCTGACTCAGCCGACCTCAGTGTCGGGGTCCCTTGG |
| | | | CCAGAGGGTCACCATCTCCTGCTCTGGAAGCACGAACAACATC |
| | | | GGTATTGTTGGTGCGAGCTGGTACCAACAGCTCCCAGGAAAGG |
| | | | CCCCTAAACTCCTCGTGTACAGTGATGGGGATCGACCGTCAGG |
| | | | GGTCCCTGACCGGTTTTCCGGCTCCAACTCTGGCAACTCAGACA |
| | | | CCCTGACCATCACTGGGCTTCAGGCTGAGGACGAGGCTGATTA |
| | | | TTACTGCCAGTCCTTTGATACCACGCTTGATGCTTACGTGTTCG |
| | | | GCTCAGGAACCCAGCTGACCGTCCTTGGTGGTGGTTCCTCTAG |
| | | | ATCTTCCTCCTCTGGTGGCGGTGGCTCGGGCGGTGGTGGGGAG |
| | | | GAGCAACTGGTGGAGTTTGGGGGAGGCCTGGTGAGGCCTGGG |
| | | | GGATCCCTGAGACTGTCCTGTGTGGCCTCTGGATTCACCTTCAG |
| | | | TAGCTATGGCATGACCTGGGTCCGTCAGTCTCCAGGGAAGGGG |
| | | | CTGCAGTGGGTCGCTACTATTAGCGATACCGGAAGCACATACT |
| | | | ACGCAGACACTGTGAAGGGCCGATTCAGCATCTCCAGAGACAA |
| | | | CGCCAAGAACACGCTGTATCTGCAGATGAACAGTCTGAGAGCC |
| | | | GACGACACGGCCCTGTATTATTGTACGAAGGACCTCGGGGTGT |
| | | | GGGGTATGGACAACTGGGGTCCTGGCACCTCACTCTTCGTGTC |
| | | | CTCA |
| 79 | scFv 36 | AA | QSVLTQPTSVSGSLGQRVTISCSGSTNNIGIVGASWYQQLPGKAPK |
| | | | LLVYSDGDRPSGVPDRFSGSNSGNSDTLTITGLQAEDEADYYCQS |
| | | | FDTTLDAYVFGSGTQLTVLGGGSSRSSSSGGGGSGGGGEEQLVEF |
| | | | GGGLVRPGGSLRLSCVASGFTFSSYGMTWVRQSPGKGLQWVATI |
| | | | SDTGSTYYADTVKGRFSISRDNAKNTLYLQMNSLRADDTALYYC |
| | | | TKDLGVWGMDNWGPGTSLFVSS |
| 37 | scFv 37 | DNA | TCCAGTGTGCTGACTCAGCCTCCCTCAGTATCAGTGTCTCTGGG |
| | | | ACAGACAGCAACCATCTCCTGCTCTGGAGAGAGTCTGAATGAC |
| | | | TATTATGCACAATGGTTCCAGCAGAAGGCAGGCCAAGTCCCTG |
| | | | TGTTGGTCATATATCGGGACACTGAGCGGCCCTCTGGGATCCCT |
| | | | GACCGATTCTCCGGCTCCAGTTCAGGGAACACACACACCCTGA |
| | | | CCATCAGCGGGGCTCGGGCCGAGGACGAGGCTGACTATTACTG |
| | | | CGAGTCAGAAGTCAGTGCTGGTGGTGCTGTATTCGGCGAAGGC |
| | | | ACCCACCTGACCGTCCTCGGTGGTGGTTCCTCTAGATCTTCCTC |
| | | | CTCTGGTGGCGGTGGCTCGGGCGGTGGTGGGGAGGTGCAGCTG |
| | | | GTGGAGACTGGGGGAGACCTGGTGAAGCCGGGGGGGTCCCTG |
| | | | AGACTGTCCTGTGTGGCCTCTGGATTCACCCTCAGAGACTACA |
| | | | GCATGACCTGGGTCCGCCAGACTCCTGAGAAGGGGCTGCAGTT |
| | | | GGTCGCAGGTATTAGCACCGGTGGAAGTACCACATACTACATA |
| | | | GACGCTGTGAAGGGCCGATTCACCATCTCCAGAGACAACGCCA |
| | | | AGAACACAGTGTATCTGCAGATGAACAGCCTGAGAGCCGAGG |
| | | | ACACGGCCATGTATCACTGTACAACCACGGCCTTAGGTTACTG |
| | | | GGGCCAGGGCACCCTGGTCACTGTCTCCTCA |
| 80 | scFv 37 | AA | SSVLTQPPSVSVSLGQTATISCSGESLNDYYAQWFQQKAGQVPVL |
| | | | VIYRDTERPSGIPDRFSGSSSGNTHTLTISGARAEDEADYYCESEVS |
| | | | AGGAVFGEGTHLTVLGGGSSRSSSSGGGGSGGGGEVQLVETGGD |
| | | | LVKPGGSLRLSCVASGFTLRDYSMTWVRQTPEKGLQLVAGISTGG |
| | | | STTYYIDAVKGRFTISRDNAKNTVYLQMNSLRAEDTAMYHCTTT |
| | | | ALGYWGQGTLVTVSS |
| 38 | scFv 38 | DNA | TCCAGTGTGCTGACTCAGCCTCCCTCAGTATCAGTCTCTCTGGG |
| | | | ACAGACAGCAACCATCTCCTGCTCTGGAGAGAGTCTGAGTGAG |
| | | | TATTATGCACAATGGTTCCAGCAGAAGGCAGGCCAAGCCCCTG |
| | | | TGTTGGTCATATATAAGGACACTGAGCGGCCCTCTGGGATCCC |
| | | | TGACCGATTCTCCGGCTCCAGTTCAGGGAACACACACACCCTG |
| | | | ACCATCAGCGGGGCTCGGGCCGAGGACGAGGCTGACTATTACT |
| | | | GCGCGTCAGCAGACAGTAGTGCTATTCCTGTGTTCGGCGGAGG |
| | | | CACCCATCTGACCGTCCTCGGCGGTGGTTCCTCTAGATCTTCCT |
| | | | CCTCTGGTGGCGGTGGCTCGGGCGGTGGTGGGGAGGTGCAGCT |
| | | | GGTGGAGTCTGGGGGAGACCTGGTGAAGCCTGGGGGGTCCCTG |
| | | | AGACTCTCCTGTGTGGCCTCAGGATTCAATTTCGGAAACTATGC |
| | | | CGTGAGCTGGGTCCGCCAGGCTCCTGGGAAGGGACTGCAGTGG |
| | | | GTCGCAGGTATCAGTAACGATGGAACTATAACAGGCTACACAG |
| | | | ATGTTGTGGAGGGCCGTTTCATCATCTCCAGAGACAATGCCAA |
| | | | GAACACGCTGTATCTACAGATGAACAGCCTGAGAGCCGAGGAC |
| | | | ATGGCTATATATTATTGTGTGGCTGGGGGCTTTCTTGACTACTG |
| | | | GGGCCAGGGAACCCTGGTCACCGTCTCCTCA |
| 81 | scFv 38 | AA | SSVLTQPPSVSVSLGQTATISCSGESLSEYYAQWFQQKAGQAPVL |
| | | | VIYKDTERPSGIPDRFSGSSSGNTHTLTISGARAEDEADYYCASAD |
| | | | SSAIPVFGGGTHLTVLGGGSSRSSSSGGGGSGGGGEVQLVESGGD |
| | | | LVKPGGSLRLSCVASGFNFGNYAVSWVRQAPGKGLQWVAGISND |
| | | | GTITGYTDVVEGRFIISRDNAKNTLYLQMNSLRAEDMAIYYCVAG |
| | | | GFLDYWGQGTLVTVSS |
| 39 | scFv 39 | DNA | CAGTCTGTGCTGACTCAGCCGCCCTCAGTGTCGGGGTCCCCTGG |
| | | | CCAGAGGGTCACCATCTCCTGCTCTGGAACCACGAAGAACATC |
| | | | GGTATAGTCGGTGCGAGTTGGTACCAACAACTCCCAGGAAAGG |
| | | | CCCCTAAACTCCTCGTTCACAGTGATGGGTTTCGACCGTCAGGG |
| | | | GTCCCTGACCGGTTTTCCGCCTCCAACTCTGGCGACTCAGACAT |
| | | | TCTGACCATCACTGGGCTTCAGGCTGAGGACGAGGCTGATTAT |
| | | | TACTGTCAGTCCTTTGATACCACGCTTGATGCTCACGTCTTCGG |
| | | | CTCAGGGACCCAGCTGACCGTCCTTGGCGGTGGTTCCTCTAGAT |
| | | | CTTCCTCCTCTGGTGGCGGTGGCTCGGGCGGTGGTGGGGAGGT |
| | | | GCCACTGGTGGAATCTGGGGGAGACCTGGTGAAGCCTGGGGG |
| | | | GTCCTTGAGACTGTCCTGTGTGGCCTCTGGATTCACCTTTAGTA |
| | | | GTTATGGCATGAGCTGGGTCCGTCAGTCTCCAGGGAAGGGGCT |
| | | | GCAGTGGGTCGCAGGTATTACCTATGATGGAAGTAATACATAC |
| | | | TACACTGACACTGTGAGGGGCCGATTCACCATCTCCAGAGACA |
| | | | ACGCCAGGAACACAGTGTATCTGCAGATGAACAGCCTGAGAGC |
| | | | CGAGGACACAGCTGTGTATTACTGTGTGGCCAGCCCCCTCTAC |
| | | | GGAACTTATGGTTTCGACTACTGGGGCCATGGCACCTCAGTCTT |
| | | | CGTGTCCTCA |
| 82 | scFv 39 | AA | QSVLTQPPSVSGSPGQRVTISCSGTTKNIGIVGASWYQQLPGKAPK |
| | | | LLVHSDGFRPSGVPDRFSASNSGDSDILTITGLQAEDEADYYCQSF |
| | | | DTTLDAHVFGSGTQLTVLGGGSSRSSSSGGGGSGGGGEVPLVESG |
| | | | GDLVKPGGSLRLSCVASGFTFSSYGMSWVRQSPGKGLQWVAGIT |
| | | | YDGSNTYYTDTVRGRFTISRDNARNTVYLQMNSLRAEDTAVYYC |
| | | | VASPLYGTYGFDYWGHGTSVFVSS |
| 40 | scFv 40 | DNA | CAGTCTGTGCTAACTCAGCCGCCCTCAGTGTCTGGGTCCCTGGG |
| | | | CCAGAAGGTCACCATCTCCTGCACTGGAAGCAGTTCCAGCGTC |
| | | | GGTACTCATTATGTGGGCTGGTACCAAATACCCCCAGGAATAG |
| | | | GCCCTAGAACCGTCATCTATAATAATGATTACCGACCTTCAGG |
| | | | GGTCCCCGATCGATTCTCTGGCTCCAAGTCAGGCAATTCAGCC |
| | | | ACCCTGACCATCTCTGGGCTCCAGGCTGAGGACGAGGCTGATT |
| | | | ATTACTGCTCATCATGGGATGACAGTCTCGGAGCTTACGTGCTC |
| | | | GGCTCAGGGACCCAGCTGACCATCCTCGGTGGTGGTTCCTCTA |
| | | | GATCTTCCTCCTCTGGTGGCGGTGGCTCGGGCGGTGGTGGGGA |
| | | | ACTCACACTGCAGGAGTCAGGGCCAGGACTGGTGAAGCCCTCA |
| | | | CAGACCCTCTCTCTCACCTGCGCTGTGTCCGGAGGCTCCGTCAT |
| | | | CAGAGGTTACTACTGGACCTGGATCCGCCAGCGCCCTGGGAGG |
| | | | GGACTGGAATGGATGGGTTACTGGGCAGGGAGTACAGATTATA |
| | | | ACCCGGCATTCCAGGGACGCATCTTCATCACTGCTGGCACGGC |
| | | | CAAGAATCAGTTCTCCCTGGAGATGACTTCCATGACCACGGAC |
| | | | GACACGGCCATATATTACTGTGTCGGATCCCTTCGGGGCGGTT |
| | | | GGCTCTTTGACAACTGGGGCCAGGGAACCCTAGTCACCGTCTC |
| | | | CTCC |
| 83 | scFv 40 | AA | QSVLTQPPSVSGSLGQKVTISCTGSSSSVGTHYVGWYQIPPGIGPR |
| | | | TVIYNNDYRPSGVPDRFSGSKSGNSATLTISGLQAEDEADYYCSS |
| | | | WDDSLGAYVLGSGTQLTILGGGSSRSSSSGGGGSGGGGELTLQES |
| | | | GPGLVKPSQTLSLTCAVSGGSVIRGYYWTWIRQRPGRGLEWMGY |
| | | | WAGSTDYNPAFQGRIFITAGTAKNQFSLEMTSMTTDDTAIYYCVG |
| | | | SLRGGWLFDNWGQGTLVTVSS |
| 41 | scFv 41 | DNA | CAGCCTGTGCTCACTCAGCCGCCCTCGGTCTCAGTGTCTCTGGG |
| | | | ACAGACAGCAACCATCTCCTGCTCTGGAGAGAGTCTGAGTAAA |
| | | | TATTATGCACAGTGGTTCCAGCAGAAGGCAGGCCAAGCCCCTG |
| | | | TCTTGGTCATATATAAGGACACTGAGCGGCCCTCTGGGATCCCT |
| | | | GACCGATTCTCTGGCTCCAGTTCAGGGAACACACACACCCTGA |
| | | | CCATCAGCGGGGCTCGGGCCGAGGACGAGGCTGACTATTACTG |
| | | | CGAGTCAGCAGTCAGTACTGAGACTGTTGTGTTCGGCGGAGGC |
| | | | ACCCATCTGACCGTCCTCGGCGGTGGTTCCTCTAGATCTTCCTC |
| | | | CTCTGGTGGCGGTGGCTCGGGCGGTGGTGGGGAGGTGCAGCTG |
| | | | GTGGAGACTGGGGGAGACCTGGTGATGCCTGCAGGGTCCCTGA |
| | | | GACTGTCTTGTGTGGCCTCTGGATTCACCTTCAGTAGTTCTGTC |
| | | | ATGAGCTGGGTCCGTCAGGCTCCTGGGAAGGGGCTGGAGTGGG |
| | | | TCGCAGGTATTAATGGTGGCGGAAGTTTTGCGGCCTACGGAGA |
| | | | CGCTGTGAAGGGCCGATTCACCATATCCAGAGACAACGCCAAG |
| | | | AACACGCTCTATCTGCAGATGAACAGCCTGAGAGCCGAGGACA |
| | | | CGGCCATGTATTACTGTGTGGGGGGCGGTTATATGGACTTCTG |
| | | | GGGTCCTGGCACCTCACTCTTCGTGTCCTCA |
| 84 | scFv 41 | AA | QPVLTQPPSVSVSLGQTATISCSGESLSKYYAQWFQQKAGQAPVL |
| | | | VIYKDTERPSGIPDRFSGSSSGNTHTLTISGARAEDEADYYCESAVS |
| | | | TETVVFGGGTHLTVLGGGSSRSSSSGGGGSGGGGEVQLVETGGDL |
| | | | VMPAGSLRLSCVASGFTFSSSVMSWVRQAPGKGLEWVAGINGGG |
| | | | SFAAYGDAVKGRFTISRDNAKNTLYLQMNSLRAEDTAMYYCVG |
| | | | GGYMDFWGPGTSLFVSS |
| 42 | scFv 42 | DNA | CAGTCTGTGCTGACTCAGCCGACCTCAGTGTCGGGGTCCCTTGG |
| | | | CCAGAGGGTCACCATTTCCTGCTCTGGAAGCACGAACGACATC |
| | | | GGTATTATTGGTGCGGCCTGGTACCAACAGCTCCCAGGGAAGG |
| | | | CCCCTAAACTCCTCCTGTACAGTAATAAGAATCGACCGTCAGG |
| | | | AGTCCCTGACCGATTTTCCGGCTCCAACTCTGGCATCTCAGCCA |
| | | | CCCTGACCATCACTGGGCTTCAGGCTGAGGACGAGGCTGATTA |
| | | | TTACTGTCAGTCTGTTGATCCCGCGCTTCATACTTACGTGTTTG |
| | | | GCTCAGGGACCCAGCTGACCATCCTCGGCGGTGGTTCCTCTAG |
| | | | ATCTTCCTCCTCTGGTGGCGGTGGCTCGGGCGGTGGTGGGGAG |
| | | | GTGCAGCTGGTGGAGATTGGGGGAGACCTTGTGAAACCTGAGG |
| | | | GGTCCCTGAGACTCTCCTGTGTGGTCTCTGGCTTCACCTTCAGT |
| | | | GGCTACGACATGAACTGGGTCCGCCAGGCTCCAGGGAAGGGG |
| | | | CTTCAGTGGGTCGCATACATTAGCAGTGATGGGAGGAGAACAG |
| | | | TTTACACAGACGCTGTGAAGGGCCGATTCACCATCTCCAGAGA |
| | | | CAATGCCAAGAACACGCTGTATCTGCAGATGAACAGCCTGAAA |
| | | | TCTGAGGACACAGCCGTATATTACTGTGTGAAGGGGGGTTGGT |
| | | | TGGACTTCTGGGGCCAGGGAACCCTGGTCACCGTCTCCTCA |
| 85 | scFv 42 | AA | QSVLTQPTSVSGSLGQRVTISCSGSTNDIGIIGAAWYQQLPGKAPK |
| | | | LLLYSNKNRPSGVPDRFSGSNSGISATLTITGLQAEDEADYYCQSV |
| | | | DPALHTYVFGSGTQLTILGGGSSRSSSSGGGGSGGGGEVQLVEIGG |
| | | | DLVKPEGSLRLSCVVSGFTFSGYDMNWVRQAPGKGLQWVAYISS |
| | | | DGRRTVYTDAVKGRFTISRDNAKNTLYLQMNSLKSEDTAVYYCV |
| | | | KGGWLDFWGQGTLVTVSS |
| 43 | scFv 43 | DNA | GATATTGTCATGACACAGAACCCACTGTCCCTGGCCGTCACCC |
| | | | CTGGAGAGCTGGCCACTATCTCCTGCAGGGCCAGTCAGAGTCT |
| | | | CCTGCACAGTGATGGAAAATCCTATTTGAATTGGTACCTGCAG |
| | | | AAGCCAGGCCAGACTCCTCGGCCGCTGATTTATGAGGCTTCCA |
| | | | GGCGTTTCTCTGGGGTCTCAGACAGGTTCAATGGCAGCGGGTC |
| | | | AGGGACAGATTTCACCCTTAAAATCAGTAGGGTGGAGGCTGAG |
| | | | GATGTTGGAGTTTATTACTGTCAGCAAGGTCTACATTTTCCTCC |
| | | | GACGTTCGGAGCAGGAACCAAGGTGGAGCTCAAAGGTGGTTCC |
| | | | TCTAGATCTTCCTCCTCTGGTGGCGGTGGCTCGGGCGGTGGTGG |
| | | | GGAGGTACAGCTGGTGGAATCTGGGGGAGACCTGGTGAAGCCT |
| | | | GGGGGGTCCCTGAGACTCTCCTGTGCAGCCTCTGGATTCACCTT |
| | | | CACTGACTACGACATGAGTTGGGTCCGCCAGGCTCCTGGGAAG |
| | | | GGGCTGCAGTGGGTCGCAGCTATTAGCTATGATGGAAGTAGCA |
| | | | CATACTACACTGACGCTGTGAAGGGCCGATTCACCATCTCCAG |
| | | | AGACAACGCCAGGAACACACTGTATCTGCAGATGAGCAGCCTG |
| | | | AGAGCCGAGGACACGGCCATGTATTACTGTGTAGTCTCGAATA |
| | | | TGAACCTCTGGGGCCATGGCACCTCAGTCTTCGTGTCCTCT |
| 86 | scFv 43 | AA | DIVMTQNPLSLAVTPGELATISCRASQSLLHSDGKSYLNWYLQKP |
| | | | GQTPRPLIYEASRRFSGVSDRFNGSGSGTDFTLKISRVEAEDVGVY |
| | | | YCQQGLHFPPTFGAGTKVELKGGSSRSSSSGGGGSGGGGEVQLVE |
| | | | SGGDLVKPGGSLRLSCAASGFTFTDYDMSWVRQAPGKGLQWVA |
| | | | AISYDGSSTYYTDAVKGRFTISRDNARNTLYLQMSSLRAEDTAMY |
| | | | YCVVSNMNLWGHGTSVFVSS |

TABLE 2
CDRs of Anti-CD19 scFvs

| SEQ | | | |
|---|---|---|---|
| ID | | | |
| NO: | Name: | Type: | Sequence: |
| 87 | scFv_01_H CDR1 | AA | GFTFSNHE |
| 88 | scFv_01_H CDR2 | AA | IYDSGSRT |
| 89 | scFv_01_H CDR3 | AA | AGGNSRGWTEFGMDY |
| 90 | scFv_01_L CDR1 | AA | NTNIGSPYD |
| | scFv_01_L CDR2 | AA | GNS |
| 91 | scFv_01_L CDR3 | AA | QSYDDSLDGRVV |
| 92 | scFv_02_H CDR1 | AA | GFSFSSYD |
| 93 | scFv_02_H CDR2 | AA | TNFDGSRT |
| 94 | scFv_02_H CDR3 | AA | ARGMGSWGASTFGY |
| 95 | scFv_02_L CDR1 | AA | RSNLGGNY |
| | scFv_02_L CDR2 | AA | AND |
| 96 | scFv_02_L CDR3 | AA | SSWDDDLGSHV |
| 97 | scFv_03_H CDR1 | AA | GFTFSNYT |
| 98 | scFv_03_H CDR2 | AA | INYDGSIT |
| 99 | scFv_03_H CDR3 | AA | VKETFMIPTE |
| 100 | scFv_03_L CDR1 | AA | HTNIGSGFD |
| | scFv_03_L CDR2 | AA | ANI |
| 101 | scFv_03_L CDR3 | AA | QSYDDNFDGHV |
| 102 | scFv_04_H CDR1 | AA | GFDFSRYG |
| 103 | scFv_04_H CDR2 | AA | ISYRGTTF |
| 104 | scFv_04_H CDR3 | AA | NSANWYVSPFDH |
| 100 | scFv_04_L CDR1 | AA | HTNIGSGFD |
| | scFv_04_L CDR2 | AA | ANI |
| 105 | scFv_04_L CDR3 | AA | QSYDDNFDYV |
| 106 | scFv_05_H CDR1 | AA | GFPFSGYS |
| 107 | scFv_05_H CDR2 | AA | INSDGSHT |
| 108 | scFv_05_H CDR3 | AA | GSHSWGNFHY |
| 109 | scFv_05_L CDR1 | AA | TNTIGVVG |
| | scFv_05_L CDR2 | AA | SDG |
| 110 | scFv_05_L CDR3 | AA | QSFDSTHNAIV |
| 111 | scFv_06_H CDR1 | AA | GFTFSDNY |
| 112 | scFv_06_H CDR2 | AA | ISTDGRGT |
| 113 | scFv_06_H CDR3 | AA | ARRSGIWGFDY |
| 114 | scFv_06_L CDR1 | AA | NTNIGSGYS |
| | scFv_06_L CDR2 | AA | GSS |
| 115 | scFv_06_L CDR3 | AA | QSYDDSLDGHAV |
| 116 | scFv_07_H CDR1 | AA | GFTFSNYG |
| 117 | scFv_07_H CDR2 | AA | INSGGSTT |
| 118 | scFv_07_H CDR3 | AA | ARVDYSSSWLVDY |
| 119 | scFv_07_L CDR1 | AA | NTNIGSGYD |
| | scFv_07_L CDR2 | AA | GNT |
| 120 | scFv_07_L CDR3 | AA | QSYDDNFDGHHV |
| 121 | scFv_08_H CDR1 | AA | GFTFSSYG |
| 122 | scFv_08_H CDR2 | AA | ISYGGGTY |
| 123 | scFv_08_H CDR3 | AA | SQWGDWGHFEY |
| 124 | scFv_08_L CDR1 | AA | RSNIGANY |
| | scFv_08_L CDR2 | AA | GYN |
| 125 | scFv_08_L CDR3 | AA | SSWDDSLRGTV |
| 126 | scFv_09_H CDR1 | AA | GFTFSSDS |
| 117 | scFv_09_H CDR2 | AA | INSGGSTT |
| 127 | scFv_09_H CDR3 | AA | AKDSGFGSWYLES |
| 128 | scFv_09_L CDR1 | AA | KTNIGSGYD |
| | scFv_09_L CDR2 | AA | GNS |
| 129 | scFv_09_L CDR3 | AA | QSFDDNVDGYV |
| 130 | scFv_10_H CDR1 | AA | GLTFNRNY |
| 131 | scFv_10_H CDR2 | AA | INPDGSTT |
| 132 | scFv_10_H CDR3 | AA | ARSSWGSFDY |
| 100 | scFv_10_L CDR1 | AA | HTNIGSGFD |
| | scFv_10_L CDR2 | AA | ANI |
| 133 | scFv_10_L CDR3 | AA | QSFDDDLDGPV |
| 134 | scFv_11_H CDR1 | AA | GFTFSDYS |
| 135 | scFv_11_H CDR2 | AA | IMTSGSTY |
| 136 | scFv_11_H CDR3 | AA | HDYWFDF |
| 100 | scFv_11_L CDR1 | AA | HTNIGSGFD |
| | scFv_11_L CDR2 | AA | ANI |
| 105 | scFv_11_L CDR3 | AA | QSYDDNFDYV |
| 137 | scFv_12_H CDR1 | AA | GFTFSSYD |
| 138 | scFv_12_H CDR2 | AA | ISYDGSST |
| 139 | scFv_12_H CDR3 | AA | AKDSGSWSHFYGLDT |
| 140 | scFv_12_L CDR1 | AA | NIGRKS |
| | scFv_12_L CDR2 | AA | ADS |
| 141 | scFv_12_L CDR3 | AA | QIWDRNINV |
| 142 | scFv_13_H CDR1 | AA | GFTFSRYY |
| 143 | scFv_13_H CDR2 | AA | ISSDGRRT |
| 144 | scFv_13_H CDR3 | AA | VREDYFDFDY |
| 100 | scFv_13_L CDR1 | AA | HTNIGSGFD |
| | scFv_13_L CDR2 | AA | ANI |
| 105 | scFv_13_L CDR3 | AA | QSYDDNFDYV |
| 145 | scFv_14_H CDR1 | AA | GFAFSGKT |
| 146 | scFv_14_H CDR2 | AA | IQHDGSRT |
| 147 | scFv_14_H CDR3 | AA | ADHPWYGPEY |
| 148 | scFv_14_L CDR1 | AA | HTNIGSGYD |
| | scFv_14_L CDR2 | AA | GNS |
| 149 | scFv_14_L CDR3 | AA | QSYDDNLNDPYV |
| 150 | scFv_15_H CDR1 | AA | GFTFNKYH |
| 151 | scFv_15_H CDR2 | AA | ITSDGSAT |
| 152 | scFv_15_H CDR3 | AA | ARDQSSWGTGNFDY |
| 153 | scFv_15_L CDR1 | AA | DTNIGNHYD |
| | scFv_15_L CDR2 | AA | GNS |
| 154 | scFv_15_L CDR3 | AA | QSYDDDLDGHIV |
| 155 | scFv_16_H CDR1 | AA | GFTLSSYW |
| 156 | scFv_16_H CDR2 | AA | ISGSGNTI |
| 157 | scFv_16_H CDR3 | AA | ATEYYWNIDN |
| 158 | scFv_16_L CDR1 | AA | SSNVGYGNY |
| | scFv_16_L CDR2 | AA | GSH |
| 159 | scFv_16_L CDR3 | AA | SSYDSTLSGYV |
| 121 | scFv_17_H CDR1 | AA | GFTFSSYG |
| 160 | scFv_17_H CDR2 | AA | INGGGSST |
| 161 | scFv_17_H CDR3 | AA | SNYYGIYWDSDY |
| 128 | scFv_17_L CDR1 | AA | KTNIGSGYD |
| | scFv_17_L CDR2 | AA | GNS |
| 162 | scFv_17_L CDR3 | AA | QSFDDNVDGYV |
| 163 | scFv_18_H CDR1 | AA | GFTLSNYG |
| 164 | scFv_18_H CDR2 | AA | IRSGGNTY |
| 123 | scFv_18_H CDR3 | AA | SQWGDWGHFEY |
| 124 | scFv_18_L CDR1 | AA | RSNIGANY |
| | scFv_18_L CDR2 | AA | GYN |
| 165 | scFv_18_L CDR3 | AA | STWDSSLNAYV |
| 106 | scFv_19_H CDR1 | AA | GFPFSGYS |
| 107 | scFv_19_H CDR2 | AA | INSDGSHT |
| 108 | scFv_19_H CDR3 | AA | GSHSWGNFHY |
| 166 | scFv_19_L CDR1 | AA | TNNIGIVG |
| | scFv_19_L CDR2 | AA | SDG |
| 167 | scFv_19_L CDR3 | AA | QSVDATHGTIL |
| 168 | scFv_20_H CDR1 | AA | GFTFSSYA |
| 169 | scFv_20_H CDR2 | AA | INSAGSTT |
| 170 | scFv_20_H CDR3 | AA | GRYYYGLDY |
| 171 | scFv_20_L CDR1 | AA | SLSKYY |
| | scFv_20_L CDR2 | AA | KDT |
| 172 | scFv_20_L CDR3 | AA | ESEVSPDTIV |
| 173 | scFv_21_H CDR1 | AA | GFTFSSFW |
| 174 | scFv_21_H CDR2 | AA | INSGGSIT |
| 175 | scFv_21_H CDR3 | AA | AKPDIYGSFDH |
| 171 | scFv_21_L CDR1 | AA | SLSKYY |
| | scFv_21_L CDR2 | AA | KDT |
| 176 | scFv_21_L CDR3 | AA | ESAVSSDTAV |
| 177 | scFv_22_H CDR1 | AA | GDSVTSSHF |
| 178 | scFv_22_H CDR2 | AA | WSDIT |
| 179 | scFv_22_H CDR3 | AA | ARYNGYVDYLDV |
| 180 | scFv_22_L CDR1 | AA | SSNVGYGDY |
| | scFv_22_L CDR2 | AA | SSS |
| 181 | scFv_22_L CDR3 | AA | SSYDTSLNGFV |
| 182 | scFv_23_H CDR1 | AA | GGSVTTSDY |
| 183 | scFv_23_H CDR2 | AA | WKGRT |
| 184 | scFv_23_H CDR3 | AA | AHWGSGNLDY |
| 185 | scFv_23_L CDR1 | AA | QSLLHSNGNTY |
| | scFv_23_L CDR2 | AA | LVS |
| 186 | scFv_23_L CDR3 | AA | GQGKQDPIT |
| 187 | scFv_24_H CDR1 | AA | GFTLSSNY |
| 188 | scFv_24_H CDR2 | AA | ISTDGSRT |
| 189 | scFv_24_H CDR3 | AA | AKNEYLAPTY |
| 100 | scFv_24_L CDR1 | AA | HTNIGSGFD |
| | scFv_24_L CDR2 | AA | ANI |
| 190 | scFv_24_L CDR3 | AA | QSYDDNVDGYV |
| 191 | scFv_25_H CDR1 | AA | GFTFSSLS |
| 192 | scFv_25_H CDR2 | AA | ISPDGTIT |
| 193 | scFv_25_H CDR3 | AA | AKSRYTDTYPFDY |
| 185 | scFv_25_L CDR1 | AA | QSLLHSNGNTY |
| | scFv_25_L CDR2 | AA | QVS |
| 194 | scFv_25_L CDR3 | AA | GQGIQYPPT |
| 195 | scFv_26_H CDR1 | AA | GFTFSSYW |
| 196 | scFv_26_H CDR2 | AA | ISDSGSST |
| 197 | scFv_26_H CDR3 | AA | ARGLGMDY |
| 171 | scFv_26_L CDR1 | AA | SLSKYY |
| | scFv_26_L CDR2 | AA | KDT |
| 198 | scFv_26_L CDR3 | AA | ESLVDSDAYM |
| 195 | scFv_27_H CDR1 | AA | GFTFSSYW |
| 196 | scFv_27_H CDR2 | AA | ISDSGSST |
| 197 | scFv_27_H CDR3 | AA | ARGLGMDY |
| 171 | scFv_27_L CDR1 | AA | SLSKYY |
| | scFv_27_L CDR2 | AA | KDT |
| 199 | scFv_27_L CDR3 | AA | HSYDENLDGYV |
| 195 | scFv_28_H CDR1 | AA | GFTFSSYW |
| 196 | scFv_28_H CDR2 | AA | ISDSGSST |
| 197 | scFv_28_H CDR3 | AA | ARGLGMDY |
| 200 | scFv_28_L CDR1 | AA | SLNKYY |
| | scFv_28_L CDR2 | AA | RDT |
| 201 | scFv_28_L CDR3 | AA | ESLVNDNTYV |
| 195 | scFv_29_H CDR1 | AA | GFTFSSYW |
| 196 | scFv_29_H CDR2 | AA | ISDSGSST |
| 197 | scFv_29_H CDR3 | AA | ARGLGMDY |
| 200 | scFv_29_L CDR1 | AA | SLNKYY |
| | scFv_29_L CDR2 | AA | KDT |
| 202 | scFv_29_L CDR3 | AA | ESVDSTDTYV |
| 195 | scFv_30_H CDR1 | AA | GFTFSSYW |
| 196 | scFv_30_H CDR2 | AA | ISDSGSST |
| 197 | scFv_30_H CDR3 | AA | ARGLGMDY |
| 171 | scFv_30_L CDR1 | AA | SLSKYY |
| | scFv_30_L CDR2 | AA | KDT |
| 190 | scFv_30_L CDR3 | AA | QSYDDNVDGYV |
| 195 | scFv_31_H CDR1 | AA | GFTFSSYW |
| 196 | scFv_31_H CDR2 | AA | ISDSGSST |
| 197 | scFv_31_H CDR3 | AA | ARGLGMDY |
| 203 | scFv_31_L CDR1 | AA | SLTKYY |
| | scFv_31_L CDR2 | AA | RDT |
| 204 | scFv_31_L CDR3 | AA | ESMDGREAYV |
| 195 | scFv_32_H CDR1 | AA | GFTFSSYW |
| 196 | scFv_32_H CDR2 | AA | ISDSGSST |
| 197 | scFv_32_H CDR3 | AA | ARGLGMDY |
| 171 | scFv_32_L CDR1 | AA | SLSKYY |
| | scFv_32_L CDR2 | AA | KDT |
| 205 | scFv_32_L CDR3 | AA | QSYDDNLDGYV |
| 206 | scFv_33_H CDR1 | AA | GFTFSHYD |
| 207 | scFv_33_H CDR2 | AA | IAYDGSNT |
| 208 | scFv_33_H CDR3 | AA | ARGLVY |
| 209 | scFv_33_L CDR1 | AA | QSLLHSDGNTY |
| | scFv_33_L CDR2 | AA | SVS |
| 210 | scFv_33_L CDR3 | AA | GQGTHLPFT |
| 211 | scFv_34_H CDR1 | AA | GLTFSDNY |
| 112 | scFv_34_H CDR2 | AA | ISTDGRGT |
| 113 | scFv_34_H CDR3 | AA | ARRSGIWGFDY |
| 114 | scFv_34_L CDR1 | AA | NTNIGSGYS |
| | scFv_34_L CDR2 | AA | GSS |
| 115 | scFv_34_L CDR3 | AA | QSYDDSLDGHAV |
| 168 | scFv_35_H CDR1 | AA | GFTFSSYA |
| 117 | scFv_35_H CDR2 | AA | INSGGSTT |
| 212 | scFv_35_H CDR3 | AA | ARWWDFEY |
| 213 | scFv_35_L CDR1 | AA | SLSEYY |
| | scFv_35_L CDR2 | AA | KDT |
| 214 | scFv_35_L CDR3 | AA | ESAVNSDGIV |
| 121 | scFv_36_H CDR1 | AA | GFTFSSYG |
| 215 | scFv_36_H CDR2 | AA | ISDTGSTY |
| 216 | scFv_36_H CDR3 | AA | KDLGVWGMDN |
| 166 | scFv_36_L CDR1 | AA | TNNIGIVG |
| | scFv_36_L CDR2 | AA | SDG |
| 217 | scFv_36_L CDR3 | AA | QSFDTTLDAYV |
| 218 | scFv_37_H CDR1 | AA | GFTLRDYS |
| 219 | scFv_37_H CDR2 | AA | ISTGGSTT |
| 220 | scFv_37_H CDR3 | AA | TTTALGY |
| 221 | scFv_37_L CDR1 | AA | SLNDYY |
| | scFv_37_L CDR2 | AA | RDT |
| 222 | scFv_37_L CDR3 | AA | ESEVSAGGAV |
| 223 | scFv_38_H CDR1 | AA | GFNFGNYA |
| 224 | scFv_38_H CDR2 | AA | ISNDGTIT |
| 225 | scFv_38_H CDR3 | AA | VAGGFLDY |
| 213 | scFv_38_L CDR1 | AA | SLSEYY |
| | scFv_38_L CDR2 | AA | KDT |
| 226 | scFv_38_L CDR3 | AA | ASADSSAIPV |
| 121 | scFv_39_H CDR1 | AA | GFTFSSYG |
| 227 | scFv_39_H CDR2 | AA | ITYDGSNT |
| 228 | scFv_39_H CDR3 | AA | VASPLYGTYGFDY |
| 229 | scFv_39_L CDR1 | AA | TKNIGIVG |
| | scFv_39_L CDR2 | AA | SDG |
| 230 | scFv_39_L CDR3 | AA | QSFDTTLDAHV |
| 231 | scFv_40_H CDR1 | AA | GGSVIRGYY |
| 232 | scFv_40_H CDR2 | AA | WAGST |
| 233 | scFv_40_H CDR3 | AA | VGSLRGGWLFDN |
| 234 | scFv_40_L CDR1 | AA | SSSVGTHY |
| | scFv_40_L CDR2 | AA | NND |
| 235 | scFv_40_L CDR3 | AA | SSWDDSLGAYV |
| 236 | scFv_41_H CDR1 | AA | GFTFSSSV |
| 237 | scFv_41_H CDR2 | AA | INGGGSFA |
| 238 | scFv_41_H CDR3 | AA | VGGGYMDF |
| 171 | scFv_41_L CDR1 | AA | SLSKYY |
| | scFv_41_L CDR2 | AA | KDT |
| 239 | scFv_41_L CDR3 | AA | ESAVSTETVV |
| 240 | scFv_42_H CDR1 | AA | GFTFSGYD |
| 143 | scFv_42_H CDR2 | AA | ISSDGRRT |
| 241 | scFv_42_H CDR3 | AA | VKGGWLDF |
| 242 | scFv_42_L CDR1 | AA | TNDIGIIG |
| | scFv_42_L CDR2 | AA | SNK |
| 243 | scFv_42_L CDR3 | AA | QSVDPALHTYV |
| 244 | scFv_43_H CDR1 | AA | GFTFTDYD |
| 138 | scFv_43_H CDR2 | AA | ISYDGSST |
| 245 | scFv_43_H CDR3 | AA | VVSNMNL |
| 246 | scFv_43_L CDR1 | AA | QSLLHSDGKSY |
| | scFv_43_L CDR2 | AA | EAS |
| 247 | scFv_43_L CDR3 | AA | QQGLHFPPT |

TABLE 3
Example Amino Acid Sequences
of Anti-CD19 Heavy and Light Chains

| SEQ | | |
|---|---|---|
| ID | | |
| NO: | Name: | Sequence: |
| 271 | scFv_01 | QAVLNQPASVSAALGQRVTISCNTNIGSPYDVQWYQQLPGKSPKTIIYG |
| | VL | NSNRPSGVPVRFSGSKSGSTATLTIAGIQAEDEADYYCQSYDDSLDGRV |
| | | VFGGGTQLTVL |
| 272 | scFv_02 | SYVLTQSPSLSGSLGQKVTISCTGSRSNLGGNYVGWYQQVPGMAPRN |
| | VL | VIYANDFRPSRIPARFSASKSGTSATLTISGLQAEDEADYFCSSWDDDL |
| | | GSHVFGSGTQLTIL |
| 273 | scFv_03 | QSVLNQPPSVSGALGQTVTISCSGSHTNIGSGFDVQWYQQLPGKSPQTL |
| | VL | VYANINRPSGVPARFSGSKSGSTATLTITGVQAEDEADYYCQSYDDNF |
| | | DGHVFGSGTQLTVL |
| 274 | scFv_04 | QSVLNQPPSVSGALGQTVTISCSGSHTNIGSGFDVQWYQQLPGKSPQTII |
| | VL | YANINRPSGVPARFSGSKSGSTATLTITGVQAEDEADYYCQSYDDNFD |
| | | YVFGSGTQLTVL |
| 275 | scFv_05 | QPVLTQPPSVSGSLGQRVTISCSGSTNTIGVVGANWYQQLPGKSPKLLV |
| | VL | YSDGVRPSGVPDRFSGSRSANSDTLTITGLQAEDEADYYCQSFDSTHN |
| | | AIVFGGGTHLTVL |
| 276 | scFv_06 | QPVLTQPPSVSGSLGQRVTISCTGTNTNIGSGYSVQWYQQLPGESPKPII |
| | VL | YGSSNRPSGVPARFSGSKSGSTGTLTITGIQAEDEADYHCQSYDDSLDG |
| | | HAVFGGGTQLTIL |
| 277 | scFv_07 | SYELTQPPSMSAALGQRVTISCTGSNTNIGSGYDVQWYQQVPGKSPTP |
| | VL | LIYGNTNRPSGVPVRFSGSKSGNTATLTITGIQAEDEADYYCQSYDDNF |
| | | DGHHVFGSGTQLTVL |
| 278 | scFv_08 | QSVLTQPASVSGSLGQKVTISCTGSRSNIGANYVGWYQQVPGIGPRTVI |
| | VL | FGYNYRPSGVPDRFSSSKSGNSATLTISGLQAEDEAEYYCSSWDDSLRG |
| | | TVFGSGTQLTVL |
| 279 | scFv_09 | QPVLTQPPSVSAALGQRVTLSCTGSKTNIGSGYDVQWYQQFPGKSPKTI |
| | VL | IYGNSNRFSGVPARFSGSKSGSIATLTITGIQAEDEADYYCQSFDDNVD |
| | | GYVFGSGTQLTIL |
| 280 | scFv_10 | QAVLNQPASVSGALGQTVTISCSGSHTNIGSGFDVQWYQQLPGKSPQTI |
| | VL | IYANINRPSGVPARFSGSKSGSTATLTITGVQAEDEADYHCQSFDDDLD |
| | | GPVFGGGTHLTVL |
| 281 | scFv_11 | QPVLTQPPSVSGALGQTVTISCSGSHTNIGSGFDVQWYQQLPGKSPQTL |
| | VL | VYANINRPSGVPARFSGSKSGSTATLTITGVQAEDEADYYCQSYDDNF |
| | | DYVFGSGTQLTVL |
| 282 | scFv_12 | SYELTQPPSATVTLRQTARLTCGGENIGRKSVQWYQQKPGQSPLLIIYA |
| | VL | DSSRPSGIPERFSGANSGNTASLTISGALAEDEADYYCQIWDRNINVFGS |
| | | GTQLTVL |
| 283 | scFv_13 | QPVLTQPPSVSGALGQTVTISCSGSHTNIGSGFDVQWYQQLPGKSPQTL |
| | VL | VYANINRPSGVPARFSGSKSGSTATLTITGVQAEDEADYYCQSYDDNF |
| | | DYVFGSGTQLTVL |
| 284 | scFv_14 | QSVLNQPPSVSAALGQRITISCTGSHTNIGSGYDVQWYQQLPGKSPRTII |
| | VL | YGNSDRPSGVPVRFSGSKSGNTATLTITGIQAEDEADYYCQSYDDNLN |
| | | DPYVFGSGTQLTVL |
| 285 | scFv_15 | SYELTQPPSVSAALGQGVTISCIGSDTNIGNHYDVQWYQQLPGKSPRTII |
| | VL | YGNSDRPSGVPGRFSGSKSGNTATLTITGIQAEDEADYHCQSYDDDLD |
| | | GHIVFGGGTHLTVL |
| 286 | scFv_16 | QSVLTQPTSVSGSLGQRVTISCTGSSSNVGYGNYVGWYQQLPGTGPRT |
| | VL | LIYGSHYRPSGVPDRFSGSSSGSSATLTISGLQAEDEADYYCSSYDSTLS |
| | | GYVFGSGTQLTIL |
| 287 | scFv_17 | QPVLTQPPSVSAALGQRVTISCTGSKTNIGSGYDVQWYQQLPGKSPKTI |
| | VL | IYGNSNRPSGVPARFSGSKSGSIATLTITGIQAEDEADYYCQSFDDNVD |
| | | GYVFGSGTQLTIL |
| 288 | scFv_18 | QSALTQTASVSGSLGQKVTISCTGSRSNIGANYVGWYQQVPGIGPRTVI |
| | VL | FGYNYRPSGVPDRFSSSKSGNSATLTISGLQAEDEAEYYCSTWDSSLNA |
| | | YVFGSGTQLTVL |
| 289 | scFv_19 | QAVLTQPASVSGPLGQRVTISCSGSTNNIGIVGAAWYQQFPGKAPKLL |
| | VL | VYSDGSRPSGVPDRFSGSNSGNSATLTISGLQAEDEADYYCQSVDATH |
| | | GTILFGGGTQLTVL |
| 290 | scFv_20 | QSVLNQPPSVSVSLGQTATISCSGESLSKYYAQWFQQKAGQVPVLVIY |
| | VL | KDTERPSGIPDRFSGSSSGNTHTLTISGARAEDEADYYCESEVSPDTIVF |
| | | GGGTHLTVL |
| 291 | scFv_21 | QPVLTQPPSVSVSLGQTATISCSGESLSKYYAQWFQQKAGQAPVLVIY |
| | VL | KDTERPSGIPDRFSGSSSGNTHTLTISGARAEDEADYYCESAVSSDTAVF |
| | | GSGTQLTVL |
| 292 | scFv_22 | QPVLTQPPSVSGSLGQRVTISCTGSSSNVGYGDYVGWYQQLPGTSPRT |
| | VL | LIYSSSSRPSGVPDRFSGSRSGSTATLTISGLQAEDEADYHCSSYDTSLN |
| | | GFVFGSGTQLTIL |
| 293 | scFv_23 | DIVMTQTPLSLSVSPGEPASISCKASQSLLHSNGNTYLYWFRQKPGQSP |
| | VL | QRLIYLVSNRDAGVPDRFSGSGSGTDFTLRISRVEADDSGIYYCGQGKQ |
| | | DPITFGEGTHLEIK |
| 294 | scFv_24 | QSVLNQPPSVSGALGQTVTISCSGSHTNIGSGFDVQWYQQLPGKSPQTL |
| | VL | VYANINRPSGVPVRFSGSKSGTTATLTITGIQAEDEADYYCQSYDDNVD |
| | | GYVFGSGTQLTVL |
| 295 | scFv_25 | DIVMTQTPLSLSVSPGETASISCKASQSLLHSNGNTYLNWFRQKPGQSP |
| | VL | QVLIYQVSNRDTGVPDRFSGSGSGTDFTLRISRVEADDAGVYYCGQGI |
| | | QYPPTFGAGTKVELK |
| 296 | scFv_26 | SSVLTQPPSVSVSLGQTATISCSGESLSKYYTQWFQQKAGQAPILVIYK |
| | VL | DTERPSGIPDRFSGSSSGNTHTLTISGARAEDEADYYCESLVDSDAYMF |
| | | GSGTQLTVL |
| 297 | scFv_27 | QPVLTQPPSVSVSLGQTATISCSGESLSKYYAQWFQQKAGQAPVLVIY |
| | VL | KDTERPSGIPDRFSGSSSGNTHTLTITGIQAEDEGDYHCHSYDENLDGY |
| | | VFGSGTQLTVL |
| 298 | scFv_28 | QSALTQPSSVSVSLGQTATISCSGESLNKYYVQWFQQKAGQVPILVIYR |
| | VL | DTERPSGIPDRFSGSSSGNTHTLTITGTRAEDEADYYCESLVNDNTYVF |
| | | GSGTQLTVL |
| 299 | scFv_29 | QSVLNQPPSVSVSLGQTATISCSGESLNKYYTQWFQQKAGQAPVLVIY |
| | VL | KDTERPSGIPDRFSGSSSGNTHTLTISGARAEDEADYYCESVDSTDTYV |
| | | FGSGTQLTIL |
| 300 | scFv_30 | QSVLTQPASVSVSLGQTATISCSGESLSKYYTQWFQQKAGQAPVLVIY |
| | VL | KDTERPSGIPDRFSGSSSGNTHTLTISGAQAEDEADYYCQSYDDNVDG |
| | | YVFGSGTQLTVL |
| 301 | scFv_31 | QSALTQPSSVSVSLGQTATISCSGESLTKYYVQWFQQKAGQAPVLVIY |
| | VL | RDTERPSGIPDRFSGSSSGNTHTLTISGARAEDEADYYCESMDGREAYV |
| | | FGSGTQLTVL |
| 302 | scFv_32 | QSVLTQPASVSVSLGQTATISCSGESLSKYYAQWFQQKAGQAPVLVIY |
| | VL | KDTERPSGIPDRFSGSSSGNTHTLTITGIQAADEADYYCQSYDDNLDGY |
| | | VFGSGTQLTVL |
| 303 | scFv_33 | DIVMTQTPLSLSVSPGEPASISCKASQSLLHSDGNTYLHWYLQKPGQSP |
| | VL | QLLIYSVSNRFTGVSDRFSGSGSGTDFTLRISRVEADDTGVYYCGQGTH |
| | | LPFTFGQGTKLEIK |
| 304 | scFv_34 | SYELTQPPSVSAALGQRVTISCTGTNTNIGSGYSVQWYQQLPGESPKPII |
| | VL | YGSSNRPSGVPARFSGSKSGSTGTLTITGIQAEDEADYHCQSYDDSLDG |
| | | HAVFGGGTQLTIL |
| 305 | scFv_35 | SSVLTQPPSVSVSLGQTATISCSGESLSEYYAQWFQQKAGQAPVLVIYK |
| | VL | DTERASGIPDRFSGSSSGNTHTLTISGARAEDEADYYCESAVNSDGIVF |
| | | GGGTHLTVL |
| 306 | scFv_36 | QSVLTQPTSVSGSLGQRVTISCSGSTNNIGIVGASWYQQLPGKAPKLLV |
| | VL | YSDGDRPSGVPDRFSGSNSGNSDTLTITGLQAEDEADYYCQSFDTTLD |
| | | AYVFGSGTQLTVL |
| 307 | scFv_37 | SSVLTQPPSVSVSLGQTATISCSGESLNDYYAQWFQQKAGQVPVLVIYR |
| | VL | DTERPSGIPDRFSGSSSGNTHTLTISGARAEDEADYYCESEVSAGGAVF |
| | | GEGTHLTVL |
| 308 | scFv_38 | SSVLTQPPSVSVSLGQTATISCSGESLSEYYAQWFQQKAGQAPVLVIYK |
| | VL | DTERPSGIPDRFSGSSSGNTHTLTISGARAEDEADYYCASADSSAIPVFG |
| | | GGTHLTVL |
| 309 | scFv_39 | QSVLTQPPSVSGSPGQRVTISCSGTTKNIGIVGASWYQQLPGKAPKLLV |
| | VL | HSDGFRPSGVPDRFSASNSGDSDILTITGLQAEDEADYYCQSFDTTLDA |
| | | HVFGSGTQLTVL |
| 310 | scFv_40 | QSVLTQPPSVSGSLGQKVTISCTGSSSSVGTHYVGWYQIPPGIGPRTVIY |
| | VL | NNDYRPSGVPDRFSGSKSGNSATLTISGLQAEDEADYYCSSWDDSLGA |
| | | YVLGSGTQLTIL |
| 311 | scFv_41 | QPVLTQPPSVSVSLGQTATISCSGESLSKYYAQWFQQKAGQAPVLVIY |
| | VL | KDTERPSGIPDRFSGSSSGNTHTLTISGARAEDEADYYCESAVSTETVVF |
| | | GGGTHLTVL |
| 312 | scFv_42 | QSVLTQPTSVSGSLGQRVTISCSGSTNDIGIIGAAWYQQLPGKAPKLLL |
| | VL | YSNKNRPSGVPDRFSGSNSGISATLTITGLQAEDEADYYCQSVDPALHT |
| | | YVFGSGTQLTIL |
| 313 | scFv_43 | DIVMTQNPLSLAVTPGELATISCRASQSLLHSDGKSYLNWYLQKPGQT |
| | VL | PRPLIYEASRRFSGVSDRENGSGSGTDFTLKISRVEAEDVGVYYCQQGL |
| | | HFPPTFGAGTKVELK |
| 314 | scFv_01 | EVQLVESGGDLVKPGGSLRLSCVASGFTFSNHEMYWVRQAPGKGLEW |
| | VH | VARIYDSGSRTRYADAVKGRFTISRDNAKNTLYLQMNSLTAEDTAVYF |
| | | CAGGNSRGWTEFGMDYWGPGTLVTVSS |
| 315 | scFv_02 | EEQLVEFGGDLVKPGGSLRLSCVASGFSFSSYDMSWVRQAPGKGLQW |
| | VH | VASTNFDGSRTYYTDAVEGRFTISRDNARNTVYLQMNSLRAEDTAVY |
| | | YCARGMGSWGASTFGYWGQGTLVTVSS |
| 316 | scFv_03 | EVQLVQSGGDLVKPGGSLRLSCVASGFTFSNYTMSWVRQAPGQGLQW |
| | VH | VAYINYDGSITYYAEAVKGRFTVSRDNAKNTLYLQMNSLRAEDTAVY |
| | | FCVKETFMIPTEWGQGTLITVSS |
| 317 | scFv_04 | EEQLVEFGGDLVKPGGSLRLSCVVSGFDFSRYGMSWVRQSPGKGLQW |
| | VH | VADISYRGTTFYADDVKGRFTVSRDSAKNTLYLEMTSLTVEDTAVYY |
| | | CANSANWYVSPFDHWGQGTLVTVSS |
| 318 | scFv_05 | EVQLVETGGDLVKPAGSLRLSCAASGFPFSGYSMTWVRQAPEKGLQL |
| | VH | VAGINSDGSHTYYTDDVKGRFTISRDNTKYILYLQMNSLRAEDTAMYY |
| | | CGSHSWGNFHYWGQGTLVTVSS |
| 319 | scFv_06 | EVQLVQSGGDLVKPGGSLRLSCVASGFTFSDNYMSWIRQVPGKGLQW |
| | VH | VSRISTDGRGTSYADAVKGRFTISRDNAKNTLYLQMNSLRDEDTAVYY |
| | | CARRSGIWGFDYWGQGALVTVSS |
| 320 | scFv_07 | EVQLVESGGDLVKPAGSLRLSCVASGFTFSNYGMNWVRQAPGKGLQ |
| | VH | WVAGINSGGSTTTYADVVKGRFTISRDNAKNTLYLQMNSLGAEDTAV |
| | | YYCARVDYSSSWLVDYWGQGTLVTVSS |
| 321 | scFv_08 | EVQLVESGGDLVKPGESLRLSCVASGFTFSSYGMSWVRQSPGKGLQW |
| | VH | VADISYGGGTYYADAVKGRFTISRDNARNTVYLQMNSLRAEDTAVYY |
| | | CASQWGDWGHFEYWGQGTLVTVSS |
| 322 | scFv_09 | EVQLVESGGDLVKPGGSLRLSCVASGFTFSSDSIHWVRQAPGKGLQWV |
| | VH | AYINSGGSTTYYADAVKGRFTISRDNARNTVYLKMNSLRAEDTAVYY |
| | | CAKDSGFGSWYLESWGQGTLVTVSP |
| 323 | scFv_10 | EVQLVQSGGDLVKPGGSLKISCVASGLTFNRNYMTWIRQAPGKGLQW |
| | VH | VSEINPDGSTTSYTDAVRGRFTISRDNAKNTLYLQMNSMRDEDTAVYY |
| | | CARSSWGSFDYWGQGTLVTVSS |
| 324 | scFv_11 | EVQLVESGGDLVKPGGSLRLSCVASGFTFSDYSMSWVRQSPGKGLQW |
| | VH | VAAIMTSGSTYYADAVKGRFTISRDNAKNTLYLQMDSLRAEDTAVYY |
| | | CAHDYWFDFWGQGTLVTVSS |
| 325 | scFv_12 | EEQLVEFGGDLVKPAGSLRLSCVASGFTFSSYDMSWVRQAPGKGLQW |
| | VH | VATISYDGSSTYYTDAVKGRFTVSRDNAKNTLYLQMNSLRAEDTALY |
| | | YCAKDSGSWSHFYGLDTWGHGTSVFVSS |
| 326 | scFv_13 | EVQLVESGGGLMKPGGSLKLSCLASGFTFSRYYMYWVRQAPGKGLQ |
| | VH | WVAQISSDGRRTYYADAVQGRFTISRDNVKNTVYLQMNSLSDEDTAV |
| | | YYCVREDYFDFDYWGQGTLVTVSS |
| 327 | scFv_14 | EVQLVQSGGDLVKPGGSLRLSCVASGFAFSGKTMQWVRQAPGKGLQ |
| | VH | WVAYIQHDGSRTSYADAVKGRFTISRDNAKNTLYLQMDSLRAEDTAV |
| | | YYCADHPWYGPEYWGQGTPVTVSS |
| 328 | scFv_15 | EVQLVQSGGDLVKPGGSLRLSCVASGFTFNKYHMSWVRQAPGKGLQ |
| | VH | WVGKITSDGSATGYADAVKGRFTISRDNAKNTFYLQMDSLRAEDTAM |
| | | YYCARDQSSWGTGNFDYWGQGTLVTVSS |
| 329 | scFv_16 | EVQLVESGGDLVKPGGSLRLSCVGSGFTLSSYWMSWVRQAPGKGLQ |
| | VH | WIAEISGSGNTIYYGDAVKGRFTISRDNAKNTLYLQMNSLRDEDTAVY |
| | | YCATEYYWNIDNWGQGTLVTVSS |
| 330 | scFv_17 | EVPLVESGGDLVKPGGSLRLSCVASGFTFSSYGMSWVRQAPGKGLQW |
| | VH | VAYINGGGSSTSYADAVRGRFTISRDNAENTLYLQMNSLRADDTAVY |
| | | YCSNYYGIYWDSDYWGQGTLVTVSS |
| 331 | scFv_18 | EVPLVESGGDLVKPGGSLRLSCVASGFTLSNYGMSWVRQSPGKGLQW |
| | VH | VAAIRSGGNTYYADAVKGRFTISRDNAKNTLYLQMNSLRAEDTAVYY |
| | | CASQWGDWGHFEYWGQGTLVTVSS |
| 332 | scFv_19 | EVQLVETGGDLVKPAGSLRLSCAASGFPFSGYSMTWVRQAPEKGLQL |
| | VH | VAGINSDGSHTYYTDDVKGRFTVSRDNTKYILYLQMNSLRVEDTAIYY |
| | | CGSHSWGNFHYWGQGTLVTVSS |
| 333 | scFv_20 | EVQLVESGGDLVKPGGSLRLSCVASGFTFSSYAMSWVRQAPGEGLQW |
| | VH | VAYINSAGSTTYYADAVKGRFTISRDNARNTVYLQMNSLRAEDTAVY |
| | | YCGRYYYGLDYWGPGTSVFVSS |
| 334 | scFv_21 | EVQLVESGGDMVKPGGSLRLSCVASGFTFSSFWMYWVRQAPGKGLQ |
| | VH | WVGGINSGGSITSYADAVKGRFTISRDNARNTLYLQMDSLRDEDTAVY |
| | | YCAKPDIYGSFDHWGQGTLVTVSS |
| 335 | scFv_22 | ELTLQESGPGLVKPAQTLSLTCTVSGDSVTSSHFWHWIRQRPGRGLEW |
| | VH | MGYWSDITIYSPTFQGRISITVDTARNQFSLHLNSVTTEDSAVYFCARY |
| | | NGYVDYLDVWGQGTLITVSS |
| 336 | scFv_23 | ELTLQESGPGLVKPSQTLSLTCVVSGGSVTTSDYWNWLRQRPGRGLEY |
| | VH | MGYWKGRTEYNPAFQGRISITADTAKNQFSLQLNSVTADDTAIYYCAH |
| | | WGSGNLDYWGPGTLVTVSS |
| 337 | scFv_24 | EVQLVQSGGXLVKPGGSLRLSCVASGFTLSSNYMDWVRQAPGKGPQ |
| | VH | WVAQISTDGSRTYYADAVKGRFTISRDTAKNTLYLQMNSLRAEDTAM |
| | | YYCAKNEYLAPTYWGQGTLVTVSS |
| 338 | scFv_25 | EEQLVEFGGDLVKPGGSLRLSCVASGFTFSSLSMNWIRQAPGKGLQWV |
| | VH | SQISPDGTITGYTDAVKGRFTISRDNAKNTLYLQMNSLRDEDTAIYYCA |
| | | KSRYTDTYPFDYWGPGTLVTVSS |
| 339 | scFv_26 | EVQLVETGGDLVKPGGSLRLSCVGSGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 340 | scFv_27 | EVQLVESGGDLVKPGGSLRLSCVGSGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 341 | scFv_28 | EVQLVETGGDLVKPGGSLRLSCVGSGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 342 | scFv_29 | EVHLVESGGDLVKPGGSLRLSCVGSGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 343 | scFv_30 | EVQLVESGGDLVKPGGSLRLSCVGSGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 344 | scFv_31 | EVQLVESGGDLVKPGGSLRLSCVASGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 345 | scFv_32 | EVQLVESGGDLVKPGGSLRLSCVGSGFTFSSYWMSWVRQAPGKGLQ |
| | VH | WIADISDSGSSTDYADAVKGRFTISRDNAKNTLYLQMNSLRAEDTAMY |
| | | YCARGLGMDYWGHGTSLFVSS |
| 346 | scFv_33 | EVQLVESGGDLVKPGGSLRLSCAASGFTFSHYDMNWVRQAPGKGLQ |
| | VH | WVASIAYDGSNTYYTDAVKGRFTISRDNAKNILFLLVNSLRAEDTAIY |
| | | YCARGLVYWGQGTLVTVSS |
| 347 | scFv_34 | EVQLVESGGDLVKPGGSLRLSCVASGLTFSDNYMSWIRQVPGKGLQW |
| | VH | VSRISTDGRGTSYADAVKGRFTISRDNAKNTLYLQMNSLRDEDTAVYY |
| | | CARRSGIWGFDYWGQGTLVTVSS |
| 348 | scFv_35 | EVQLVESGGDLVKPAGSLRLSCVTSGFTFSSYAMSWVRQAPGKGLQW |
| | VH | VGSINSGGSTTSYADAVKGRFTISRDNAKNTVYLQMNSLRAEDTAVYY |
| | | CARWWDFEYWGQGTLVTVSS |
| 349 | scFv_36 | EEQLVEFGGGLVRPGGSLRLSCVASGFTFSSYGMTWVRQSPGKGLQW |
| | VH | VATISDTGSTYYADTVKGRFSISRDNAKNTLYLQMNSLRADDTALYYC |
| | | TKDLGVWGMDNWGPGTSLFVSS |
| 350 | scFv_37 | EVQLVETGGDLVKPGGSLRLSCVASGFTLRDYSMTWVRQTPEKGLQL |
| | VH | VAGISTGGSTTYYIDAVKGRFTISRDNAKNTVYLQMNSLRAEDTAMYH |
| | | CTTTALGYWGQGTLVTVSS |
| 351 | scFv_38 | EVQLVESGGDLVKPGGSLRLSCVASGFNFGNYAVSWVRQAPGKGLQ |
| | VH | WVAGISNDGTITGYTDVVEGRFIISRDNAKNTLYLQMNSLRAEDMAIY |
| | | YCVAGGFLDYWGQGTLVTVSS |
| 352 | scFv_39 | EVPLVESGGDLVKPGGSLRLSCVASGFTFSSYGMSWVRQSPGKGLQW |
| | VH | VAGITYDGSNTYYTDTVRGRFTISRDNARNTVYLQMNSLRAEDTAVY |
| | | YCVASPLYGTYGFDYWGHGTSVFVSS |
| 353 | scFv_40 | ELTLQESGPGLVKPSQTLSLTCAVSGGSVIRGYYWTWIRQRPGRGLEW |
| | VH | MGYWAGSTDYNPAFQGRIFITAGTAKNQFSLEMTSMTTDDTAIYYCV |
| | | GSLRGGWLFDNWGQGTLVTVSS |
| 354 | scFv_41 | EVQLVETGGDLVMPAGSLRLSCVASGFTFSSSVMSWVRQAPGKGLEW |
| | VH | VAGINGGGSFAAYGDAVKGRFTISRDNAKNTLYLQMNSLRAEDTAMY |
| | | YCVGGGYMDFWGPGTSLFVSS |
| 355 | scFv_42 | EVQLVEIGGDLVKPEGSLRLSCVVSGFTFSGYDMNWVRQAPGKGLQW |
| | VH | VAYISSDGRRTVYTDAVKGRFTISRDNAKNTLYLQMNSLKSEDTAVYY |
| | | CVKGGWLDFWGQGTLVTVSS |
| 356 | scFv_43 | EVQLVESGGDLVKPGGSLRLSCAASGFTFTDYDMSWVRQAPGKGLQ |
| | VH | WVAAISYDGSSTYYTDAVKGRFTISRDNARNTLYLQMSSLRAEDTAM |
| | | YYCVVSNMNLWGHGTSVFVSS |

### CD19 Targeting

In some aspects, the binding polypeptides, the antibody or antigen-binding fragments thereof described in the present disclosure can bind to human CD19. The human CD19 antigen, also known as B-lymphocyte surface antigen B4, B4, and CVID3 is a 95 kD type I transmembrane glycoprotein that is a member of the immunoglobulin gene superfamily. The CD19 protein is encoded by a the ˜7 kb (I) 19 gene located in humans on the short arm of chromosome 16. The CD19 protein has two N-terminal extracellular Ig-like domains separated by a non-Ig-like domain, a single hydrophobic transmembrane domain, and a large C-terminal cytoplasmic domain. CD19 has no significant homologies with other known human proteins.

Expression of CD19 is restricted largely to B cell lymphocytes and some follicular dendritic cells, where it is a reliable marker for pre-B cells and mature B cells. However, CD19 expression diminishes during terminal B cell differentiation into antibody-secreting plasma cells. CD 19 plays two roles in B cell activation: as an adaptor protein to recruit other cytoplasmic signaling proteins to the plasma membrane, and as a complex with several membrane proteins including complement receptor type 2 (CD21) and tetraspanin (CD81), which together act to reduce the activation threshold for antigen-initiated B cell activation.

Though not typically essential for carcinogenesis and tumor growth, CD19 expression is highly conserved on most B cell tumors, including acute lymphoblastic leukemias (ALLs), chronic lymphocytic leukemias (CLLs), and B cell lymphomas where it likely contributes to overall lymphomagenic signaling. Indeed, the majority of B cell malignancies express CD19 at normal to high levels. Given its selective B cell expression and close association with B cell lymphomas and leukemias, CD19 has been a target of cancer immunotherapies seeking to treat these types of malignancies, including targeting by monoclonal antibodies, antibody-drug conjugates, immunotoxin conjugates, bi-specific T cell engagers (BiTEs), and chimeric antigen receptors (CARs) with varying levels of clinical success.

CD19-directed CAR-T cells are also showing promise outside of cancer for counteracting “unwanted immunity” in cases of autoantibody-mediated autoimmune disease such as for the treatment of systemic lupus erythematosus and other autoimmune diseases and for prevention of alloantibody-mediated solid organ transplant immune incompatibility due to the presence of donor-specific HLA antibodies. With respect to the latter application, clinical trials are currently underway which use CD19-directed CAR-T cells in combination with BCMA-directed CAR-T cells in order to eliminate alloantibody producing B cells and plasma cells, respectively, to treat alloimmunized patients who are in need of renal transplant.

The CD19-specfic antibodies and antigen-binding fragments (e.g., scFvs) disclosed herein can be used to target CD19 expressed on autoantibody-producing B cells as well as a variety of malignant and pathogenic B cells and thereby serve as a B-cell targeting immunotherapy.

### Chimeric Antigen Receptors

The present invention provides compositions and methods for modified immune cells or precursor cells thereof, e.g., modified T cells, comprising a chimeric antigen receptor (CAR) having affinity for human CD19. A subject CAR of the invention comprises an antigen binding domain (e.g., CD19 binding domain), a transmembrane domain, a costimulatory signaling domain, and an intracellular signaling domain. A subject CAR of the invention may optionally comprise a hinge domain. Accordingly, a subject CAR of the invention comprises an antigen binding domain (e.g., CD19 binding domain), a hinge domain, a transmembrane domain, a costimulatory signaling domain, and an intracellular signaling domain. In some embodiments, each of the domains of a subject CAR is separated by a linker.

The antigen binding domain may be operably linked to another domain of the CAR, such as the transmembrane domain, the costimulatory signaling domain or the intracellular signaling domain, each described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding a costimulatory signaling domain.

The antigen binding domains described herein, including antibodies or antigen-binding fragments thereof, scFvs, and the like, can be combined with any of the transmembrane domains, any of the costimulatory signaling domains, any of the intracellular signaling domains, or any of the other domains described herein that may be included in a CAR of the present invention.

In one aspect, the invention includes a chimeric antigen receptor (CAR) that specifically binds CD19, comprising: a CD19-specific antigen binding domain, optionally a hinge domain, a transmembrane domain, a costimulatory signaling domain, and an intracellular signaling domain.

In one aspect, the invention includes a chimeric antigen receptor (CAR) that specifically binds CD19, comprising: a CD19-specific antigen binding domain, optionally a hinge domain, a transmembrane domain, and an intracellular signaling domain.

In one exemplary embodiment, the invention includes a chimeric antigen receptor (CAR) that specifically binds CD19, comprising: a CD19-specific antigen binding domain comprising a heavy chain variable (VH) domain and a light chain variable (VL) domain, wherein the VH domain comprises three heavy chain complementarity determining regions, wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 8, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and wherein the VL domain comprises a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247; a transmembrane domain; and a KIR2DS2 signaling domain.

In some embodiments, a genetically modified immune cell (e.g., T cell) or precursor cell thereof of the present invention comprises a chimeric antigen receptor (CAR) having affinity for human CD19. In some embodiments, a genetically modified immune cell (e.g., T cell) or precursor cell thereof of the present invention comprises a chimeric antigen receptor (CAR) having affinity for human CD19.

In certain embodiments, the genetically modified cell is a T cell.

Accordingly, in one exemplary embodiment, provided herein is a genetically modified T cell comprising a chimeric antigen receptor (CAR) that specifically binds human CD19, comprising: a human CD19 specific antigen binding domain, an optional hinge domain, a transmembrane domain, a costimulatory signaling domain, and an intracellular signaling domain.

In another exemplary embodiment, provided herein is a genetically modified T cell comprising a chimeric antigen receptor (CAR) that specifically binds human CD19, comprising: a human CD19 specific antigen binding domain, an optional hinge domain, a transmembrane domain, and a KIR2DS2 signaling domain.

Accordingly, in one exemplary embodiment, the invention includes a is a genetically modified T cell comprising chimeric antigen receptor (CAR) that specifically binds human CD19, comprising: a CD19-specific antigen binding domain comprising a heavy chain variable (VH) domain and a light chain variable (VL) domain, wherein the VH domain comprises three heavy chain complementarity determining regions, wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 8, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and wherein the VL domain comprises a light chain variable region that comprises three light chain complementarity determining regions, wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247; a transmembrane domain; and a KIR2DS2 signaling domain.

In certain embodiments of the invention, the antigen binding domain of the CAR is encoded by a nucleic acid sequence comprising the nucleotide sequence of SEQ ID NOs: 1-43. In certain embodiments of the invention, the antigen-binding domain of the CAR comprises the amino acid sequence of SEQ ID NOs: 44-86.

In some embodiments, the nucleic acid encoding an antigen binding domain of a CAR encodes an HCDR1, HCDR2, HCDR3, LCDR1, and LCDR3 which comprise the respective amino acid sequences set forth in:
SEQ ID NOs: 87, 88, 89, 90, and 91 and wherein LCDR2 comprises the amino acid sequence GNS;
SEQ ID NOs: 92, 93, 94, 95, and 96, and wherein LCDR2 comprises the amino acid sequence AND;
SEQ ID NOs: 97, 98, 99, 100, and 101, and wherein LCDR2 comprises the amino acid sequence ANI;
SEQ ID NOs: 102, 103, 104, 100, and 105, and wherein LCDR2 comprises the amino acid sequence ANI;
SEQ ID NOs: 106, 107, 108, 109, and 110, and wherein LCDR2 comprises the amino acid sequence SDG;
SEQ ID NOs: 111, 112, 113, 114, and 115, and wherein LCDR2 comprises the amino acid sequence GSS;
SEQ ID NOs: 116, 117, 118, 119, and 120, and wherein LCDR2 comprises the amino acid sequence GNT;
SEQ ID NOs: 121, 122, 123, 124, and 125, and wherein LCDR2 comprises the amino acid sequence GYN;
SEQ ID NOs: 126, 117, 127, 128, and 129, and wherein LCDR2 comprises the amino acid sequence GNS;
SEQ ID NOs: 130, 131, 132, 100, and 133, and wherein LCDR2 comprises the amino acid sequence ANI;
SEQ ID NOs: 134, 135, 136, 100, and 105, and wherein LCDR2 comprises the amino acid sequence ANI;
SEQ ID NOs: 137, 138, 139, 140, and 141, and wherein LCDR2 comprises the amino acid sequence ADS;
SEQ ID NOs: 142, 143, 144, 100, and 105, and wherein LCDR2 comprises the amino acid sequence ANI;
SEQ ID NOs: 145, 146, 147, 148, and 149, and wherein LCDR2 comprises the amino acid sequence GNS;
SEQ ID NOs: 150, 151, 152, 153, and 154, and wherein LCDR2 comprises the amino acid sequence GNS;
SEQ ID NOs: 155, 156, 157, 158, and 159, and wherein LCDR2 comprises the amino acid sequence GSH;
SEQ ID NOs: 121, 160, 161, 128, and 162, and wherein LCDR2 comprises the amino acid sequence GNS;
SEQ ID NOs: 163, 164, 123, 124, and 165, and wherein LCDR2 comprises the amino acid sequence GYN;
SEQ ID NOs: 106, 107, 108, 166, and 167, and wherein LCDR2 comprises the amino acid sequence SDG;
SEQ ID NOs: 168, 169, 170, 171, and 172, and wherein LCDR2 comprises the amino acid sequence KDT;
SEQ ID NOs: 173, 174, 175, 171, and 176, and wherein LCDR2 comprises the amino acid sequence KDT;
SEQ ID NOs: 177, 178, 179, 180, and 181, and wherein LCDR2 comprises the amino acid sequence SSS;
SEQ ID NOs: 182, 183, 184, 185, and 186, and wherein LCDR2 comprises the amino acid sequence LVS;
SEQ ID NOs: 187, 188, 189, 100, and 190, and wherein LCDR2 comprises the amino acid sequence ANI;
SEQ ID NOs: 191, 192, 193, 185, and 194, and wherein LCDR2 comprises the amino acid sequence QVS;
SEQ ID NOs: 195, 196, 197, 171, and 198, and wherein LCDR2 comprises the amino acid sequence KDT;
SEQ ID NOs: 195, 196, 197, 171, and 199, and wherein LCDR2 comprises the amino acid sequence KDT;
SEQ ID NOs: 195, 196, 197, 200, and 201, and wherein LCDR2 comprises the amino acid sequence RDT;
SEQ ID NOs: 195, 196, 197, 200, and 202, and wherein LCDR2 comprises the amino acid sequence KDT;
SEQ ID NOs: 195, 196, 197, 171, and 190, and wherein LCDR2 comprises the amino acid sequence KDT;
SEQ ID NOs: 195, 196, 197, 203, and 204, and wherein LCDR2 comprises the amino acid sequence RDT;
SEQ ID NOs: 195, 196, 197, 171, and 205, and wherein LCDR2 comprises the amino acid sequence KDT;
SEQ ID NOs: 206, 207, 208, 209, and 210, and wherein LCDR2 comprises the amino acid sequence SVS;
SEQ ID NOs: 211, 112, 113, 114, and 115, and wherein LCDR2 comprises the amino acid sequence GSS;
SEQ ID NOs: 168, 117, 212, 213, and 214, and wherein LCDR2 comprises the amino acid sequence KDT;
SEQ ID NOs: 121, 215, 216, 166, and 217, and wherein LCDR2 comprises the amino acid sequence SDG;
SEQ ID NOs: 218, 219, 220, 221, and 222, and wherein LCDR2 comprises the amino acid sequence RDT;
SEQ ID NOs: 223, 224, 225, 213, and 226, and wherein LCDR2 comprises the amino acid sequence KDT;
SEQ ID NOs: 121, 227, 228, 229, and 230, and wherein LCDR2 comprises the amino acid sequence SDG;
SEQ ID NOs: 231, 232, 233, 234, and 235, and wherein LCDR2 comprises the amino acid sequence NND;
SEQ ID NOs: 236, 237, 238, 171, and 239, and wherein LCDR2 comprises the amino acid sequence KDT;
SEQ ID NOs: 240, 143, 241, 242, and 243, and wherein LCDR2 comprises the amino acid sequence SNK; or
SEQ ID NOs: 244, 138, 245, 246, and 247, and wherein LCDR2 comprises the amino acid sequence EAS.

Sequences of individual domains of the CAR are found in Table 4.

Accordingly, a subject CAR may be a CAR having affinity for CD19, comprising a CD19 binding domain comprising the amino acid sequence set forth in SEQ ID NOs: 44-86. A subject CD19 CAR may further comprise a leader sequence comprising an amino acid sequence set forth in SEQ ID NO: 357. A subject CD19 CAR may further comprise a myc tag domain comprising an amino acid sequence set forth in SEQ ID NO: 264. A subject CD19 CAR may further comprise a transmembrane and intracellular signaling domain comprising an amino acid sequence set forth in SEQ ID NO: 266.

### NK Cell Receptor CARs

In some aspects, the invention of the current disclosure provides a CAR which shares functional and structural properties with a NK cell immune-function receptor (or NKR). NKRs and NKR-CARs are described herein, e.g., in the section below. As is discussed below, a variety of NKRs can serve as the basis for an NKR-CAR.

### NK Cell Immune-Function Receptors (NKRS) and NK Cells

As disclosed herein, NK cell immune-function receptor (or NKR) refers to an endogenous naturally occurring transmembrane protein expressed in NK cells, which can engage with a ligand on an antigen presenting cell and modulate an NK cell immune-function response, e.g., the cytolytic activity or cytokine secretion of the NK cell. NK cells are mononuclear cells that develop in the bone marrow from lymphoid progenitors and are typically identified by the expression of the cluster determinants (CDs) CD 16, CD56, and/or CD57, as well as the absence of the alpha/beta or gamma/delta TCR complex on the cell surface. Functionally, NK cells possess the ability to bind to and kill target cells that fail to express “self major histocompatibility complex (MHC)/human leukocyte antigen (HLA) proteins; and the ability to kill tumor cells or other diseased cells that express ligands for activating NK receptors. NK cells are characterized by their ability to bind and kill 5 several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Upon activation by interferons and/or cytokines, NK cells mediate the lysis of tumor cells and of cells infected with intracellular pathogens by mechanisms that require direct, physical contacts between the NK cell and the target cell. Lysis of target cells involves the release of cytotoxic granules from the NK cell onto the surface of the bound target, and effector proteins such as perforin and granzyme B that penetrate the target plasma membrane and induce apoptosis or programmed cell death. Normal, healthy cells are protected from lysis by NK cells. NK cell activity is regulated by a complex mechanism that involves both stimulating and inhibitory signals.

Briefly, the lytic activity of NK cells is regulated by various cell surface receptors that transduce either positive or negative intracellular signals upon interaction with ligands on the target cell. The balance between positive and negative signals transmitted via these receptors determine whether or not a target cell is lysed (killed) by a NK cell. NK cell stimulatory signals can be mediated by Natural Cytotoxicity Receptors (NCR) such as NKp30, NKp44, and NKp46; as well as NKG2C receptors, NKG2D receptors, certain activating killer cell immunoglobulin-like receptors (KIRs), and other activating NK receptors (Lanier, Annual Review of Immunology 2005; 23:225-74). NK cell inhibitory signals can be mediated by receptors like Ly49, CD94/NKG2A, as well as certain inhibitory KIRs, which recognize major histocompatibility complex (MHC) class I molecules (Karre et al., Nature 1986; 319:675-8; Ohlen et al, Science 1989; 246:666-8). These inhibitory receptors bind to polymorphic determinants of MHC class I molecules (including HLA class I) present on other cells and inhibit NK cell-mediated lysis.

### KIR-CARS

In certain embodiments, the current disclosure provides a chimeric antigen receptor (CAR) molecule comprising an antigen binding domain and a killer cell immunoglobulin-like receptor domain (KIR-CAR). In one embodiment, the KIR-CAR of the invention is expressed on the surface of an immune effector cell, e.g., a T cell or a NK cell.

KIRs, referred to as killer cell immunoglobulin-like receptors, have been characterized in humans and non-human primates, and are polymorphic type 1 trans-membrane molecules present on certain subsets of lymphocytes, including NK cells and some T cells. KIRs interact with determinants in the alpha 1 and 2 domains of the MHC class I molecules and, as described elsewhere herein, distinct KIRs are either stimulatory or inhibitory for NK cells.

NKR-CARs described herein include KIR-CARs, which share functional and structural properties with KIRs.

KIRs are a family of cell surface proteins found on NK cells, which regulate the killing function of these cells by interacting with MHC class I molecules, which are expressed on all cell types. This interaction allows them to detect virally infected cells or tumor cells. Most KIRs are inhibitory, meaning that their recognition of MHC suppresses the cytotoxic activity of the NK cell that expresses them. Only a limited number of KIRs have the ability to activate cells. The KIR gene family has at least 15 gene loci (KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, and two pseudogenes, KIR2DP1 and KIR3DP1) encoded within a 100-200 Kb region of the Leukocyte Receptor Complex (LRC) located on chromosome 19 (19q13.4). The LRC constitutes a large, 1 Mb, and dense cluster of rapidly evolving immune genes which contains genes encoding other cell surface molecules with distinctive lg-like extra-cellular domains. In addition, the extended LRC contains genes encoding the trans membrane adaptor molecules DAPI0 and DAP12.

KIR genes vary in length from 4 to 16 Kb (full genomic sequence) and can contain four to nine exons. KIR genes are classified as belonging to one of three groups according to their structural features: (1) Type I KIR2D genes, which encode two extra-cellular domain proteins with a D1 and D2 conformation; (2) The structurally divergent Type II KIR2D genes which encode two extra-cellular domain proteins with a DO and D2 conformation; and finally (3) KIR3D genes encoding proteins with three extra-cellular lg-like domains (DO, D1 and D2). Type I KIR2D genes, which include the pseudogene KIR2DP1 as well as KIR2DL1-3 and KIR2DS 1-5 genes, possess eight exons as well as a pseudoexon 3 sequence. This pseudoexon is inactivated in Type I KIR2D. In some cases, this is due to a nucleotide substitution located on the intron 2-exon 3 splice-site where its nucleotide sequence exhibits a high-degree of identity to KIR3D exon 3 sequences and possesses a characteristic three base pair deletion. In other cases, a premature stop codon initiates differential splicing of exon 3. Within the Type I KIR2D group of genes, KIR2DL1 and KIR2DL2 share a common deletion in exon 7 distinguishing them from all other KIR in this exon, which results in a shorter exon 7 sequence. Similarly, within Type I KIR2D, KIR2DL1-3 differ from KIR2DS1-5 only in the length of their cytoplasmic tail encoding region in exon 9. The KIR2DP1 pseudogene structure differs from that of KIR2DL1-3 in that the former has a shorter exon 4 sequence, due to a single base pair deletion.

Type II KIR2D genes include KIR2DL4 and KIR2DL5. Unlike KIR3D and Type I KIR2D, Type II KIR2D characteristically have deleted the region corresponding to exon 4 in all other KIR. Additionally, Type II KIR2D genes differ from Type I KIR2D genes in that the former possess a translated exon 3, while Type I KIR2D genes have an untranslated pseudoexon 3 sequence in its place. Within the Type II KIR2D genes, KIR2DL4 is further differentiated from KIR2DL5 (as well as from other KIR genes) by the length of its exon 1 sequence. In KIR2DL4, exon 1 was found to be six nucleotides longer and to possess an initiation codon different from those present in the other KIR genes. This initiation codon is in better agreement with the Kozak transcription initiation consensus sequence' than the second potential initiation codon in KIR2DL4 that corresponds to the initiation codon present in other KIR genes.

KIR3D genes possess nine exons and include the structurally related KIR3DL1, KIR3DS1, KIR3DL2 and KIR3DL3 genes. KIR3DL2 nucleotide sequences are the longest of all KIR genes and span 16,256 bp in full genomic sequences and 1,368 bp in cDNA. Within the KIR3D group, the four KIR genes differ in the length of the region encoding the cytoplasmic tail in exon 9. The length of the cytoplasmic tail of KIR proteins can vary from 14 amino acid residues long (in some KIR3DS 1 alleles) to 108 amino acid residues long (in KIR2DL4 proteins). Additionally, KIR3DS1 differs from KIR3DL1 or KIR3DL2 in that the former has a shorter exon 8 sequence. KIR3DL3 differs from other KIR sequences in that it completely lacks exon 6. The most extreme KIR gene structure difference observed was that of KIR3DP1. This gene fragment completely lacks exons 6 through 9, and occasionally also exon 2. The remaining portions of the gene which are present (exon 1, 3, 4 and 5) share a high level of sequence identity to other KIR3D sequences, in particular to KIR3DL3 sequences.

KIR proteins possess characteristic lg-like domains on their extracellular regions, which in some KIR proteins are involved in HLA class I ligand binding. They also possess transmembrane and cytoplasmic regions which are functionally relevant as they define the type of signal which is transduced to the NK cell. KIR proteins can have two or three lg-like domains (hence KIR2D or KIR3D) as well as short or long cytoplasmic tails (represented as KIR2DS or KIR2DL). Two domain KIR proteins are subdivided into two groups depending on the origin of the membrane distal lg-like domains present. Type I KIR2D proteins (KIR2DL1, KIR2DL2, KIR2DL3, KIR2DS1, KIR2DS2, KIR2DS3, KIR2DS4 and KIR2DS5) possess a membrane-distal lg-like domain similar in origin to the KIR3D DI lg-like domain but lack a DO domain. This DI lg-like domain is encoded mainly by the fourth exon of the corresponding KIR genes. The Type II KIR2D proteins, KIR2DL4 and KIR2DL5, possess a membrane-distal lg-like domain of similar sequence to the DO domain present in KIR3D proteins, however, Type II KIR2D lack a DI domain. Long cytoplasmic tails usually contain two Immune Tyrosine-based Inhibitory Motifs (ITIM) which transduce inhibitory signals to the NK cell. Short cytoplasmic tails possess a positively charged amino acid residue in their transmembrane region which allows them to associate with a DAP12 signaling molecule capable of generating an activation signal Exceptions to this is KIR2DL4, which contains only one N-terminus ITIM. In addition, KIR2DL4 also possesses a charged residue (arginine) in its transmembrane domain, a feature which allows this receptor to elicit both inhibitory and activating signals. KIR control the response of human NK cells by delivering inhibitory or activating signals upon recognition of MHC class I ligands on the surface of potential target cells.

KIR proteins vary in length from 306 to 456 amino acid residues. Although the differences in protein length are mostly the consequence of the number of lg-like domains present, cytoplasmic region length diversity is also an influencing factor. The leader peptide of most KIR proteins is 21 amino acid residues long. However, the presence of a different initiation codon generates a correspondingly longer leader peptide in KIR2DL4 proteins. The DO lg-like domain present in Type II KIR2D proteins and KIR3D proteins is approximately 96 amino acid residues in length. The DI domain of Type I KIR2D and of KIR3D proteins is 102 amino acid residues long, while the D2 domain of all KIR proteins is 98 amino acid residues long. The length of the stem region varies from the 24 amino acid residues present in most KIR proteins, to only seven amino acid residues in the divergent KIR3DL3 protein. The transmembrane region is 20 amino acid residues long for most proteins, but one residue shorter on KIR2DL1 and KIR2DL2 proteins as a result of a three base pair deletion in exon 7. Finally, the cytoplasmic region of KIR proteins exhibits greater length variations, ranging from 23 amino acid residues in some KIR3DS 1 alleles to the 96 amino acid residues present in KIR3DL2 proteins.

Amino acid sequences for human KIR polypeptides (Homo sapiens) are available in the KIR NCBI database, see e.g., accession number NP_037421.2 (GI: 134268644), NP_703144.2 (GI: 46488946), NP_001229796.1 (GI: 338968852), NP_001229796.1 (GI: 338968852), NP_006728.2 (GI: 134268642), NP_065396.1 (GI: 11968154), NP_001018091.1 (GI: 66267727), NP_001077008.1 (GI: 134133244), NP_036444.1 (GI: 6912472), NP_055327.1 (GI: 7657277), NP_056952.2 (GI: 71143139), NP_036446.3 (GI: 116517309), NP_001074239.1 (GI: 124107610), NP_002246.5 (GI: 124107606), NP_001074241.1 (GI: 124107604), NP_036445.1 (GI: 6912474).

The nomenclature for KIRs is based upon the number of extracellular domains (KIR2D and KIR3D having two and three extracellular lg-domains, respectively) and whether the cytoplasmic tail is long (KIR2DL or KIR3DL) or short (KIR2DS or KIR3DS). The presence or KIR absence of a given is variable from one NK cell to another within the NK population present in a single individual. Among humans, there is also a relatively high level of polymorphism of KIR genes, with certain KIR genes being present in some, but not all individuals. The expression of KIR alleles on NK cells is stochastically regulated, meaning that, in a given individual, a given lymphocyte may express one, two, or more different KIRs, depending on the genotype of the individual. The NK cells of a single individual typically express different combinations of KIRs, providing a repertoire of NK cells with different specificities for MHC class I molecules. Certain KIR gene products cause stimulation of lymphocyte activity when bound to an appropriate ligand. The activating KIRs all have a short cytoplasmic tail with a charged trans-membrane residue that associates with an adapter molecule having an Immunoreceptor Tyrosine-based Activation Motifs (ITAMs) which transduce stimulatory signals to the NK cell. By contrast, inhibitory KIRs have a long cytoplasmic tail containing Immunoreceptor Tyrosine-based Inhibitory Motif (ITIM), which transduce inhibitory signals to the NK cell upon engagement of their MHC class I ligands. The known inhibitory KIRs include members of the KIR2DL and KIR3DL subfamilies. Inhibitory KIRs having two lg domains (KIR2DL) recognize HLA-C allotypes: KIR2DL2 (formerly designated p58.2) and the closely related, allelic gene product KIR2DL3 both recognize “group 1” HLA-C allotypes (including HLA-Cwl,-3,-7, and -8), whereas KIR2DL1 (p58.1) recognizes “group 2” HLA-C allotypes (such as HLA-Cw2,-4,-5, and -6). The recognition by KIR2DL1 is dictated by the presence of a Lys residue at position 80 of HLA-C alleles. KIR2DL2 and KIR2DL3 recognition is dictated by the presence of an Asn residue at position 80 in HLA-C. Importantly, the great majority of HLA-C alleles have either an Asn or a Lys residue at position 80. Therefore, KIR2DL1,-2, and -3 collectively recognize essentially all HLA-C allotypes found in humans. One KIR with three lg domains, KIR3DL1 (p70), recognizes an epitope shared by HLA-Bw4 alleles. Finally, KIR3DL2 (p140), a homodimer of molecules with three lg domains, recognizes HLA-A3 and -HLA-A11.

However, the invention should not be limited to inhibitory KIRs comprising a cytoplasmic tail containing ITIM. Rather, any inhibitory protein having a cytoplasmic domain that is associated with an inhibitory signal can be used in the construction of the CARs of the invention. Non-limiting examples of an inhibitory protein include but are not limited CTLA-4, PD-1, and the like. These proteins are known to inhibit T cell activation.

Accordingly, in some aspects, the invention of the current disclosure provides a KIR-CAR comprising an extracellular domain that comprises a target-specific binding element, otherwise referred to as an antigen binding domain, fused to a KIR or fragment thereof. In one embodiment, the KIR is an activating KIR that comprises a short cytoplasmic tail that associates with an adapter molecule having an Immunoreceptor Tyrosine-based Activation Motifs (ITAMs) which transduce stimulatory signals to the NK cell (referred elsewhere herein as actKIR-CAR). In one embodiment, the KIR is an inhibitory KIR that comprises a long cytoplasmic tail containing Immunoreceptor Tyrosine based Inhibitory Motif (ITIM), which transduces inhibitory signals (referred to herein as inhKIR-CAR). In some instances, it is desirable to remove the hinge region for the activating KIRs when construction an actKIR-CAR. This is because the invention is partly based on the discovery that an activating KIR CAR in which the KIR2DS2 hinge was removed to generate the KIR2S CAR, this KIRS2 CAR exhibited enhanced cytolytic activity compared to an actKIR-CAR comprising a full-length wildtype KIR2DS2.

In some embodiments, a KIR-CAR comprises an antigen binding domain and a KIR transmembrane domain. In some embodiments, a KIR-CAR comprises an antigen binding domain and a KIR intracellular domain, e.g., an inhKIR intracellular domain. KIR D domain, as that term is used herein, refers to a DO, DI, or D2 domain of a KIR. KIR D domain, as that term is used herein, refers to a polypeptide domain having structural and functional properties of a D domain of a KIR. KIR DO domain, as that term is used herein, refers to a DO domain of a KIR. In some embodiments the KIR DO domain of a KIR-CAR has at least 70, 80, 85, 90, 95, or 99% homology with a reference sequence, e.g., a naturally occurring KIR DO domain or a KIR DO domain described herein. In some embodiments the KIR DO domain of a KIR-CAR differs at no more than 15, 10, 5, 2, or 1% of its residues from a reference sequence, e.g., a naturally occurring KIR DO domain or a KIR DO domain described herein. In some embodiments the KIR DO domain of a KIR-CAR differs at no more than 5, 4, 3, 2 or 1 residue from a reference sequence, e.g., a naturally occurring KIR DO domain or a KIR DO domain described herein. In some embodiments the KIR DO domain of a KIR-CAR does not differ from, or shares 100% homology with, a reference sequence, e.g., a naturally occurring KIR DO domain or a KIR DO domain described herein. KIR DI domain, as that term is used herein, refers to a polypeptide domain having structural and functional properties of a DI domain of a KIR. In an embodiment the KIR DI domain of a KIR-CAR has at least 70, 80, 85, 90, 95, or 99% homology with a reference sequence, e.g., a naturally occurring KIR DI domain or a KIR DI domain described herein. In some embodiments the KIR DI domain of a KIR-CAR differs at no more than 15, 10, 5, 2, or 1% of its residues from a reference sequence, e.g., a naturally occurring KIR DI domain or a KIR DI domain described herein. In some embodiments the KIR DI domain of a KIR-CAR differs at no more than 5, 4, 3, 2 or 1 residue from a reference sequence, e.g., a naturally occurring KIR DO domain or a KIR DI domain described herein. In some embodiments the KIR DI domain of a KIR-CAR does not differ from, or shares 100% homology with, a reference sequence, e.g., a naturally occurring KIR DI domain or a KIR DI domain described herein.

KIR D2 domain, as that term is used herein, refers to a polypeptide domain having structural and functional properties of a D2 domain of a KIR. In an embodiment the KIR D2 domain of a KIR-CAR has at least 70, 80, 85, 90, 95, or 99% homology with a reference sequence, e.g., a naturally occurring KIR D2 domain or a KIR D2 domain described herein. In embodiments the KIR D2 domain of a KIR-CAR differs at no more than 15, 10, 5, 2, or 1% of its residues from a reference sequence, e.g., a naturally occurring KIR D2 domain or a KIR D2 domain described herein. In some embodiments the KIR D2 domain of a KIR-CAR differs at no more than 5, 4, 3, 2 or 1 residue from a reference sequence, e.g., a naturally occurring KIR D2 domain or a KIR D2 domain described herein. In some embodiments the KIR D2 domain of a KIR-CAR does not differ from, or shares 100% homology with, a reference sequence, e.g., a naturally occurring KIR D2 domain or a KIR D2 domain described herein.

KIR hinge or stem domain, as that term is used herein, refers to a polypeptide domain having structural and functional properties of a hinge or stem domain of a KIR. In some embodiments the KIR hinge or stem domain of a KIR-CAR has at least 70, 80, 85, 90, 95, or 99% homology with a reference sequence, e.g., a naturally occurring KIR hinge or stem domain or a KIR hinge or stem domain described herein. In some embodiments the KIR hinge or stem domain of a KIR-CAR differs at no more than 15, 10, 5, 2, or 1% of its residues from a reference sequence, e.g., a naturally occurring KIR hinge or stem domain or a KIR hinge or stem domain described herein. In some embodiments the KIR hinge or stem domain of a KIR-CAR differs at no more than 5, 4, 3, 2 or 1 residue from a reference sequence, e.g., a naturally occurring KIR hinge or stem domain or a KIR hinge or stem domain described herein. In some embodiments the KIR hinge or stem domain of a KIR-CAR does not differ from, or shares 100% homology with, a reference sequence, e.g., a naturally occurring KIR hinge or stem domain or a KIR hinge or stem domain described herein.

KIR transmembrane domain, as that term is used herein, refers to a polypeptide domain having structural and functional properties of a transmembrane domain of a KIR. In some embodiments the KIR transmembrane domain of a KIR-CAR has at least 70, 80, 85, 90, 95, or 99% homology with a reference sequence, e.g., a naturally occurring KIR transmembrane domain or a KIR transmembrane domain described herein. In some embodiments the KIR transmembrane domain of a KIR-CAR differs at no more than 15, 10, 5, 2, or 1% of its residues from a reference sequence, e.g., a naturally occurring KIR transmembrane domain or a KIR In transmembrane domain described herein. In some embodiments the KIR transmembrane domain of a KIR-CAR differs at no more than 5, 4, 3, 2 or 1 residue from a reference sequence, e.g., a naturally occurring KIR transmembrane domain or a KIR transmembrane domain described herein. In some embodiments the KIR transmembrane domain of a KIR-CAR does not differ from, or shares 100% homology with, a reference sequence, e.g., a naturally occurring KIR transmembrane domain or a KIR transmembrane domain described herein.

KIR intracellular domain, as that term is used herein, refers to a polypeptide domain having structural and functional properties of an intracellular domain of a KIR. KIR intracellular domains comprise inhibitory KIR intracellular domains (referred to herein as inhKIR intracellular domains) and activating KIR intracellular domains (referred to herein as actKIR intracellular domains). In an embodiment the inhKIR intracellular domain comprises an ITIM sequence. In an embodiment the KIR intracellular domain of a KIR-CAR has at least 70, 80, 85, 90, 95, or 99% homology with a reference sequence, e.g., a naturally occurring KIR intracellular domain or a KIR intracellular domain of a intracellular domain described herein. In some embodiments the KIR-CAR differs at no more than 15, 10, 5, 2, or 1% of its residues from a reference sequence, e.g., a naturally occurring KIR intracellular domain or a KIR intracellular domain described herein. In some embodiments the KIR intracellular domain of a KIR-CAR differs at no more than 5, 4, 3, 2 or 1 residue from a reference sequence, e.g., a naturally occurring KIR intracellular domain or a KIR intracellular domain described herein. In some embodiments the KIR intracellular domain of a KIR-CAR does not differ from, or shares 100% homology with, a reference sequence, e.g., a naturally occurring KIR intracellular domain or a KIR intracellular domain described herein.

### NORs

NKR-CARs described herein include NCR-CARs, which share functional and structural properties with NCRs.

Natural killer (NK) cells are cytotoxic lymphoid cells specialized in destroying tumors and virus-infected cells. Unlike cytotoxic T lymphocytes, NK cells do not express antigen-specific receptors. The recognition of transformed cells occurs via the association of a multitude of cell-surface receptors with surface markers on the target cell. The NK cell surface receptors can be distinguished according to whether they activate or inhibit NK cell-mediated cytotoxicity. Numerous interactions between different receptors appear to lead to the formation of synapses between NK and target cells. The integration of activating and inhibiting signals at the synapse dictates whether or not the NK cells exert their cytolytic function on the target cell. Among the activating receptors, the family of lg-like molecules is termed natural cytotoxicity receptors (NCRs). These natural cytotoxicity receptors include NKp30, NKp44 and NKp46 molecules. The NCRs are key activating receptors for NK cells in tumor cell recognition. All three NCRs are involved in the clearance of both tumor and virus-infected cells. In the latter, the antiviral activity is initiated by the interaction of NKp44 with hemagglutinin of influenza virus or Sendai virus. NKp46 targets virus-infected cells by binding to influenza virus hemagglutinin or Sendai virus hemagglutinin-neuraminidase. In contrast, it has been shown that NK cell-mediated cytotoxicity is inhibited by binding of NKp30 to the human cytomegaloviral protein pp65 (see, e.g., Amon, et. al., Nat. Immunol. (2005) 6:515-523).

Amino acid sequences for human NCR polypeptides (Homo sapiens) are available in the NCBI database, see e.g., accession number NP_004819.2 (GI: 153945782), 014931.1 (GI: 47605770), 095944.2 (GI: 251757303), 076036.1 (GI: 47605775), NP_001138939.1 (GI: 224586865), and/or NP_001138938.1 (GI: 224586860).

In an embodiment, a NCR-CAR comprises an antigen binding domain and a NCR transmembrane domain. In some embodiments, a KIR-CAR comprises an antigen binding domain and a NCR intracellular domain.

NCR extracellular domain, as that term is used herein, refers to a polypeptide domain having structural and functional properties of an extracellular domain of a NCR. In an embodiment the NCR extracellular domain of a NCR-CAR has at least 70, 80, 85, 90, 95, or 99% homology with a reference sequence, e.g., a naturally occurring NCR extracellular domain or a NCR extracellular domain described herein. In some embodiments the NCR extracellular domain of a NCR-CAR differs at no more than 15, 10, 5, 2, or 1% of its residues from a reference sequence, e.g., a naturally occurring NCR extracellular domain or a NCR extracellular domain described herein. In some embodiments the NCR extracellular domain of a NCR-CAR differs at no more than 5, 4, 3, 2 or 1 residue from a reference sequence, e.g., a naturally occurring NCR extracellular domain or a NCR extracellular domain described herein. In some embodiments the NCR extracellular domain of a NCR-CAR does not differ from, or shares 100% homology with, a reference sequence, e.g., a naturally occurring NCR extracellular domain or a NCR extracellular domain described herein.

NCR hinge or stem domain, as that term is used herein, refers to a polypeptide domain having structural and functional properties of a hinge or stem domain of a NCR. In an embodiment the NCR hinge or stem domain of a NCR-CAR has at least 70, 80, 85, 90, 95, or 99% homology with a reference sequence, e.g., a naturally occurring NCR hinge or stem domain or a NCR hinge or stem domain described herein. In some embodiments the NCR hinge or stem domain of a NCR-CAR differs at no more than 15, 10, 5, 2, or 1% of its residues from a reference sequence, e.g., a naturally occurring NCR hinge or stem domain or a NCR hinge or stem domain described herein. In some embodiments the NCR hinge or stem domain of a NCR-CAR differs at no more than 5, 4, 3, 2 or 1 residue from a reference sequence, e.g., a naturally occurring NCR hinge or stem domain or a NCR hinge or stem domain described herein. In embodiments the NCR hinge or stem domain of a NCR-CAR does not differ from, or shares 100% homology with a reference sequence, e.g., a naturally occurring NCR hinge or stem domain or a NCR hinge or stem domain described herein.

NCR transmembrane domain, as that term is used herein, refers to a polypeptide domain having structural and functional properties of a transmembrane domain of a NCR. In an embodiment the NCR transmembrane domain of a NCR-CAR has at least 70, 80, 85, 90, 95, or 99% homology with a reference sequence, e.g., a naturally occurring NCR transmembrane domain or a NCR transmembrane domain described herein. In some embodiments the NCR transmembrane domain of a NCR-CAR differs at no more than 15, 10, 5, 2, or 1% of its residues from a reference sequence, e.g., a naturally occurring NCR transmembrane domain or a NCR transmembrane domain described herein. In some embodiments the NCR transmembrane domain of a NCR-CAR differs at no more than 5, 4, 3, 2 or 1 residue from a reference sequence, e.g., a naturally occurring NCR transmembrane domain or a NCR transmembrane domain described herein. In some embodiments the NCR transmembrane domain of a NCR-CAR does not differ from, or shares 100% homology with, a reference sequence, e.g., a naturally occurring NCR transmembrane domain or a NCR transmembrane domain described herein.

NCR intracellular domain, as that term is used herein, refers to a polypeptide domain having structural and functional properties of an intracellular domain of a NCR. In some embodiments the NCR intracellular domain of a NCR-CAR has at least 70, 80, 85, 90, 95, or 99% homology with a reference sequence, e.g., a naturally occurring NCR intracellular domain or a NCR intracellular domain described herein. In some embodiments the NCR intracellular domain of a NCR-CAR differs at no more than 15, 10, 5, 2, or 1% of its residues from a reference sequence, e.g., a naturally occurring NCR intracellular domain or a NCR intracellular domain described herein. In some embodiments the NCR intracellular domain of a NCR-CAR differs at no more than 5, 4, 3, 2 or 1 residue from a reference sequence, e.g., a naturally occurring NCR intracellular domain or a NCR intracellular domain described herein. In some embodiments the NCR intracellular domain of a NCR-CAR does not differ from, or shares 100% homology with, a reference sequence, e.g., a naturally occurring NCR intracellular domain or a NCR intracellular domain described herein.

### SLAM Receptors

NKR-CARs described herein include SLAMF-CARs, which share functional and structural properties with SLAMFs.

The signaling lymphocyte activation molecule (SLAM) family of immune cell receptors is closely related to the CD2 family of the immunoglobulin (lg) superfamily of molecules. The SLAM family (SLAMF) currently includes nine members named SLAM, CD48, CD229, 2B4, CD84, NTB-A, CRACC, BLAME, and CD2F-10. In general, SLAM molecules possess two to four extracellular lg domains, a transmembrane segment, and an intracellular tyrosine-rich region. The molecules are differentially expressed on a variety of immune cell types. Several are self-ligands, and SLAM has been identified as the human measles virus receptor. Several small SH2-containing adaptor proteins are known to associate with the intracellular domains of SLAM family members and modulate receptor signaling including SH2DIA (also known as SLAM-associated protein [SAP]) and SH2D1B (also known as EAT2). For example, in T and NK cells, activated SLAM family receptors become tyrosine phosphorylated and recruit the adaptor SAP and subsequently the Src kinase Fyn. The ensuing signal transduction cascade influences the outcome of T cell-antigen presenting cell and NK cell-target cell interactions.

Amino acid sequences for human SLAM receptor polypeptides (Homo sapiens) are available in the NCBI database, see e.g., accession number NP_057466.1 (GI: 7706529), NP_067004.3 (GI: 19923572), NP_003028.1 (GI: 4506969), NP_001171808.1 (GI: 296434285), NP_001171643.1 (GI: 296040491), NP_001769.2 (GI: 21361571), NP_254273.2 (GI: 226342990), NP_064510.1 (GI: 9910342) and/or NP_002339.2 (GI: 55925578)

In some embodiments, a SLAMF-CAR comprises an antigen binding domain and a SLAMF transmembrane domain. In an embodiment, a SLAMF-CAR comprises an antigen binding domain and a SLAMF intracellular domain.

The term SLAMF extracellular domain, as used herein, refers to a polypeptide domain having structural and functional properties of an extracellular domain of a SLAMF. In an embodiment the SLAMF extracellular domain of a SLAMF-CAR has at least 70, 80, 85, 90, 95, or 99% homology with a reference sequence, e.g., a naturally occurring SLAMF extracellular domain or a SLAMF extracellular domain described herein. In some embodiments the SLAMF extracellular domain of a SLAMF-CAR differs at no more than 15, 10, 5, 2, or 1% of its residues from a reference sequence, e.g., a naturally occurring SLAMF extracellular domain or a SLAMF extracellular domain described herein. In some embodiments the SLAMF extracellular domain of a SLAMF-CAR differs at no more than 5, 4, 3, 2 or 1 residue from a reference sequence, e.g., a naturally occurring SLAMF extracellular domain or a SLAMF extracellular domain described herein. In some embodiments the SLAMF extracellular domain of a SLAMF-CAR does not differ from, or shares 100% homology with, a reference sequence, e.g., a naturally occurring SLAMF extracellular domain or a SLAMF extracellular domain described herein. SLAMF hinge or stem domain, as that term is used herein, refers to a polypeptide domain having structural and functional properties of a hinge or stem domain of a SLAMF. In an embodiment the SLAMF hinge or stem domain of a SLAMF-CAR has at least 70, 80, 85, 90, 95, or 99% homology with a reference sequence, e.g., a naturally occurring SLAMF hinge or stem domain or a SLAMF hinge or stem domain described herein. In some embodiments the SLAMF hinge or stem domain of a SLAMF-CAR differs at no more than 15, 10, 5, 2, or 1% of its residues from a reference sequence, e.g., a naturally occurring SLAMF hinge or stem domain or a SLAMF hinge or stem domain described herein. In some embodiments the SLAMF hinge or stem domain of a SLAMF-CAR differs at no more than 5, 4, 3, 2 or 1 residue from a reference sequence, e.g., a naturally occurring SLAMF hinge or stem domain or a SLAMF hinge or stem domain described herein. In some embodiments the SLAMF hinge or stem domain of a SLAMF-CAR does not differ from, or shares 100% homology with, a reference sequence, e.g., a naturally occurring SLAMF hinge or stem domain or a SLAMF hinge or stem domain described herein. SLAMF transmembrane domain, as that term is used herein, refers to a polypeptide domain having structural and functional properties of a transmembrane domain of a SLAMF. In an embodiment the SLAMF transmembrane domain of a SLAMF-CAR has at least 70, 80, 85, 90, 95, or 99% homology with a reference sequence, e.g., a naturally occurring SLAMF transmembrane domain or a SLAMF transmembrane domain described herein. In some embodiments the SLAMF transmembrane domain of a SLAMF-CAR differs at no more than 15, 10, 5, 2, or 1% of its residues from a reference sequence, e.g., a naturally occurring SLAMF transmembrane domain or a SLAMF transmembrane domain described herein. In some embodiments the SLAMF transmembrane domain of a SLAMF-CAR differs at no more than 5, 4, 3, 2 or 1 residue from a reference sequence, e.g., a naturally occurring SLAMF transmembrane domain or a SLAMF transmembrane domain described herein. In some embodiments the SLAMF transmembrane domain of a SLAMF-CAR does not differ from, or shares 100% homology with, a reference sequence, e.g., a naturally occurring SLAMF transmembrane domain or a SLAMF transmembrane domain described herein.

SLAMF intracellular domain, as that term is used herein, refers to a polypeptide domain having structural and functional properties of an intracellular domain of a SLAMF. In an embodiment the SLAMF intracellular domain of a SLAMF-CAR has at least 70, 80, 85, 90, 95, or 99% homology with a reference sequence, e.g., a naturally occurring SLAMF intracellular domain or a SLAMF intracellular domain described herein. In some embodiments the SLAMF intracellular domain of a SLAMF-CAR differs at no more than 15, 10, 5, 2, or 1% of its residues from a reference sequence, e.g., a naturally occurring SLAMF intracellular domain or a SLAMF intracellular domain described herein. In some embodiments the SLAMF intracellular domain of a SLAMF-CAR differs at no more than 5, 4, 3, 2 or 1 residue from a reference sequence, e.g., a naturally occurring SLAMF intracellular domain or a SLAMF intracellular domain described herein. In some embodiments the SLAMF intracellular domain of a SLAMF-CAR does not differ from, or shares 100% homology with, a reference sequence, e.g., a naturally occurring SLAMF intracellular domain or a SLAMF intracellular domain described herein.

### Fic-Binding Receptors

NKR-CARs described herein include CARs based on the Fc receptors, FcR-CARs, e.g., CD16-CARs, and CD64-CARs, which share functional and structural properties with CD16 and CD64.

Upon activation, NK cells produce cytokines and chemokines abundantly and at the same time exhibit potent cytolytic activity. Activation of NK cells can occur through the direct binding of NK cell receptors to ligands on the target cell, as seen with direct tumor cell killing, or through the crosslinking of the Fe receptor (CD 16; FcyRIII) by binding to the Fe portion of antibodies bound to an antigen-bearing cell. This CD16 engagement (CD16 crosslinking) initiates NK cell responses via intracellular signals that are generated through one, or both, of the CD16-associated adaptor chains, FcRy or CD3. Triggering of CD16 leads to phosphorylation of the y or s chain, which in turn recruits tyrosine kinases, syk, and ZAP-70, initiating a cascade of signal transduction leading to rapid and potent effector functions. The most well-known effector function is the release of cytoplasmic granules carrying toxic proteins to kill nearby target cells through the process of antibody-dependent cellular cytotoxicity. CD16 crosslinking also results in the production of cytokines and chemokines that, in turn, activate and orchestrate a series of immune responses.

However, unlike T and B lymphocytes, NK cells are thought to have only a limited capacity for target recognition using germline-encoded activation receptors (Bottino et al., Curr Top Microbial Immunol. 298:175-182 (2006); Stewart et al., Cun-Top Micro biol Immunol. 298:1-21 (2006)). NK cells express the activating Fe receptor CD 16, which recognizes IgG-coated target cells, thereby broadening target recognition (Ravetch & Bolland, Annu Rev Immunol. 19:275-290 (2001); Lanier Nat. Immunol. 9 (5): 495-502 (2008); Bryceson Long, Curr Opin Immunol. 20 (3): 344-352 (2008)). The expression and signal transduction activity of several NK cell activation receptors requires physically associated adaptors, which transduce signals through immunoreceptor tyrosine-based activation motifs (ITAMs). Among these adaptors, FcRy and CD3 (chains can associate with CD16 and natural cytotoxicity receptors (NCRs) as either disulfide-linked homodimers or heterodimers, and these chains have been thought to be expressed by all mature NK cells. Amino acid sequence for CD16 (Homo sapiens) is available in the NCBI database, see, e.g., accession number NP_000560.5 (GI: 50726979), NP_001231682.1 (GI: 348041254)

In some embodiments, a FcR-CAR comprises an antigen binding domain and a FcR transmembrane domain. In some embodiments, a FcR-CAR comprises an antigen binding domain and a FcR intracellular domain.

CD 16 extracellular domain, as that term is used herein, refers to a polypeptide domain having structural and functional properties of an extracellular domain of a CD16. In some embodiments, the CD16 extracellular domain of a CD16-CAR has at least 70, 80, 85, 90, 95, or 99% homology with a reference sequence, e.g., a naturally occurring CD16 extracellular domain or a CD16 extracellular domain described herein. In some embodiments the CD16 extracellular domain of a CD16-CAR differs at no more than 15, 10, 5, 2, or 1% of its residues from a reference sequence, e.g., a naturally occurring CD16 extracellular domain or a CD16 extracellular domain described herein. In some embodiments the CD16 extracellular domain of a CD16-CAR differs at no more than 5, 4, 3, 2 or 1 residue from a reference sequence, e.g., a naturally occurring CD16 extracellular domain or a CD16 extracellular domain described herein.

In some embodiments, the CD 16 extracellular domain of a CD16-CAR does not differ from, or shares 100% homology with a reference sequence, e.g., a naturally occurring CD16 extracellular domain or a CD16 extracellular domain described herein.

CD16 hinge or stem domain, as that term is used herein, refers to a polypeptide domain having structural and functional properties of a hinge or stem domain of a CD16. In an embodiment the CD16 hinge or stem domain of a CD16-CAR has at least 70, 80, 85, 90, 95, or 99% homology with a reference sequence, e.g., a naturally occurring CD16 hinge or stem domain or a CD 16 hinge or stem domain described herein. In some embodiments the CD16 hinge or stem domain of a CD16-CAR differs at no more than 15, 10, 5, 2, or 1% of its residues from a reference sequence, e.g., a naturally occurring CD16 hinge or stem domain or a CD16 hinge or stem domain described herein. In some embodiments the CD16 hinge or stem domain of a CD16-CAR differs at no more than 5, 4, 3, 2 or 1 residue from a reference sequence, e.g., a naturally occurring CD16 hinge or stem domain or a CD16 hinge or stem domain described herein. In some embodiments the CD16 hinge or stem domain of a CD16-CAR does not differ from, or shares 100% homology with, a reference sequence, e.g., a naturally occurring CD16 hinge or stem domain or a CD 16 hinge or stem domain described herein.

CD16 transmembrane domain, as that term is used herein, refers to a polypeptide domain having structural and functional properties of a transmembrane domain of a CD16. In an embodiment the CD16 transmembrane domain of a CD16-CAR has at least 70, 80, 85, 90, 95, or 99% homology with a reference sequence, e.g., a naturally occurring CD16 transmembrane domain or a CD16 transmembrane domain described herein. In some embodiments the CD16 transmembrane domain of a CD16-CAR differs at no more than 15, 10, 5, 2, or 1% of its residues from a reference sequence, e.g., a naturally occurring CD16 transmembrane domain or a CD16 transmembrane domain described herein. In some embodiments the CD16 transmembrane domain of a CD 16-CAR differs at no more than 5, 4, 3, 2 or 1 residue from a reference sequence, e.g., a naturally occurring CD16 transmembrane domain or a CD16 transmembrane domain described herein. In some embodiments the CD16 transmembrane domain of a CD16-CAR does not differ from, or shares 100% homology with, a reference sequence, e.g., a naturally occurring CD16 transmembrane domain or a CD16 transmembrane domain described herein.

CD16 intracellular domain, as that term is used herein, refers to a polypeptide domain having structural and functional properties of an intracellular domain of a CD16. In an embodiment the CD16 intracellular domain of a CD16-CAR has at least 70, 80, 85, 90, 95, or 99% homology with a reference sequence, e.g., a naturally occurring CD16 intracellular domain or a CD 16 intracellular domain described herein. In some embodiments the CD 16 intracellular domain of a CD16-CAR differs at no more than 15, 10, 5, 2, or 1% of its residues from a reference sequence, e.g., a naturally occurring CD16 intracellular domain or a CD16 intracellular domain described herein. In some embodiments the CD16 intracellular domain of a CD16-CAR differs at no more than 5, 4, 3, 2 or 1 residue from a reference sequence, e.g., a naturally occurring CD16 intracellular domain or a CD16 intracellular domain described herein. In some embodiments the CD16 intracellular domain of a CD16-CAR does not differ from, or shares 100% homology with, a reference sequence, e.g., a naturally occurring CD16 intracellular domain or a CD16 intracellular domain described herein.

CD64 extracellular domain, as that term is used herein, refers to a polypeptide domain having structural and functional properties of an extracellular domain of a CD64. In an embodiment the CD64 extracellular domain of a CD64-CAR has at least 70, 80, 85, 90, 95, or 99% homology with a reference sequence, e.g., a naturally occurring CD64 extracellular domain or a CD64 extracellular domain described herein. In some embodiments the CD64 extracellular domain of a CD64-CAR differs at no more than 15, 10, 5, 2, or 1% of its residues from a reference sequence, e.g., a naturally occurring CD64 extracellular domain or a CD64 extracellular domain described herein. In some embodiments the CD64 extracellular domain of a CD64-CAR differs at no more than 5, 4, 3, 2 or 1 residue from a reference sequence, e.g., a naturally occurring CD64 extracellular domain or a CD64 extracellular domain described herein. In some embodiments the CD64 extracellular domain of a CD64-CAR does not differ from, or shares 100% homology with, a reference sequence, e.g., a naturally occurring CD64 extracellular domain or a CD64 extracellular domain described herein.

CD64 transmembrane domain, as that term is used herein, refers to a polypeptide domain having structural and functional properties of a transmembrane domain of a CD64. In an embodiment the CD64 transmembrane domain of a CD64-CAR has at least 70, 80, 85, 90, 95, or 99% homology with a reference sequence, e.g., a naturally occurring CD64 transmembrane domain or a CD64 transmembrane domain described herein. In some embodiments the CD64 transmembrane domain of a CD64-CAR differs at no more than 15, 10, 5, 2, or 1% of its residues from a reference sequence, e.g., a naturally occurring CD64 transmembrane domain or a CD64 transmembrane domain described herein. In some embodiments the CD64 transmembrane domain of a CD64-CAR differs at no more than 5, 4, 3, 2 or 1 residue from a reference sequence, e.g., a naturally occurring CD64 transmembrane domain or a CD64 transmembrane domain described herein. In some embodiments the CD64 transmembrane domain of a CD64-CAR does not differ from, or shares 100% homology with, a reference sequence, e.g., a naturally occurring CD64 transmembrane domain or a CD64 transmembrane domain described herein.

CD64 intracellular domain, as that term is used herein, refers to a polypeptide domain having structural and functional properties of an intracellular domain of a CD64. In an embodiment the CD64 intracellular domain of a CD64-CAR has at least 70, 80, 85, 90, 95, or 99% homology with a reference sequence, e.g., a naturally occurring CD64 intracellular domain or a CD64 intracellular domain described herein. In some embodiments the CD64 intracellular domain of a CD64-CAR differs at no more than 15, 10, 5, 2, or 1% of its residues from a reference sequence, e.g., a naturally occurring CD64 intracellular domain or a CD64 intracellular domain described herein. In some embodiments the CD64 intracellular domain of a CD64-CAR differs at no more than 5, 4, 3, 2 or 1 residue from a reference sequence, e.g., a naturally occurring CD64 intracellular domain or a CD64 intracellular domain described herein. In some embodiments the CD64 intracellular domain of a CD64-CAR does not differ from, or shares 100% homology with, a reference sequence, e.g., a naturally occurring CD64 intracellular domain or a CD64 intracellular domain described herein.

### Ly49 and Related Killer Cell Lectin-Like Receptors

NKR-CARs described herein include Ly49-CARs, which share functional and structural properties with Ly49.

The Ly49 receptors derive from at least 23 identified genes (Ly49A-W) in mice. These receptors share many of the same roles in mouse NK cells and T cells as that played by KIRs in humans despite their different structure (type II integral membrane proteins of the C-type lectin superfamily), and they also contain a considerable degree of genetic variation like human KIRs. The remarkable functional similarity between Ly49 and KIR receptors suggests that these groups of receptors have evolved independently yet convergently to perform the same physiologic functionals in NK cells and T cells.

Like KIRs in humans, different Ly49 receptors recognize different MHC class I alleles and are differentially expressed on subsets of NK cells. The original prototypic Ly49 receptors, Ly49A and Ly49C possess a cytoplasmic domain bearing two immunotyrosine-based inhibitory motifs (ITIM) similar to inhibitory KIRs such as KIR2DL3. These domains have been identified to recruit the phosphatase, SHP-1, and like the inhibitory KIRs, serve to limit the activation of NK cells and T cells. In addition to the inhibitory Ly49 molecules, several family members such as Ly49D and Ly49H have lost the IT IM-containing domains and have instead acquired the capacity to interact with the signaling adaptor molecule, DAP12 similar to the activating KIRs such as KIR2DS2 in humans.

Amino acid sequences for Ly49 family members are available in the NCBI database, see e.g., accession numbers AAF82184.1 (GI: 9230810), AAF99547.1 (GI: 9801837), NP_034778.2 (GI: 133922593), NP_034779.1 (GI: 6754462), NP_001095090.1 (GI: 197333718), NP 034776.1 (GI: 21327665), AAKl 1559.1 (GI: 13021834) and/or NP_038822.3 (GI: 9256549).

In some embodiments, a Ly49-CAR comprises an antigen binding domain and a Ly49 transmembrane domain. In an embodiment, a Ly49-CAR comprises an antigen binding domain and a Ly49 intracellular domain.

Ly49 extracellular domain, as that term is used herein, refers to a polypeptide domain having structural and functional properties of the extracellular domain of a Ly49. In some embodiments the Ly49 extracellular domain of a Ly49-CAR has at least 70, 80, 85, 90, 95, or 99% homology with a reference sequence, e.g., a naturally occurring Ly49 extracellular domain or a Ly49 extracellular domain described herein. In some embodiments the Ly49 extracellular domain of a Ly49-CAR differs at no more than 15, 10, 5, 2, or 1% of its residues from a reference sequence, e.g., a naturally occurring Ly49 extracellular domain or a Ly49 extracellular domain described herein. In some embodiments the Ly49 extracellular domain of a Ly49-CAR differs at no more than 5, 4, 3, 2 or 1 residue from a reference sequence, e.g., a naturally occurring Ly49 extracellular domain or a Ly49 extracellular domain described herein. In some embodiments the Ly49 extracellular domain of a Ly49-CAR does not differ from, or shares 100% homology with, a reference sequence, e.g., a naturally occurring Ly49 extracellular domain or a Ly49 extracellular domain described herein.

Ly49 hinge or stem domain, as that term is used herein, refers to a polypeptide domain having structural and functional properties of a hinge or stem domain of a Ly49. In some embodiments the Ly49 hinge or stem domain of a Ly49-CAR has at least 70, 80, 85, 90, 95, or 99% homology with a reference sequence, e.g., a naturally occurring Ly49 hinge or stem domain or a Ly49 hinge or stem domain described herein. In some embodiments the Ly49 hinge or stem domain of a Ly49-CAR differs at no more than 15, 10, 5, 2, or 1% of its residues from a reference sequence, e.g., a naturally occurring Ly49 hinge or stem domain or a Ly49 hinge or stem domain described herein. In some embodiments the Ly49 hinge or stem domain of a Ly49-CAR differs at no more than 5, 4, 3, 2 or 1 residue from a reference sequence, e.g., a naturally occurring Ly49 hinge or stem domain or a Ly49 hinge or stem domain described herein. In some embodiments the Ly49 hinge or stem domain of a Ly49-CAR does not differ from, or shares 100% homology with a reference sequence, e.g., a naturally occurring Ly49 hinge or stem domain or a Ly49 hinge or stem domain described herein.

Ly49 transmembrane domain, as that term is used herein, refers to a polypeptide domain having structural and functional properties of a transmembrane domain of a Ly49. In some embodiments the Ly49 transmembrane domain of a Ly49-CAR has at least 70, 80, 85, 90, 95, or 99% homology with a reference sequence, e.g., a naturally occurring Ly49 transmembrane domain or a Ly49 transmembrane domain described herein. In some embodiments the Ly49 transmembrane domain of a Ly49-CAR differs at no more than 15, 10, 5, 2, or 1% of its residues from a reference sequence, e.g., a naturally occurring Ly49 transmembrane domain or a Ly49 transmembrane domain described herein. In some embodiments, the Ly49 transmembrane domain of a Ly49-CAR differs at no more than 5, 4, 3, 2 or 1 residue from a reference sequence, e.g., a naturally occurring Ly49 transmembrane domain or a Ly49 transmembrane domain described herein. In some embodiments, the Ly49 transmembrane domain of a Ly49-CAR does not differ from, or shares 100% homology with, a reference sequence, e.g., a naturally occurring Ly49 transmembrane domain or a Ly49 transmembrane domain described herein.

Ly49 intracellular domain, as that term is used herein, refers to a polypeptide domain having structural and functional properties of an intracellular domain of a Ly49. In some embodiments the Ly49 intracellular domain of a Ly49-CAR has at least 70, 80, 85, 90, 95, or 99% homology with a reference sequence, e.g., a naturally occurring Ly49 intracellular domain or a Ly49 intracellular domain described herein. In some embodiments the Ly49 intracellular domain of a LY49-CAR differs at no more than 15, 10, 5, 2, or 1% of its residues from a reference sequence, e.g., a naturally occurring Ly49 intracellular domain or a Ly49 intracellular domain described herein. In some embodiments the Ly49 intracellular domain of a Ly49-CAR differs at no more than 5, 4, 3, 2 or 1 residue from a reference sequence, e.g., a naturally occurring Ly49 intracellular domain or a Ly49 intracellular domain described herein. In some embodiments, the Ly49 intracellular domain of a Ly49-CAR does not differ from, or shares 100% homology with, a reference sequence, e.g., a naturally occurring Ly49 intracellular domain or a Ly49 intracellular domain described herein.

### Intracellular Signaling Domains or Adaptor Molecules

Some NKR-CARs interact with other molecules, e.g., molecules comprising an intracellular signaling domain, e.g., an ITAM. In an embodiment an intracellular signaling domain is DAP12.

DAP12 is so named because of its structural features, and presumed function. Certain cell surface receptors lack intrinsic functionality, which hypothetically may interact with another protein partner suggested to be a 12 kD protein. The mechanism of the signaling may involve an ITAM signal.

DAP12 was identified from sequence databases based upon a hypothesized relationship to CD3 (see Olcese, et al. (1997) J. Immunol. 158:5083-5086), the presence of an ITAM sequence (see Thomas (1995) J. Exp. Med. 181:1953-1956), certain size predictions (see Olcese; and Takase, et al. (1997) J. Immunol. 159:741-747, and other features. In particular, the transmembrane domain was hypothesized to contain a charged residue, which would allow a salt bridge with the corresponding transmembrane segments of its presumed receptor partners, KIR CD94 protein, and possibly other similar proteins. See Daeron, et al. (1995) Immunity 3:635-646.

In fact, many of the known KIR, MIR, ILT, and CD94/NKG2 receptor molecules may actually function with an accessory protein which is part of the functional receptor complex. See Olcese, et al. (1997) J. Immunol. 158:5083-5086; and Takase, et al. (1997) J. Immunol. 159:741-747.

A DAP12 domain, as that term is used herein, refers to a polypeptide domain having structural and functional properties of a cytoplasmic domain of a DAP12, and will typically include an ITAM domain. In an embodiment a DAP 12 domain of a KIR-CAR has at least 70, 80, 85, 90, 95, or 99% homology with a reference sequence, e.g., a naturally occurring DAP 12 or a DAP 12 described herein. In some embodiments, the DAP12 domain of a KIR-CAR differs at no more than 15, 10, 5, 2, or 1% of its residues from a reference sequence, e.g., a naturally occurring DAP12 or a DAP12 described herein. In some embodiments, the DAP12 domain of a KIR-CAR differs at no more than 5, 4, 3, 2 or 1 residue from a reference sequence, e.g., a naturally occurring DAP12 or a DAP12 described herein. In some embodiments, the DAP12 domain of a KIR-CAR does not differ from, or shares 100% homology with, a reference sequence, e.g., a naturally occurring DAP12 or a DAP12 described herein.

The DAP10 was identified partly by its homology to the DAP12, and other features. In particular, in contrast to the DAP12, which exhibits an ITAM activation motif, the DAP10 exhibits an ITIM inhibitory motif. The MDL-1 was identified by its functional association with DAP12.

The functional interaction between, e.g., DAP12 or DAP10, and its accessory receptor may allow use of the structural combination in receptors which normally are not found in a truncated receptor form. Thus, the mechanism of signaling through such accessory proteins as the DAP12 and DAP10 allow for interesting engineering of other KIR-like receptor complexes, e.g., with the KIR, MIR, ILT, and CD94 NKG2 type receptors. Truncated forms of intact receptors may be constructed which interact with a DAP12 or DAP10 to form a functional signaling complex.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g., a tumor associated antigen) on a target cell (e.g., a cancer cell). The target antigen may include any type of protein, or epitope thereof, associated with the target cell. For example, the CAR may comprise affinity to a target antigen on a target cell that indicates a particular status of the target cell.

In certain embodiments, the CAR of the invention comprises an antigen binding domain that binds to hCD19. In certain embodiments, the antigen binding domain of the invention comprises an antibody or fragment thereof, that binds to a hCD19 molecule. In certain exemplary embodiments, the antigen binding domain is an scFv antibody that binds to hCD19. The choice of antigen binding domain depends upon the type and number of antigens that are present on the surface of a target cell. For example, the antigen binding domain may be chosen to recognize an antigen that acts as a cell surface marker on a target cell associated with a particular status of the target cell.

As described herein, a CAR of the present disclosure having affinity for a specific target antigen on a target cell may comprise a target-specific binding domain. In some embodiments, the target-specific binding domain is a human target-specific binding domain, e.g., the target-specific binding domain is of human origin. In an exemplary embodiment, a CAR of the present disclosure having affinity for hCD19 on a target cell may comprise a hCD19 binding domain. In some embodiments, the hCD19 binding domain is a canine hCD19 binding domain, e.g., the hCD19 binding domain is of canine origin. In some embodiments, the hCD19 binding domain is a humanized CD19 binding domain. In some embodiments, the hCD19 binding domain is a human CD19 binding domain, e.g., the hCD19 binding domain is of human origin.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In another embodiment, the antigen binding domain of the CAR is selected from the group consisting of an anti-hCD19 antibody and a fragment thereof. In some embodiments, the antigen binding domain is selected from the group consisting of an antibody, an antigen binding fragment (Fab), and a single-chain variable fragment (scFv). In some embodiments, a hCD19 binding domain of the present invention is selected from the group consisting of a hCD19-specific antibody, a hCD19-specific Fab, and a hCD19-specific scFv. In one embodiment, a hCD19 binding domain is a hCD19-specific antibody. In one embodiment, a hCD19 binding domain is a hCD19-specific Fab. In one embodiment, a hCD19 binding domain is a hCD19-specific scFv.

As used herein, the term “single-chain variable fragment” or “scFv” is a fusion protein of the variable regions of the heavy (VH) and light chains (VL) of an immunoglobulin (e.g., mouse or human) covalently linked to form a VH: VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., hCD19 binding domain) comprises an scFv having the configuration from N-terminus to C-terminus, VH-linker-VL. In some embodiments, the antigen binding domain (e.g., hCD19 binding domain) comprises an scFv having the configuration from N-terminus to C-terminus, VL-linker-VH. Those of skill in the art would be able to select the appropriate configuration for use in the present invention.

In some embodiments, the hCD 19 binding domain is derived from an scFv specific for human CD19 as disclosed elsewhere herein. Accordingly, a CAR of the present disclosure comprises a hCD19 binding domain derived from an scFv disclosed elsewhere herein.

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In one embodiment, the hCD19 binding domain comprises a light chain variable region comprising an amino acid sequence set forth in SEQ ID NOs: 271-313. The light chain variable region of the hCD19 binding domain comprises three light chain complementarity-determining regions (CDRs). As used herein, a “complementarity-determining region” or “CDR” refers to a region of the variable chain of an antigen binding molecule that binds to a specific antigen. Accordingly, a hCD19 binding domain may comprise a light chain variable region that comprises a LCDR1 comprising an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; a LCDR2 comprising an amino acid sequence selected from the group consisting of ADS, AND, ANI, EAS, GNS, GNT, GSH, GSS, GYN, KDT, LVS, NND, QVS, RDT, SDG, SNK, SSS, and SVS; and a LCDR3 comprising an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

In one embodiment, the hCD19 binding domain comprises a heavy chain variable region comprising an amino acid sequence set forth in SEQ ID NOs: 314-356. A hCD19 binding domain may comprise a heavy chain variable region that comprises a hCDR1 comprising an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245.

In some embodiments, the hCD19 binding domain comprises an HCDR1, HCDR2, HCDR3, LCDR1, and LCDR3 which comprise the respective amino acid sequences set forth in:
SEQ ID NOs: 87, 88, 89, 90, and 91 and wherein LCDR2 comprises the amino acid sequence GNS;
SEQ ID NOs: 92, 93, 94, 95, and 96, and wherein LCDR2 comprises the amino acid sequence AND;
SEQ ID NOs: 97, 98, 99, 100, and 101, and wherein LCDR2 comprises the amino acid sequence ANI;
SEQ ID NOs: 102, 103, 104, 100, and 105, and wherein LCDR2 comprises the amino acid sequence ANI;
SEQ ID NOs: 106, 107, 108, 109, and 110, and wherein LCDR2 comprises the amino acid sequence SDG;
SEQ ID NOs: 111, 112, 113, 114, and 115, and wherein LCDR2 comprises the amino acid sequence GSS;
SEQ ID NOs: 116, 117, 118, 119, and 120, and wherein LCDR2 comprises the amino acid sequence GNT;
SEQ ID NOs: 121, 122, 123, 124, and 125, and wherein LCDR2 comprises the amino acid sequence GYN;
SEQ ID NOs: 126, 117, 127, 128, and 129, and wherein LCDR2 comprises the amino acid sequence GNS;
SEQ ID NOs: 130, 131, 132, 100, and 133, and wherein LCDR2 comprises the amino acid sequence ANI;
SEQ ID NOs: 134, 135, 136, 100, and 105, and wherein LCDR2 comprises the amino acid sequence ANI;
SEQ ID NOs: 137, 138, 139, 140, and 141, and wherein LCDR2 comprises the amino acid sequence ADS;
SEQ ID NOs: 142, 143, 144, 100, and 105, and wherein LCDR2 comprises the amino acid sequence ANI;
SEQ ID NOs: 145, 146, 147, 148, and 149, and wherein LCDR2 comprises the amino acid sequence GNS;
SEQ ID NOs: 150, 151, 152, 153, and 154, and wherein LCDR2 comprises the amino acid sequence GNS;
SEQ ID NOs: 155, 156, 157, 158, and 159, and wherein LCDR2 comprises the amino acid sequence GSH;
SEQ ID NOs: 121, 160, 161, 128, and 162, and wherein LCDR2 comprises the amino acid sequence GNS;
SEQ ID NOs: 163, 164, 123, 124, and 165, and wherein LCDR2 comprises the amino acid sequence GYN;
SEQ ID NOs: 106, 107, 108, 166, and 167, and wherein LCDR2 comprises the amino acid sequence SDG;
SEQ ID NOs: 168, 169, 170, 171, and 172, and wherein LCDR2 comprises the amino acid sequence KDT;
SEQ ID NOs: 173, 174, 175, 171, and 176, and wherein LCDR2 comprises the amino acid sequence KDT;
SEQ ID NOs: 177, 178, 179, 180, and 181, and wherein LCDR2 comprises the amino acid sequence SSS;
SEQ ID NOs: 182, 183, 184, 185, and 186, and wherein LCDR2 comprises the amino acid sequence LVS;
SEQ ID NOs: 187, 188, 189, 100, and 190, and wherein LCDR2 comprises the amino acid sequence ANI;
SEQ ID NOs: 191, 192, 193, 185, and 194, and wherein LCDR2 comprises the amino acid sequence QVS;
SEQ ID NOs: 195, 196, 197, 171, and 198, and wherein LCDR2 comprises the amino acid sequence KDT;
SEQ ID NOs: 195, 196, 197, 171, and 199, and wherein LCDR2 comprises the amino acid sequence KDT;
SEQ ID NOs: 195, 196, 197, 200, and 201, and wherein LCDR2 comprises the amino acid sequence RDT;
SEQ ID NOs: 195, 196, 197, 200, and 202, and wherein LCDR2 comprises the amino acid sequence KDT;
SEQ ID NOs: 195, 196, 197, 171, and 190, and wherein LCDR2 comprises the amino acid sequence KDT;
SEQ ID NOs: 195, 196, 197, 203, and 204, and wherein LCDR2 comprises the amino acid sequence RDT;
SEQ ID NOs: 195, 196, 197, 171, and 205, and wherein LCDR2 comprises the amino acid sequence KDT;
SEQ ID NOs: 206, 207, 208, 209, and 210, and wherein LCDR2 comprises the amino acid sequence SVS;
SEQ ID NOs: 211, 112, 113, 114, and 115, and wherein LCDR2 comprises the amino acid sequence GSS;
SEQ ID NOs: 168, 117, 212, 213, and 214, and wherein LCDR2 comprises the amino acid sequence KDT;
SEQ ID NOs: 121, 215, 216, 166, and 217, and wherein LCDR2 comprises the amino acid sequence SDG;
SEQ ID NOs: 218, 219, 220, 221, and 222, and wherein LCDR2 comprises the amino acid sequence RDT;
SEQ ID NOs: 223, 224, 225, 213, and 226, and wherein LCDR2 comprises the amino acid sequence KDT;
SEQ ID NOs: 121, 227, 228, 229, and 230, and wherein LCDR2 comprises the amino acid sequence SDG;
SEQ ID NOs: 231, 232, 233, 234, and 235, and wherein LCDR2 comprises the amino acid sequence NND;
SEQ ID NOs: 236, 237, 238, 171, and 239, and wherein LCDR2 comprises the amino acid sequence KDT;
SEQ ID NOs: 240, 143, 241, 242, and 243, and wherein LCDR2 comprises the amino acid sequence SNK; or
SEQ ID NOs: 244, 138, 245, 246, and 247, and wherein LCDR2 comprises the amino acid sequence EAS.

In one embodiment, the hCD19 binding domain comprises a heavy chain variable region comprising the amino acid sequence set forth in SEQ ID NO: 314 and a light chain variable region comprising the amino acid sequence set forth in SEQ ID NO: 271.

In one embodiment, the hCD19 binding domain comprises a heavy chain variable region comprising the amino acid sequence set forth in SEQ ID NO: 315 and a light chain variable region comprising the amino acid sequence set forth in SEQ ID NO: 272.

In one embodiment, the hCD19 binding domain comprises a heavy chain variable region comprising the amino acid sequence set forth in SEQ ID NO: 316 and a light chain variable region comprising the amino acid sequence set forth in SEQ ID NO: 273.

In one embodiment, the hCD19 binding domain comprises a heavy chain variable region comprising the amino acid sequence set forth in SEQ ID NO: 317 and a light chain variable region comprising the amino acid sequence set forth in SEQ ID NO: 274.

In one embodiment, the hCD19 binding domain comprises a heavy chain variable region comprising the amino acid sequence set forth in SEQ ID NO: 318 and a light chain variable region comprising the amino acid sequence set forth in SEQ ID NO: 275.

In one embodiment, the hCD19 binding domain comprises a heavy chain variable region comprising the amino acid sequence set forth in SEQ ID NO: 319 and a light chain variable region comprising the amino acid sequence set forth in SEQ ID NO: 276.

In one embodiment, the hCD19 binding domain comprises a heavy chain variable region comprising the amino acid sequence set forth in SEQ ID NO: 320 and a light chain variable region comprising the amino acid sequence set forth in SEQ ID NO: 277.

In one embodiment, the hCD19 binding domain comprises a heavy chain variable region comprising the amino acid sequence set forth in SEQ ID NO: 321 and a light chain variable region comprising the amino acid sequence set forth in SEQ ID NO: 278.

In one embodiment, the hCD19 binding domain comprises a heavy chain variable region comprising the amino acid sequence set forth in SEQ ID NO: 322 and a light chain variable region comprising the amino acid sequence set forth in SEQ ID NO: 279.

In one embodiment, the hCD19 binding domain comprises a heavy chain variable region comprising the amino acid sequence set forth in SEQ ID NO: 323 and a light chain variable region comprising the amino acid sequence set forth in SEQ ID NO: 280.

In one embodiment, the hCD19 binding domain comprises a heavy chain variable region comprising the amino acid sequence set forth in SEQ ID NO: 324 and a light chain variable region comprising the amino acid sequence set forth in SEQ ID NO: 281.

In one embodiment, the hCD19 binding domain comprises a heavy chain variable region comprising the amino acid sequence set forth in SEQ ID NO: 325 and a light chain variable region comprising the amino acid sequence set forth in SEQ ID NO: 282.

In one embodiment, the hCD19 binding domain comprises a heavy chain variable region comprising the amino acid sequence set forth in SEQ ID NO: 326 and a light chain variable region comprising the amino acid sequence set forth in SEQ ID NO: 283.

In one embodiment, the hCD19 binding domain comprises a heavy chain variable region comprising the amino acid sequence set forth in SEQ ID NO: 327 and a light chain variable region comprising the amino acid sequence set forth in SEQ ID NO: 284.

In one embodiment, the hCD19 binding domain comprises a heavy chain variable region comprising the amino acid sequence set forth in SEQ ID NO: 328 and a light chain variable region comprising the amino acid sequence set forth in SEQ ID NO: 285.

In one embodiment, the hCD19 binding domain comprises a heavy chain variable region comprising the amino acid sequence set forth in SEQ ID NO: 329 and a light chain variable region comprising the amino acid sequence set forth in SEQ ID NO: 286.

In one embodiment, the hCD19 binding domain comprises a heavy chain variable region comprising the amino acid sequence set forth in SEQ ID NO: 330 and a light chain variable region comprising the amino acid sequence set forth in SEQ ID NO: 287.

In one embodiment, the hCD19 binding domain comprises a heavy chain variable region comprising the amino acid sequence set forth in SEQ ID NO: 331 and a light chain variable region comprising the amino acid sequence set forth in SEQ ID NO: 288.

In one embodiment, the hCD19 binding domain comprises a heavy chain variable region comprising the amino acid sequence set forth in SEQ ID NO: 332 and a light chain variable region comprising the amino acid sequence set forth in SEQ ID NO: 289.

In one embodiment, the hCD19 binding domain comprises a heavy chain variable region comprising the amino acid sequence set forth in SEQ ID NO: 333 and a light chain variable region comprising the amino acid sequence set forth in SEQ ID NO: 290.

In one embodiment, the hCD19 binding domain comprises a heavy chain variable region comprising the amino acid sequence set forth in SEQ ID NO: 334 and a light chain variable region comprising the amino acid sequence set forth in SEQ ID NO: 291.

In one embodiment, the hCD19 binding domain comprises a heavy chain variable region comprising the amino acid sequence set forth in SEQ ID NO: 335 and a light chain variable region comprising the amino acid sequence set forth in SEQ ID NO: 292.

In one embodiment, the hCD19 binding domain comprises a heavy chain variable region comprising the amino acid sequence set forth in SEQ ID NO: 336 and a light chain variable region comprising the amino acid sequence set forth in SEQ ID NO: 293.

In one embodiment, the hCD19 binding domain comprises a heavy chain variable region comprising the amino acid sequence set forth in SEQ ID NO: 337 and a light chain variable region comprising the amino acid sequence set forth in SEQ ID NO: 294.

In one embodiment, the hCD19 binding domain comprises a heavy chain variable region comprising the amino acid sequence set forth in SEQ ID NO: 338 and a light chain variable region comprising the amino acid sequence set forth in SEQ ID NO: 295.

In one embodiment, the hCD19 binding domain comprises a heavy chain variable region comprising the amino acid sequence set forth in SEQ ID NO: 339 and a light chain variable region comprising the amino acid sequence set forth in SEQ ID NO: 296.

In one embodiment, the hCD19 binding domain comprises a heavy chain variable region comprising the amino acid sequence set forth in SEQ ID NO: 340 and a light chain variable region comprising the amino acid sequence set forth in SEQ ID NO: 297.

In one embodiment, the hCD19 binding domain comprises a heavy chain variable region comprising the amino acid sequence set forth in SEQ ID NO: 341 and a light chain variable region comprising the amino acid sequence set forth in SEQ ID NO: 298.

In one embodiment, the hCD19 binding domain comprises a heavy chain variable region comprising the amino acid sequence set forth in SEQ ID NO: 342 and a light chain variable region comprising the amino acid sequence set forth in SEQ ID NO: 299.

In one embodiment, the hCD19 binding domain comprises a heavy chain variable region comprising the amino acid sequence set forth in SEQ ID NO: 343 and a light chain variable region comprising the amino acid sequence set forth in SEQ ID NO: 300.

In one embodiment, the hCD19 binding domain comprises a heavy chain variable region comprising the amino acid sequence set forth in SEQ ID NO: 344 and a light chain variable region comprising the amino acid sequence set forth in SEQ ID NO: 301.

In one embodiment, the hCD19 binding domain comprises a heavy chain variable region comprising the amino acid sequence set forth in SEQ ID NO: 345 and a light chain variable region comprising the amino acid sequence set forth in SEQ ID NO: 302.

In one embodiment, the hCD19 binding domain comprises a heavy chain variable region comprising the amino acid sequence set forth in SEQ ID NO: 346 and a light chain variable region comprising the amino acid sequence set forth in SEQ ID NO: 303.

In one embodiment, the hCD19 binding domain comprises a heavy chain variable region comprising the amino acid sequence set forth in SEQ ID NO: 347 and a light chain variable region comprising the amino acid sequence set forth in SEQ ID NO: 304.

In one embodiment, the hCD19 binding domain comprises a heavy chain variable region comprising the amino acid sequence set forth in SEQ ID NO: 348 and a light chain variable region comprising the amino acid sequence set forth in SEQ ID NO: 305.

In one embodiment, the hCD19 binding domain comprises a heavy chain variable region comprising the amino acid sequence set forth in SEQ ID NO: 349 and a light chain variable region comprising the amino acid sequence set forth in SEQ ID NO: 306.

In one embodiment, the hCD19 binding domain comprises a heavy chain variable region comprising the amino acid sequence set forth in SEQ ID NO: 350 and a light chain variable region comprising the amino acid sequence set forth in SEQ ID NO: 307.

In one embodiment, the hCD19 binding domain comprises a heavy chain variable region comprising the amino acid sequence set forth in SEQ ID NO: 351 and a light chain variable region comprising the amino acid sequence set forth in SEQ ID NO: 308.

In one embodiment, the hCD19 binding domain comprises a heavy chain variable region comprising the amino acid sequence set forth in SEQ ID NO: 352 and a light chain variable region comprising the amino acid sequence set forth in SEQ ID NO: 309.

In one embodiment, the hCD19 binding domain comprises a heavy chain variable region comprising the amino acid sequence set forth in SEQ ID NO: 353 and a light chain variable region comprising the amino acid sequence set forth in SEQ ID NO: 310.

In one embodiment, the hCD19 binding domain comprises a heavy chain variable region comprising the amino acid sequence set forth in SEQ ID NO: 354 and a light chain variable region comprising the amino acid sequence set forth in SEQ ID NO: 311.

In one embodiment, the hCD19 binding domain comprises a heavy chain variable region comprising the amino acid sequence set forth in SEQ ID NO: 355 and a light chain variable region comprising the amino acid sequence set forth in SEQ ID NO: 312.

In one embodiment, the hCD19 binding domain comprises a heavy chain variable region comprising the amino acid sequence set forth in SEQ ID NO: 356 and a light chain variable region comprising the amino acid sequence set forth in SEQ ID NO: 313.

Tolerable variations of the hCD19 binding domain will be known to those of skill in the art, while maintaining specific binding to hCD19. For example, in some embodiments the hCD19 binding domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 44-86.

In some embodiments, the hCD19 binding domain is encoded by a nucleic acid sequence comprising the nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleotide sequence set forth in SEQ ID NOs: 1-43.

The antigen binding domain may be operably linked to another domain of the CAR, such as the transmembrane domain or the costimulatory signaling domain, both described elsewhere herein. In one embodiment, a nucleic acid encoding the antigen binding domain is operably linked to a nucleic acid encoding a transmembrane domain and a nucleic acid encoding a costimulatory signaling domain.

The antigen binding domains described herein, such as the antibody or fragment thereof that binds to hCD19, can be combined with any of the transmembrane domains described herein, any of the intracellular domains or cytoplasmic domains described herein, or any of the other domains described herein that may be included in the CAR.

TABLE 4
CAR Construct Features and Exemplary Anti-CD19 CARs

| SEQ | | |
|---|---|---|
| ID | | |
| NO: | Name: | Sequence: |
| 262 | scFv KIR | ATGGCCTTACCAGTGACCGCCTTGCTCCTGCCGCTGGCCTTGCTGCTC |
| | construct | CACGCCGCCAGGCCGGGATCCXGCTAGCGAGCAGAAATTAATCTCA |
| | DNA | GAGGAGGACCTGCCTAGCGGTGGCGGAGGTTCTGGAGGTGGGGGTT |
| | | CCTCACCCACTGAACCAAGCTCCAAAACCGGTAACCCCAGACACCTG |
| | | CATGTTCTGATTGGGACCTCAGTGGTCAAAATCCCTTTCACCATCCTC |
| | | CTCTTCTTTCTCCTTCATCGCTGGTGCTCCAACAAAAAAAATGCTGCT |
| | | GTAATGGACCAAGAGCCTGCAGGGAACAGAACAGTGAACAGCGAGG |
| | | ATTCTGATGAACAAGACCATCAGGAGGTGTCATACGCATAA |
| | | (X indicates scFv insertion site) |
| 263 | scFv KIR | MALPVTALLLPLALLLHAARPGSXASEQKLISEEDLPSGGGGSGGGGSSP |
| | construct | TEPSSKTGNPRHLHVLIGTSVVKIPFTILLFFLLHRWCSNKKNAAVMDQE |
| | AA | PAGNRTVNSEDSDEQDHQEVSYA |
| | | (X indicates scFv insertion site) |
| 264 | Myc tag | GAGCAGAAATTAATCTCAGAGGAGGACCTG |
| 260 | Linker | SGGGGSGGGGS |
| 265 | TM/KIR2 | CCCACTGAACCAAGCTCCAAAACCGGTAACCCCAGACACCTGCATGT |
| | DS2 | TCTGATTGGGACCTCAGTGGTCAAAATCCCTTTCACCATCCTCCTCTT |
| | domains | CTTTCTCCTTCATCGCTGGTGCTCCAACAAAAAAAATGCTGCTGTAAT |
| | | GGACCAAGAGCCTGCAGGGAACAGAACAGTGAACAGCGAGGATTCT |
| | | GATGAACAAGACCATCAGGAGGTGTCATACGCA |
| 266 | TM/KIR2 | PTEPSSKTGNPRHLHVLIGTSVVKIPFTILLFFLLHRWCSNKKNAAVMDQ |
| | DS2 | EPAGNRTVNSEDSDEQDHQEVSYA |
| | domains | |
| 357 | CD8 | MALPVTALLLPLALLLHAARP |
| | Leader | |
| | Sequence | |
| 370 | scFv_01 | MALPVTALLLPLALLLHAARPGSQAVLNQPASVSAALGQRVTISC |
| | CAR | NTNIGSPYDVQWYQQLPGKSPKTIIYGNSNRPSGVPVRFSGSKSGS |
| | | TATLTIAGIQAEDEADYYCQSYDDSLDGRVVFGGGTQLTVLGGG |
| | | SSRSSSSGGGGSGGGGEVQLVESGGDLVKPGGSLRLSCVASGFTF |
| | | SNHEMYWVRQAPGKGLEWVARIYDSGSRTRYADAVKGRFTISR |
| | | DNAKNTLYLQMNSLTAEDTAVYFCAGGNSRGWTEFGMDYWGP |
| | | GTLVTVSSASEQKLISEEDLPSGGGGSGGGGSSPTEPSSKTGNPRH |
| | | LHVLIGTSVVKIPFTILLFFLLHRWCSNKKNAAVMDQEPAGNRTV |
| | | NSEDSDEQDHQEVSYA |
| 371 | scFv_02 | MALPVTALLLPLALLLHAARPGSSYVLTQSPSLSGSLGQKVTISCT |
| | CAR | GSRSNLGGNYVGWYQQVPGMAPRNVIYANDFRPSRIPARFSASK |
| | | SGTSATLTISGLQAEDEADYFCSSWDDDLGSHVFGSGTQLTILGG |
| | | GSSRSSSSGGGGSGGGGEEQLVEFGGDLVKPGGSLRLSCVASGFS |
| | | FSSYDMSWVRQAPGKGLQWVASTNFDGSRTYYTDAVEGRFTISR |
| | | DNARNTVYLQMNSLRAEDTAVYYCARGMGSWGASTFGYWGQG |
| | | TLVTVSSASEQKLISEEDLPSGGGGSGGGGSSPTEPSSKTGNPRHL |
| | | HVLIGTSVVKIPFTILLFFLLHRWCSNKKNAAVMDQEPAGNRTVN |
| | | SEDSDEQDHQEVSYA |
| 372 | scFv_03 | MALPVTALLLPLALLLHAARPGSQSVLNQPPSVSGALGQTVTISC |
| | CAR | SGSHTNIGSGFDVQWYQQLPGKSPQTLVYANINRPSGVPARFSGS |
| | | KSGSTATLTITGVQAEDEADYYCQSYDDNFDGHVFGSGTQLTVL |
| | | GGGSSRSSSSGGGGSGGGGEVQLVQSGGDLVKPGGSLRLSCVAS |
| | | GFTFSNYTMSWVRQAPGQGLQWVAYINYDGSITYYAEAVKGRF |
| | | TVSRDNAKNTLYLQMNSLRAEDTAVYFCVKETFMIPTEWGQGTL |
| | | ITVSSASEQKLISEEDLPSGGGGSGGGGSSPTEPSSKTGNPRHLHVL |
| | | IGTSVVKIPFTILLFFLLHRWCSNKKNAAVMDQEPAGNRTVNSED |
| | | SDEQDHQEVSYA |
| 373 | scFv_04 | MALPVTALLLPLALLLHAARPGSQSVLNQPPSVSGALGQTVTISC |
| | CAR | SGSHTNIGSGFDVQWYQQLPGKSPQTIIYANINRPSGVPARFSGSK |
| | | SGSTATLTITGVQAEDEADYYCQSYDDNFDYVFGSGTQLTVLGG |
| | | GSSRSSSSGGGGSGGGGEEQLVEFGGDLVKPGGSLRLSCVVSGFD |
| | | FSRYGMSWVRQSPGKGLQWVADISYRGTTFYADDVKGRFTVSR |
| | | DSAKNTLYLEMTSLTVEDTAVYYCANSANWYVSPFDHWGQGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 374 | scFv_05 | MALPVTALLLPLALLLHAARPGSQPVLTQPPSVSGSLGQRVTISCS |
| | CAR | GSTNTIGVVGANWYQQLPGKSPKLLVYSDGVRPSGVPDRFSGSR |
| | | SANSDTLTITGLQAEDEADYYCQSFDSTHNAIVFGGGTHLTVLGG |
| | | GSSRSSSSGGGGSGGGGEVQLVETGGDLVKPAGSLRLSCAASGFP |
| | | FSGYSMTWVRQAPEKGLQLVAGINSDGSHTYYTDDVKGRFTISR |
| | | DNTKYILYLQMNSLRAEDTAMYYCGSHSWGNFHYWGQGTLVT |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 375 | scFv_06 | MALPVTALLLPLALLLHAARPGSQPVLTQPPSVSGSLGQRVTISCT |
| | CAR | GTNTNIGSGYSVQWYQQLPGESPKPIIYGSSNRPSGVPARFSGSKS |
| | | GSTGTLTITGIQAEDEADYHCQSYDDSLDGHAVFGGGTQLTILGG |
| | | GSSRSSSSGGGGSGGGGEVQLVQSGGDLVKPGGSLRLSCVASGFT |
| | | FSDNYMSWIRQVPGKGLQWVSRISTDGRGTSYADAVKGRFTISR |
| | | DNAKNTLYLQMNSLRDEDTAVYYCARRSGIWGFDYWGQGALV |
| | | TVSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 376 | scFv_07 | MALPVTALLLPLALLLHAARPGSSYELTQPPSMSAALGQRVTISC |
| | CAR | TGSNTNIGSGYDVQWYQQVPGKSPTPLIYGNTNRPSGVPVRFSGS |
| | | KSGNTATLTITGIQAEDEADYYCQSYDDNFDGHHVFGSGTQLTV |
| | | LGGGSSRSSSSGGGGSGGGGEVQLVESGGDLVKPAGSLRLSCVAS |
| | | GFTFSNYGMNWVRQAPGKGLQWVAGINSGGSTTTYADVVKGRF |
| | | TISRDNAKNTLYLQMNSLGAEDTAVYYCARVDYSSSWLVDYWG |
| | | QGTLVTVSSASEQKLISEEDLPSGGGGSGGGGSSPTEPSSKTGNPR |
| | | HLHVLIGTSVVKIPFTILLFFLLHRWCSNKKNAAVMDQEPAGNRT |
| | | VNSEDSDEQDHQEVSYA |
| 377 | scFv_08 | MALPVTALLLPLALLLHAARPGSQSVLTQPASVSGSLGQKVTISC |
| | CAR | TGSRSNIGANYVGWYQQVPGIGPRTVIFGYNYRPSGVPDRESSSK |
| | | SGNSATLTISGLQAEDEAEYYCSSWDDSLRGTVFGSGTQLTVLGG |
| | | GSSRSSSSGDGGSGGGGEVQLVESGGDLVKPGESLRLSCVASGFT |
| | | FSSYGMSWVRQSPGKGLQWVADISYGGGTYYADAVKGRFTISR |
| | | DNARNTVYLQMNSLRAEDTAVYYCASQWGDWGHFEYWGQGT |
| | | LVTVSSASEQKLISEEDLPSGGGGSGGGGSSPTEPSSKTGNPRHLH |
| | | VLIGTSVVKIPFTILLFFLLHRWCSNKKNAAVMDQEPAGNRTVNS |
| | | EDSDEQDHQEVSYA |
| 378 | scFv_09 | MALPVTALLLPLALLLHAARPGSQPVLTQPPSVSAALGQRVTLSC |
| | CAR | TGSKTNIGSGYDVQWYQQFPGKSPKTIIYGNSNRFSGVPARFSGS |
| | | KSGSIATLTITGIQAEDEADYYCQSFDDNVDGYVFGSGTQLTILGG |
| | | GSSRSSSSGGGGSGGGGEVQLVESGGDLVKPGGSLRLSCVASGFT |
| | | FSSDSIHWVRQAPGKGLQWVAYINSGGSTTYYADAVKGRFTISR |
| | | DNARNTVYLKMNSLRAEDTAVYYCAKDSGFGSWYLESWGQGT |
| | | LVTVSPASEQKLISEEDLPSGGGGSGGGGSSPTEPSSKTGNPRHLH |
| | | VLIGTSVVKIPFTILLFFLLHRWCSNKKNAAVMDQEPAGNRTVNS |
| | | EDSDEQDHQEVSYA |
| 379 | scFv_10 | MALPVTALLLPLALLLHAARPGSQAVLNQPASVSGALGQTVTISC |
| | CAR | SGSHTNIGSGFDVQWYQQLPGKSPQTIIYANINRPSGVPARFSGSK |
| | | SGSTATLTITGVQAEDEADYHCQSFDDDLDGPVFGGGTHLTVLG |
| | | GGSSRSSSSGGGGSGGGGEVQLVQSGGDLVKPGGSLKISCVASGL |
| | | TFNRNYMTWIRQAPGKGLQWVSEINPDGSTTSYTDAVRGRFTISR |
| | | DNAKNTLYLQMNSMRDEDTAVYYCARSSWGSFDYWGQGTLVT |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 380 | scFv_11 | MALPVTALLLPLALLLHAARPGSQPVLTQPPSVSGALGQTVTISCS |
| | CAR | GSHTNIGSGFDVQWYQQLPGKSPQTLVYANINRPSGVPARFSGSK |
| | | SGSTATLTITGVQAEDEADYYCQSYDDNFDYVFGSGTQLTVLGG |
| | | GSSRSSSSGGGGSGGGGEVQLVESGGDLVKPGGSLRLSCVASGFT |
| | | FSDYSMSWVRQSPGKGLQWVAAIMTSGSTYYADAVKGRFTISRD |
| | | NAKNTLYLQMDSLRAEDTAVYYCAHDYWFDFWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 381 | scFv_12 | MALPVTALLLPLALLLHAARPGSSYELTQPPSATVTLRQTARLTC |
| | CAR | GGENIGRKSVQWYQQKPGQSPLLIIYADSSRPSGIPERFSGANSGN |
| | | TASLTISGALAEDEADYYCQIWDRNINVFGSGTQLTVLGGGSSRS |
| | | SSSGGGGSGGGGEEQLVEFGGDLVKPAGSLRLSCVASGFTFSSYD |
| | | MSWVRQAPGKGLQWVATISYDGSSTYYTDAVKGRFTVSRDNAK |
| | | NTLYLQMNSLRAEDTALYYCAKDSGSWSHFYGLDTWGHGTSVF |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 382 | scFv_13 | MALPVTALLLPLALLLHAARPGSQPVLTQPPSVSGALGQTVTISCS |
| | CAR | GSHTNIGSGFDVQWYQQLPGKSPQTLVYANINRPSGVPARFSGSK |
| | | SGSTATLTITGVQAEDEADYYCQSYDDNFDYVFGSGTQLTVLGG |
| | | GSSRSSSSGDGGSGGGGEVQLVESGGGLMKPGGSLKLSCLASGFT |
| | | FSRYYMYWVRQAPGKGLQWVAQISSDGRRTYYADAVQGRFTIS |
| | | RDNVKNTVYLQMNSLSDEDTAVYYCVREDYFDFDYWGQGTLV |
| | | TVSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 383 | scFv_14 | MALPVTALLLPLALLLHAARPGSQSVLNQPPSVSAALGQRITISCT |
| | CAR | GSHTNIGSGYDVQWYQQLPGKSPRTIIYGNSDRPSGVPVRFSGSK |
| | | SGNTATLTITGIQAEDEADYYCQSYDDNLNDPYVFGSGTQLTVLG |
| | | GGSSRSSSSGGGGSGGGGEVQLVQSGGDLVKPGGSLRLSCVASG |
| | | FAFSGKTMQWVRQAPGKGLQWVAYIQHDGSRTSYADAVKGRFT |
| | | ISRDNAKNTLYLQMDSLRAEDTAVYYCADHPWYGPEYWGQGTP |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 384 | scFv_15 | MALPVTALLLPLALLLHAARPGSSYELTQPPSVSAALGQGVTISCI |
| | CAR | GSDTNIGNHYDVQWYQQLPGKSPRTIIYGNSDRPSGVPGRFSGSK |
| | | SGNTATLTITGIQAEDEADYHCQSYDDDLDGHIVFGGGTHLTVLG |
| | | GGSSRSSSSGGGGSGGGGEVQLVQSGGDLVKPGGSLRLSCVASG |
| | | FTFNKYHMSWVRQAPGKGLQWVGKITSDGSATGYADAVKGRFT |
| | | ISRDNAKNTFYLQMDSLRAEDTAMYYCARDQSSWGTGNFDYWG |
| | | QGTLVTVSSASEQKLISEEDLPSGGGGSGGGGSSPTEPSSKTGNPR |
| | | HLHVLIGTSVVKIPFTILLFFLLHRWCSNKKNAAVMDQEPAGNRT |
| | | VNSEDSDEQDHQEVSYA |
| 385 | scFv_16 | MALPVTALLLPLALLLHAARPGSQSVLTQPTSVSGSLGQRVTISCT |
| | CAR | GSSSNVGYGNYVGWYQQLPGTGPRTLIYGSHYRPSGVPDRFSGSS |
| | | SGSSATLTISGLQAEDEADYYCSSYDSTLSGYVFGSGTQLTILGGG |
| | | SSRSSSSGGGGSGGGGEVQLVESGGDLVKPGGSLRLSCVGSGFTL |
| | | SSYWMSWVRQAPGKGLQWIAEISGSGNTIYYGDAVKGRFTISRD |
| | | NAKNTLYLQMNSLRDEDTAVYYCATEYYWNIDNWGQGTLVTV |
| | | SSASEQKLISEEDLPSGGGGSGGGGSSPTEPSSKTGNPRHLHVLIGT |
| | | SVVKIPFTILLFFLLHRWCSNKKNAAVMDQEPAGNRTVNSEDSDE |
| | | QDHQEVSYA |
| 386 | scFv_17 | MALPVTALLLPLALLLHAARPGSQPVLTQPPSVSAALGQRVTISC |
| | CAR | TGSKTNIGSGYDVQWYQQLPGKSPKTIIYGNSNRPSGVPARFSGS |
| | | KSGSIATLTITGIQAEDEADYYCQSFDDNVDGYVFGSGTQLTILGG |
| | | GSSRSSSSGGGGSGGGGEVPLVESGGDLVKPGGSLRLSCVASGFT |
| | | FSSYGMSWVRQAPGKGLQWVAYINGGGSSTSYADAVRGRFTISR |
| | | DNAENTLYLQMNSLRADDTAVYYCSNYYGIYWDSDYWGQGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 387 | scFv_18 | MALPVTALLLPLALLLHAARPGSQSALTQTASVSGSLGQKVTISC |
| | CAR | TGSRSNIGANYVGWYQQVPGIGPRTVIFGYNYRPSGVPDRESSSK |
| | | SGNSATLTISGLQAEDEAEYYCSTWDSSLNAYVFGSGTQLTVLGG |
| | | GSSRSSSSGGGGSGGGGEVPLVESGGDLVKPGGSLRLSCVASGFT |
| | | LSNYGMSWVRQSPGKGLQWVAAIRSGGNTYYADAVKGRFTISR |
| | | DNAKNTLYLQMNSLRAEDTAVYYCASQWGDWGHFEYWGQGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 388 | scFv_19 | MALPVTALLLPLALLLHAARPGSQAVLTQPASVSGPLGQRVTISC |
| | CAR | SGSTNNIGIVGAAWYQQFPGKAPKLLVYSDGSRPSGVPDRFSGSN |
| | | SGNSATLTISGLQAEDEADYYCQSVDATHGTILFGGGTQLTVLGG |
| | | GSSRSSSSGGGGSGGGGEVQLVETGGDLVKPAGSLRLSCAASGFP |
| | | FSGYSMTWVRQAPEKGLQLVAGINSDGSHTYYTDDVKGRFTVSR |
| | | DNTKYILYLQMNSLRVEDTAIYYCGSHSWGNFHYWGQGTLVTV |
| | | SSASEQKLISEEDLPSGGGGSGGGGSSPTEPSSKTGNPRHLHVLIGT |
| | | SVVKIPFTILLFFLLHRWCSNKKNAAVMDQEPAGNRTVNSEDSDE |
| | | QDHQEVSYA |
| 389 | scFv_20 | MALPVTALLLPLALLLHAARPGSQSVLNQPPSVSVSLGQTATISCS |
| | CAR | GESLSKYYAQWFQQKAGQVPVLVIYKDTERPSGIPDRFSGSSSGN |
| | | THTLTISGARAEDEADYYCESEVSPDTIVFGGGTHLTVLGGGSSRS |
| | | SSSGGGGSGGGGEVQLVESGGDLVKPGGSLRLSCVASGFTFSSYA |
| | | MSWVRQAPGEGLQWVAYINSAGSTTYYADAVKGRFTISRDNAR |
| | | NTVYLQMNSLRAEDTAVYYCGRYYYGLDYWGPGTSVFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 390 | scFv_21 | MALPVTALLLPLALLLHAARPGSQPVLTQPPSVSVSLGQTATISCS |
| | CAR | GESLSKYYAQWFQQKAGQAPVLVIYKDTERPSGIPDRFSGSSSGN |
| | | THTLTISGARAEDEADYYCESAVSSDTAVFGSGTQLTVLGGGSSR |
| | | SSSSGGGGSGGGGEVQLVESGGDMVKPGGSLRLSCVASGFTFSSF |
| | | WMYWVRQAPGKGLQWVGGINSGGSITSYADAVKGRFTISRDNA |
| | | RNTLYLQMDSLRDEDTAVYYCAKPDIYGSFDHWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 391 | scFv_22 | MALPVTALLLPLALLLHAARPGSQPVLTQPPSVSGSLGQRVTISCT |
| | CAR | GSSSNVGYGDYVGWYQQLPGTSPRTLIYSSSSRPSGVPDRFSGSRS |
| | | GSTATLTISGLQAEDEADYHCSSYDTSLNGFVFGSGTQLTILGGGS |
| | | SRSSSSGGGGSGGGGELTLQESGPGLVKPAQTLSLTCTVSGDSVT |
| | | SSHFWHWIRQRPGRGLEWMGYWSDITIYSPTFQGRISITVDTARN |
| | | QFSLHLNSVTTEDSAVYFCARYNGYVDYLDVWGQGTLITVSSAS |
| | | EQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQD |
| | | HQEVSYA |
| 392 | scFv_23 | MALPVTALLLPLALLLHAARPGSDIVMTQTPLSLSVSPGEPASISC |
| | CAR | KASQSLLHSNGNTYLYWFRQKPGQSPQRLIYLVSNRDAGVPDRF |
| | | SGSGSGTDFTLRISRVEADDSGIYYCGQGKQDPITFGEGTHLEIKG |
| | | GSSRSSSSGGGGSGGGGELTLQESGPGLVKPSQTLSLTCVVSGGS |
| | | VTTSDYWNWLRQRPGRGLEYMGYWKGRTEYNPAFQGRISITAD |
| | | TAKNQFSLQLNSVTADDTAIYYCAHWGSGNLDYWGPGTLVTVS |
| | | SASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 393 | scFv_24 | MALPVTALLLPLALLLHAARPGSQSVLNQPPSVSGALGQTVTISC |
| | CAR | SGSHTNIGSGFDVQWYQQLPGKSPQTLVYANINRPSGVPVRFSGS |
| | | KSGTTATLTITGIQAEDEADYYCQSYDDNVDGYVFGSGTQLTVL |
| | | GGGSSRSSSSGGGGSGGGGEVQLVQSGGXLVKPGGSLRLSCVAS |
| | | GFTLSSNYMDWVRQAPGKGPQWVAQISTDGSRTYYADAVKGRF |
| | | TISRDTAKNTLYLQMNSLRAEDTAMYYCAKNEYLAPTYWGQGT |
| | | LVTVSSASEQKLISEEDLPSGGGGSGGGGSSPTEPSSKTGNPRHLH |
| | | VLIGTSVVKIPFTILLFFLLHRWCSNKKNAAVMDQEPAGNRTVNS |
| | | EDSDEQDHQEVSYA |
| 394 | scFv_25 | MALPVTALLLPLALLLHAARPGSDIVMTQTPLSLSVSPGETASISC |
| | CAR | KASQSLLHSNGNTYLNWFRQKPGQSPQVLIYQVSNRDTGVPDRF |
| | | SGSGSGTDFTLRISRVEADDAGVYYCGQGIQYPPTFGAGTKVELK |
| | | GGSSRSSSSGGGGSGGGGEEQLVEFGGDLVKPGGSLRLSCVASGF |
| | | TFSSLSMNWIRQAPGKGLQWVSQISPDGTITGYTDAVKGRFTISR |
| | | DNAKNTLYLQMNSLRDEDTAIYYCAKSRYTDTYPFDYWGPGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 395 | scFv_26 | MALPVTALLLPLALLLHAARPGSSSVLTQPPSVSVSLGQTATISCS |
| | CAR | GESLSKYYTQWFQQKAGQAPILVIYKDTERPSGIPDRFSGSSSGNT |
| | | HTLTISGARAEDEADYYCESLVDSDAYMFGSGTQLTVLGGGSSRS |
| | | SSSGGGGSGGGGEVQLVETGGDLVKPGGSLRLSCVGSGFTFSSY |
| | | WMSWVRQAPGKGLQWIADISDSGSSTDYADAVKGRFTISRDNA |
| | | KNTLYLQMNSLRAEDTAMYYCARGLGMDYWGHGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 396 | scFv_27 | MALPVTALLLPLALLLHAARPGSQPVLTQPPSVSVSLGQTATISCS |
| | CAR | GESLSKYYAQWFQQKAGQAPVLVIYKDTERPSGIPDRFSGSSSGN |
| | | THTLTITGIQAEDEGDYHCHSYDENLDGYVFGSGTQLTVLGGGSS |
| | | RSSSSGGGGSGGGGEVQLVESGGDLVKPGGSLRLSCVGSGFTFSS |
| | | YWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 397 | scFv_28 | MALPVTALLLPLALLLHAARPGSQSALTQPSSVSVSLGQTATISCS |
| | CAR | GESLNKYYVQWFQQKAGQVPILVIYRDTERPSGIPDRFSGSSSGN |
| | | THTLTITGTRAEDEADYYCESLVNDNTYVFGSGTQLTVLGGGSSR |
| | | SSSSGGGGSGGGGEVQLVETGGDLVKPGGSLRLSCVGSGFTFSSY |
| | | WMSWVRQAPGKGLQWIADISDSGSSTDYADAVKGRFTISRDNA |
| | | KNTLYLQMNSLRAEDTAMYYCARGLGMDYWGHGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 398 | scFv_29 | MALPVTALLLPLALLLHAARPGSQSVLNQPPSVSVSLGQTATISCS |
| | CAR | GESLNKYYTQWFQQKAGQAPVLVIYKDTERPSGIPDRFSGSSSGN |
| | | THTLTISGARAEDEADYYCESVDSTDTYVFGSGTQLTILGGGSSRS |
| | | SSSGGGGSGGGGEVHLVESGGDLVKPGGSLRLSCVGSGFTFSSY |
| | | WMSWVRQAPGKGLQWIADISDSGSSTDYADAVKGRFTISRDNA |
| | | KNTLYLQMNSLRAEDTAMYYCARGLGMDYWGHGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 399 | scFv_30 | MALPVTALLLPLALLLHAARPGSQSVLTQPASVSVSLGQTATISCS |
| | CAR | GESLSKYYTQWFQQKAGQAPVLVIYKDTERPSGIPDRFSGSSSGN |
| | | THTLTISGAQAEDEADYYCQSYDDNVDGYVFGSGTQLTVLGGGS |
| | | SRSSSSGGGGSGGGGEVQLVESGGDLVKPGGSLRLSCVGSGFTFS |
| | | SYWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 400 | scFv_31 | MALPVTALLLPLALLLHAARPGSQSALTQPSSVSVSLGQTATISCS |
| | CAR | GESLTKYYVQWFQQKAGQAPVLVIYRDTERPSGIPDRFSGSSSGN |
| | | THTLTISGARAEDEADYYCESMDGREAYVFGSGTQLTVLGGGSS |
| | | RSSSSGGGGSGGGGEVQLVESGGDLVKPGGSLRLSCVASGFTFSS |
| | | YWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 401 | scFv_32 | MALPVTALLLPLALLLHAARPGSQSVLTQPASVSVSLGQTATISCS |
| | CAR | GESLSKYYAQWFQQKAGQAPVLVIYKDTERPSGIPDRFSGSSSGN |
| | | THTLTITGIQAADEADYYCQSYDDNLDGYVFGSGTQLTVLGGGS |
| | | SRSSSSGGGGSGGGGEVQLVESGGDLVKPGGSLRLSCVGSGFTFS |
| | | SYWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 402 | scFv_33 | MALPVTALLLPLALLLHAARPGSDIVMTQTPLSLSVSPGEPASISC |
| | CAR | KASQSLLHSDGNTYLHWYLQKPGQSPQLLIYSVSNRFTGVSDRFS |
| | | GSGSGTDFTLRISRVEADDTGVYYCGQGTHLPFTFGQGTKLEIKG |
| | | GSSRSSSSGGGGSGGGGEVQLVESGGDLVKPGGSLRLSCAASGFT |
| | | FSHYDMNWVRQAPGKGLQWVASIAYDGSNTYYTDAVKGRFTIS |
| | | RDNAKNILFLLVNSLRAEDTAIYYCARGLVYWGQGTLVTVSSAS |
| | | EQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQD |
| | | HQEVSYA |
| 403 | scFv_34 | MALPVTALLLPLALLLHAARPGSSYELTQPPSVSAALGQRVTISCT |
| | CAR | GTNTNIGSGYSVQWYQQLPGESPKPIIYGSSNRPSGVPARFSGSKS |
| | | GSTGTLTITGIQAEDEADYHCQSYDDSLDGHAVFGGGTQLTILGG |
| | | GSSRSSSSGGGGSGGGGEVQLVESGGDLVKPGGSLRLSCVASGLT |
| | | FSDNYMSWIRQVPGKGLQWVSRISTDGRGTSYADAVKGRFTISR |
| | | DNAKNTLYLQMNSLRDEDTAVYYCARRSGIWGFDYWGQGTLVT |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 404 | scFv_35 | MALPVTALLLPLALLLHAARPGSSSVLTQPPSVSVSLGQTATISCS |
| | CAR | GESLSEYYAQWFQQKAGQAPVLVIYKDTERASGIPDRFSGSSSGN |
| | | THTLTISGARAEDEADYYCESAVNSDGIVFGGGTHLTVLGGGSSR |
| | | SSSSGGGGSGGGGEVQLVESGGDLVKPAGSLRLSCVTSGFTFSSY |
| | | AMSWVRQAPGKGLQWVGSINSGGSTTSYADAVKGRFTISRDNA |
| | | KNTVYLQMNSLRAEDTAVYYCARWWDFEYWGQGTLVTVSSAS |
| | | EQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQD |
| | | HQEVSYA |
| 405 | scFv_36 | MALPVTALLLPLALLLHAARPGSQSVLTQPTSVSGSLGQRVTISCS |
| | CAR | GSTNNIGIVGASWYQQLPGKAPKLLVYSDGDRPSGVPDRFSGSNS |
| | | GNSDTLTITGLQAEDEADYYCQSFDTTLDAYVFGSGTQLTVLGG |
| | | GSSRSSSSGGGGSGGGGEEQLVEFGGGLVRPGGSLRLSCVASGFT |
| | | FSSYGMTWVRQSPGKGLQWVATISDTGSTYYADTVKGRFSISRD |
| | | NAKNTLYLQMNSLRADDTALYYCTKDLGVWGMDNWGPGTSLF |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 406 | scFv_37 | MALPVTALLLPLALLLHAARPGSSSVLTQPPSVSVSLGQTATISCS |
| | CAR | GESLNDYYAQWFQQKAGQVPVLVIYRDTERPSGIPDRFSGSSSGN |
| | | THTLTISGARAEDEADYYCESEVSAGGAVFGEGTHLTVLGGGSSR |
| | | SSSSGGGGSGGGGEVQLVETGGDLVKPGGSLRLSCVASGFTLRD |
| | | YSMTWVRQTPEKGLQLVAGISTGGSTTYYIDAVKGRFTISRDNAK |
| | | NTVYLQMNSLRAEDTAMYHCTTTALGYWGQGTLVTVSSASEQK |
| | | LISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEV |
| | | SYA |
| 407 | scFv_38 | MALPVTALLLPLALLLHAARPGSSSVLTQPPSVSVSLGQTATISCS |
| | CAR | GESLSEYYAQWFQQKAGQAPVLVIYKDTERPSGIPDRFSGSSSGN |
| | | THTLTISGARAEDEADYYCASADSSAIPVFGGGTHLTVLGGGSSR |
| | | SSSSGGGGSGGGGEVQLVESGGDLVKPGGSLRLSCVASGFNFGN |
| | | YAVSWVRQAPGKGLQWVAGISNDGTITGYTDVVEGRFIISRDNA |
| | | KNTLYLQMNSLRAEDMAIYYCVAGGFLDYWGQGTLVTVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 408 | scFv_39 | MALPVTALLLPLALLLHAARPGSQSVLTQPPSVSGSPGQRVTISCS |
| | CAR | GTTKNIGIVGASWYQQLPGKAPKLLVHSDGFRPSGVPDRFSASNS |
| | | GDSDILTITGLQAEDEADYYCQSFDTTLDAHVFGSGTQLTVLGGG |
| | | SSRSSSSGGGGSGGGGEVPLVESGGDLVKPGGSLRLSCVASGFTF |
| | | SSYGMSWVRQSPGKGLQWVAGITYDGSNTYYTDTVRGRFTISRD |
| | | NARNTVYLQMNSLRAEDTAVYYCVASPLYGTYGFDYWGHGTSV |
| | | FVSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 409 | scFv_40 | MALPVTALLLPLALLLHAARPGSQSVLTQPPSVSGSLGQKVTISCT |
| | CAR | GSSSSVGTHYVGWYQIPPGIGPRTVIYNNDYRPSGVPDRFSGSKSG |
| | | NSATLTISGLQAEDEADYYCSSWDDSLGAYVLGSGTQLTILGGGS |
| | | SRSSSSGGGGSGGGGELTLQESGPGLVKPSQTLSLTCAVSGGSVIR |
| | | GYYWTWIRQRPGRGLEWMGYWAGSTDYNPAFQGRIFITAGTAK |
| | | NQFSLEMTSMTTDDTAIYYCVGSLRGGWLFDNWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 410 | scFv_41 | MALPVTALLLPLALLLHAARPGSQPVLTQPPSVSVSLGQTATISCS |
| | CAR | GESLSKYYAQWFQQKAGQAPVLVIYKDTERPSGIPDRFSGSSSGN |
| | | THTLTISGARAEDEADYYCESAVSTETVVFGGGTHLTVLGGGSSR |
| | | SSSSGGGGSGGGGEVQLVETGGDLVMPAGSLRLSCVASGFTFSSS |
| | | VMSWVRQAPGKGLEWVAGINGGGSFAAYGDAVKGRFTISRDNA |
| | | KNTLYLQMNSLRAEDTAMYYCVGGGYMDFWGPGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 411 | scFv_42 | MALPVTALLLPLALLLHAARPGSQSVLTQPTSVSGSLGQRVTISCS |
| | CAR | GSTNDIGIIGAAWYQQLPGKAPKLLLYSNKNRPSGVPDRFSGSNS |
| | | GISATLTITGLQAEDEADYYCQSVDPALHTYVFGSGTQLTILGGGS |
| | | SRSSSSGGGGSGGGGEVQLVEIGGDLVKPEGSLRLSCVVSGFTFS |
| | | GYDMNWVRQAPGKGLQWVAYISSDGRRTVYTDAVKGRFTISRD |
| | | NAKNTLYLQMNSLKSEDTAVYYCVKGGWLDFWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 412 | scFv_43 | MALPVTALLLPLALLLHAARPGSDIVMTQNPLSLAVTPGELATISC |
| | CAR | RASQSLLHSDGKSYLNWYLQKPGQTPRPLIYEASRRFSGVSDRFN |
| | | GSGSGTDFTLKISRVEAEDVGVYYCQQGLHFPPTFGAGTKVELKG |
| | | GSSRSSSSGGGGSGGGGEVQLVESGGDLVKPGGSLRLSCAASGFT |
| | | FTDYDMSWVRQAPGKGLQWVAAISYDGSSTYYTDAVKGRFTISR |
| | | DNARNTLYLQMSSLRAEDTAMYYCVVSNMNLWGHGTSVFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |

### Transmembrane Domain

With respect to the transmembrane domain, the CAR of the present invention (e.g., hCD19 CAR) can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

In some embodiments, the transmembrane domain comprises a KIR transmembrane. In some embodiments, the KIR transmembrane domain is selected from the group consisting of a KIR2DS2 transmembrane domain, a KIR2DS2 transmembrane domain, a KIR2DL3 transmembrane domain, a KIR2DL1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL1 transmembrane domain, a KIR3DS1 transmembrane domain, a KIR3DL2 transmembrane domain, a KIR3DL3 transmembrane domain, a KIR2DP1 transmembrane domain, and a KIR3DP1 transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3 (2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G) n, glycine-serine polymers (including, for example, (GS)ₙ, (GSGGS)ₙ (SEQ ID NO: 346) and (GGGS)ₙ (SEQ ID NO: 347), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine and is much less restricted than residues with longer side chains (see, e.g., Scheraga, Rev. Computational. Chem. (1992) 2:73-142). Exemplary hinge regions can comprise amino acid sequences including, but not limited to, GGSG (SEQ ID NO: 348), GGSGG (SEQ ID NO: 349), GSGSG (SEQ ID NO: 350), GSGGG (SEQ ID NO: 351), GGGSG (SEQ ID NO: 352), GSSSG (SEQ ID NO: 353), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87 (1): 162-166; and Huck et al., Nucleic Acids Res. (1986) 14 (4): 1779-1789. As non-limiting examples, an immunoglobulin hinge region can include one of the following amino acid sequences: DKTHT (SEQ ID NO: 358); CPPC (SEQ ID NO: 359); CPEPKSCDTPPPCPR (SEQ ID NO: 360) (see, e.g., Glaser et al., J. Biol. Chem. (2005) 280:41494-41503); ELKTPLGDTTHT (SEQ ID NO: 361); KSCDKTHTCP (SEQ ID NO: 362); KCCVDCP (SEQ ID NO: 363); KYGPPCP (SEQ ID NO: 364); EPKSCDKTHTCPPCP (SEQ ID NO: 365) (human IgG1 hinge); ERKCCVECPPCP (SEQ ID NO: 366) (human IgG2 hinge); ELKTPLGDTTHTCPRCP (SEQ ID NO: 367) (human IgG3 hinge); SPNMVPHAHHAQ (SEQ ID NO: 368) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally occurring) hinge region. For example, His229 of human IgG1 hinge can be substituted with Tyr, so that the hinge region comprises the sequence EPKSCDKTYTCPPCP (SEQ ID NO: 369); see, e.g., Yan et al., J. Biol. Chem. (2012) 287:5891-5897. In one embodiment, the hinge region can comprise an amino acid sequence derived from human CD8, or a variant thereof.

The transmembrane domains described herein, such as a transmembrane region of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

Accordingly, a subject CAR of the present disclosure may comprise any of the transmembrane domains, hinge domains, or spacer domains described herein.

### Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a KIR2DS2 domain. In certain embodiments, the intracellular domain comprises a costimulatory signaling domain and an intracellular signaling domain.

In one embodiment, the intracellular domain of the CAR comprises a costimulatory signaling domain which includes any portion of one or more co-stimulatory molecules, such as at least one signaling domain from CD2, CD3, CD8, CD27, CD28, OX40, ICOS, 4-1BB, PD-1, any derivative or variant thereof, any synthetic sequence thereof that has the same functional capability, and any combination thereof.

Examples of the intracellular signaling domain include, without limitation, the (chain of the T cell receptor complex or any of its homologs, e.g., n chain, FesRly and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma Rlla, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33 (6): 651-653). Additionally, intracellular signaling domains may include signaling domains used by NK and NKT cells (see, e.g., Hermanson and Kaufman, Front. Immunol. (2015) 6:195) such as signaling domains of NKp30 (B7-H6) (see, e.g., Zhang et al., J. Immunol. (2012) 189 (5): 2290-2299), and DAP 12 (see, e.g., Topfer et al., J. Immunol. (2015) 194 (7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent).

In some embodiments, the intracellular signaling domain is based on an NK cell receptor (a NKR), e.g., a KIR-CAR, a NCR-CAR, a SLAMF-CAR, a FcR-CAR, or a Ly49-CAR is provided. In one embodiment, the invention provides a type of chimeric antigen receptor (CAR) wherein the CAR is termed a “KIR-CAR”, which is a CAR design comprising a component of a receptor found on natural killer (NK) cells. In one embodiment, the NK receptor includes but is not limited to a killer cell immunoglobulin-like receptor (KIR). KIRs can function as an activating KIR or an inhibiting KIR. One advantage of the KIR-CARs is that a KIR-CAR provides a method for regulating cytotoxic cell, e.g., T cell, specificity to control off-target activity of the engineered T cell. In some instances, the KIR-CARs of the invention do not require co-stimulation to proliferate. KIR-CARs can deliver a signal through an adaptor protein, e.g., an ITAM containing adaptor protein. In one embodiment, the KIR-CARs of the invention comprise an activating KIR which delivers its signal through an interaction with the immunotyrosine-based activation motif (ITAM) containing membrane protein, DAP12 that is mediated by residues within the transmembrane domains of these proteins.

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCERIG (also known as FCRG; Fc epsilon receptor I gamma chain; Fc receptor gamma-chain; Fc-epsilon RI-gamma; FcR gamma; FceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR.

In some embodiments, the intracellular domain comprises a KIR signaling domain. In some embodiments, the KIR signaling domain is selected from the group consisting of a KIR2DS2 signaling domain, a KIR2DS2 signaling domain, a KIR2DL3 signaling domain, a KIR2DL 1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain, and a KIR3DP1 signaling domain.

In one embodiment, the intracellular domain of a subject CAR comprises a KIR2DS2 intracellular signaling domain comprising the amino acid sequence set forth in SEQ ID NO: 266.

Tolerable variations of the intracellular domain that maintain specific activity will be known to those of skill in the art. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 266.

### Modified Immune Cells

The present invention provides a modified immune cell or precursor cell thereof (e.g., a modified T cell, a modified NK cell, a modified NKT cell), comprising a subject CAR. Accordingly, such modified cells possess the specificity directed by the CAR that is expressed therein. For example, a modified cell of the present invention comprising a hCD19 CAR possesses specificity for hCD19 on a target cell.

Any modified cell comprising a CAR comprising any antigen binding domain, any hinge, any transmembrane domain, any intracellular costimulatory domain, and any intracellular signaling domain described herein is envisioned, and can readily be understood and made by a person of skill in the art in view of the disclosure herein.

In some embodiments, the modified cell is an immune cell or precursor cell thereof. In an exemplary embodiment, the modified cell is a T cell. In an exemplary embodiment, the modified cell is an autologous cell. In an exemplary embodiment, the modified cell is an autologous immune cell or precursor cell thereof. In an exemplary embodiment, the modified cell is an autologous T cell.

### Nucleic Acids and Expression Vectors

The present disclosure provides an isolated nucleic acid encoding a polypeptide. The nucleic acid of the present disclosure can comprise a polynucleotide sequence encoding any one of the binding polypeptides, scFvs, CARs, or any fragments thereof disclosed herein.

One aspect of the present disclosure includes an isolated nucleic acid encoding a binding polypeptide comprising an antigen-binding domain that specifically binds human CD19.

In certain embodiments, the nucleic acid comprises an antigen binding domain comprising a heavy chain variable region that comprises three heavy chain complementarity determining regions (HCDRs) and a light chain variable region that comprises three light chain complementarity determining regions (LCDRs). HCDR1 comprises the amino acid sequences (SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, or 244), and/or HCDR2 comprises the amino acid sequences (SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, or 237), and/or HCDR3 comprises the amino acid sequences (SEQ ID NO: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, or 245) and/or LCDR1 comprises the amino acid sequence (SEQ ID NOs: 90, 95, 100, 109, 114, 119, 124, 128, 140, 148, 153, 158, 166, 171, 180, 185, 200, 203, 209, 213, 221, 229, 234, 242, or 246), and/or LCDR2 comprises the amino acid sequence (ADS, AND, ANI, EAS, GNS, GNT, GSH, GSS, GYN, KDT, LVS, NND, QVS, RDT, SDG, SNK, SSS, or SVS), and/or LCDR3 comprises the amino acid sequence (SEQ ID NO: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, or 247).

Also provided is an isolated nucleic acid encoding a single-chain variable fragment (scFv) comprising a heavy chain variable region that comprises three heavy chain complementarity determining regions (HCDRs) and a light chain variable region that comprises three light chain complementarity determining regions (LCDRs). HCDR1 comprises the amino acid sequences (SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, or 244), and/or HCDR2 comprises the amino acid sequences (SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, or 237), and/or HCDR3 comprises the amino acid sequences (SEQ ID NO: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, or 245) and/or LCDR1 comprises the amino acid sequence (SEQ ID NOs: 90, 95, 100, 109, 114, 119, 124, 128, 140, 148, 153, 158, 166, 171, 180, 185, 200, 203, 209, 213, 221, 229, 234, 242, or 246), and/or LCDR2 comprises the amino acid sequence (ADS, AND, ANI, EAS, GNS, GNT, GSH, GSS, GYN, KDT, LVS, NND, QVS, RDT, SDG, SNK, SSS, or SVS), and/or LCDR3 comprises the amino acid sequence (SEQ ID NO: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, or 247).

In some embodiments, the nucleic acid encoding an scFv encodes an HCDR1, HCDR2, HCDR3, LCDR1, and LCDR3 which comprise the respective amino acid sequences set forth in:
SEQ ID NOs: 87, 88, 89, 90, and 91 and wherein LCDR2 comprises the amino acid sequence GNS;
SEQ ID NOs: 92, 93, 94, 95, and 96, and wherein LCDR2 comprises the amino acid sequence AND;
SEQ ID NOs: 97, 98, 99, 100, and 101, and wherein LCDR2 comprises the amino acid sequence ANI;
SEQ ID NOs: 102, 103, 104, 100, and 105, and wherein LCDR2 comprises the amino acid sequence ANI;
SEQ ID NOs: 106, 107, 108, 109, and 110, and wherein LCDR2 comprises the amino acid sequence SDG;
SEQ ID NOs: 111, 112, 113, 114, and 115, and wherein LCDR2 comprises the amino acid sequence GSS;
SEQ ID NOs: 116, 117, 118, 119, and 120, and wherein LCDR2 comprises the amino acid sequence GNT;
SEQ ID NOs: 121, 122, 123, 124, and 125, and wherein LCDR2 comprises the amino acid sequence GYN;
SEQ ID NOs: 126, 117, 127, 128, and 129, and wherein LCDR2 comprises the amino acid sequence GNS;
SEQ ID NOs: 130, 131, 132, 100, and 133, and wherein LCDR2 comprises the amino acid sequence ANI;
SEQ ID NOs: 134, 135, 136, 100, and 105, and wherein LCDR2 comprises the amino acid sequence ANI;
SEQ ID NOs: 137, 138, 139, 140, and 141, and wherein LCDR2 comprises the amino acid sequence ADS;
SEQ ID NOs: 142, 143, 144, 100, and 105, and wherein LCDR2 comprises the amino acid sequence ANI;
SEQ ID NOs: 145, 146, 147, 148, and 149, and wherein LCDR2 comprises the amino acid sequence GNS;
SEQ ID NOs: 150, 151, 152, 153, and 154, and wherein LCDR2 comprises the amino acid sequence GNS;
SEQ ID NOs: 155, 156, 157, 158, and 159, and wherein LCDR2 comprises the amino acid sequence GSH;
SEQ ID NOs: 121, 160, 161, 128, and 162, and wherein LCDR2 comprises the amino acid sequence GNS;
SEQ ID NOs: 163, 164, 123, 124, and 165, and wherein LCDR2 comprises the amino acid sequence GYN;
SEQ ID NOs: 106, 107, 108, 166, and 167, and wherein LCDR2 comprises the amino acid sequence SDG;
SEQ ID NOs: 168, 169, 170, 171, and 172, and wherein LCDR2 comprises the amino acid sequence KDT;
SEQ ID NOs: 173, 174, 175, 171, and 176, and wherein LCDR2 comprises the amino acid sequence KDT;
SEQ ID NOs: 177, 178, 179, 180, and 181, and wherein LCDR2 comprises the amino acid sequence SSS;
SEQ ID NOs: 182, 183, 184, 185, and 186, and wherein LCDR2 comprises the amino acid sequence LVS;
SEQ ID NOs: 187, 188, 189, 100, and 190, and wherein LCDR2 comprises the amino acid sequence ANI;
SEQ ID NOs: 191, 192, 193, 185, and 194, and wherein LCDR2 comprises the amino acid sequence QVS;
SEQ ID NOs: 195, 196, 197, 171, and 198, and wherein LCDR2 comprises the amino acid sequence KDT;
SEQ ID NOs: 195, 196, 197, 171, and 199, and wherein LCDR2 comprises the amino acid sequence KDT;
SEQ ID NOs: 195, 196, 197, 200, and 201, and wherein LCDR2 comprises the amino acid sequence RDT;
SEQ ID NOs: 195, 196, 197, 200, and 202, and wherein LCDR2 comprises the amino acid sequence KDT;
SEQ ID NOs: 195, 196, 197, 171, and 190, and wherein LCDR2 comprises the amino acid sequence KDT;
SEQ ID NOs: 195, 196, 197, 203, and 204, and wherein LCDR2 comprises the amino acid sequence RDT;
SEQ ID NOs: 195, 196, 197, 171, and 205, and wherein LCDR2 comprises the amino acid sequence KDT;
SEQ ID NOs: 206, 207, 208, 209, and 210, and wherein LCDR2 comprises the amino acid sequence SVS;
SEQ ID NOs: 211, 112, 113, 114, and 115, and wherein LCDR2 comprises the amino acid sequence GSS;
SEQ ID NOs: 168, 117, 212, 213, and 214, and wherein LCDR2 comprises the amino acid sequence KDT;
SEQ ID NOs: 121, 215, 216, 166, and 217, and wherein LCDR2 comprises the amino acid sequence SDG;
SEQ ID NOs: 218, 219, 220, 221, and 222, and wherein LCDR2 comprises the amino acid sequence RDT;
SEQ ID NOs: 223, 224, 225, 213, and 226, and wherein LCDR2 comprises the amino acid sequence KDT;
SEQ ID NOs: 121, 227, 228, 229, and 230, and wherein LCDR2 comprises the amino acid sequence SDG;
SEQ ID NOs: 231, 232, 233, 234, and 235, and wherein LCDR2 comprises the amino acid sequence NND;
SEQ ID NOs: 236, 237, 238, 171, and 239, and wherein LCDR2 comprises the amino acid sequence KDT;
SEQ ID NOs: 240, 143, 241, 242, and 243, and wherein LCDR2 comprises the amino acid sequence SNK; or
SEQ ID NOs: 244, 138, 245, 246, and 247, and wherein LCDR2 comprises the amino acid sequence EAS.

Also provided is an isolated nucleic acid encoding a single-chain variable fragment (scFv) comprising a polynucleotide sequence set forth in SEQ ID NOs: 1-43. Also provided is an isolated nucleic acid encoding a single-chain variable fragment (scFv) consisting of a polynucleotide sequence set forth in SEQ ID NOs: 1-43.

Tolerable variations of the nucleic acid sequences will be known to those of skill in the art. For example, in some embodiments the nucleic acid encoding a single-chain variable fragment comprises a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the nucleotide sequences set forth in SEQ ID NOs: 1-43.

In certain embodiments, a nucleic acid of the present disclosure comprises a first polynucleotide sequence and a second polynucleotide sequence. The first and second polynucleotide sequence can be connected by a linker. For example, in certain embodiments the heavy chain variable region and the light chain variable region of an scFv are connected by a linker. In certain embodiments, the nucleic acid comprises from 5′ to 3′ the first polynucleotide sequence, the linker, and the second polynucleotide sequence. In certain embodiments, the nucleic acid comprises, from 5′ to 3′, the second polynucleotide sequence, the linker, and the first polynucleotide sequence.

Another aspect of the present disclosure provides a vector comprising any one of the isolated nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell can be of eukaryotic, prokaryotic, mammalian, or bacterial origin. Non-limiting examples of cells that can be used to express the anti-CD19 antibodies or antigen-binding fragments of scFv disclosed herein include Human embryonic kidney (HEK) cell lines (e.g., HEK293), Chinese hamster ovary (CHO) cell lines, Baby hamster kidney (BHK) cell lines, COS cell lines, Madin Darby canine kidney (MDCK) cell line, and HeLa cell lines. In some cases, the host cell is a Chinese Hamster Ovary cell.

A method of producing an antibody, a binding polypeptide or scFv that binds to human CD19 is also provided herein, wherein the method comprises culturing the host cell. In some embodiments, the method further comprises incubating the host cell in a cell culture medium under conditions sufficient to allow expression and secretion of the anti-CD19 antibody or antigen-binding fragment thereof or the scFv described herein.

In some embodiments, a nucleic acid of the present disclosure can be operably linked to a transcriptional control element, e.g., a promoter, and enhancer, etc. Suitable promoter and enhancer elements are known to those of skill in the art.

In certain embodiments, the nucleic acid is in operable linkage with a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GALI promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADCI promoter, a TRPI promoter, a URA3 promoter, a LEU2 promoter, an ENO promoter, a TPI promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a tre promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173 (1): 86-93; Alpuche-Aranda et al., Proc. Natl. Acad. Sci. USA (1992) 89 (21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma 70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein--Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuL V promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch can make use of a Cre-lox-mediated recombination (see, e.g., Fuhrmann-Benzakein, et al., Proc. Natl. Acad. Sci. USA (2000) 28: e99, the present disclosure of which is incorporated herein by reference). Any suitable combination of recombinase, endonuclease, ligase, recombination sites, etc. known to the art can be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the present disclosures of which are incorporated herein by reference.

A nucleic acid of the present disclosure can be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host can be present. Suitable expression vectors include, but are not limited to, viral vectors (e.g., viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10: 641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) can be used to introduce the nucleic acid into a host cell. Accordingly, an expression vector (e.g., a lentiviral vector) of the present disclosure can comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In one embodiment, the vector further comprises an elongation-factor-1-alpha promoter (EF-1α promoter). Use of an EF-1α promoter can increase the efficiency in expression of downstream transgenes. Physiologic promoters (e.g., an EF-1α promoter) can be less likely to induce integration mediated genotoxicity and can abrogate the ability of the retroviral vector to transform stem cells. Other physiological promoters suitable for use in a vector (e.g., lentiviral vector) are known to those of skill in the art and can be incorporated into a vector of the present disclosure. In some embodiments, the vector (e.g., lentiviral vector) further comprises a non-requisite cis acting sequence that can improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and can be incorporated into a vector (e.g., lentiviral vector) of the present disclosure. In some embodiments, the vector further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present disclosure further comprises a WPRE sequence. Various posttranscriptional regulator elements are known to those of skill in the art and can be incorporated into a vector (e.g., lentiviral vector) of the present disclosure. A vector of the present disclosure can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present disclosure includes a 3′ U3 deleted LTR. Accordingly, a vector (e.g., lentiviral vector) of the present disclosure can comprise any combination of the elements described herein to enhance the efficiency of functional expression of transgenes. For example, a vector (e.g., lentiviral vector) of the present disclosure can comprise a WPRE sequence, cPPT sequence, RRE sequence, 5′LTR, 3′ U3 deleted LTR′ in addition to a nucleic acid encoding for a CAR.

Vectors of the present disclosure can be self-inactivating vectors. As used herein, the term “self-inactivating vector” refers to vectors in which the 3′ LTR enhancer promoter region (U3 region) has been modified (e.g., by deletion or substitution). A self-inactivating vector can prevent viral transcription beyond the first round of viral replication. Consequently, a self-inactivating vector can be capable of infecting and then integrating into a host genome (e.g., a mammalian genome) only once, and cannot be passed further. Accordingly, self-inactivating vectors can greatly reduce the risk of creating a replication-competent virus.

In some embodiments, a nucleic acid of the present disclosure can be RNA, e.g., in vitro synthesized RNA. Methods for in vitro synthesis of RNA are known to those of skill in the art; any known method can be used to synthesize RNA comprising a sequence encoding a polypeptide of the present disclosure. Methods for introducing RNA into a host cell are known in the art. See, e.g., Zhao et al. Cancer Res. (2010) 15:9053. Introducing RNA comprising a nucleotide sequence encoding a polypeptide of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

In order to assess the expression of a polypeptide or portions thereof, the expression vector to be introduced into a cell can also contain either a selectable marker gene or a reporter gene, or both, to facilitate identification and selection of expressing cells from the population of cells sought to be transfected or infected through viral vectors. In some embodiments, the selectable marker can be carried on a separate piece of DNA and used in a co-transfection procedure. Both selectable markers and reporter genes can be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes can include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82).

In some embodiments, a nucleic acid of the present disclosure is provided for the production of a polypeptide as described herein, e.g., in a host cell. In some embodiments, a nucleic acid of the present disclosure provides for amplification of the polypeptide-encoding nucleic acid.

### Immunoconjugates

The present disclosure also provides immunoconjugates comprising any of the anti-hCD19 antibodies or antigen-binding fragments, or the scFvs, or the CARs disclosed herein. In some embodiments, the immunoconjugate comprises an antibody or antigen-binding fragments, or the scFv disclosed linked to an agent. In some embodiments, the immunoconjugate comprises a bispecific molecule disclosed herein linked to an agent. The agent can be a therapeutic agent. The agent can be a diagnostic agent.

For diagnostic agents, appropriate agents can include detectable labels. Non-limiting examples of detectable labels include radioisotopes, for whole body imaging, and radioisotopes, enzymes, fluorescent labels and other suitable antibody tags for sample testing. The detectable labels that can be linked to any anti-hCD19 antibody or antigen binding fragment thereof or scFv described herein can be any of the various types used currently in the field of in vitro diagnostics, including particulate labels including metal sols such as colloidal gold, isotopes such as ¹²⁵I or ^{99m}Tc presented for instance with a peptidic chelating agent of the N2S2, N3S or N4 type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1, 2 dioxetane substrates such as adamantyl methoxy phosphoryloxy phenyl dioxetane (AMPPD), disodium 3-(4-(methoxyspiro {1,2-dioxetane-3,2′-(5′-chloro) tricyclo {3.3.1.1 3, 7} decan}-4-yl) phenyl phosphate (CSPD), as well as CDP and CDP-STAR® or other luminescent substrates well-known to those in the art, for example the chelates of suitable lanthanides such as Terbium (III) and Europium (III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

In some embodiments, conjugation methods result in linkages which are substantially (or nearly) non-immunogenic, e.g., peptide-(i.e., amide-), sulfide-, (sterically hindered), disulfide-, hydrazone-, and ether linkages. These linkages are nearly non-immunogenic and show reasonable stability within serum; see, e.g., Senter, P. D., Curr. Opin. Chem. Biol. 13 (2009) 235-244; WO 2009/059278; WO 95/17886).

Depending on the biochemical nature of the agent and the anti-CD19 antibody or the scFv or the CAR, different conjugation strategies can be employed. In case the agent is naturally occurring or recombinant of between 50 to 500 amino acids, there are standard procedures in textbooks describing the chemistry for synthesis of protein conjugates, which can be easily followed by the skilled artisan (see, e.g., Hackenberger, C. P. R., and Schwarzer, D., Angew. Chem. Int. Ed. Engl. 47 (2008) 10030-10074). In some embodiments the reaction of a maleinimido agent with a cysteine residue within the antibody or the agent is used. This is an especially suited coupling chemistry in case e.g., a Fab or Fab′-fragment of an antibody is used. Alternatively, in some embodiments, coupling to the C-terminal end of the antibody or agent is performed. C-terminal modification of a protein, e.g., of a Fab-fragment, can be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al. Chem. Biol. 15 (2008) 128-136; and Protease-catalyzed formation of C—N bonds is used by Bordusa, F., Highlights in Bioorganic Chemistry (2004) 389-403). Site specific reaction and covalent coupling can also be achieved by the selective reaction of terminal amino acids with appropriate modifying reagents.

The reactivity of an N-terminal cysteine with benzonitriles (see Ren, H. et al., Angew. Chem. Int. Ed. Engl. 48 (2009) 9658-9662) can be used to achieve a site-specific covalent coupling.

Native chemical ligation can also rely on C-terminal cysteine residues (Taylor, E. Vogel; Imperiali, B, Nucleic Acids and Molecular Biology (2009), 22 (Protein Engineering), 65-96). U.S. Pat. No. 6,437,095 B1 describes a conjugation method which is based on the faster reaction of a cysteine within a stretch of negatively charged amino acids with a cysteine located in a stretch of positively charged amino acids.

The agent can also be a synthetic peptide or peptide mimic. In case a polypeptide is chemically synthesized, amino acids with orthogonal chemical reactivity can be incorporated during such synthesis (see e.g., de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

In some embodiments, the agent attached to an anti-CD19 antibody or scFv is selected from the group consisting of a binding moiety, a labeling moiety, and a biologically active moiety.

Anti-CD19 antibodies and scFvs described herein can also be conjugated to a therapeutic agent to form an immunoconjugate such as an antibody-drug conjugate (ADC). Suitable therapeutic agents include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. In some embodiments, the linker is a peptidyl linker such as Val-Cit, Ala-Val, Val-Ala-Val, Lys-Lys, Pro-Val-Gly-Val-Val (SEQ ID NO: 78), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038658; WO 07/051081; WO 07/059404; WO 08/083312; and WO 08/103693; U.S. Patent Publications 20060024317; 20060004081; and 20060247295, the contents of which are herein incorporated by reference.

Anti-CD19 antibodies or scFv described herein, can also be used for detecting CD19 protein in tissues or tissue samples. The antibodies can be used, e.g., in an ELISA assay or in flow cytometry. In some embodiments, an anti-CD19 antibody or scFv is contacted with cells, e.g., cells in a tissue, for a time appropriate for specific binding to occur, and then a reagent, e.g., an antibody that detects the anti-CD19 antibody, is added. Exemplary methods for detecting CD19 in a sample (cell or tissue sample) comprise (i) contacting a sample with an anti-CD19 antibody or scFv, for a time sufficient for allowing specific binding of the anti-CD19 antibody or scFv to CD19 in the sample, and (2) contacting the sample with a detection reagent, e.g., an antibody, that specifically binds to the anti-CD19 antibody or scFv to thereby detect CD19 bound by the anti-CD19 antibody or scFv. Wash steps can be included after the incubation with the antibody and/or detection reagent. Anti-CD19 antibodies or scFv for use in these methods do not have to be linked to a label or detection agents, as a separate detection agent can be used.

### Methods of Generating Modified Immune Cells

The present invention provides methods for producing/generating a modified immune cell or precursor cell thereof (e.g., a T cell/NK cell/NKT cell). The cells are generally engineered by introducing a nucleic acid encoding a subject CAR (e.g., hCD19 CAR). In some embodiments, the CAR is accompanied by a gene modification system that is capable of modifying the expression or sequence of the endogenous hCD19 gene.

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12 (8): 861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR of the invention is introduced into a cell by an expression vector. Expression vectors comprising a nucleic acid encoding a subject CAR (e.g., CD19 CAR) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7 (20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

The present invention also provides genetically engineered cells which include and stably express a subject CAR of the present disclosure. In some embodiments, the genetically engineered cells are genetically engineered T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In one embodiment, the genetically engineered cells are autologous cells.

Modified cells (e.g., comprising a subject CAR) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electro transfer and/or hydrodynamic delivery) and/or particle-based methods (e.g., impalefection, using a gene gun and/or magnetofection). Transfected cells expressing a subject CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells and/or NKT cells. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity-based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed, and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed, and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker) or express relatively low levels (marker^{low}) of one or more markers. For example, in certain aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In certain exemplary embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4+ or CD8+selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations. In some embodiments, combining TCM-enriched CD8+ T cells and CD4+ T cells further enhances efficacy.

In some embodiments, memory T cells are present in both CD62L+ and CD62L-subsets of CD8+peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and/or a CD8+T population is enriched for central memory (TCM) cells. In some embodiments, the enrichment for central memory T (TCM) cells is based on positive or high surface expression of CD45RO, CD62L, CCR7, CD28, CD3, and/or CD 127; in certain aspects, it is based on negative selection for cells expressing or highly expressing CD45RA and/or granzyme B. In certain aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in certain aspects are carried out simultaneously and in other aspects are carried out sequentially, in either order. In some embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+T lymphocytes are CD45RO-, CD45RA+, CD62L+, CD4+ T cells. In some embodiments, central memory CD4+ cells are CD62L+ and CD45RO+. In some embodiments, effector CD4+ cells are CD62L- and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4 cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of Immune Cells

Whether prior to or after modification of cells to express a subject CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005, the relevant contents of which are incorporated by reference. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30 (8): 3975-3977; Haanen et al., J. Exp. Med. (1999) 190 (9): 1319-1328; and Garland et al., J. Immunol. Methods (1999) 227 (1-2): 53-63).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3, and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore, the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetylcysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

### Methods of Treatment

The antibodies or antigen binding fragments thereof, scFvs, and modified immune cells (e.g., T cells) described herein may be included in a composition for immunotherapy. The composition may include a pharmaceutical composition and further include a pharmaceutically acceptable carrier. A therapeutically effective amount of the pharmaceutical composition comprising the antibodies or antigen binding fragments thereof, scFvs, and modified T cells may be administered.

In one aspect, the invention includes a method for adoptive cell transfer therapy comprising administering to a subject in need thereof a modified T cell of the present invention. In another aspect, the invention includes a method of treating a disease or condition in a subject comprising administering to a subject in need thereof a population of modified T cells.

Also included is a method of treating cancer in a subject in need thereof, comprising administering to the subject an effective amount of a modified immune cell of the invention of the current disclosure, or a modified immune cell generated by the methods of the invention of the current disclosure, thereby treating the cancer. In some embodiments, the immune cell comprises an isolated nucleic acid encoding an antibody, scFv, or CAR of the invention of the current disclosure. In some embodiments, the modified immune cell is selected from the group consisting of a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell), and a dendritic cell. In some embodiments, the antibody, scFv, or CAR of the invention has a binding specificity for human CD19 (hCD19). In some embodiments, the cancer is a hematologic cancer. In some embodiments, the cancer is associated with the expression of CD19. In some embodiments, the CD19 is expressed on tumor cells. In some embodiments, the cancer is selected from the group consisting of Burkitt lymphoma, chronic lymphocytic leukemia (CLL), acute lymphoblastic leukemia (ALL), B-cell lymphoma, and B-cell leukemia. In some embodiments, the subject is a human.

Also provided is a method of treating an autoimmune disorder or disease in a subject in need thereof, comprising administering to the subject an effective amount of the modified immune cell of the invention of the current disclosure or a modified immune cell generated by the methods of the current disclosure, thereby treating the autoimmune disorder. In some embodiments, the autoimmune disorder is antibody mediated. In some embodiments, administration of the modified immune cell depletes autoimmune B cells or B cells and plasma cells secreting antibodies and autoreactive and pathogenic antibodies. In some embodiments, the autoimmune disorder is selected from the group consisting of rheumatoid arthritis, Systemic Lupus Erythematosus (SLE), Idiopathic/Autoimmune Thrombocytopenia Purpura (ITP), pemphigus-related disorders, diabetes, scleroderma, myasthenia gravis, multiple sclerosis, vasculitis, and autoimmune hemolytic anemia, among others.

Also provided is a method of treating an alloantibody-mediated condition or disorder in a subject in need thereof, comprising administering to the subject an effective amount of the modified immune cell of the invention of the current disclosure or a modified immune cell generated by the methods of the current disclosure, thereby treating the alloantibody-mediated condition or disorder. Examples of alloantibody-mediated conditions or disorders include, but are not limited to, solid organ transplant immune incompatibility, resistance to enzyme replacement therapy or acute or delayed hemolytic reactions to transfusion, and chronic alloantibody mediated rejection (CAMR), among others. In some embodiments, the alloantibody-mediated condition or disorder is antibody mediated. In some embodiments, administration of the modified immune cell depletes alloantibody-producing B cells or B cells and plasma cells secreting antibodies and alloreactive and pathogenic antibodies. In some embodiments, the alloantibody-mediated condition or disorder includes the production of alloantibodies, for example antibodies specific for therapeutic or transplanted cells or tissues which are allogenic with respect to the subject. In some embodiments, the alloantibody-mediated condition or disorder includes the production of antibodies against otherwise therapeutic molecules, proteins, lipids, and polysaccharides or combinations thereof. In these cases, the administration of the modified immune cell depletes B cells that generate alloantibodies. In some embodiments, the alloantibody condition or disorder is selected from the group consisting of organ transplant immune incompatibility hemophilia (hemophilia A or B), lysosomal storage diseases (LSD), urea cycle disorder, adenosine deaminase deficiency, neuronal ceroid lipofuscinoses (NCL) (inclusive of infant, juvenile, and adult NCL; e.g., Batten disease), hyperammonemia, and chronic graft-versus-host disease (cGvHD). Examples of lysosomal storage diseases include, without limitation, glycogen storage disease (e.g., type II (Pompe disease)), Gaucher's disease, Niemann-Pick disease, Fabry's disease, and mucopolysaccharidosis (e.g., MPS I, MPS II, MPS VI, etc.), among others.

In one aspect, the present disclosure provides a method for treating a disease or condition in a subject in need thereof comprising administering to the subject the antibodies, binding polypeptides, and scFvs described herein, or a composition comprising any one of these, or an engineered immune cell or precursor thereof comprising a CAR described herein. The composition can include a pharmaceutical composition and further include a pharmaceutically acceptable carrier. A therapeutically effective amount of the pharmaceutical composition can be administered to the subject.

In certain embodiments, described herein are the antibodies, binding polypeptides, and scFvs described herein, or a composition comprising any one of these, or a modified immune cell or precursor thereof comprising a CAR described herein, for use as a medicament.

In certain embodiments, described herein are the antibodies, binding polypeptides, and scFvs described herein, or a composition comprising any one of these, or a modified immune cell or precursor thereof comprising a CAR described herein for use as a medicament for the treatment of cancer or a B cell-mediated autoimmune condition.

In certain embodiments, described herein is use of the antibodies, binding polypeptides, and scFvs described herein, or a composition comprising any one of these, or a modified immune cell or precursor thereof comprising a CAR described herein for the manufacture of a medicament.

In certain embodiments, described herein is use of the antibodies, binding polypeptides, and scFvs described herein, or a composition comprising any one of these, or a modified immune cell or precursor thereof comprising a CAR described herein for the manufacture of a medicament for the treatment of cancer or B cell-mediated autoimmune condition.

Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

In some embodiments, the method comprises administering to the subject an engineered immune effector cell comprising an isolated CAR comprising an antigen binding domain comprising a heavy chain variable region comprising an amino acid sequence at least 80%, 85%, 90%, 95%, 96%, 97%, 98%, 99%, or 100% identical to SEQ ID NOs: 314-356 and a light chain variable region comprising an amino acid sequence at least 80%, 85%, 90%, 95%, 96%, 97%, 98%, 99%, or 100% identical to SEQ ID NOs: 271-313. In certain embodiments, the immune effector cell is a T cell.

In certain embodiments, the cancer is associated with expression of CD19. In certain embodiments, the CD19 is expressed on a malignant immune cell of the subject. In certain embodiments, the malignant immune cell is a B lymphocyte. In certain embodiments, the malignant B cells is associated with a B cell lymphoma or leukemia. In certain embodiments the B cell lymphoma or leukemia includes acute lymphoblastic leukemia (ALL), chronic lymphocytic leukemias (CLL), Burkitt lymphoma, B cell leukemias, and B cell lymphomas.

In certain embodiments, the method further comprises administering one or more additional therapeutics or interventions. There is no limitation on such additional therapeutics or interventions, which can include any therapeutic agents or small molecule drugs that is helpful for treating the subject in need thereof. In some embodiments, the additional therapeutics or interventions are administered with the antibody or antigen-binding fragments thereof or the scFv or engineered immune effector cell described herein, or the bispecific molecule or the immunoconjugates comprising these, as a combination therapy. Non-limiting examples of additional therapeutics or interventions include chemotherapy (e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine), radiation therapy, immunotherapy, and other targeted therapy.

Compositions of the present disclosure can be administered in dosages and routes and at times to be determined in appropriate pre-clinical and clinical experimentation and trials. Compositions can be administered multiple times at dosages within these ranges. Administration of the compositions can be combined with other methods useful to treat the desired disease or condition as determined by those of skill in the art. In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, therapeutically acceptable amount is between about 0.1 mg/kg and about 50 mg/kg. In certain embodiments, therapeutically acceptable amount is between about 1 mg/kg and about 40 mg/kg. In certain embodiments, therapeutically acceptable amount is between about 1 mg/kg and about 20 mg/kg. In certain embodiments, therapeutically acceptable amount is between about 1 mg/kg and about 10 mg/kg. In certain embodiments, therapeutically acceptable amount is between about 5 mg/kg and about 30 mg/kg. In certain embodiments, therapeutically acceptable amount is between about 5 mg/kg and about 20 mg/kg. Therapeutically effective amounts include amounts sufficient to ameliorate one or more symptoms associated with the disease or affliction to be treated.

In terms of the present disclosure, prophylactic, palliative, symptomatic and/or curative treatments may represent separate aspects of the disclosure. An anti-CD19 antibody or antigen-binding fragments thereof or scFv or CAR or modified immune cell or precursor thereof comprising a CAR disclosed herein can be administered parenterally, such as intravenously, such as intramuscularly, such as subcutaneously. Alternatively, an antibody or antigen-binding fragments thereof or scFv or CAR or modified immune cell or precursor thereof comprising a CAR of the present disclosure can be administered via a non-parenteral route, such as orally or topically. An antibody of the invention can be administered prophylactically. An antibody or antigen-binding fragments thereof or scFv or CAR or modified immune cell or precursor thereof comprising a CAR of the present disclosure can be administered therapeutically (on demand).

### Pharmaceutical Compositions, Kits, and Methods of Making the Compositions

Also provided are pharmaceutical compositions comprising any one of the binding polypeptides, scFvs, antibodies, or the antigen-binding fragments disclosed herein. Among the compositions are pharmaceutical compositions and formulations for administration, such as for treatment of a disease or disorder. Also provided are therapeutic methods for administering the pharmaceutical compositions to subjects, e.g., canines.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular composition and/or by the method of administration. Accordingly, there are a variety of suitable formulations. For example, the pharmaceutical composition can contain preservatives. Suitable preservatives can include, for example, methylparaben, propylparaben, sodium benzoate, and benzalkonium chloride. In some aspects, a mixture of two or more preservatives is used. The preservatives or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in an amount effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

In certain embodiments the anti-CD19 antibodies or antigen-binding fragments thereof or scFv or CAR or modified immune cell or precursor thereof comprising an anti-CD19 CAR of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration.

In certain embodiments, described herein is a method of making a composition for treating cancer, sepsis or septic shock, or chronic infection (e.g., viral infections), comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

While the present disclosure has been described with reference to the specific embodiments thereof, it should be understood by those skilled in the art that various changes can be made, and equivalents can be substituted without departing from the true spirit and scope of the present disclosure. It will be readily apparent to those skilled in the art that other suitable modifications and adaptations of the methods described herein can be made using suitable equivalents without departing from the scope of the embodiments disclosed herein. In addition, many modifications can be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### Definitions

In the detailed description, certain specific details are set forth in order to provide a thorough understanding of various embodiments. However, one skilled in the art will understand that the embodiments provided can be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The articles “a” and “an” are used herein to refer to one or to more than one (i.e., to at least one) of the grammatical object of the article. By way of example, “an element” means one element or more than one element.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a dog or canine.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules comprising two heavy chain and two light chain polypeptides. Each polypeptide chain contains three complementarity-determining regions (CDRs), which bind to the antigen and defines the antibody's antigen specificity.

As used herein, the term “antibody” and “antibodies” can also include polypeptides or polypeptide complexes derived from full-length antibodies. These polypeptide complexes can be naturally occurring or constructed from single chain antibodies or antibody fragments and retain an antigen-specific binding ability. The antibodies of the present disclosure can exist in a variety of forms including, for example, polyclonal antibodies, monoclonal antibodies, Fv, Fab and F(ab′)₂, as well as single chain antibodies, scFv, caninized antibodies, canine antibodies, humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). For preparation of suitable antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies of this disclosure. Also, transgenic mice, or other organisms such as other mammals, can be used to express humanized or human antibodies as well as caninized or canine antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding can be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

A “linker” herein is also referred to as “linker sequence,” “spacer,” “tethering sequence,” or grammatical equivalents thereof. A “linker” as referred herein connects two distinct molecules that by themselves possess target binding, catalytic activity, or are naturally expressed and assembled as separate polypeptides, or comprise separate domains of the same polypeptide. For example, two distinct binding moieties or a heavy-chain/light-chain pair. A number of strategies can be used to covalently link molecules together. Linkers described herein can be utilized to join a light chain variable region and a heavy chain variable region in an scFv molecule; or can be used to tether an scFv or other antigen binding fragment on the N- or C-terminus of an antibody heavy chain; or the N- or C-terminus of a light chain to create a bispecific or multi-specific binding molecule. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide can predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length can be used. Useful linkers include glycine-serine polymers, including for example (GS)n, (GSGGS)n (SEQ ID NO: 248), (GGGGS)n (SEQ ID NO: 250), and (GGGS)n (SEQ ID NO: 249), where n is an integer of at least one, glycine-alanine polymers, alanine-serine polymers, and other flexible linkers. Exemplary, linkers for linking antibody fragments or single chain variable fragments can include AAEPKSS (SEQ ID NO: 267), AAEPKSSDKTHTCPPCP (SEQ ID NO: 268), GGGG (SEQ ID NO: 269), or GGGGDKTHTCPPCP (SEQ ID NO: 270). Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR can vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In certain embodiments, the CDRs of the antibodies described herein can be defined by IMGT method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single V_{H} or V_{L} domain can be sufficient to confer antigen-binding specificity. Furthermore, antibodies that bind a particular antigen can be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

An “epitope” refers to the binding determinant of an antibody or fragment described herein minimally necessary for specific binding of the antibody or fragment thereof to a target antigen. When the target antigen is a polypeptide, the epitope will be a continuous or discontinuous epitope. A continuous epitope is formed by one region of the target antigen, while a discontinuous epitope can be formed from two or more separate regions. A discontinuous epitope, for example, can form when a target antigen adopts a tertiary structure that brings two amino acid sequences together and forms a three-dimensional structure bound by the antibody. When the target antigen is a polypeptide, the epitope will generally be a plurality of amino acids linked into a polypeptide chain. A continuous epitope can comprise 5, 6, 7, 8, 9, 10, 11, 12, 13, 14, 15, 16, 17, 18, 19, or 20 contiguous amino acids. While an epitope can comprise a contiguous polymer of amino acids, not every amino acid of the polymer can be contacted by an amino acid residue of the antibody. Such non-contacted amino acids will still comprise part of the epitope as they can be important for the structure and linkage of the contacted amino acids. The skilled artisan can determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

The term “antibody fragment” refers to a polypeptide comprising or derived from a portion of an intact antibody and comprises the antigen-binding determining variable regions of an intact antibody. Examples of antibody fragments include, but are not limited to, Fab, Fab′, F(ab′)₂, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multi-specific antibodies formed from antibody fragments. Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., polypeptide linkers, and/or those that are not produced by enzyme digestion of a naturally occurring intact antibody. In some aspects, the antibody fragments are scFvs.

A Fab or Fab fragment contains the constant domain of the light chain and the first constant domain (CH1) of the heavy chain. Fab′ or Fab′ fragments differ from Fab fragments by the addition of a few residues at the carboxyl terminus of the heavy chain CH1 domain including one or more cysteines from the antibody hinge region. Fab′ fragments are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fragment crystallizable (Fc) region of an intact antibody, clear more rapidly from the circulation of animals, and can have less nonspecific tissue binding than an intact antibody. “Fv” fragment is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VHVL dimer. In some cases, the six CDRs confer target binding specificity to the antibody. However, in some cases. even a single variable domain (or half of an Fv comprising only three CDRs specific for a target) can have the ability to recognize and bind target. Single domain antibodies (sdAb)/single-chain fragments are composed of a single VH or VL domain which exhibit exhibits sufficient affinity to an antigen. The antibody fragment also includes a canine antibody or a caninized antibody or a portion of a canine antibody or a caninized antibody. A scFv (single-chain Fv) refers to antibody binding fragments that comprise the VH and VL domains of an antibody, where these domains are present in a single polypeptide chain. Generally, the scFv polypeptide further comprises a polypeptide linker between the VH and VL domains which enables the scFv to form a structure favorable for target binding.

The term “diabodies” refers to small antibody fragments prepared by constructing scFv fragments with short linkers (about 5-10 residues) between the VH and VL domains such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the VH and VL domains of the two antibodies are present on different polypeptide chains.

The term “linear antibodies” generally refers to the antibodies comprise comprising a pair of tandem Fd segments (VH-CH1-VH-CH1) which, together with complementary light chain polypeptides, form a pair of antigen binding regions. Linear antibodies can be bispecific or monospecific.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in some antibody molecules in their naturally occurring conformations. k and 1 light chains refer to the two major antibody light chain isotypes.

The term “synthetic antibody” as used herein, is means an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the present disclosure can be replaced with other amino acid residues from the same side chain family and the altered antibody can be tested for the ability to bind human CD19 using the functional assays described herein.

“Co-stimulatory ligand,” as the term is used herein, includes a molecule expressed by an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to cell-surface molecules expressed by T cells that specifically bind with co-stimulatory ligands expressed by antigen-presenting cells (APCs), thereby providing a “secondary signal” which, in combination with the “primary signal” delivered through MHC/HLA-antigen interactions with the T Cell Receptor (TCR) results in optimal T cell activation including, but not limited to, cytokine production and proliferation. Co-stimulatory molecules include, but are not limited to CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

The term “dysregulated” when used in the context of the level of expression or activity of CD 19 refers to the level of expression or activity that is different from the expression level or activity of CD19 or it's its ligands in an otherwise identical healthy animal, organism, tissue, cell or component thereof. The term “dysregulated” also refers to the altered regulation of the level of expression and activity of CD19 compared to the regulation in an otherwise identical healthy animal, organism, tissue, cell or component thereof.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

“Effective amount” or “therapeutically effective amount” are used interchangeably herein, and refer to an amount of a compound, formulation, material, or composition, as described herein effective to achieve a particular biological result. Such results can include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” and “chimeric” forms of non-human (e.g., canine) antibodies are immunoglobulins, immunoglobulin chains or fragments thereof (such as Fv, Fab, Fab′, F(ab′)₂ or other antigen-binding subsequences of antibodies) which contain minimal sequences derived from non-human immunoglobulin. For the most part, humanized and chimeric antibodies are human immunoglobulins (recipient antibody) in which residues from a complementary-determining region (CDR) of the recipient are replaced by residues from a CDR of a non-human species (donor antibody) such as mouse, rat, canine or rabbit having the desired specificity, affinity, and capacity. In some instances, Fv framework region (FR) residues of the human immunoglobulin are replaced by corresponding non-human residues. Furthermore, humanized, and chimeric antibodies can comprise residues which are found neither in the recipient antibody nor in the imported CDR or framework sequences. These modifications are made to further refine and optimize antibody performance. In general, the humanized and chimeric antibody will comprise substantially all of at least one, and typically two, variable domains, in which all or substantially all of the CDR regions correspond to those of a non-human immunoglobulin and all or substantially all of the FR regions are those of a human immunoglobulin sequence. The chimeric antibody optimally also will comprise at least a portion of an immunoglobulin constant region (Fc), typically that of a human immunoglobulin.

As used herein, an “instructional material” includes a publication, a recording, a diagram, or any other medium of expression which can be used to communicate the usefulness of the compositions and methods of the present disclosure. The instructional material of the kit of the present disclosure can, for example, be affixed to a container which contains the nucleic acid, peptide, and/or composition of the present disclosure or be shipped together with a container which contains the nucleic acid, peptide, and/or composition. Alternatively, the instructional material can be shipped separately from the container with the intention that the instructional material and the compound be used cooperatively by the recipient.

“Identity” as used herein refers to the percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or can be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA can also include introns to the extent that the nucleotide sequence encoding the protein can in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence can be the core promoter sequence and in other instances, this sequence can also include an enhancer sequence and other regulatory elements which are required for expression of the gene product. The promoter/regulatory sequence can, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell. An example of a “cell surface receptor” is human GFRa4.

“Single chain antibodies” refer to antibodies formed by recombinant DNA techniques in which immunoglobulin heavy and light chain fragments are linked to each other using an engineered span of amino acids to recapitulate the Fv region of an antibody as a single polypeptide. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The term “subject” is intended to include living organisms in which an immune response can be elicited (e.g., mammals). A “subject” or “patient,” as used therein, can be a human or non-human mammal. Non-human mammals include, for example, livestock and pets, such as ovine, bovine, porcine, canine, feline and murine mammals. In some embodiments, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

The term “specifically binds,” as used herein, is means an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

The term “stimulation” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell and/or on a tumor cell.

A “stimulatory ligand,” as used herein, means a ligand that when present on an antigen presenting cell (e.g., an aAPC, a dendritic cell, a B-cell, and the like) or a tumor cell, can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a super-agonist anti-CD28 antibody, and a super-agonist anti-CD2 antibody.

Ranges: throughout this present disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

The present disclosure is further described in detail by reference to the following experimental examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teaching provided herein. While preferred embodiments of the present invention have been shown and described herein, it is understood by those skilled in the art that such embodiments are provided by way of example only. Numerous variations, changes, and substitutions will now occur to those skilled in the art without departing from the present disclosure. It should be understood that various alternatives to the embodiments of the present disclosure described herein can be employed in practicing the present disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Without further description, it is believed that one of ordinary skill in the art can, using the preceding description and the following illustrative examples, make and utilize the compounds of the present disclosure and practice the claimed methods. The following working examples, therefore, specifically point out some embodiments of the present disclosure.

The materials and methods used in the following examples are now described.

Anti-CD) 19 discovery campaign. ScFv discovery was performed by panning (i.e., selection) of human CD19-specific phage particles from a large naïve/non-immune canine phage display antibody library. To maintain proper conformation of CD19 molecules, CD19 protein with a biotinylated AviTag™ (Acrobiosystems, Newark, DE) was used and captured to streptavidin-coated 96-well microplates. To prevent the capture of streptavidin binders, the library was pre-absorbed against wells coated just with streptavidin. After negatively selected phage were positively selected on antigen-coated wells, unbound phage were washed away, bound phage were eluted with low pH, and pH-neutralized phage eluates were amplified overnight in bacterial culture (Rader et al., 2001, Phage Display: A Laboratory Manual, Cold Spring Harbor Press, NY, 10.01-10.20). This sequence of negative/positive selection and overnight amplification was performed a total of 4 times with increased stringency of selection in panning rounds 2, 3, and 4 (10 washes to remove unbound phage vs. 5 washes in panning round 1).

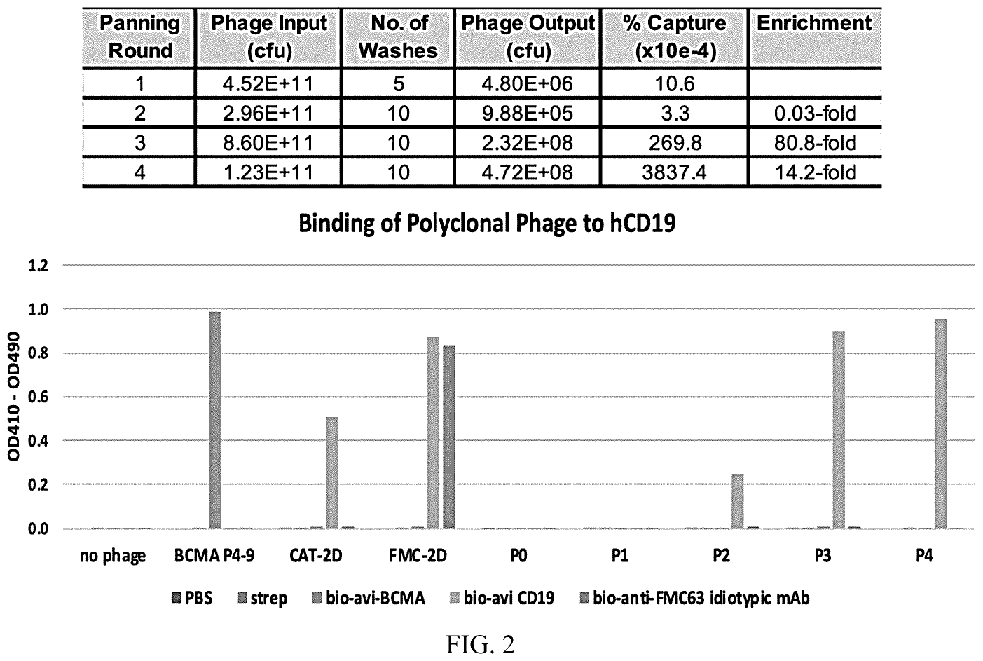

Enrichment of anti-CD19 scFvs. The fraction of phage bound to the CD19 target (ratio of number of phage in panning output vs. input) after each round of panning was determined as well as the binding reactivity of phage libraries prior to selection and after each round of panning as measured by phage ELISA. As shown in FIG. 2 (top), there was marked enrichment in the capture of phage beginning in panning round 3. This can be seen in the phage ELISA in FIG. 2 (bottom) where microplate wells were coated with human CD19 or controls (streptavidin alone or streptavidin loaded with biotinylated-AviTag™-human BCMA (Acrobiosystems) or a biotinylated anti-idiotypic antibody to FMC63 (Acrobiosystems), a murine anti-human CD19 antibody used in a number of FDA-approved CD19-directed CAR-T therapies including tisagenlecleucel).

Target-specific activation of (′ARs. CD19 scFv CAR-transduced Jurkat-NFAT-GFP (JNG) reporter cells were incubated with K562, K562 CD19 target cells at an effector-to-target (E:T) ratio of 1:1 or with OKT3 as positive control. 24 h after cells were harvested and stained for the activation marker CD69. JNG cell activation was analyzed by flow cytometry gating on CD69 and GFP double positive population. Percentages of activation of each scFv and FMC63 CAR-JNG (positive control) were determined.

CD19 CAR expression in human T cells. Fresh isolated human T cells from healthy donors obtained from the Human Immunology Core at the University of Pennsylvania were transduced with lentiviral vector supernatants encoding the CD19 scFv 18 or scFv 19 CARs, and CAR expression was evaluated using anti-myc tag antibody by flow cytometry. T cells transduced with a CD19-FMC63 CAR (FMC63) or CAT vector (CAR-T vector expressing the anti-CD19 CAT scFv; Ghorashian et al (2019) Nat Med 25:1408-1414) were used as positive controls. Mock-transduced T cells (NTD) were used as a negative control.

Expansion of CAR-expressing T cells. Primary human T cells from healthy volunteer donors were activated using anti-CD3/anti-CD28 coated Dynabeads (ThermoFisher, Waltham, MA) at a bead: cell ratio of 3:1 and cultured in RPMI1640 with 10% fetal bovine serum (FBS), 10 mM HEPES, and 1% penicillin/streptomycin. 24 h after activation, the T cells were transduced with CD19-CAR lentiviruses at an MOI of 3. T cells were then expanded in media containing IL-7 and IL-15 and measured every other day by coulter counter (Beckman, Chaska, MN) until they reached a cellular volume of approximately 400 fL, prior to cryopreservation.

Assessing CAR T cell function. The CD19 scFv CAR T cells were co-cultured with target cells in duplicates at an effector-target (E: T) ratio of 30:1 for 16 h at 37° C. and 5% CO2. K562-CD19 specific target cells and NALM6 target cells were used as positive control and K562 no target cells were used as negative control. The supernatants were collected, diluted 1:2 and subjected to IFNy detection by sandwich ELISA assay using Human IFN-gamma DuoSet ELISA kit (R&D Systems, Minneapolis MN)) according to the manufacturer's instructions.

To assess antigen-specific killing of a CD19 expressing cell line by CD19 CART cells, ⁵¹Cr labeled K562 or K562 CD 19 target cells were co-cultured with effector CD19 scFv CAR T cells or no transduced T cells as a negative control at different effector/target ratios (30:1, 10:1, 3:1 and 1:1). Supernatants were collected at 4-hrs, and the amount of ⁵¹Cr released from the labeled target cells was measured on a MicroBeta reader (Perkin Elmer, Waltham, MA). Target cells incubated in medium alone or with 1% SDS were used to determine spontaneous(S) or maximum (M) ⁵¹Cr release. Percentage of specific lysis was calculated as follow: 100× (cpm experimental release-cpm S release)/(cpm M release-cpm S release).

Treatment of tumor-bearing mice with CAR-T cells. NSG mice engrafted with NALM6 cells were treated with 0.5×10⁶, 1×10⁶, or 2×10⁶ CD19 scFv CAR T cells at day 5 following tumor cell injection. Leukemia progression was monitored by bioluminescence of ventral and dorsal mouse areas twice per week. Dorsal photon emission from NALM6 click beetle luciferase (CBG)-positive tumors are shown with individual animals depicted in each graph. Total human T cell engraftment in peripheral blood from the indicated mice groups was quantified using TruCount tubes following manufactured instructions. The absolute count of the cell population (A) was obtained with the following formula: A=X/Y×N/V, where X represents the number of positive cell events, Y represents the number of bead events, N represents the number of beads per test and V represents the test volume.

### Example 1: Discovery and Characterization of anti-Human CD19 scFvs

CD19, a cell-surface molecule on B lymphocytes, has been exploited as a target for the treatment of acute leukemias and lymphomas as exemplified by a number of recently FDA-approved CD19-directed chimeric antigen receptor (CAR) T cell drugs, most notably tisagenlecleucel marketed as Kymriah® by Novartis. CD19-directed CAR-T cells are also showing promise outside of cancer for counteracting “unwanted immunity” in cases of autoantibody-mediated autoimmune disease such as for the treatment of lupus erythematosus and for prevention of alloantibody-mediated solid organ transplant immune incompatibility due to the presence of donor-specific HLA antibodies. With respect to the latter application, clinical trials are currently underway which use CD19-directed CAR-T cells in combination with BCMA-directed CAR-T cells in order to eliminate alloantibody producing B cells and plasma cells, respectively, to treat alloimmunized patients who are in need of renal transplant. There is also a scientific desire to investigate new CAR-T cell designs that employ KIR-based signaling mechanisms vs. those that employ conventional CD3-zeta (CD3-zeta as used in all FDA-approved CAR-T drugs).

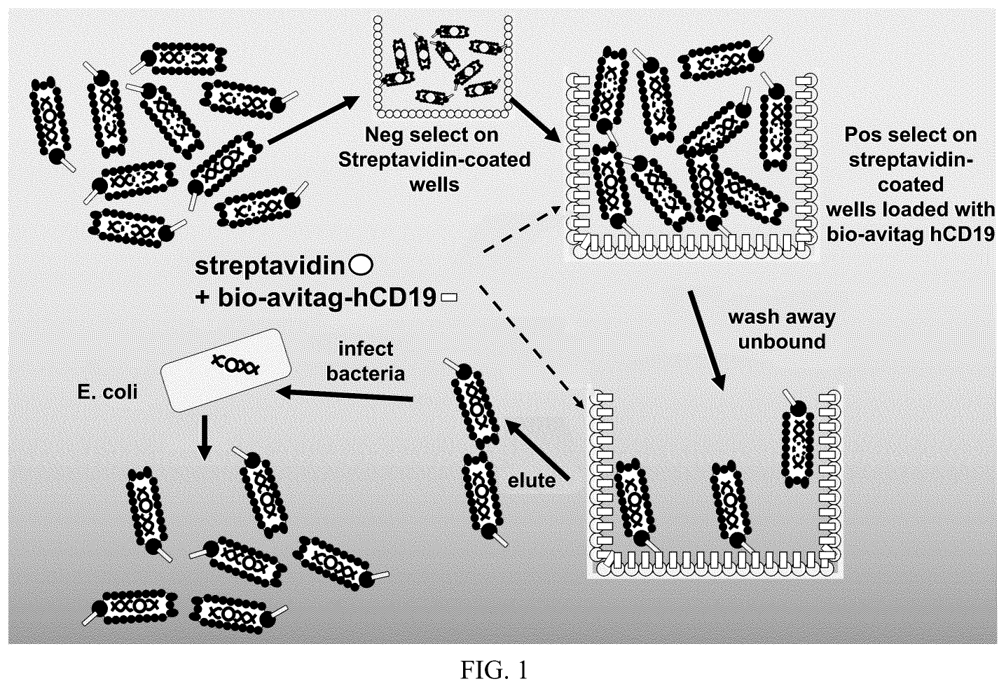

FIG. 1 presents an overview of the anti-CD19 discovery campaign performed by panning (i.e., selection) of human CD19-specific phage particles from a large naïve/non-immune canine phage display antibody library which has been used previously to isolate scFv directed against a number of other target antigens. In order to maintain proper conformation of CD19 molecules, CD19 protein with a biotinylated AviTag™ was used and captured to streptavidin-coated 96-well microplates. To prevent the capture of streptavidin-only binders, the library was pre-absorbed against wells coated just with streptavidin. After negatively selected phage were positively selected on antigen-coated wells, unbound phage were washed away, bound phage were eluted with low pH, and pH-neutralized phage eluates were amplified overnight in bacterial culture. This sequence of negative/positive selection and overnight amplification was performed a total of 4 times with increased stringency of selection in panning rounds 2, 3, and 4 (10 washes to remove unbound phage vs. 5 washes in panning round 1).

In order to assess library enrichment for anti-CD19 scFvs, the fraction of phage bound to the CD19 target (ratio of number of phage in panning output vs. input) after each round of panning was determined as well as the binding reactivity of phage libraries prior to selection and after each round of panning as measured by phage ELISA. As shown in FIG. 2 (top), there was marked enrichment in the capture of phage beginning in panning round 3. This can be seen in the phage ELISA in FIG. 2 (bottom) where microplate wells were coated with human CD19 or controls (streptavidin alone or streptavidin loaded with biotinylated-AviTag™-human BCMA or a biotinylated anti-idiotypic antibody to FMC63, a murine anti-human CD19 antibody used in a number of FDA-approved CD19-directed CAR-T therapies including tisagenlecleucel). After coating wells, phage samples were applied, unbound phage washed away, and bound phage were detected with an HRP-conjugated anti-M13 antibody and HRP substrate. Phage samples comprised polyclonal phage from the original unpanned scFv phage library (P0) and panned libraries (P1, P2, P3, and P4). Negative control phage included anti-BCMA-expressing phage (clone P4-9 from an unrelated project), and positive control phage comprised FMC-2D (a.k.a. FMC63) and CAT-2D, another murine anti-human CD19 antibody also used in CAR T therapy with patients (Ghorashian et al. (2019) Nature Medicine 25:1408-1414). Results for P0 through P4 phage samples show enrichment for anti-human CD19 binding phage. Positive and negative control well coatings and phage showed expected results. Only FMC63 reacts with its anti-idiotypic antibody as expected.

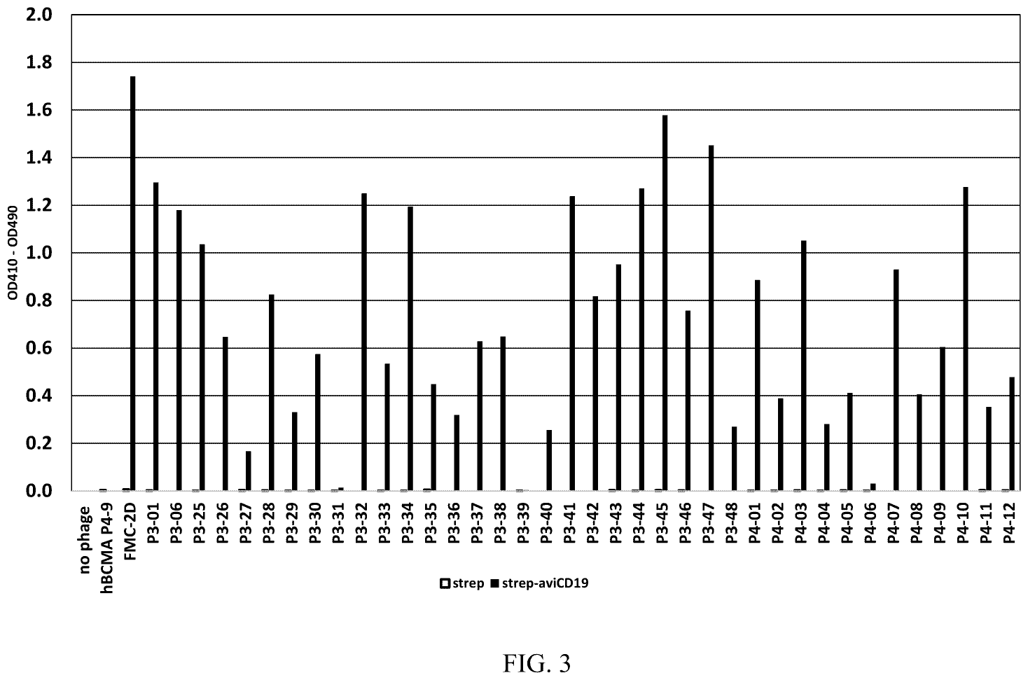

To identify unique human CD19-binding monoclonal scFv from the panned libraries, individual phage antibody clones were expanded, assessed for binding to CD19 by ELISA, and subjected to nucleotide sequencing to identify those clones that were unique. A total of 84 scFvs were randomly selected from the panned libraries (70 from P3 and 14 from P4). 73 of the 84 clones were positive for CD19 binding (60 of 70 from P3 and 13 of 14 from P4). 68 of the 73 positive clones had evaluative nucleotide sequences. Of those 68, there were 43 unique scFv nucleotide sequences identified. FIG. 3 depicts typical ELISA results for a cohort of mostly positive clones (38 of the 84 randomly selected clones are shown in this example). Tables 5 and 6 tabulate binding and genetic characterizations for the 43 unique scFvs listed in Column 1. Column 2 lists the specific names of the 68 positive clones that led to the 43 unique antibodies. Column 3 presents a semi-quantitative evaluation of binding strength to CD19 as expressed by a ratio of the binding of the test scFv clone to the binding of FMC63 run as a standard in each ELISA assay. Tables 5 and 6 also list the heavy chain (μ or γ) and light chain (κ or λ) isotypes and the heavy (VH, D, JH) and light chain (VL and JL) gene segments from which the scFv were derived as determined using the canine germline immunoglobulin database in IMGT (The International Immunogenetics Information System) and search tool IMGT/V-QUEST (Brochet et al. (2008) Nucl Acids Res 36: W503-508). The results show broad diversity in genetic origin of the 43 unique scFv along with heavy chain CDR3 segments of varying length. Nearly all light chains were lambda light even though the initial unpanned library comprised equal representations of kappa and lambda comprising scFvs.

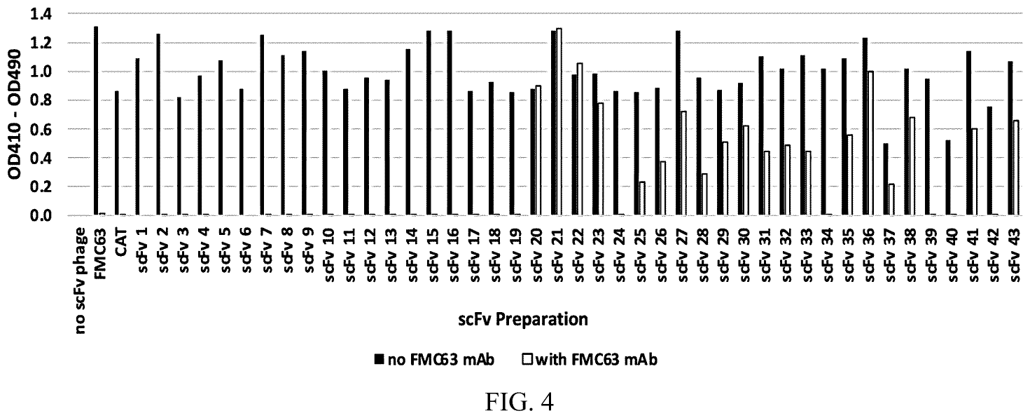

FIG. 4 shows ELISA results of an experiment designed to bin the 43 unique scFvs into those that might share the same (or related) epitope with FMC63. ELISA wells coated with streptavidin and biotinylated-AviTag™-CD19 were first incubated with high concentrations of FMC63 IgG (100-fold higher than saturating levels) and then dilute phage clones expressing the scFvs were added. HRP-conjugated antiphage antibody was used to assess phage-scFv binding. Controls were run without pre-addition/incubation with FMC63. FIG. 4 shows typical results for the clones that are blocked byFMC63 (e.g., scFv 1, 4, 7, etc.), those that do not appear to be blocked at all (e.g., scFv 20, 21, 22, etc.), and those that appear partially blocked (e.g., scFv 31, 33, etc.). For each scFv-phage tested, an ELISA OD value for the scFv-phage binding in the absence of FMC63 IgG was chosen to be approximately 1.0 in order to increase the sensitivity of binding in the presence of FMC63 IgG. That the binding ofFMC63 expressed on phage was blocked by FMC63 IgG serves as a positive control for the assay. The CD19 murine antibody CAT expressed on phage was also blocked by FMC63 IgG which is consistent with publications that FMC63 and CAT share the same CD19 epitope (Ghorashian et al. (2019) Nature Medicine 25:1408-1414).

TABLE 5
Binding and genetic characterizations for the 43 unique scFvs

| Unique | | ELISA hCD19 | μ or | κ or |
|---|---|---|---|---|
| scFv No. | scFv Clone Names | (ratio FMC63) | γ | λ |
| 1 | P3-15, P3-20, P3-32, P3-45, | 0.84 | γ | λ |
| | P3-49, P4-03, P4-10 | | | |
| 2 | P3-21, P3-22, P3-26, P3-50 | 0.49 | γ | λ |
| 3 | P3-05 | 0.32 | γ | λ |
| 4 | P3-06 | 0.77 | γ | λ |
| 5 | P3-14 | 0.48 | μ | λ |
| 6 | P3-28 | 0.47 | μ | λ |
| 7 | P3-47 | 0.83 | γ | λ |
| 8 | P3-16 | 0.41 | γ | λ |
| 9 | P3-41 | 0.71 | γ | λ |
| 10 | P3-01 | 0.77 | γ | λ |
| 11 | P3-57 | 0.31 | γ | λ |
| 12 | P3-48 | 0.16 | γ | λ |
| 13 | P3-36, P3-38 | 0.28 | γ | λ |
| 14 | P3-58 | 0.59 | μ | λ |
| 15 | P3-59A | 0.99 | γ | λ |
| 16 | P3-33, P4-01 | 0.41 | μ | λ |
| 17 | P4-05A, P4-05B | 0.66 | μ | λ |
| 18 | P4-12 | 0.27 | γ | λ |
| 19 | P3-35 | 0.26 | μ | λ |
| 20 | P3-12 | 0.47 | μ | λ |
| 21 | P3-04, P3-34, P4-07 | 0.71 | μ | λ |
| 22 | P3-09, P3-11, P3-23, | 0.21 | γ | λ |
| | P3-55, P4-04 | | | |
| 23 | P3-37 | 0.36 | μ | κ |
| 24 | P3-60 | 0.91 | μ | λ |
| 25 | P3-40 | 0.15 | μ | κ |
| 26 | P3-03 | 0.65 | γ | λ |
| 27 | P3-18 | 0.35 | γ | λ |
| 28 | P3-30 | 0.33 | γ | λ |
| 29 | P3-51 | 0.83 | γ | λ |
| 30 | P4-02 | 0.22 | γ | λ |
| 31 | P4-09 | 0.35 | γ | λ |
| 32 | P4-11 | 0.20 | γ | λ |
| 33 | P3-07 | 0.43 | γ | κ |
| 34 | P3-44 | 0.73 | μ | λ |
| 35 | P4-08 | 0.23 | μ | λ |
| 36 | P3-25 | 0.60 | γ | λ |
| 37 | P3-27 | 0.10 | γ | λ |
| 38 | P3-17 | 0.64 | γ | λ |
| 39 | P3-29 | 0.19 | γ | λ |
| 40 | P3-59B | 0.14 | γ | λ |
| 41 | P3-08A, P3-08B, P3-46 | 0.47 | μ | λ |
| 42 | P3-13 | 0.14 | μ | λ |
| 43 | P3-02, P3-19, P3-24, | 0.57 | γ | κ |
| | P3-42, P3-54, P3-56 | | | |

TABLE 6
Binding and genetic characterizations
for the 43 unique scFvs, continued

| | | | | | Canine Germline | | | |
|---|---|---|---|---|---|---|---|---|
| Unique | Canine Germline Heavy Chain Gene | | | | Light Chain Gene | | | KIR CAR-T |
| scFv | | | | HC- | | | LC- | Construct |
| No. | VH | D | JH | CDR3 | VL | JL | CDR3 | No. |
| 1 | Canlupfam | Canlupfam | Canlupfam | AGGNSRG | Canlupfam | Canlupfam | QSYDDS | TR.CAN.C |
| | IGHV3-8*01 | IGHD5*01 | IGHJ4*01 | WTEFGMD | IGLV1- | IGLJ5*01 | LDGRVV | D19.01.M |
| | | | | Y (SEQ ID | 103*01 | | (SEQ ID | |
| | | | | NO: 89) | | | NO: 91) | |
| 2 | Canlupfam | Canlupfam | Canlupfam | ARGMGSW | Canlupfam | Canlupfam | SSWDDD | TR.CAN.C |
| | IGHV3-41*01 | IGHD5*01 | IGHJ4*01 | GASTFGY | IGLV1- | IGLJ2*01 | LGSHV | D19.02.M |
| | | | | (SEQ ID NO: | 48*01 | | (SEQ ID | |
| | | | | 94) | | | NO: 96) | |
| 3 | Canlupfam | Canlupfam | Canlupfam | VKETFMIPT | Canlupfam | Canlupfam | QSYDDN | TR.CAN.C |
| | IGHV3-69*01 | IGHD4*01 | IGHJ4*01 | E (SEQ ID | IGLV1- | IGLJ4*01 | FDGHV | D19.03.M |
| | | | | NO: 99) | 103*01 | | (SEQ ID | |
| | | | | | | | NO: 101) | |
| 4 | Canlupfam | Canlupfam | Canlupfam | NSANWYV | Canlupfam | Canlupfam | QSYDDN | TR.CAN.C |
| | IGHV3-38*01 | IGHD4*01 | IGHJ4*01 | SPFDH | IGLV1- | IGLJ4*01 | FDYV | D19.04.M |
| | | | | (SEQ ID | 103*01 | | (SEQ ID | |
| | | | | NO: 104) | | | NO: 105) | |
| 5 | Canlupfam | Canlupfam | Canlupfam | GSHSWGNF | Canlupfam | Canlupfam | QSFDST | TR.CAN.C |
| | IGHV3-19*01 | IGHD5*01 | IGHJ4*01 | HY (SEQ ID | IGLV1- | IGLJ5*01 | HNAIV | D19.05.M |
| | | | | NO: 108) | 162*01 | | (SEQ ID | |
| | | | | | | | NO: 110) | |
| 6 | Canlupfam | Canlupfam | Canlupfam | ARRSGIWG | Canlupfam | Canlupfam | QSYDDS | TR.CAN.C |
| | IGHV3-2*01 | IGHD5*01 | IGHJ4*01 | FDY (SEQ | IGLV1- | IGLJ2*01 | LDGHAV | D19.06.M |
| | | | | ID NO: 113) | 103*01 | | (SEQ ID | |
| | | | | | | | NO: 115) | |
| 7 | Canlupfam | Canlupfam | Canlupfam | ARVDYSSS | Canlupfam | Canlupfam | QSYDDN | TR.CAN.C |
| | IGHV3-19*01 | IGHD4*01 | IGHJ4*01 | WLVDY | IGLV1- | IGLJ4*01 | FDGHHV | D19.07.M |
| | | | | (SEQ ID | 103*01 | | (SEQ ID | |
| | | | | NO: 118) | | | NO: 120) | |
| 8 | Canlupfam | Canlupfam | Canlupfam | SQWGDWG | Canlupfam | Canlupfam | SSWDDS | TR.CAN.C |
| | IGHV3-69*01 | IGHD5*01 | IGHJ4*01 | HFEY | IGLV1- | IGLJ4*01 | LRGTV | D19.08.M |
| | | | | (SEQ ID | 48*01 | | (SEQ ID | |
| | | | | NO: 123) | | | NO: 125) | |
| 9 | Canlupfam | Canlupfam | Canlupfam | AKDSGFGS | Canlupfam | Canlupfam | QSFDDN | TR.CAN.C |
| | IGHV3-69*01 | IGHD4*01 | IGHJ4*01 | WYLES | IGLV1- | IGLJ4*01 | VDGYV | D19.09.M |
| | | | | (SEQ ID | 103*01 | | (SEQ ID | |
| | | | | NO: 127) | | | NO: 129) | |
| 10 | Canlupfam | Canlupfam | Canlupfam | ARSSWGSF | Canlupfam | Canlupfam | QSFDDD | TR.CAN.C |
| | IGHV3-2*01 | IGHD5*01 | IGHJ4*01 | DY | IGLV1- | IGLJ3*01 | LDGPV | D19.10.M |
| | | | | (SEQ ID | 103*01 | | (SEQ ID | |
| | | | | NO: 132) | | | NO: 133) | |
| 11 | Canlupfam | Canlupfam | Canlupfam | HDYWFDF | Canlupfam | Canlupfam | QSYDDN | TR.CAN.C |
| | IGHV3-38*01 | IGHD1*01 | IGHJ2*01 | (SEQ ID | IGLV1- | IGLJ4*01 | FDYV | D19.11.M |
| | | | | NO: 136) | 103*01 | | (SEQ ID | |
| | | | | | | | NO: 105) | |
| 12 | Canlupfam | Canlupfam | Canlupfam | AKDSGSWS | Canlupfam | Canlupfam | QIWDRN | TR.CAN.C |
| | IGHV3-41*01 | IGHD5*01 | IGHJ6*01 | HFYGLDT | IGLV3- | IGLJ4*01 | INV (SEQ | D19.12.M |
| | | | | (SEQ ID | 11*01 | | ID NO: | |
| | | | | NO: 139) | | | 141) | |
| 13 | Canlupfam | Canlupfam | Canlupfam | VREDYFDF | Canlupfam | Canlupfam | QSYDDN | TR.CAN.C |
| | IGHV3-67*01 | IGHD1*01 | IGHJ4*01 | DY | IGLV1- | IGLJ4*01 | FDYV | D19.13.M |
| | | | | (SEQ ID | 103*01 | | (SEQ ID | |
| | | | | NO: 144) | | | NO: 105) | |
| 14 | Canlupfam | Canlupfam | Canlupfam | ADHPWYG | Canlupfam | Canlupfam | QSYDDN | TR.CAN.C |
| | IGHV3-5*01 | IGHD1*01 | IGHJ4*01 | PEY | IGLV1- | IGLJ4*01 | LNDPYV | D19.14.M |
| | | | | (SEQ ID | 103*01 | | (SEQ ID | |
| | | | | NO: 147) | | | NO: 149) | |
| 15 | Canlupfam | Canlupfam | Canlupfam | ARDQSSWG | Canlupfam | Canlupfam | QSYDDD | TR.CAN.C |
| | IGHV3-5*01 | IGHD5*01 | IGHJ4*01 | TGNFDY | IGLV1- | IGLJ5*01 | LDGHIV | D19.15.M |
| | | | | (SEQ ID | 103*01 | | (SEQ ID | |
| | | | | NO: 152) | | | NO: 154) | |
| 16 | Canlupfam | Canlupfam | Canlupfam | ATEYYWNI | Canlupfam | Canlupfam | SSYDSTL | TR.CAN.C |
| | IGHV3-26*01 | IGHD2*01 | IGHJ4*01 | DN | IGLV1- | IGLJ4*01 | SGYV | D19.16.M |
| | | | | (SEQ ID | 84*01 | | (SEQ ID | |
| | | | | NO: 157) | | | NO: 159) | |
| 17 | Canlupfam | Canlupfam | Canlupfam | SNYYGIYW | Canlupfam | Canlupfam | QSFDDN | TR.CAN.C |
| | IGHV3-5*01 | IGHD2*01 | IGHJ4*01 | DSDY | IGLVI- | IGLJ4*01 | VDGYV | D19.17.M |
| | | | | (SEQ ID | 103*01 | | (SEQ ID | |
| | | | | NO: 161) | | | NO: 162) | |
| 18 | Canlupfam | Canlupfam | Canlupfam | SQWGDWG | Canlupfam | Canlupfam | STWDSS | TR.CAN.C |
| | IGHV3-38*01 | IGHD5*01 | IGHJ4*01 | HFEY | IGLV1- | IGLJ4*01 | LNAYV | D19.18.M |
| | | | | (SEQ ID | 48*01 | | (SEQ ID | |
| | | | | NO: 123) | | | NO: 165) | |
| 19 | Canlupfam | Canlupfam | Canlupfam | GSHSWGNF | Canlupfam | Canlupfam | QSVDAT | TR.CAN.C |
| | IGHV3-19*01 | IGHD5*01 | IGHJ4*01 | HY | IGLV1- | IGLJ5*01 | HGTIL | D19.19.M |
| | | | | (SEQ ID | 141*01 | | (SEQ ID | |
| | | | | NO: 108) | | | NO: 167) | |
| 20 | Canlupfam | unassigned | Canlupfam | GRYYYGLD | Canlupfam | Canlupfam | ESEVSPD | TR.CAN.C |
| | IGHV3-69*01 | | IGHJ6*01 | Y | IGLV3- | IGLJ5*01 | TIV | D19.20.M |
| | | | | (SEQ ID | 27*01 | | (SEQ | |
| | | | | NO: 170) | | | ID NO: | |
| | | | | | | | 172) | |
| 21 | Canlupfam | Canlupfam | Canlupfam | AKPDIYGSF | Canlupfam | Canlupfam | ESAVSS | TR.CAN.C |
| | IGHV3-19*01 | IGHD3*01 | IGHJ4*01 | DH | IGLV3- | IGLJ4*01 | DTAV | D19.21.M |
| | | | | (SEQ ID | 27*01 | | (SEQ ID | |
| | | | | NO: 175) | | | NO: 176) | |
| 22 | Canlupfam | Canlupfam | Canlupfam | ARYNGYV | Canlupfam | Canlupfam | SSYDTSL | TR.CAN.C |
| | IGHV4-1*01 | IGHD3*01 | IGHJ2*01 | DYLDV | IGLV1- | IGLJ2*01 | NGFV | D19.22.M |
| | | | | (SEQ ID | 138*01 | | (SEQ ID | |
| | | | | NO: 179) | | | NO: 181) | |
| 23 | Canlupfam | Canlupfam | Canlupfam | AHWGSGN | Canlupfam | Canlupfam | GQGKQD | |
| | IGHV4-1*01 | IGHD5*01 | IGHJ4*01 | LDY | IGKV2- | IGKJ5*01 | PIT (SEQ | |
| | | | | (SEQ ID | 11*01 | | ID NO: | |
| | | | | NO: 184) | | | 186) | |
| 24 | Canlupfam | Canlupfam | Canlupfam | AKNEYLAP | Canlupfam | Canlupfam | QSYDDN | |
| | IGHV3-67*01 | IGHD1*01 | IGHJ3*01 | TY | IGLV1- | IGLJ4*01 | VDGYV | |
| | | | | (SEQ ID | 103*01 | | (SEQ ID | |
| | | | | NO: 189) | | | NO: 190) | |
| 25 | Canlupfam | Canlupfam | Canlupfam | AKSRYTDT | Canlupfam | Canlupfam | GQGIQY | |
| | IGHV3-2*01 | IGHD3*01 | IGHJ4*01 | YPFDY | IGKV2- | IGKJ1*01 | PPT (SEQ | |
| | | | | (SEQ ID | 7*01 | | ID NO: | |
| | | | | NO: 193) | | | 194) | |
| 26 | Canlupfam | unassigned | Canlupfam | ARGLGMD | Canlupfam | Canlupfam | ESLVDS | |
| | IGHV3-26*01 | | IGHJ6*01 | Y | IGLV3- | IGLJ4*01 | DAYM | |
| | | | | (SEQ ID | 27*01 | | (SEQ ID | |
| | | | | NO: 197) | | | NO: 198) | |
| 27 | Canlupfam | unassigned | Canlupfam | ARGLGMD | Canlupfam | Canlupfam | HSYDEN | |
| | IGHV3-26*01 | | IGHJ6*01 | Y | IGLV3- | IGLJ4*01 | LDGYV | |
| | | | | (SEQ ID | 27*01 | | (SEQ ID | |
| | | | | NO: 197) | | | NO: 199) | |
| 28 | Canlupfam | unassigned | Canlupfam | ARGLGMD | Canlupfam | Canlupfam | ESLVND | |
| | IGHV3-26*01 | | IGHJ6*01 | Y | IGLV3- | IGLJ4*01 | NTYV | |
| | | | | (SEQ ID | 27*01 | | (SEQ ID | |
| | | | | NO: 197) | | | NO: 201) | |
| 29 | Canlupfam | unassigned | Canlupfam | ARGLGMD | Canlupfam | Canlupfam | ESVDST | |
| | IGHV3-26*01 | | IGHJ6*01 | Y | IGLV3- | IGLJ4*01 | DTYV | |
| | | | | (SEQ ID | 27*01 | | (SEQ ID | |
| | | | | NO: 197) | | | NO: 202) | |
| 30 | Canlupfam | unassigned | Canlupfam | ARGLGMD | Canlupfam | Canlupfam | QSYDDN | |
| | IGHV3-26*01 | | IGHJ6*01 | Y | IGLV3- | IGLJ4*01 | VDGYV | |
| | | | | (SEQ ID | 27*01 | | (SEQ ID | |
| | | | | NO: 197) | | | NO: 205) | |
| 31 | Canlupfam | unassigned | Canlupfam | ARGLGMD | Canlupfam | Canlupfam | ESMDGR | |
| | IGHV3-26*01 | | IGHJ6*01 | Y | IGLV3- | IGLJ4*01 | EAYV | |
| | | | | (SEQ ID | 27*01 | | (SEQ ID | |
| | | | | NO: 197) | | | NO: 204) | |
| 32 | Canlupfam | unassigned | Canlupfam | ARGLGMD | Canlupfam | Canlupfam | QSYDDN | |
| | IGHV3-26*01 | | IGHJ6*01 | Y | IGLV3- | IGLJ4*01 | LDGYV | |
| | | | | (SEQ ID | 27*01 | | (SEQ ID | |
| | | | | NO: 197) | | | NO: 205) | |
| 33 | Canlupfam | Canlupfam | Canlupfam | ARGLVY | Canlupfam | Canlupfam | GQGTHL | |
| | IGHV3-41*01 | IGHD4*01 | IGHJ4*01 | (SEQ ID | IGKV2- | IGKJ3*01 | PFT (SEQ | |
| | | | | NO: 208) | 4*02 | | ID NO: | |
| | | | | | | | 210) | |
| 34 | Canlupfam | Canlupfam | Canlupfam | ARRSGIWG | Canlupfam | Canlupfam | QSYDDS | |
| | IGHV3-2*01 | IGHD5*01 | IGHJ4*01 | FDY | IGLV1- | IGLJ2*01 | LDGHAV | |
| | | | | (SEQ ID | 103*01 | | (SEQ ID | |
| | | | | NO: 113) | | | NO: 115) | |
| 35 | Canlupfam | Canlupfam | Canlupfam | ARWWDFE | Canlupfam | Canlupfam | ESAVNS | |
| | IGHV3-19*01 | IGHD5*01 | IGHJ4*01 | Y | IGLV3- | IGLJ5*01 | DGIV | |
| | | | | (SEQ ID | 27*01 | | (SEQ ID | |
| | | | | NO: 212) | | | NO: 214) | |
| 36 | Canlupfam | Canlupfam | Canlupfam | KDLGVWG | Canlupfam | Canlupfam | QSFDTTL | |
| | IGHV3-35*01 | IGHD5*01 | IGHJ6*01 | MDN | IGLV1- | IGLJ4*01 | DAYV | |
| | | | | (SEQ ID | 162*01 | | (SEQ ID | |
| | | | | NO: 216) | | | NO: 217) | |
| 37 | Canlupfam | unassigned | Canlupfam | TTTALGY | Canlupfam | Canlupfam | ESEVSA | |
| | IGHV3-19*01 | | IGHJ3*01 | (SEQ ID | IGLV3- | IGLJ6*01 | GGAV | |
| | | | | NO: 220) | 27*01 | | (SEQ ID | |
| | | | | | | | NO: 222) | |
| 38 | Canlupfam | Canlupfam | Canlupfam | VAGGFLDY | Canlupfam | Canlupfam | ASADSS | |
| | IGHV3-41*01 | IGHD5*01 | IGHJ4*01 | (SEQ ID | IGLV3- | IGLJ3*01 | AIPV | |
| | | | | NO: 225) | 27*01 | | (SEQ ID | |
| | | | | | | | NO: 226) | |
| 39 | Canlupfam | Canlupfam | Canlupfam | VASPLYGT | Canlupfam | Canlupfam | QSFDTTL | |
| | IGHV3-41*01 | IGHD1*01 | IGHJ6*01 | YGFDY | IGLV1- | IGLJ4*01 | DAHV | |
| | | | | (SEQ ID | 162*01 | | (SEQ ID | |
| | | | | NO: 228) | | | NO: 230) | |
| 40 | Canlupfam | Canlupfam | Canlupfam | VASPLYGT | Canlupfam | Canlupfam | QSFDTTL | |
| | IGHV3-47*01 | IGHD1*01 | IGHJ6*01 | YGFDY | IGLV1- | IGLJ4*01 | DAYV | |
| | | | | (SEQ ID | 162*01 | | (SEQ ID | |
| | | | | NO: 228) | | | NO: 217) | |
| 41 | Canlupfam | Canlupfam | Canlupfam | VGGGYMD | Canlupfam | Canlupfam | ESAVSTE | |
| | IGHV3-19*01 | IGHD5*01 | IGHJ6*01 | F (SEQ ID | IGLV3- | IGLJ5*01 | TVV | |
| | | | | NO: 238) | 27*01 | | (SEQ ID | |
| | | | | | | | NO: 239) | |
| 42 | Canlupfam | Canlupfam | Canlupfam | VKGGWLD | Canlupfam | Canlupfam | QSVDPA | |
| | IGHV3-18*01 | IGHD5*01 | IGHJ4*01 | F (SEQ ID | IGLV1- | IGLJ4*01 | LHTYV | |
| | | | | NO: 241) | 141*01 | | (SEQ ID | |
| | | | | | | | NO: 243) | |
| 43 | Canlupfam | Canlupfam | Canlupfam | VVSNMNL | Canlupfam | Canlupfam | QQGLHF | |
| | IGHV3-41*01 | IGHD3*01 | IGHJ6*01 | (SEQ ID | IGKV2- | IGKJ1*01 | PPT (SEQ | |
| | | | | NO: 245) | 16*01 | | ID NO: | |
| | | | | | | | 247) | |

### Example 2: In Vitro and In Vivo Functional Characterization of CD19-CARs

The studies presented elsewhere herein identified a number of human CD19-specific scFvs and confirmed their binding specificity. Twenty of these scFvs were selected to be modified into CD19-specific chimeric antigen receptor (CAR) constructs which can be expressed in immune effector cells (e.g., T cells) in order to provide antigen-specific immunotherapy. The CAR constructs created in these studies comprised NK cell-derived KIR signaling domains.

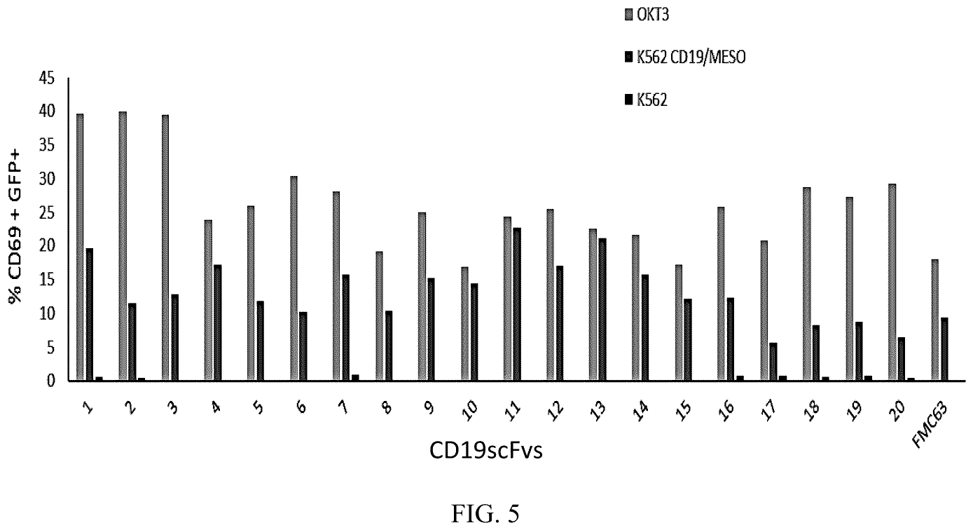

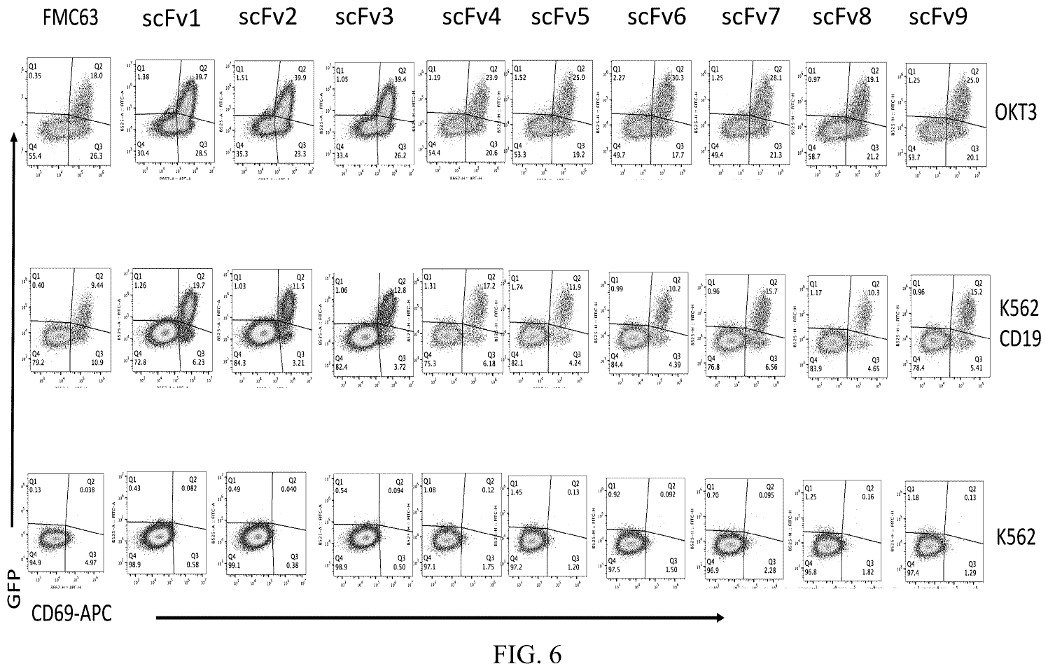

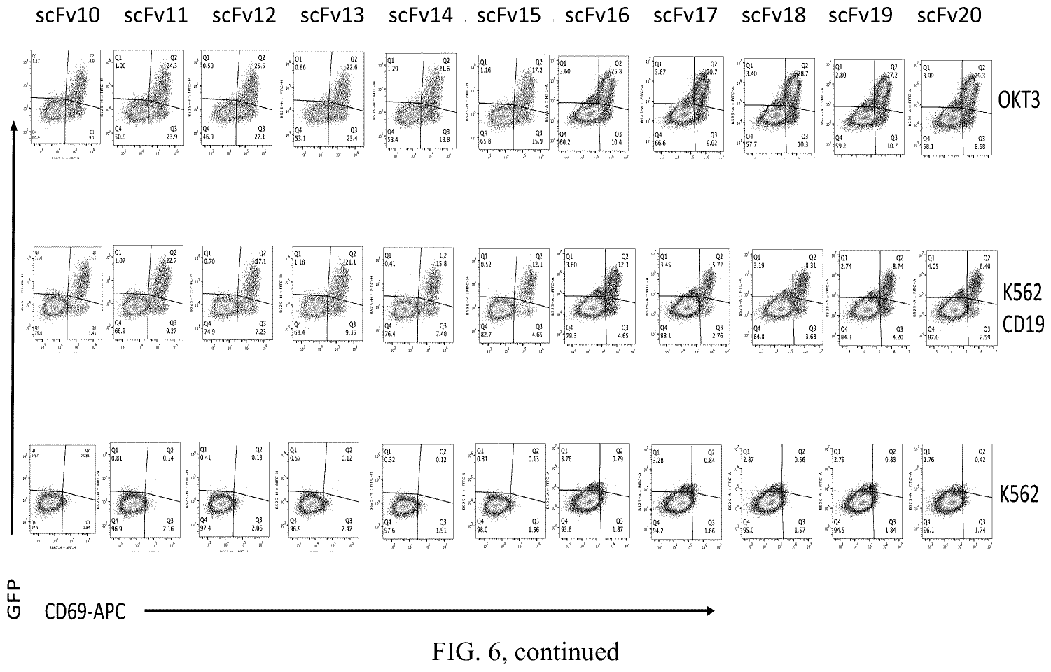

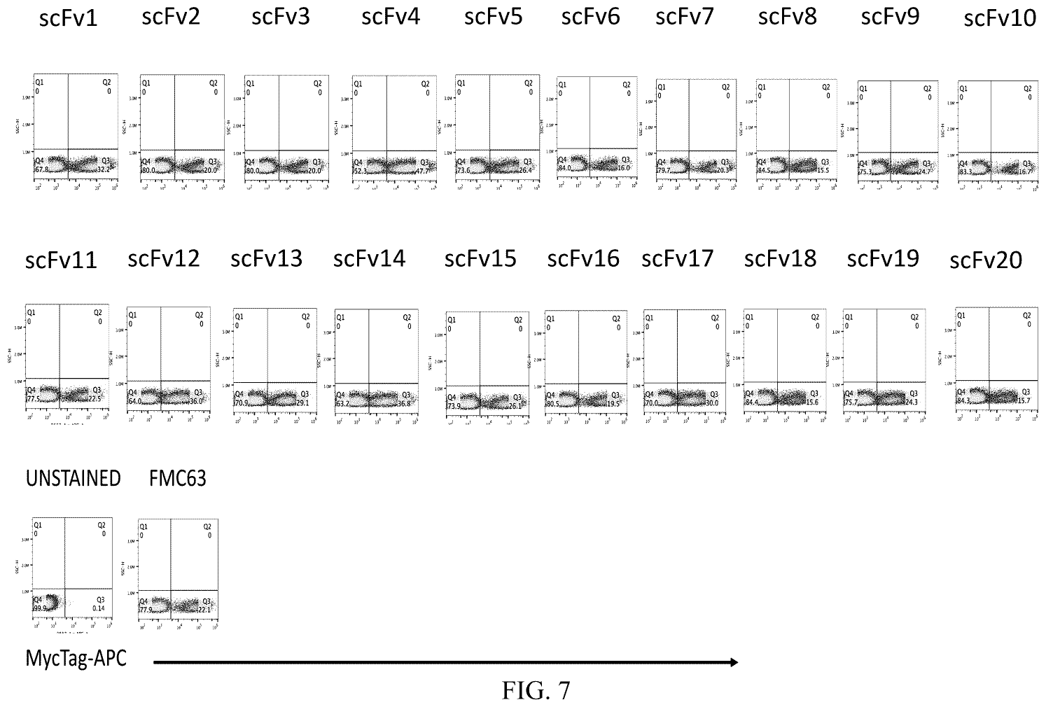

As an initial study, the newly derived CD19 scFv-CARs were transduced into Jurkat-NFAT-GFP (JNG) cells. These cells possess GFP marker genes operably linked to the NFAT promoter, such that activation of the CAR signaling domain drives expression of the GFP gene. Transfected cells were then incubated with CD19-expressing K562 cells. Non-CD19-expressing K562 cells were used as negative controls and T cells stimulated with anti-CD3 antibody (OKT3) were used as a positive control. Twenty-four hours after cells were harvested and stained for the activation marker CD69 and GFP expression (FIG. 6). FIG. 5 is a graph of the results of this study. Expression of T cell activation status was also verified by flow cytometry following staining with an antibody specific for CD69, and co-expression of CD69 and GFP for each CAR construct possesses. The transduction efficiency of each CAR transduced JNG cells was measured by flow cytometry followed by staining with anti-myc antibody (FIG. 7).

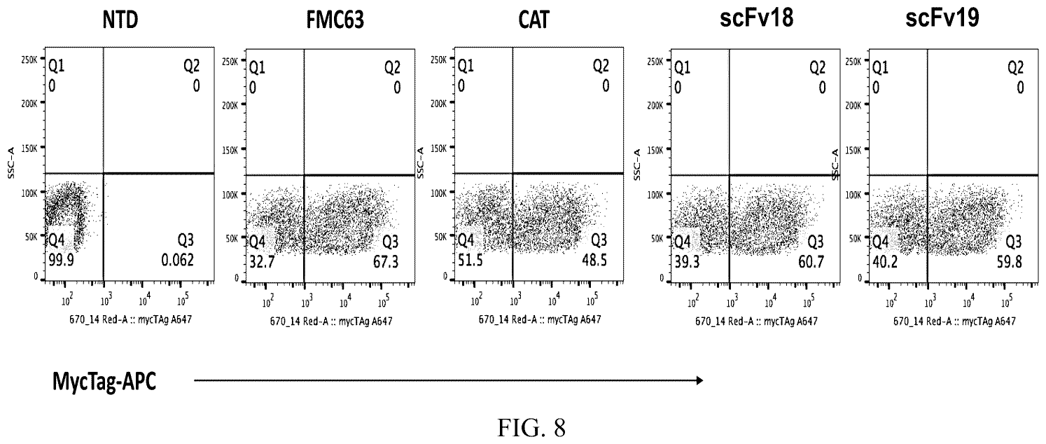

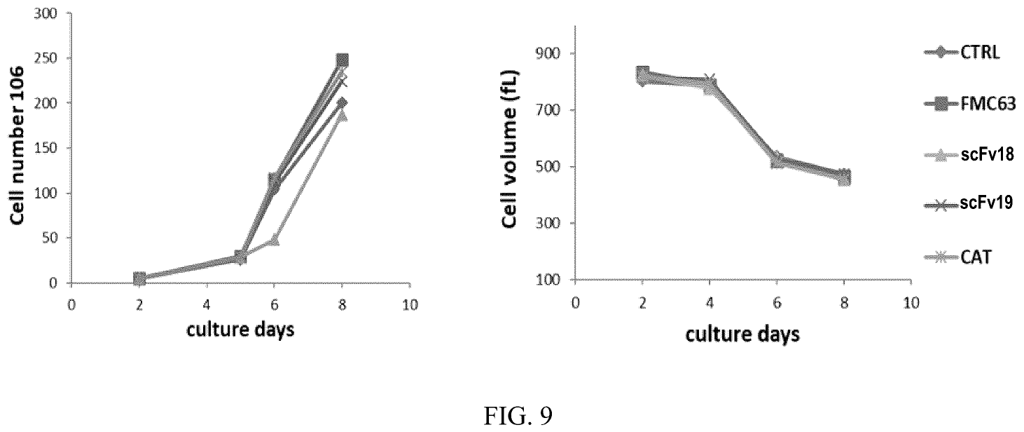

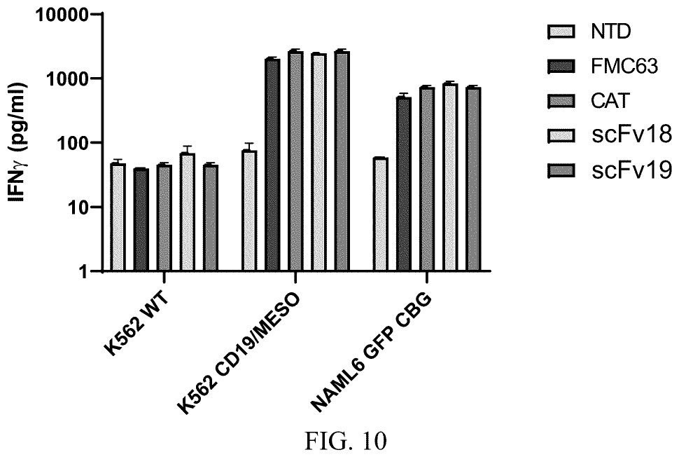

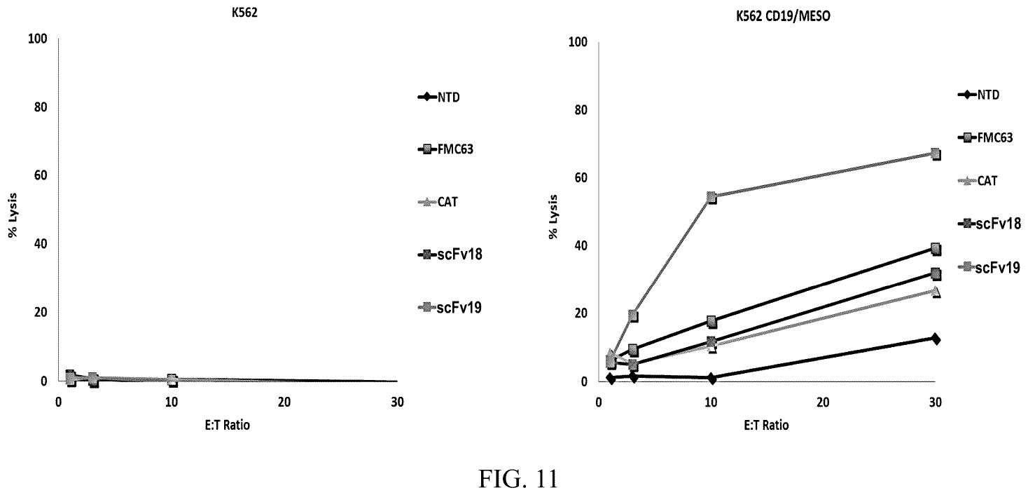

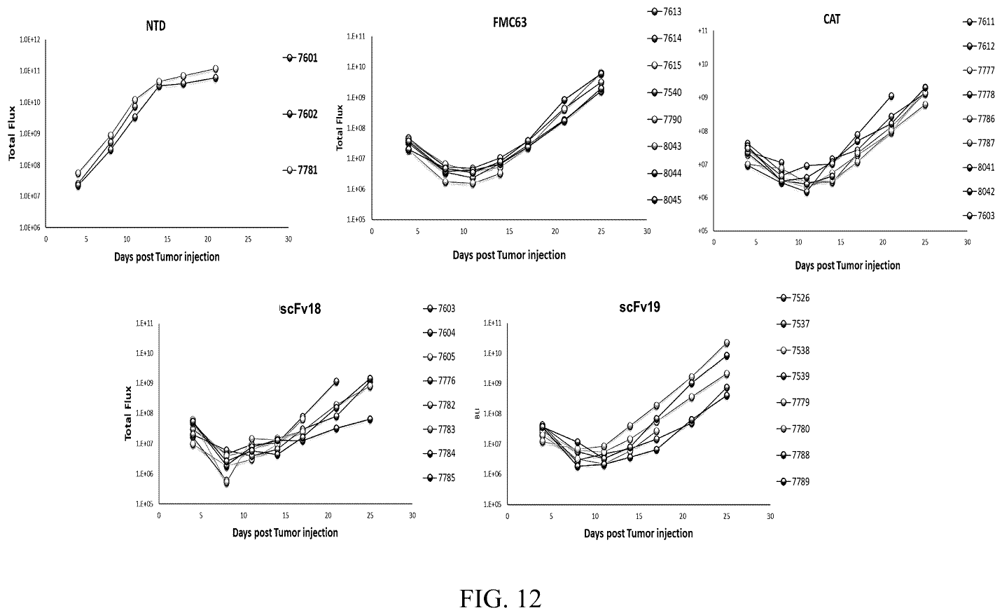

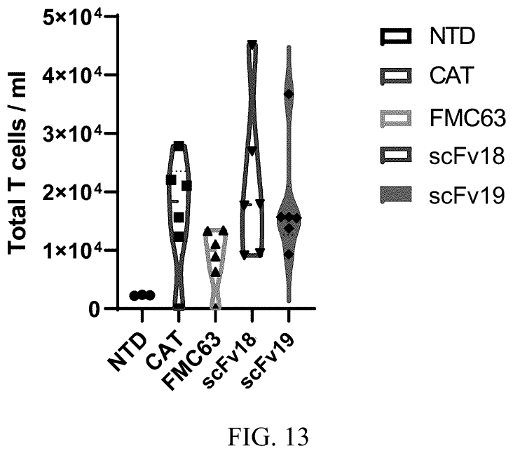

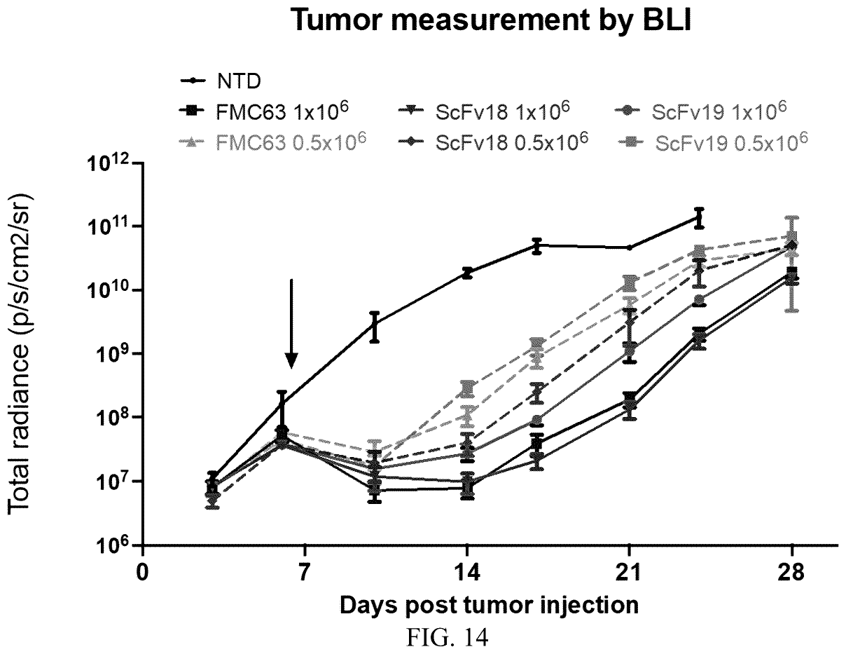

As a result of these studies, two scFvs were selected for further in vivo studies: scFv 18 and scFv 19. Expression of scFv 18 and scFv 19 CARs in human donor T cells was first confirmed (FIG. 9), as well as the ability of CAR-expressing T cells to proliferate and expand similar to control and FMC63 CAR expressing T cells following anti-CD3/anti-CD28 bead-stimulation (FIG. 9). The transduction efficiency was measured in human T cells (FIG. 8). The ability of scFv 18 and scFv 19 CAR expressing T cells to generate IFNy upon co-culture with target cells was then assessed and found to be similar to other positive control CAR constructs FMC63 and CAT (FIG. 10). Further in vitro cytotoxicity studies confirmed the ability of scFv 18 and scFv 19 CAR expressing T cells to kill CD19-expressing target cells (FIG. 11). A mouse xenograft tumor model was then used to verify in vivo activity of CAR-expressing T cells. Mice were first implanted with NALM6 tumor cells before receiving adoptive transfers of CD19 CAR T cells. Both scFv 18 and scFv 19 CAR T cells demonstrated a temporary regression in tumor growth, indicating in vivo tumor recognition (FIG. 12). To confirm the survival and engraftment of transferred CAR T cells, peripheral blood of tumor bearing animals was assessed for the number of human T cells per unit volume. Both scFv 18 and scFv 19 CAR T cells expanded to engraft into recipient animals (FIG. 13). A follow-up to the study presented in FIG. 12 was then conducted in order to observe the effect of different doses of CAR T cells on tumor growth. Here, immunocompromised NOD-scid IL2Rγc^{-/-} (NSG) mice were injected intravenously with 2×10⁶ luciferase-expressing NALM6-CBG (CD19-positive) tumor cells. Five days later, animals were randomized, and then injected intravenously via the tail vein with a low dose (0.5×10⁶) or high dose (1×10⁶) of scFv 18 CAR-T cells (scFv 18, n=8 for low dose, n=8 for high dose), or Clone 19 CAR-T cells (scFv19, n=8 for low dose 0.5×10⁶, n=8 for high dose 1×10⁶), or positive control FMC63 CAR-T cells (FMC63, n=8 for low dose 0.5×10⁶, n=8 for high dose 1×10⁶). For a negative control, one group mice received non-transduced (NTD) T cells (n=2, high dose 1×106). NALM6 tumor growth was then followed over the next 23 days via bioluminescence imaging (BLI) using a Xenogen IVIS Imaging System. Results from this study demonstrated a clear dose-dependent anti-tumor effect for each scFv with a significantly greater delay in tumor growth in animals receiving the high dose vs those that received the low dose or NTD control T cells (FIG. 14).

### Enumerated Embodiments

The following enumerated embodiments are provided, the numbering of which is not to be construed as designating levels of importance.

Embodiment 1 provides an antibody or antigen-binding fragment thereof that specifically binds to human CD19, wherein the antibody or antigen-binding fragment thereof comprises:
(a) a heavy chain complementarity determining region 1 (HCDR1), comprising the amino acid sequence set forth in SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, or 244;
(b) a heavy chain complementarity determining region 2 (HCDR2), comprising the amino acid sequence set forth in SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, or 237;
(c) a heavy chain complementarity determining region 3 (HCDR3), comprising the amino acid sequence set forth in SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, or 245;
(d) a light chain complementarity determining region 1 (LCDR1), comprising the amino acid sequence set forth in SEQ ID NOs: 90, 95, 100, 109, 114, 119, 124, 128, 140, 148, 153, 158, 166, 171, 180, 185, 200, 203, 209, 213, 221, 229, 234, 242, or 246;
(e) a light chain complementarity determining region 2 (LCDR2), comprising the amino acid sequence set forth in SEQ ID NOs: ADS, AND, ANI, EAS, GNS, GNT, GSH, GSS, GYN, KDT, LVS, NND, QVS, RDT, SDG, SNK, SSS, or SVS; or
(f) a light chain complementarity determining region 3 (LCDR3), comprising the amino acid sequence set forth in SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, or 247.

Embodiment 2 provides the antibody or antigen-binding fragment thereof of embodiment 1, wherein HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

Embodiment 3 provides the antibody or antigen-binding fragment thereof of embodiment 1, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to an amino acid sequence set forth in SEQ ID NOs: 314-356; and/or
(b) a light chain variable region comprising an amino acid sequence having at least 80%, 85%, 90%, 95%, 96%, 97%, 98%, 99% identity to an amino acid sequence set forth in SEQ ID NOs: 271-313.

Embodiment 4 provides the antibody or antigen-binding fragment thereof of embodiment 1, wherein the antibody or antigen-binding fragment thereof comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 5 provides an antibody or antigen-binding fragment thereof that specifically binds to human CD19, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to an amino acid sequence set forth in SEQ ID NOs: 314-356; and
(b) a light chain variable region comprising an amino acid sequence having at least 80%, 85%, 90%, 95%, 96%, 97%, 98%, 99% identity to the amino acid sequence set forth in SEQ ID NOs: 271-313.

Embodiment 6 provides the antibody or antigen-binding fragment thereof of embodiment 5, wherein the antibody or antigen-binding fragment thereof comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(1) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 7 provides an antibody or antigen-binding fragment thereof that specifically binds to CD19, wherein the antibody or antigen-binding fragment thereof comprises:
(a) a heavy chain variable region comprising an amino acid sequence set forth in SEQ ID NOs: 314-356; and
(b) a light chain variable region comprising an amino acid sequence set forth in SEQ ID NOs: 271-313.

Embodiment 8 provides the antibody or antigen-binding fragment thereof of any one of embodiments 1-7, wherein the antibody or antigen-binding fragment thereof is selected from the group consisting of a full-length antibody, a single-chain variable fragment (scFv), a sc(Fv)2, a dsFv, a Fab, a Fab′, a (Fab′)2, and a diabody.

Embodiment 9 provides the antibody or antigen-binding fragment thereof of any one of embodiments 1-8, wherein the antigen-binding fragment comprises an scFv.

Embodiment 10 provides a single-chain variable fragment (scFv) that specifically binds to human CD19, comprising:
(a) a heavy chain variable region that comprises an HCDR1, HCDR2, and HCDR3, wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises an amino acid sequence selected from the group consisting of SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises an, LCDR1, LCDR2, and LCDR3, wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises an amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247, wherein the heavy chain variable region and the light chain variable region are connected by a linker.

Embodiment 11 provides the scFv of embodiment 10, wherein HCDR1, HCDR2, HCDR3, LCDR1, and LCDR3 comprise the respective amino acid sequences set forth in: (a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

Embodiment 12 provides a single-chain variable fragment (scFv) specifically binds to CD19, comprising:
(a) a heavy chain variable region comprising an amino acid sequence set forth in SEQ ID NOs: 314-356; and
(b) a light chain variable region comprising an amino acid sequence set forth in SEQ ID NOs: 271-313, wherein the heavy chain variable region and the light chain variable region are connected by a linker.

Embodiment 13 provides the scFv of embodiment 12, wherein the scFv comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 14 provides a single chain variable fragment (scFv) specifically binds to CD19, comprising an amino acid sequence set forth in SEQ ID NOs: 44-86.

Embodiment 15 provides a chimeric antigen receptor (CAR) comprising the scFv of any one of embodiments 10-14.

Embodiment 16 provides a chimeric antigen receptor (CAR) comprising an antigen-binding domain specific for human CD19 (hCD19), a transmembrane domain, and an intracellular signaling domain, wherein the CAR comprises:
(a) a heavy chain variable region that comprises heavy chain complementarity determining region 1 (HCDR1), HCDR2, and HCDR3, wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises an amino acid sequence selected from the group consisting of SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises light chain complementarity determining region 1 (LCDR1), LCDR2, and LCDR3, wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises an amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

Embodiment 17 provides the CAR of embodiment 16, wherein the HCDR1, HCDR2, HCDR3, LCDR1, LCDR2, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

Embodiment 18 provides the CAR of embodiment 16, wherein the antigen-binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(1) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 19 provides the CAR of embodiment 16, wherein the antigen binding domain comprises a heavy chain variable region comprising an amino acid sequence having at least 95%-99% identity to an amino acid sequence of any of the heavy chain variable regions set forth in SEQ ID NOs: 314-356.

Embodiment 20 provides the CAR of embodiment 16, wherein the antigen binding domain comprises a heavy chain variable region comprising an amino acid sequence of any of the heavy chain variable regions set forth in SEQ ID NOs: 314-356.

Embodiment 21 provides the CAR of embodiment 16, wherein the antigen binding domain comprises a heavy chain variable region consisting of an amino acid sequence of any of the heavy chain variable regions set forth in SEQ ID NOs: 314-356.

Embodiment 22 provides the CAR of embodiment 16, wherein the antigen binding domain comprises a light chain variable region comprising an amino acid sequence having at least 95-99% identity to an amino acid sequence of any of the light chain variable regions set forth in SEQ ID NOs: 271-313.

Embodiment 23 provides the CAR of embodiment 16, wherein the antigen binding domain comprises a light chain variable region comprising an amino acid sequence of any of the light chain variable regions set forth in SEQ ID NOs: 271-313.

Embodiment 24 provides the CAR of embodiment 16, wherein the antigen binding domain consists of a light chain variable region consisting of an amino acid sequence of any of the light chain variable regions set forth in SEQ ID NOs: 271-313.

Embodiment 25 provides a chimeric antigen receptor (CAR) comprising an antigen binding domain, a transmembrane domain, and an intracellular domain, wherein the antigen binding domain comprises a heavy chain variable region comprising any of the amino acid sequences set forth in SEQ ID NO: 314-356; and a light chain variable region comprising any of the amino acid sequences set forth in SEQ ID NO: 271-313.

Embodiment 26 provides the CAR of embodiment 25, wherein the antigen-binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(1) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 27 provides a chimeric antigen receptor (CAR) comprising an antigen binding domain, a transmembrane domain, and an intracellular signaling domain, wherein the antigen binding domain comprises any one of the amino acid sequences set forth in SEQ ID NOs: 44-86.

Embodiment 28 provides the CAR of any one of embodiments 16-27, wherein the intracellular signaling domain is derived from a human NK cell receptor (NKR), and wherein the CAR is an NKR-CAR.

Embodiment 29 provides the CAR of embodiment 28, wherein the NKR domain is selected from the group consisting of an actKIR domain, an NCR domain, a SLAMF domain, an FcR domain, a CD16 domain, a CD64 domain, an actLy49 domain, and a an inhLy49 domain.

Embodiment 30 provides the CAR of embodiment 28, wherein the intracellular signaling domain is a KIR signaling domain selected from the group consisting of a KIR2DS2 signaling domain, a KIR2DS2 signaling domain, a KIR2DL3 signaling domain, a KIR2DL1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain, and a KIR3DP1 signaling domain.

Embodiment 31 provides the CAR of embodiment 28, wherein the transmembrane domain is a KIR transmembrane domain selected from the group consisting of a KIR2DS2 transmembrane domain, a KIR2DS2 transmembrane domain, a KIR2DL3 transmembrane domain, a KIR2DL1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL1 transmembrane domain, a KIR3DS1 transmembrane domain, a KIR3DL2 transmembrane domain, a KIR3DL3 transmembrane domain, a KIR2DP1 transmembrane domain, and a KIR3DP1 transmembrane domain.

Embodiment 32 provides the CAR of embodiment 28, wherein transmembrane domain can bind the transmembrane domain of DAP12

Embodiment 33 provides the CAR of any one of embodiments 28-32, wherein the KIR signaling domain comprises the amino acid sequence set forth in SEQ ID NO: 266.

Embodiment 34 provides the CAR of any one of embodiments 28-32, wherein the CAR comprises an amino acid sequence set forth in SEQ ID NOs: 370-412.

Embodiment 35 provides an NK cell receptor (NKR)-CAR complex, comprising an NKR-CAR comprising the amino acid sequence set forth in any one of SEQ ID NOs: 370-412 and an adaptor molecule.

Embodiment 36 provides the NKR-CAR complex of embodiment 29, wherein the adaptor molecule is DAP12 or FcεRγ.

Embodiment 37 provides the NKR-CAR complex of embodiment 29 or 30, wherein the NKR-CAR interacts with the adaptor molecule upon binding of the antigen binding domain of the NKR-CAR to human CD19.

Embodiment 38 provides an NKR-CAR comprising:
(a) an antigen-binding domain that binds specifically to human CD19 (hCD19), and one or both of;
(b) an NKR transmembrane domain; or
(c) an NKR intracellular signaling domain.

Embodiment 39 provides the NKR-CAR of embodiment 38, wherein the antigen binding domain comprises:
(a) an antigen binding domain comprising: a heavy chain variable region that comprises three heavy chain complementarity determining regions (HCDRs), wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

Embodiment 40 provides the NKR-CAR of embodiment 39, wherein HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

Embodiment 41 provides the NKR-CAR of embodiment 38, wherein the antigen binding domain comprises a heavy chain variable region comprising any of the amino acid sequences set forth in SEQ ID NO: 314-356; and a light chain variable region comprising any of the amino acid sequences set forth in SEQ ID NO: 271-313.

Embodiment 42 provides the NKR-CAR of embodiment 41, wherein the antigen-binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(1) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(11) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 43 provides the NKR-CAR of embodiment 38, wherein the NKR intracellular signaling domain is selected from the group consisting of an actKIR domain, an NCR domain, a SLAMF domain, an FcR domain, a CD16 domain, a CD64 domain, an actLy49 domain, and an inhLy49 domain.

Embodiment 44 provides the NKR-CAR of any one of embodiments 35-43, wherein the NKR-CAR is a KIR-CAR.

Embodiment 45 provides the NKR-CAR of embodiment 38, wherein the intracellular signaling domain is a KIR signaling domain selected from the group consisting of a KIR2DS2 signaling domain, a KIR2DS2 signaling domain, a KIR2DL3 signaling domain, a KIR2DL1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain, and a KIR3DP1 signaling domain.

Embodiment 46 provides the NKR-CAR of embodiment 38, wherein the transmembrane domain is a KIR transmembrane domain selected from the group consisting of a KIR2DS2 transmembrane domain, a KIR2DS2 transmembrane domain, a KIR2DL3 transmembrane domain, a KIR2DL1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL1 transmembrane domain, a KIR3DS1 transmembrane domain, a KIR3DL2 transmembrane domain, a KIR3DL3 transmembrane domain, a KIR2DP1 transmembrane domain, and a KIR3DP1 transmembrane domain.

Embodiment 47 provides the NKR-CAR of any one of embodiments 38-46, wherein transmembrane domain is capable of binding and/or activating DAP12 through the transmembrane domain of DAP12.

Embodiment 48 provides the NKR-CAR of any one of embodiments 38-46, wherein the KIR signaling domain comprises the amino acid sequence set forth in SEQ ID NO: 266.

Embodiment 49 provides the NKR-CAR of any one of embodiments 38-46, wherein the CAR comprising an amino acid sequence set forth in SEQ ID NOs: 370-412.

Embodiment 50 provides a chimeric antigen receptor (CAR) comprising:
(a) an antigen binding domain comprising: a heavy chain variable region that comprises three heavy chain complementarity determining regions (HCDRs), wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247;
(b) a transmembrane domain; and
(c) a KIR2DS2 intracellular signaling domain.

Embodiment 51 provides the CAR of embodiment 50, wherein HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

Embodiment 52 provides a chimeric antigen receptor (CAR) comprising:
(a) an antigen binding domain comprising: a heavy chain variable region comprising any of the amino acid sequences set forth in SEQ ID NOs: 314-356; and a light chain variable region comprising any of the amino acid sequences set forth in SEQ ID NOs: 271-313;
(b) a transmembrane domain; and
(c) a KIR2DS2 intracellular signaling domain.

Embodiment 53 provides the CAR of embodiment 52, wherein the antigen binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(1) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(o) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 54 provides a chimeric antigen receptor (CAR) comprising:
(a) the CD8 leader amino acid sequence set forth in SEQ ID NO: 357;
(b) an scFv amino acid sequence set forth in SEQ ID NOs: 44-86;
(c) the myc tag amino acid sequence set forth in SEQ ID NO: 264; and
(d) the transmembrane/KIR2DS2 signaling domain amino acid sequence set forth in SEQ ID NO: 266.

Embodiment 55 provides the CAR of any one of embodiments 50-54, wherein the CAR comprises an amino acid sequence set forth in SEQ ID NOs: 370-412.

Embodiment 56 provides an isolated nucleic acid encoding the antibody or antigen-binding fragment thereof of any one of embodiments 1-9 or the scFv of any one of embodiments 10-14.

Embodiment 57 provides an isolated nucleic acid encoding the CAR of any one of embodiments 15-32 and 50-55, or the NKR-CAR of any one of embodiments 33-46.

Embodiment 58 provides an isolated nucleic acid encoding an antibody or antigen-binding fragment thereof, wherein the nucleic acid antibody or antigen-binding fragment thereof comprises a nucleic acid having at least 80%, 85%, 90%, 95%, 96%, 96%, 97%, 98%, 99% identity to the polynucleotide sequence set forth in SEQ ID NOs: 1-43.

Embodiment 59 provides an isolated nucleic acid encoding an antibody or antigen-binding fragment thereof, wherein the nucleic acid antibody or antigen-binding fragment thereof comprises a polynucleotide sequence set forth in SEQ ID NOs: 1-43.

Embodiment 60 provides the isolated nucleic acid of embodiment 58 or 59, wherein the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

Embodiment 61 provides an isolated nucleic acid encoding a single-chain variable fragment (scFv) comprising a polynucleotide sequence set forth in SEQ ID NOs: 1-43.

Embodiment 62 provides the isolated nucleic acid of any one of embodiments 56-61, wherein the antibody or antigen-binding fragment thereof or the scFv or the antigen-binding domain of the CAR or NKR-CAR specifically binds to human CD19.

Embodiment 63 provides an isolated nucleic acid encoding a chimeric antigen receptor (CAR) comprising an antigen binding domain, a transmembrane domain, and an intracellular signaling domain, wherein the antigen binding domain comprises:
(a) a heavy chain variable region comprising a nucleic acid encoding any of the amino acid sequences set forth in SEQ ID NOs: 314-356; and
(b) a light chain variable region comprising a nucleic acid encoding any of the amino acid sequences set forth in SEQ ID NOs: 271-313.

Embodiment 64 provides the isolated nucleic acid of embodiment 63, wherein the antigen binding domain comprises:
(a) a heavy chain variable region comprising the amino acid sequence set forth in SEQ ID NO: 314 and a light chain variable region comprising the amino acid sequence set forth in SEQ ID NO: 271;
(b) a heavy chain variable region comprising the amino acid sequence set forth in SEQ ID NO: 315 and a light chain variable region comprising the amino acid sequence set forth in SEQ ID NO: 272;
(c) a heavy chain variable region comprising the amino acid sequence set forth in SEQ ID NO: 316 and a light chain variable region comprising the amino acid sequence set forth in SEQ ID NO: 273;
(d) a heavy chain variable region comprising the amino acid sequence set forth in SEQ ID NO: 317 and a light chain variable region comprising the amino acid sequence set forth in SEQ ID NO: 274;
(e) a heavy chain variable region comprising the amino acid sequence set forth in SEQ ID NO: 318 and a light chain variable region comprising the amino acid sequence set forth in SEQ ID NO: 275;
(f) a heavy chain variable region comprising the amino acid sequence set forth in SEQ ID NO: 319 and a light chain variable region comprising the amino acid sequence set forth in SEQ ID NO: 276;
(g) a heavy chain variable region comprising the amino acid sequence set forth in SEQ ID NO: 320 and a light chain variable region comprising the amino acid sequence set forth in SEQ ID NO: 277;
(h) a heavy chain variable region comprising the amino acid sequence set forth in SEQ ID NO: 321 and a light chain variable region comprising the amino acid sequence set forth in SEQ ID NO: 278;
(i) a heavy chain variable region comprising the amino acid sequence set forth in SEQ ID NO: 322 and a light chain variable region comprising the amino acid sequence set forth in SEQ ID NO: 279;
(j) a heavy chain variable region comprising the amino acid sequence set forth in SEQ ID NO: 323 and a light chain variable region comprising the amino acid sequence set forth in SEQ ID NO: 280;
(k) a heavy chain variable region comprising the amino acid sequence set forth in SEQ ID NO: 324 and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282;
(m) a heavy chain variable region comprising the amino acid sequence set forth in SEQ ID NO: 326 and a light chain variable region comprising the amino acid sequence set forth in SEQ ID NO: 283;
(n) a heavy chain variable region comprising the amino acid sequence set forth in SEQ ID NO: 327 and a light chain variable region comprising the amino acid sequence set forth in SEQ ID NO: 284;
(0) a heavy chain variable region comprising the amino acid sequence set forth in SEQ ID NO: 328 and a light chain variable region comprising the amino acid sequence set forth in SEQ ID NO: 285;
(p) a heavy chain variable region comprising the amino acid sequence set forth in SEQ ID NO: 329 and a light chain variable region comprising the amino acid sequence set forth in SEQ ID NO: 286;
(q) a heavy chain variable region comprising the amino acid sequence set forth in SEQ ID NO: 330 and a light chain variable region comprising the amino acid sequence set forth in SEQ ID NO: 287;
(r) a heavy chain variable region comprising the amino acid sequence set forth in SEQ ID NO: 331 and a light chain variable region comprising the amino acid sequence set forth in SEQ ID NO: 288;
(s) a heavy chain variable region comprising the amino acid sequence set forth in SEQ ID NO: 332 and a light chain variable region comprising the amino acid sequence set forth in SEQ ID NO: 289;
(t) a heavy chain variable region comprising the amino acid sequence set forth in SEQ ID NO: 333 and a light chain variable region comprising the amino acid sequence set forth in SEQ ID NO: 290;
(u) a heavy chain variable region comprising the amino acid sequence set forth in SEQ ID NO: 334 and a light chain variable region comprising the amino acid sequence set forth in SEQ ID NO: 291;
(v) a heavy chain variable region comprising the amino acid sequence set forth in SEQ ID NO: 335 and a light chain variable region comprising the amino acid sequence set forth in SEQ ID NO: 292;
(w) a heavy chain variable region comprising the amino acid sequence set forth in SEQ ID NO: 336 and a light chain variable region comprising the amino acid sequence set forth in SEQ ID NO: 293;
(x) a heavy chain variable region comprising the amino acid sequence set forth in SEQ ID NO: 337 and a light chain variable region comprising the amino acid sequence set forth in SEQ ID NO: 294;
(y) a heavy chain variable region comprising the amino acid sequence set forth in SEQ ID NO: 338 and a light chain variable region comprising the amino acid sequence set forth in SEQ ID NO: 295;
(z) a heavy chain variable region comprising the amino acid sequence set forth in SEQ ID NO: 339 and a light chain variable region comprising the amino acid sequence set forth in SEQ ID NO: 296;
(aa) a heavy chain variable region comprising the amino acid sequence set forth in SEQ ID NO: 340 and a light chain variable region comprising the amino acid sequence set forth in SEQ ID NO: 297;
(bb) a heavy chain variable region comprising the amino acid sequence set forth in SEQ ID NO: 341 and a light chain variable region comprising the amino acid sequence set forth in SEQ ID NO: 298;
(cc) a heavy chain variable region comprising the amino acid sequence set forth in SEQ ID NO: 342 and a light chain variable region comprising the amino acid sequence set forth in SEQ ID NO: 299;
(dd) a heavy chain variable region comprising the amino acid sequence set forth in SEQ ID NO: 343 and a light chain variable region comprising the amino acid sequence set forth in SEQ ID NO: 300;
(ee) a heavy chain variable region comprising the amino acid sequence set forth in SEQ ID NO: 344 and a light chain variable region comprising the amino acid sequence set forth in SEQ ID NO: 301;
(ff) a heavy chain variable region comprising the amino acid sequence set forth in SEQ ID NO: 345 and a light chain variable region comprising the amino acid sequence set forth in SEQ ID NO: 302;
(gg) a heavy chain variable region comprising the amino acid sequence set forth in SEQ ID NO: 346 and a light chain variable region comprising the amino acid sequence set forth in SEQ ID NO: 303;
(hh) a heavy chain variable region comprising the amino acid sequence set forth in SEQ ID NO: 347 and a light chain variable region comprising the amino acid sequence set forth in SEQ ID NO: 304;
(ii) a heavy chain variable region comprising the amino acid sequence set forth in SEQ ID NO: 348 and a light chain variable region comprising the amino acid sequence set forth in SEQ ID NO: 305;
(jj) a heavy chain variable region comprising the amino acid sequence set forth in SEQ ID NO: 349 and a light chain variable region comprising the amino acid sequence set forth in SEQ ID NO: 306;
(kk) a heavy chain variable region comprising the amino acid sequence set forth in SEQ ID NO: 350 and a light chain variable region comprising the amino acid sequence set forth in SEQ ID NO: 307;
(ll) a heavy chain variable region comprising the amino acid sequence set forth in SEQ ID NO: 351 and a light chain variable region comprising the amino acid sequence set forth in SEQ ID NO: 308;
(mm) a heavy chain variable region comprising the amino acid sequence set forth in SEQ ID NO: 352 and a light chain variable region comprising the amino acid sequence set forth in SEQ ID NO: 309;
(nn) a heavy chain variable region comprising the amino acid sequence set forth in SEQ ID NO: 353 and a light chain variable region comprising the amino acid sequence set forth in SEQ ID NO: 310;
(oo) a heavy chain variable region comprising the amino acid sequence set forth in SEQ ID NO: 354 and a light chain variable region comprising the amino acid sequence set forth in SEQ ID NO: 311;
(pp) a heavy chain variable region comprising the amino acid sequence set forth in SEQ ID NO: 355 and a light chain variable region comprising the amino acid sequence set forth in SEQ ID NO: 312; or
(qq) a heavy chain variable region comprising the amino acid sequence set forth in SEQ ID NO: 356 and a light chain variable region comprising the amino acid sequence set forth in SEQ ID NO: 313.

Embodiment 65 provides an isolated nucleic acid encoding a chimeric antigen receptor (CAR) comprising an antigen binding domain, a transmembrane domain, and an intracellular signaling domain, wherein the antigen binding domain is encoded by any one of the nucleotide polynucleotide sequences set forth in SEQ ID NOs: 1-43.

Embodiment 66 provides the isolated nucleic acid of any one of embodiments 63-65, wherein the intracellular signaling domain comprises an intracellular domain comprising one or more cytoplasmic signaling domains of a human NK cell KIR receptor.

Embodiment 67 provides the isolated nucleic acid of embodiment 66, wherein the KIR signaling domain is comprises a KIR2DS2 signaling domain.

Embodiment 68 provides the isolated nucleic acid of embodiment 67, wherein the KIR signaling domain is encoded by a polynucleotide sequence comprising the sequence set forth in SEQ ID NO: 265.

Embodiment 69 provides a vector comprising the isolated nucleic acid of any one of embodiments 56-68.

Embodiment 70 provides the vector of embodiment 69, wherein the vector is an expression vector.

Embodiment 71 provides the vector of embodiment 69 or 70, wherein the vector is selected from the group consisting of a DNA vector, an RNA vector, a plasmid, a lentiviral vector, an adenoviral vector, an adeno-associated viral vector, and a retroviral vector.

Embodiment 72 provides a host cell comprising the isolated nucleic acid of any one of embodiments 56-68 or the vector of any one of embodiments 69-71.

Embodiment 73 provides the host cell of embodiment 72, wherein the host cell is of eukaryotic or prokaryotic origin.

Embodiment 74 provides the host cell of embodiment 72, wherein the host cell is of mammalian origin.

Embodiment 75 provides the host cell of embodiment 72, wherein the host cell is of bacterial origin.

Embodiment 76 provides the host cell of embodiment 70, wherein the host cell is a Chinese Hamster Ovary cell.

Embodiment 77 provides a modified immune cell or precursor cell thereof, comprising a chimeric antigen receptor (CAR) comprising an antigen binding domain, a transmembrane domain, and an intracellular domain, wherein the antigen binding domain comprises:
(a) a heavy chain variable region that comprises three heavy chain complementarity determining regions (HCDRs), wherein HCDR1 comprises an amino acid sequence selected from the group consisting of SEQ ID NOs: 87, 92, 97, 102, 106, 111, 116, 121, 126, 130, 134, 137, 142, 145, 150, 155, 163, 168, 173, 177, 182, 187, 191, 195, 206, 211, 218, 223, 231, 236, 240, and 244; HCDR2 comprises an amino acid sequence selected from the group consisting of SEQ ID NOs: 88, 93, 98, 103, 107, 112, 117, 122, 131, 135, 138, 143, 146, 151, 156, 160, 164, 169, 174, 178, 183, 188, 192, 196, 207, 215, 219, 224, 227, 232, and 237; and HCDR3 comprises and amino acid sequence selected from the group comprising SEQ ID NOs: 89, 94, 99, 104, 108, 113, 118, 123, 127, 132, 136, 139, 144, 147, 152, 157, 161, 170, 175, 179, 184, 189, 193, 197, 208, 212, 216, 220, 225, 228, 233, 238, 241, and 245; and
(b) a light chain variable region that comprises three light chain complementarity determining regions (LCDRs), wherein LCDR1 comprises an amino acid sequence selected from the group consisting of SEQ ID NOs: 90, 95, 100, 109, 114, 119, 124, 128, 140, 148, 153, 158, 166, 171, 180, 185, 200, 203, 209, 213, 221, 229, 234, 242, and 246; LCDR2 comprises and amino acid sequence selected from the group consisting of SEQ ID NOs: ADS, AND, ANI, EAS, GNS, GNT, GSH, GSS, GYN, KDT, LVS, NND, QVS, RDT, SDG, SNK, SSS, and SVS; and LCDR3 comprises an amino acid sequence selected from the group consisting of SEQ ID NOs: 91, 96, 101, 105, 110, 115, 120, 125, 129, 133, 141, 149, 154, 159, 162, 165, 167, 172, 176, 181, 186, 190, 194, 198, 199, 201, 202, 204, 205, 210, 214, 217, 222, 226, 230, 235, 239, 243, and 247.

Embodiment 78 provides the modified immune cell of embodiment 77, wherein HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff) SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

Embodiment 79 provides the modified immune cell of embodiment 77, wherein the CAR binds human CD19.

Embodiment 80 provides the modified immune cell of embodiment 77, wherein the CAR comprises an antigen binding domain selected from the group consisting of an antibody, an scFv, and a Fab.

Embodiment 81 provides the modified immune cell of embodiment 77, wherein the CAR comprises an intracellular domain comprising cytoplasmic signaling domains of a human NK cell KIR receptor.

Embodiment 82 provides the modified immune cell of embodiment 81, wherein the KIR signaling domain comprises a KIR2DS2 signaling domain.

Embodiment 83 provides the modified immune cell of embodiment 77, wherein the modified cell is an autologous cell.

Embodiment 84 provides the modified immune cell of embodiment 77, wherein the modified cell is an allogeneic cell.

Embodiment 85 provides the modified immune cell of embodiment 77, wherein the modified cell is a cell isolated from a human subject.

Embodiment 86 provides the modified immune cell of embodiment 77, wherein the modified cell is a modified T cell.

Embodiment 87 provides a pharmaceutical composition comprising the antibody or antigen-binding fragment thereof of any one of embodiments 1-9, the scFv of any one of embodiments 10-14, or the CAR of any one of embodiments 15-32 and 47-52, or the NKR-CAR of any one of embodiments 33-46, or the isolated nucleic acid of any one of embodiments 53-65, the vector of any one of embodiments 66-68, or the modified immune cell of any one of embodiments 74-83, and a pharmaceutically acceptable excipient, carrier, or diluent.

Embodiment 88 provides a method for generating a modified immune cell or precursor cell thereof, comprising introducing into the immune or precursor cell an isolated nucleic acid encoding the chimeric antigen receptor (CAR) of any one of embodiments 15-32 and 47-52 or the NKR-CAR of any one of embodiments 33-46.

Embodiment 89 provides the method of embodiment 88, wherein the modified immune cell is a T cell.

Embodiment 90 provides a method of treating cancer in a subject in need thereof, comprising administering to the subject an effective amount of the modified immune cell of embodiments 74-83 or a modified immune cell generated by the method any one of embodiments 85-86, thereby treating the cancer.

Embodiment 91 provides the method of embodiment 90, wherein the cancer is a hematologic cancer.

Embodiment 92 provides the method of embodiment 90, wherein the cancer is associated with the expression of CD19.

Embodiment 93 provides the method of embodiment 92, wherein the CD19 is expressed on tumor cells.

Embodiment 94 provides the method of embodiment 90, wherein the cancer is selected from the group consisting of Burkitt lymphoma, chronic lymphocytic leukemia (CLL), acute lymphoblastic leukemia (ALL), B-cell lymphoma, and B-cell leukemia.

Embodiment 95 provides the method of embodiment 90, wherein the subject is a human.

Embodiment 96 provides a method of treating an autoimmune disorder or disease in a subject in need thereof, comprising administering to the subject an effective amount of the modified immune cell of embodiments 74-83 or a modified immune cell generated by the method any one of embodiments 85-86, thereby treating the autoimmune disorder.

Embodiment 97 provides the method of embodiment 96, wherein the autoimmune disorder is antibody mediated.

Embodiment 98 provides the method of embodiment 96, wherein administration of the modified immune cell depletes autoimmune B cells.

Embodiment 99 provides the method of embodiment 96, wherein the autoimmune disorder is selected from the group consisting of rheumatoid arthritis, systemic lupus erythematosus (SLE), lupus nephritis (LN), idiopathic/autoimmune thrombocytopenia purpura (ITP), idiopathic thrombotic thrombocytopeniaurpura (TTP), pemphigus-related disorders, diabetes, and scleroderma, myasthenia gravis, multiple sclerosis, vasculitis, and autoimmune hemolytic anemia.

Embodiment 100 provides a method of treating an alloantibody-mediated disorder or disease in a subject in need thereof, comprising administering to the subject an effective amount of the modified immune cell of Embodiments 77-86 or a modified immune cell generated by the method any one of Embodiments 88-89, thereby treating the alloantibody-mediated disorder or disease.

Embodiment 101 provides the method of Embodiment 100, wherein the administration of the modified immune cell depletes B cells that produce alloantibodies.

Embodiment 102 provides the method of Embodiment 100, wherein the alloantibody-mediated disorder is selected from the group consisting of solid organ transplant immune incompatibility, resistance to enzyme replacement therapy or acute or delayed hemolytic reactions to transfusion, and chronic alloantibody mediated rejection (CAMR), organ transplant immune incompatibility hemophilia (hemophilia A or B), lysosomal storage diseases (LSD), urea cycle disorder, adenosine deaminase deficiency, neuronal ceroid lipofuscinoses (NCL), hyperammonemia, and chronic graft-versus-host disease (cGvHD).

Embodiment 103 provides the method of Embodiment 102, wherein the lysosomal storage disease is selected from the group consisting of glycogen storage disease, Gaucher's disease, Niemann-Pick disease, Fabry's disease, and mucopolysaccharidosis (MPS) I, MPS II, and MPS VI.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure can be devised by others skilled in the art without departing from the true spirit and scope of the present disclosure. The appended claims are intended to be construed to include all such embodiments and equivalent variations.

What is claimed is:

1. An antibody or antigen-binding fragment thereof that specifically binds to human CD19, comprising a heavy chain variable region that comprises heavy chain complementarity determining region 1 (HCDR1), HCDR2, and HCDR3 and a light chain variable region that comprises light chain complementarity determining region 1 (LCDR1), LCDR2, and LCDR3, wherein HCDR1, HCDR2, HCDR3, LCDR1, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

2. The antibody or antigen-binding fragment thereof of claim 1, wherein the antibody or antigen-binding fragment thereof comprises:
(a) a heavy chain variable region comprising the amino acid sequence set forth in SEQ ID NO: 314 or comprising an amino acid sequence having at least 80%, 85%, 90%, 95%, 96%, 96%, 97%, 98%, or 99% identity to the amino acid set forth in SEQ ID NO: 314; and a light chain variable region comprising the amino acid sequence set forth in SEQ ID NO: 271 or comprising an amino acid sequence having at least 80%, 85%, 90%, 95%, 96%, 96%, 97%, 98%, or 99% identity to the amino acid set forth in SEQ ID NO: 271;
(b) a heavy chain variable region comprising the amino acid sequence set forth in SEQ ID NO: 315 or comprising an amino acid sequence having at least 80%, 85%, 90%, 95%, 96%, 96%, 97%, 98%, or 99% identity to the amino acid set forth in SEQ ID NO: 315; and a light chain variable region comprising the amino acid sequence set forth in SEQ ID NO: 272 or comprising an amino acid sequence having at least 80%, 85%, 90%, 95%, 96%, 96%, 97%, 98%, or 99% identity to the amino acid set forth in SEQ ID NO: 272;
(c) a heavy chain variable region comprising the amino acid sequence set forth in SEQ ID NO: 316 or comprising an amino acid sequence having at least 80%, 85%, 90%, 95%, 96%, 96%, 97%, 98%, or 99% identity to the amino acid set forth in SEQ ID NO: 316; and a light chain variable region comprising the amino acid sequence set forth in SEQ ID NO: 273 or comprising an amino acid sequence having at least 80%, 85%, 90%, 95%, 96%, 96%, 97%, 98%, or 99% identity to the amino acid set forth in SEQ ID NO: 273;
(d) a heavy chain variable region comprising the amino acid sequence set forth in SEQ ID NO: 317 or comprising an amino acid sequence having at least 80%, 85%, 90%, 95%, 96%, 96%, 97%, 98%, or 99% identity to the amino acid set forth in SEQ ID NO: 317; and a light chain variable region comprising the amino acid sequence set forth in SEQ ID NO: 274 or comprising an amino acid sequence having at least 80%, 85%, 90%, 95%, 96%, 96%, 97%, 98%, or 99% identity to the amino acid set forth in SEQ ID NO: 274;
(e) a heavy chain variable region comprising the amino acid sequence set forth in SEQ ID NO: 318 or comprising an amino acid sequence having at least 80%, 85%, 90%, 95%, 96%, 96%, 97%, 98%, or 99% identity to the amino acid set forth in SEQ ID NO: 318; and a light chain variable region comprising the amino acid sequence set forth in SEQ ID NO: 275 or comprising an amino acid sequence having at least 80%, 85%, 90%, 95%, 96%, 96%, 97%, 98%, or 99% identity to the amino acid set forth in SEQ ID NO: 275;
(f) a heavy chain variable region comprising the amino acid sequence set forth in SEQ ID NO: 319 or comprising an amino acid sequence having at least 80%, 85%, 90%, 95%, 96%, 96%, 97%, 98%, or 99% identity to the amino acid set forth in SEQ ID NO: 319; and a light chain variable region comprising the amino acid sequence set forth in SEQ ID NO: 276 or comprising an amino acid sequence having at least 80%, 85%, 90%, 95%, 96%, 96%, 97%, 98%, or 99% identity to the amino acid set forth in SEQ ID NO: 276;
(g) a heavy chain variable region comprising the amino acid sequence set forth in SEQ ID NO: 320 or comprising an amino acid sequence having at least 80%, 85%, 90%, 95%, 96%, 96%, 97%, 98%, or 99% identity to the amino acid set forth in SEQ ID NO: 320; and a light chain variable region comprising the amino acid sequence set forth in SEQ ID NO: 277 or comprising an amino acid sequence having at least 80%, 85%, 90%, 95%, 96%, 96%, 97%, 98%, or 99% identity to the amino acid set forth in SEQ ID NO: 277;
(h) a heavy chain variable region comprising the amino acid sequence set forth in SEQ ID NO: 321 or comprising an amino acid sequence having at least 80%, 85%, 90%, 95%, 96%, 96%, 97%, 98%, or 99% identity to the amino acid set forth in SEQ ID NO: 321; and a light chain variable region comprising the amino acid sequence set forth in SEQ ID NO: 278 or comprising an amino acid sequence having at least 80%, 85%, 90%, 95%, 96%, 96%, 97%, 98%, or 99% identity to the amino acid set forth in SEQ ID NO: 278;
(i) a heavy chain variable region comprising the amino acid sequence set forth in SEQ ID NO: 322 or comprising an amino acid sequence having at least 80%, 85%, 90%, 95%, 96%, 96%, 97%, 98%, or 99% identity to the amino acid set forth in SEQ ID NO: 322; and a light chain variable region comprising the amino acid sequence set forth in SEQ ID NO: 279 or comprising an amino acid sequence having at least 80%, 85%, 90%, 95%, 96%, 96%, 97%, 98%, or 99% identity to the amino acid set forth in SEQ ID NO: 279;
(j) a heavy chain variable region comprising the amino acid sequence set forth in SEQ ID NO: 323 or comprising an amino acid sequence having at least 80%, 85%, 90%, 95%, 96%, 96%, 97%, 98%, or 99% identity to the amino acid set forth in SEQ ID NO: 323; and a light chain variable region comprising the amino acid sequence set forth in SEQ ID NO: 280 or comprising an amino acid sequence having at least 80%, 85%, 90%, 95%, 96%, 96%, 97%, 98%, or 99% identity to the amino acid set forth in SEQ ID NO: 280;
(k) a heavy chain variable region comprising the amino acid sequence set forth in SEQ ID NO: 324 or comprising an amino acid sequence having at least 80%, 85%, 90%, 95%, 96%, 96%, 97%, 98%, or 99% identity to the amino acid set forth in SEQ ID NO: 324; and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282 or comprising an amino acid sequence having at least 80%, 85%, 90%, 95%, 96%, 96%, 97%, 98%, or 99% identity to the amino acid set forth in SEQ ID NO: 282;
(m) a heavy chain variable region comprising the amino acid sequence set forth in SEQ ID NO: 326 or comprising an amino acid sequence having at least 80%, 85%, 90%, 95%, 96%, 96%, 97%, 98%, or 99% identity to the amino acid set forth in SEQ ID NO: 326; and a light chain variable region comprising the amino acid sequence set forth in SEQ ID NO: 283 or comprising an amino acid sequence having at least 80%, 85%, 90%, 95%, 96%, 96%, 97%, 98%, or 99% identity to the amino acid set forth in SEQ ID NO: 283;
(n) a heavy chain variable region comprising the amino acid sequence set forth in SEQ ID NO: 327 or comprising an amino acid sequence having at least 80%, 85%, 90%, 95%, 96%, 96%, 97%, 98%, or 99% identity to the amino acid set forth in SEQ ID NO: 327; and a light chain variable region comprising the amino acid sequence set forth in SEQ ID NO: 284 or comprising an amino acid sequence having at least 80%, 85%, 90%, 95%, 96%, 96%, 97%, 98%, or 99% identity to the amino acid set forth in SEQ ID NO: 284;
(o) a heavy chain variable region comprising the amino acid sequence set forth in SEQ ID NO: 328 or comprising an amino acid sequence having at least 80%, 85%, 90%, 95%, 96%, 96%, 97%, 98%, or 99% identity to the amino acid set forth in SEQ ID NO: 328; and a light chain variable region comprising the amino acid sequence set forth in SEQ ID NO: 285 or comprising an amino acid sequence having at least 80%, 85%, 90%, 95%, 96%, 96%, 97%, 98%, or 99% identity to the amino acid set forth in SEQ ID NO: 285;
(p) a heavy chain variable region comprising the amino acid sequence set forth in SEQ ID NO: 329 or comprising an amino acid sequence having at least 80%, 85%, 90%, 95%, 96%, 96%, 97%, 98%, or 99% identity to the amino acid set forth in SEQ ID NO: 329; and a light chain variable region comprising the amino acid sequence set forth in SEQ ID NO: 286 or comprising an amino acid sequence having at least 80%, 85%, 90%, 95%, 96%, 96%, 97%, 98%, or 99% identity to the amino acid set forth in SEQ ID NO: 286;
(q) a heavy chain variable region comprising the amino acid sequence set forth in SEQ ID NO: 330 or comprising an amino acid sequence having at least 80%, 85%, 90%, 95%, 96%, 96%, 97%, 98%, or 99% identity to the amino acid set forth in SEQ ID NO: 330; and a light chain variable region comprising the amino acid sequence set forth in SEQ ID NO: 287 or comprising an amino acid sequence having at least 80%, 85%, 90%, 95%, 96%, 96%, 97%, 98%, or 99% identity to the amino acid set forth in SEQ ID NO: 287;
(r) a heavy chain variable region comprising the amino acid sequence set forth in SEQ ID NO: 331 or comprising an amino acid sequence having at least 80%, 85%, 90%, 95%, 96%, 96%, 97%, 98%, or 99% identity to the amino acid set forth in SEQ ID NO: 331; and a light chain variable region comprising the amino acid sequence set forth in SEQ ID NO: 288 or comprising an amino acid sequence having at least 80%, 85%, 90%, 95%, 96%, 96%, 97%, 98%, or 99% identity to the amino acid set forth in SEQ ID NO: 288;
(s) a heavy chain variable region comprising the amino acid sequence set forth in SEQ ID NO: 332 or comprising an amino acid sequence having at least 80%, 85%, 90%, 95%, 96%, 96%, 97%, 98%, or 99% identity to the amino acid set forth in SEQ ID NO: 332; and a light chain variable region comprising the amino acid sequence set forth in SEQ ID NO: 289 or comprising an amino acid sequence having at least 80%, 85%, 90%, 95%, 96%, 96%, 97%, 98%, or 99% identity to the amino acid set forth in SEQ ID NO: 289;
(t) a heavy chain variable region comprising the amino acid sequence set forth in SEQ ID NO: 333 or comprising an amino acid sequence having at least 80%, 85%, 90%, 95%, 96%, 96%, 97%, 98%, or 99% identity to the amino acid set forth in SEQ ID NO: 333; and a light chain variable region comprising the amino acid sequence set forth in SEQ ID NO: 290 or comprising an amino acid sequence having at least 80%, 85%, 90%, 95%, 96%, 96%, 97%, 98%, or 99% identity to the amino acid set forth in SEQ ID NO: 290;
(u) a heavy chain variable region comprising the amino acid sequence set forth in SEQ ID NO: 334 or comprising an amino acid sequence having at least 80%, 85%, 90%, 95%, 96%, 96%, 97%, 98%, or 99% identity to the amino acid set forth in SEQ ID NO: 334; and a light chain variable region comprising the amino acid sequence set forth in SEQ ID NO: 291 or comprising an amino acid sequence having at least 80%, 85%, 90%, 95%, 96%, 96%, 97%, 98%, or 99% identity to the amino acid set forth in SEQ ID NO: 291;
(v) a heavy chain variable region comprising the amino acid sequence set forth in SEQ ID NO: 335 or comprising an amino acid sequence having at least 80%, 85%, 90%, 95%, 96%, 96%, 97%, 98%, or 99% identity to the amino acid set forth in SEQ ID NO: 335; and a light chain variable region comprising the amino acid sequence set forth in SEQ ID NO: 292 or comprising an amino acid sequence having at least 80%, 85%, 90%, 95%, 96%, 96%, 97%, 98%, or 99% identity to the amino acid set forth in SEQ ID NO: 292;
(w) a heavy chain variable region comprising the amino acid sequence set forth in SEQ ID NO: 336 or comprising an amino acid sequence having at least 80%, 85%, 90%, 95%, 96%, 96%, 97%, 98%, or 99% identity to the amino acid set forth in SEQ ID NO: 336; and a light chain variable region comprising the amino acid sequence set forth in SEQ ID NO: 293 or comprising an amino acid sequence having at least 80%, 85%, 90%, 95%, 96%, 96%, 97%, 98%, or 99% identity to the amino acid set forth in SEQ ID NO: 293;
(x) a heavy chain variable region comprising the amino acid sequence set forth in SEQ ID NO: 337 or comprising an amino acid sequence having at least 80%, 85%, 90%, 95%, 96%, 96%, 97%, 98%, or 99% identity to the amino acid set forth in SEQ ID NO: 337; and a light chain variable region comprising the amino acid sequence set forth in SEQ ID NO: 294 or comprising an amino acid sequence having at least 80%, 85%, 90%, 95%, 96%, 96%, 97%, 98%, or 99% identity to the amino acid set forth in SEQ ID NO: 294;
(y) a heavy chain variable region comprising the amino acid sequence set forth in SEQ ID NO: 338 or comprising an amino acid sequence having at least 80%, 85%, 90%, 95%, 96%, 96%, 97%, 98%, or 99% identity to the amino acid set forth in SEQ ID NO: 338; and a light chain variable region comprising the amino acid sequence set forth in SEQ ID NO: 295 or comprising an amino acid sequence having at least 80%, 85%, 90%, 95%, 96%, 96%, 97%, 98%, or 99% identity to the amino acid set forth in SEQ ID NO: 295;
(z) a heavy chain variable region comprising the amino acid sequence set forth in SEQ ID NO: 339 or comprising an amino acid sequence having at least 80%, 85%, 90%, 95%, 96%, 96%, 97%, 98%, or 99% identity to the amino acid set forth in SEQ ID NO: 339; and a light chain variable region comprising the amino acid sequence set forth in SEQ ID NO: 296 or comprising an amino acid sequence having at least 80%, 85%, 90%, 95%, 96%, 96%, 97%, 98%, or 99% identity to the amino acid set forth in SEQ ID NO: 296;
(aa) a heavy chain variable region comprising the amino acid sequence set forth in SEQ ID NO: 340 or comprising an amino acid sequence having at least 80%, 85%, 90%, 95%, 96%, 96%, 97%, 98%, or 99% identity to the amino acid set forth in SEQ ID NO: 340; and a light chain variable region comprising the amino acid sequence set forth in SEQ ID NO: 297 or comprising an amino acid sequence having at least 80%, 85%, 90%, 95%, 96%, 96%, 97%, 98%, or 99% identity to the amino acid set forth in SEQ ID NO: 297;
(bb) a heavy chain variable region comprising the amino acid sequence set forth in SEQ ID NO: 341 or comprising an amino acid sequence having at least 80%, 85%, 90%, 95%, 96%, 96%, 97%, 98%, or 99% identity to the amino acid set forth in SEQ ID NO: 341; and a light chain variable region comprising the amino acid sequence set forth in SEQ ID NO: 298 or comprising an amino acid sequence having at least 80%, 85%, 90%, 95%, 96%, 96%, 97%, 98%, or 99% identity to the amino acid set forth in SEQ ID NO: 298;
(cc) a heavy chain variable region comprising the amino acid sequence set forth in SEQ ID NO: 342 or comprising an amino acid sequence having at least 80%, 85%, 90%, 95%, 96%, 96%, 97%, 98%, or 99% identity to the amino acid set forth in SEQ ID NO: 342; and a light chain variable region comprising the amino acid sequence set forth in SEQ ID NO: 299 or comprising an amino acid sequence having at least 80%, 85%, 90%, 95%, 96%, 96%, 97%, 98%, or 99% identity to the amino acid set forth in SEQ ID NO: 299;
(dd) a heavy chain variable region comprising the amino acid sequence set forth in SEQ ID NO: 343 or comprising an amino acid sequence having at least 80%, 85%, 90%, 95%, 96%, 96%, 97%, 98%, or 99% identity to the amino acid set forth in SEQ ID NO: 343; and a light chain variable region comprising the amino acid sequence set forth in SEQ ID NO: 300 or comprising an amino acid sequence having at least 80%, 85%, 90%, 95%, 96%, 96%, 97%, 98%, or 99% identity to the amino acid set forth in SEQ ID NO: 300;
(ee) a heavy chain variable region comprising the amino acid sequence set forth in SEQ ID NO: 344 or comprising an amino acid sequence having at least 80%, 85%, 90%, 95%, 96%, 96%, 97%, 98%, or 99% identity to the amino acid set forth in SEQ ID NO: 344; and a light chain variable region comprising the amino acid sequence set forth in SEQ ID NO: 301 or comprising an amino acid sequence having at least 80%, 85%, 90%, 95%, 96%, 96%, 97%, 98%, or 99% identity to the amino acid set forth in SEQ ID NO: 301;
(ff) a heavy chain variable region comprising the amino acid sequence set forth in SEQ ID NO: 345 or comprising an amino acid sequence having at least 80%, 85%, 90%, 95%, 96%, 96%, 97%, 98%, or 99% identity to the amino acid set forth in SEQ ID NO: 345; and a light chain variable region comprising the amino acid sequence set forth in SEQ ID NO: 302 or comprising an amino acid sequence having at least 80%, 85%, 90%, 95%, 96%, 96%, 97%, 98%, or 99% identity to the amino acid set forth in SEQ ID NO: 302;
(gg) a heavy chain variable region comprising the amino acid sequence set forth in SEQ ID NO: 346 or comprising an amino acid sequence having at least 80%, 85%, 90%, 95%, 96%, 96%, 97%, 98%, or 99% identity to the amino acid set forth in SEQ ID NO: 346; and a light chain variable region comprising the amino acid sequence set forth in SEQ ID NO: 303 or comprising an amino acid sequence having at least 80%, 85%, 90%, 95%, 96%, 96%, 97%, 98%, or 99% identity to the amino acid set forth in SEQ ID NO: 303;
(hh) a heavy chain variable region comprising the amino acid sequence set forth in SEQ ID NO: 347 or comprising an amino acid sequence having at least 80%, 85%, 90%, 95%, 96%, 96%, 97%, 98%, or 99% identity to the amino acid set forth in SEQ ID NO: 347; and a light chain variable region comprising the amino acid sequence set forth in SEQ ID NO: 304 or comprising an amino acid sequence having at least 80%, 85%, 90%, 95%, 96%, 96%, 97%, 98%, or 99% identity to the amino acid set forth in SEQ ID NO: 304;
(ii) a heavy chain variable region comprising the amino acid sequence set forth in SEQ ID NO: 348 or comprising an amino acid sequence having at least 80%, 85%, 90%, 95%, 96%, 96%, 97%, 98%, or 99% identity to the amino acid set forth in SEQ ID NO: 348; and a light chain variable region comprising the amino acid sequence set forth in SEQ ID NO: 305 or comprising an amino acid sequence having at least 80%, 85%, 90%, 95%, 96%, 96%, 97%, 98%, or 99% identity to the amino acid set forth in SEQ ID NO: 305;
(jj) a heavy chain variable region comprising the amino acid sequence set forth in SEQ ID NO: 349 or comprising an amino acid sequence having at least 80%, 85%, 90%, 95%, 96%, 96%, 97%, 98%, or 99% identity to the amino acid set forth in SEQ ID NO: 349; and a light chain variable region comprising the amino acid sequence set forth in SEQ ID NO: 306 or comprising an amino acid sequence having at least 80%, 85%, 90%, 95%, 96%, 96%, 97%, 98%, or 99% identity to the amino acid set forth in SEQ ID NO: 306;
(kk) a heavy chain variable region comprising the amino acid sequence set forth in SEQ ID NO: 350 or comprising an amino acid sequence having at least 80%, 85%, 90%, 95%, 96%, 96%, 97%, 98%, or 99% identity to the amino acid set forth in SEQ ID NO: 350; and a light chain variable region comprising the amino acid sequence set forth in SEQ ID NO: 307 or comprising an amino acid sequence having at least 80%, 85%, 90%, 95%, 96%, 96%, 97%, 98%, or 99% identity to the amino acid set forth in SEQ ID NO: 307;
(ll) a heavy chain variable region comprising the amino acid sequence set forth in SEQ ID NO: 351 or comprising an amino acid sequence having at least 80%, 85%, 90%, 95%, 96%, 96%, 97%, 98%, or 99% identity to the amino acid set forth in SEQ ID NO: 351; and a light chain variable region comprising the amino acid sequence set forth in SEQ ID NO: 308 or comprising an amino acid sequence having at least 80%, 85%, 90%, 95%, 96%, 96%, 97%, 98%, or 99% identity to the amino acid set forth in SEQ ID NO: 308;
(mm) a heavy chain variable region comprising the amino acid sequence set forth in SEQ ID NO: 352 or comprising an amino acid sequence having at least 80%, 85%, 90%, 95%, 96%, 96%, 97%, 98%, or 99% identity to the amino acid set forth in SEQ ID NO: 352; and a light chain variable region comprising the amino acid sequence set forth in SEQ ID NO: 309 or comprising an amino acid sequence having at least 80%, 85%, 90%, 95%, 96%, 96%, 97%, 98%, or 99% identity to the amino acid set forth in SEQ ID NO: 309;
(nn) a heavy chain variable region comprising the amino acid sequence set forth in SEQ ID NO: 353 or comprising an amino acid sequence having at least 80%, 85%, 90%, 95%, 96%, 96%, 97%, 98%, or 99% identity to the amino acid set forth in SEQ ID NO: 353; and a light chain variable region comprising the amino acid sequence set forth in SEQ ID NO: 310 or comprising an amino acid sequence having at least 80%, 85%, 90%, 95%, 96%, 96%, 97%, 98%, or 99% identity to the amino acid set forth in SEQ ID NO: 310;
(oo) a heavy chain variable region comprising the amino acid sequence set forth in SEQ ID NO: 354 or comprising an amino acid sequence having at least 80%, 85%, 90%, 95%, 96%, 96%, 97%, 98%, or 99% identity to the amino acid set forth in SEQ ID NO: 354; and a light chain variable region comprising the amino acid sequence set forth in SEQ ID NO: 311 or comprising an amino acid sequence having at least 80%, 85%, 90%, 95%, 96%, 96%, 97%, 98%, or 99% identity to the amino acid set forth in SEQ ID NO: 311;
(pp) a heavy chain variable region comprising the amino acid sequence set forth in SEQ ID NO: 355 or comprising an amino acid sequence having at least 80%, 85%, 90%, 95%, 96%, 96%, 97%, 98%, or 99% identity to the amino acid set forth in SEQ ID NO: 355; and a light chain variable region comprising the amino acid sequence set forth in SEQ ID NO: 312 or comprising an amino acid sequence having at least 80%, 85%, 90%, 95%, 96%, 96%, 97%, 98%, or 99% identity to the amino acid set forth in SEQ ID NO: 312; or
(qq) a heavy chain variable region comprising the amino acid sequence set forth in SEQ ID NO: 356 or comprising an amino acid sequence having at least 80%, 85%, 90%, 95%, 96%, 96%, 97%, 98%, or 99% identity to the amino acid set forth in SEQ ID NO: 356; and a light chain variable region comprising the amino acid sequence set forth in SEQ ID NO: 313 or comprising an amino acid sequence having at least 80%, 85%, 90%, 95%, 96%, 96%, 97%, 98%, or 99% identity to the amino acid set forth in SEQ ID NO: 313.

3. The antibody or antigen-binding fragment thereof of claim 1, wherein the antibody or antigen-binding fragment thereof is selected from the group consisting of a full-length antibody, a single-chain variable fragment (scFv), a sc(Fv)2, a dsFv, a Fab, a Fab′, a (Fab′)2, and a diabody.

4. The antibody or antigen-binding fragment thereof of claim 1, wherein the antigen-binding fragment comprises an scFv.

5. A single-chain variable fragment (scFv) that comprises the antigen-binding fragment of claim 1, wherein the heavy chain variable region and the light chain variable region are connected by a linker.

6. The single chain variable fragment (scFv) of claim 5, comprising an amino acid sequence set forth in SEQ ID NOs: 44-86.

7. A chimeric antigen receptor (CAR) comprising the single-chain variable fragment (scFv) of claim 5.

8. A chimeric antigen receptor (CAR) comprising an antigen-binding domain specific for human CD19 (hCD19), a transmembrane domain, and an intracellular signaling domain, wherein the CAR comprises a heavy chain variable region that comprises heavy chain complementarity determining region 1 (HCDR1), HCDR2, and HCDR3 and a light chain variable region that comprises light chain complementarity determining region 1 (LCDR1), LCDR2, and LCDR3, and wherein the HCDR1, HCDR2, HCDR3, LCDR1, LCDR2, and LCDR3 comprise the respective amino acid sequences set forth in:
(a) SEQ ID NOs: 87, 88, 89, 90, and 91 and wherein LCDR2 comprises the amino acid sequence GNS;
(b) SEQ ID NOs: 92, 93, 94, 95, and 96, and wherein LCDR2 comprises the amino acid sequence AND;
(c) SEQ ID NOs: 97, 98, 99, 100, and 101, and wherein LCDR2 comprises the amino acid sequence ANI;
(d) SEQ ID NOs: 102, 103, 104, 100, and 105, and wherein LCDR2 comprises the amino acid sequence ANI;
(e) SEQ ID NOs: 106, 107, 108, 109, and 110, and wherein LCDR2 comprises the amino acid sequence SDG;
(f) SEQ ID NOs: 111, 112, 113, 114, and 115, and wherein LCDR2 comprises the amino acid sequence GSS;
(g) SEQ ID NOs: 116, 117, 118, 119, and 120, and wherein LCDR2 comprises the amino acid sequence GNT;
(h) SEQ ID NOs: 121, 122, 123, 124, and 125, and wherein LCDR2 comprises the amino acid sequence GYN;
(i) SEQ ID NOs: 126, 117, 127, 128, and 129, and wherein LCDR2 comprises the amino acid sequence GNS;
(j) SEQ ID NOs: 130, 131, 132, 100, and 133, and wherein LCDR2 comprises the amino acid sequence ANI;
(k) SEQ ID NOs: 134, 135, 136, 100, and 105, and wherein LCDR2 comprises the amino acid sequence ANI;
(l) SEQ ID NOs: 137, 138, 139, 140, and 141, and wherein LCDR2 comprises the amino acid sequence ADS;
(m) SEQ ID NOs: 142, 143, 144, 100, and 105, and wherein LCDR2 comprises the amino acid sequence ANI;
(n) SEQ ID NOs: 145, 146, 147, 148, and 149, and wherein LCDR2 comprises the amino acid sequence GNS;
(o) SEQ ID NOs: 150, 151, 152, 153, and 154, and wherein LCDR2 comprises the amino acid sequence GNS;
(p) SEQ ID NOs: 155, 156, 157, 158, and 159, and wherein LCDR2 comprises the amino acid sequence GSH;
(q) SEQ ID NOs: 121, 160, 161, 128, and 162, and wherein LCDR2 comprises the amino acid sequence GNS;
(r) SEQ ID NOs: 163, 164, 123, 124, and 165, and wherein LCDR2 comprises the amino acid sequence GYN;
(s) SEQ ID NOs: 106, 107, 108, 166, and 167, and wherein LCDR2 comprises the amino acid sequence SDG;
(t) SEQ ID NOs: 168, 169, 170, 171, and 172, and wherein LCDR2 comprises the amino acid sequence KDT;
(u) SEQ ID NOs: 173, 174, 175, 171, and 176, and wherein LCDR2 comprises the amino acid sequence KDT;
(v) SEQ ID NOs: 177, 178, 179, 180, and 181, and wherein LCDR2 comprises the amino acid sequence SSS;
(w) SEQ ID NOs: 182, 183, 184, 185, and 186, and wherein LCDR2 comprises the amino acid sequence LVS;
(x) SEQ ID NOs: 187, 188, 189, 100, and 190, and wherein LCDR2 comprises the amino acid sequence ANI;
(y) SEQ ID NOs: 191, 192, 193, 185, and 194, and wherein LCDR2 comprises the amino acid sequence QVS;
(z) SEQ ID NOs: 195, 196, 197, 171, and 198, and wherein LCDR2 comprises the amino acid sequence KDT;
(aa) SEQ ID NOs: 195, 196, 197, 171, and 199, and wherein LCDR2 comprises the amino acid sequence KDT;
(bb) SEQ ID NOs: 195, 196, 197, 200, and 201, and wherein LCDR2 comprises the amino acid sequence RDT;
(cc) SEQ ID NOs: 195, 196, 197, 200, and 202, and wherein LCDR2 comprises the amino acid sequence KDT;
(dd) SEQ ID NOs: 195, 196, 197, 171, and 190, and wherein LCDR2 comprises the amino acid sequence KDT;
(ee) SEQ ID NOs: 195, 196, 197, 203, and 204, and wherein LCDR2 comprises the amino acid sequence RDT;
(ff SEQ ID NOs: 195, 196, 197, 171, and 205, and wherein LCDR2 comprises the amino acid sequence KDT;
(gg) SEQ ID NOs: 206, 207, 208, 209, and 210, and wherein LCDR2 comprises the amino acid sequence SVS;
(hh) SEQ ID NOs: 211, 112, 113, 114, and 115, and wherein LCDR2 comprises the amino acid sequence GSS;
(ii) SEQ ID NOs: 168, 117, 212, 213, and 214, and wherein LCDR2 comprises the amino acid sequence KDT;
(jj) SEQ ID NOs: 121, 215, 216, 166, and 217, and wherein LCDR2 comprises the amino acid sequence SDG;
(kk) SEQ ID NOs: 218, 219, 220, 221, and 222, and wherein LCDR2 comprises the amino acid sequence RDT;
(ll) SEQ ID NOs: 223, 224, 225, 213, and 226, and wherein LCDR2 comprises the amino acid sequence KDT;
(mm) SEQ ID NOs: 121, 227, 228, 229, and 230, and wherein LCDR2 comprises the amino acid sequence SDG;
(nn) SEQ ID NOs: 231, 232, 233, 234, and 235, and wherein LCDR2 comprises the amino acid sequence NND;
(oo) SEQ ID NOs: 236, 237, 238, 171, and 239, and wherein LCDR2 comprises the amino acid sequence KDT;
(pp) SEQ ID NOs: 240, 143, 241, 242, and 243, and wherein LCDR2 comprises the amino acid sequence SNK; or
(qq) SEQ ID NOs: 244, 138, 245, 246, and 247, and wherein LCDR2 comprises the amino acid sequence EAS.

9. The CAR of claim 8, wherein the antigen-binding domain comprises:
(a) a heavy chain variable region comprising the amino acid sequence set forth in SEQ ID NO: 314 or comprising an amino acid sequence having at least 80%, 85%, 90%, 95%, 96%, 96%, 97%, 98%, or 99% identity to the amino acid set forth in SEQ ID NO: 314; and a light chain variable region comprising the amino acid sequence set forth in SEQ ID NO: 271 or comprising an amino acid sequence having at least 80%, 85%, 90%, 95%, 96%, 96%, 97%, 98%, or 99% identity to the amino acid set forth in SEQ ID NO: 271;
(b) a heavy chain variable region comprising the amino acid sequence set forth in SEQ ID NO: 315 or comprising an amino acid sequence having at least 80%, 85%, 90%, 95%, 96%, 96%, 97%, 98%, or 99% identity to the amino acid set forth in SEQ ID NO: 315; and a light chain variable region comprising the amino acid sequence set forth in SEQ ID NO: 272 or comprising an amino acid sequence having at least 80%, 85%, 90%, 95%, 96%, 96%, 97%, 98%, or 99% identity to the amino acid set forth in SEQ ID NO: 272;
(c) a heavy chain variable region comprising the amino acid sequence set forth in SEQ ID NO: 316 or comprising an amino acid sequence having at least 80%, 85%, 90%, 95%, 96%, 96%, 97%, 98%, or 99% identity to the amino acid set forth in SEQ ID NO: 316; and a light chain variable region comprising the amino acid sequence set forth in SEQ ID NO: 273 or comprising an amino acid sequence having at least 80%, 85%, 90%, 95%, 96%, 96%, 97%, 98%, or 99% identity to the amino acid set forth in SEQ ID NO: 273;
(d) a heavy chain variable region comprising the amino acid sequence set forth in SEQ ID NO: 317 or comprising an amino acid sequence having at least 80%, 85%, 90%, 95%, 96%, 96%, 97%, 98%, or 99% identity to the amino acid set forth in SEQ ID NO: 317; and a light chain variable region comprising the amino acid sequence set forth in SEQ ID NO: 274 or comprising an amino acid sequence having at least 80%, 85%, 90%, 95%, 96%, 96%, 97%, 98%, or 99% identity to the amino acid set forth in SEQ ID NO: 274;
(e) a heavy chain variable region comprising the amino acid sequence set forth in SEQ ID NO: 318 or comprising an amino acid sequence having at least 80%, 85%, 90%, 95%, 96%, 96%, 97%, 98%, or 99% identity to the amino acid set forth in SEQ ID NO: 318; and a light chain variable region comprising the amino acid sequence set forth in SEQ ID NO: 275 or comprising an amino acid sequence having at least 80%, 85%, 90%, 95%, 96%, 96%, 97%, 98%, or 99% identity to the amino acid set forth in SEQ ID NO: 275;
(f) a heavy chain variable region comprising the amino acid sequence set forth in SEQ ID NO: 319 or comprising an amino acid sequence having at least 80%, 85%, 90%, 95%, 96%, 96%, 97%, 98%, or 99% identity to the amino acid set forth in SEQ ID NO: 319; and a light chain variable region comprising the amino acid sequence set forth in SEQ ID NO: 276 or comprising an amino acid sequence having at least 80%, 85%, 90%, 95%, 96%, 96%, 97%, 98%, or 99% identity to the amino acid set forth in SEQ ID NO: 276;
(g) a heavy chain variable region comprising the amino acid sequence set forth in SEQ ID NO: 320 or comprising an amino acid sequence having at least 80%, 85%, 90%, 95%, 96%, 96%, 97%, 98%, or 99% identity to the amino acid set forth in SEQ ID NO: 320; and a light chain variable region comprising the amino acid sequence set forth in SEQ ID NO: 277 or comprising an amino acid sequence having at least 80%, 85%, 90%, 95%, 96%, 96%, 97%, 98%, or 99% identity to the amino acid set forth in SEQ ID NO: 277;
(h) a heavy chain variable region comprising the amino acid sequence set forth in SEQ ID NO: 321 or comprising an amino acid sequence having at least 80%, 85%, 90%, 95%, 96%, 96%, 97%, 98%, or 99% identity to the amino acid set forth in SEQ ID NO: 321; and a light chain variable region comprising the amino acid sequence set forth in SEQ ID NO: 278 or comprising an amino acid sequence having at least 80%, 85%, 90%, 95%, 96%, 96%, 97%, 98%, or 99% identity to the amino acid set forth in SEQ ID NO: 278;
(i) a heavy chain variable region comprising the amino acid sequence set forth in SEQ ID NO: 322 or comprising an amino acid sequence having at least 80%, 85%, 90%, 95%, 96%, 96%, 97%, 98%, or 99% identity to the amino acid set forth in SEQ ID NO: 322; and a light chain variable region comprising the amino acid sequence set forth in SEQ ID NO: 279 or comprising an amino acid sequence having at least 80%, 85%, 90%, 95%, 96%, 96%, 97%, 98%, or 99% identity to the amino acid set forth in SEQ ID NO: 279;
(j) a heavy chain variable region comprising the amino acid sequence set forth in SEQ ID NO: 323 or comprising an amino acid sequence having at least 80%, 85%, 90%, 95%, 96%, 96%, 97%, 98%, or 99% identity to the amino acid set forth in SEQ ID NO: 323; and a light chain variable region comprising the amino acid sequence set forth in SEQ ID NO: 280 or comprising an amino acid sequence having at least 80%, 85%, 90%, 95%, 96%, 96%, 97%, 98%, or 99% identity to the amino acid set forth in SEQ ID NO: 280;
(k) a heavy chain variable region comprising the amino acid sequence set forth in SEQ ID NO: 324 or comprising an amino acid sequence having at least 80%, 85%, 90%, 95%, 96%, 96%, 97%, 98%, or 99% identity to the amino acid set forth in SEQ ID NO: 324; and a light chain variable region comprising the amino acid sequence set forth in SEQ ID NO: 281;
(l) a heavy chain variable region comprising the amino acid sequence set forth in SEQ ID NO: 325 and a light chain variable region comprising the amino acid sequence set forth in SEQ ID NO: 282 or comprising an amino acid sequence having at least 80%, 85%, 90%, 95%, 96%, 96%, 97%, 98%, or 99% identity to the amino acid set forth in SEQ ID NO: 282;
(m) a heavy chain variable region comprising the amino acid sequence set forth in SEQ ID NO: 326 or comprising an amino acid sequence having at least 80%, 85%, 90%, 95%, 96%, 96%, 97%, 98%, or 99% identity to the amino acid set forth in SEQ ID NO: 326; and a light chain variable region comprising the amino acid sequence set forth in SEQ ID NO: 283 or comprising an amino acid sequence having at least 80%, 85%, 90%, 95%, 96%, 96%, 97%, 98%, or 99% identity to the amino acid set forth in SEQ ID NO: 283;
(n) a heavy chain variable region comprising the amino acid sequence set forth in SEQ ID NO: 327 or comprising an amino acid sequence having at least 80%, 85%, 90%, 95%, 96%, 96%, 97%, 98%, or 99% identity to the amino acid set forth in SEQ ID NO: 327; and a light chain variable region comprising the amino acid sequence set forth in SEQ ID NO: 284 or comprising an amino acid sequence having at least 80%, 85%, 90%, 95%, 96%, 96%, 97%, 98%, or 99% identity to the amino acid set forth in SEQ ID NO: 284;
(o) a heavy chain variable region comprising the amino acid sequence set forth in SEQ ID NO: 328 or comprising an amino acid sequence having at least 80%, 85%, 90%, 95%, 96%, 96%, 97%, 98%, or 99% identity to the amino acid set forth in SEQ ID NO: 328; and a light chain variable region comprising the amino acid sequence set forth in SEQ ID NO: 285 or comprising an amino acid sequence having at least 80%, 85%, 90%, 95%, 96%, 96%, 97%, 98%, or 99% identity to the amino acid set forth in SEQ ID NO: 285;
(p) a heavy chain variable region comprising the amino acid sequence set forth in SEQ ID NO: 329 or comprising an amino acid sequence having at least 80%, 85%, 90%, 95%, 96%, 96%, 97%, 98%, or 99% identity to the amino acid set forth in SEQ ID NO: 329; and a light chain variable region comprising the amino acid sequence set forth in SEQ ID NO: 286 or comprising an amino acid sequence having at least 80%, 85%, 90%, 95%, 96%, 96%, 97%, 98%, or 99% identity to the amino acid set forth in SEQ ID NO: 286;
(q) a heavy chain variable region comprising the amino acid sequence set forth in SEQ ID NO: 330 or comprising an amino acid sequence having at least 80%, 85%, 90%, 95%, 96%, 96%, 97%, 98%, or 99% identity to the amino acid set forth in SEQ ID NO: 330; and a light chain variable region comprising the amino acid sequence set forth in SEQ ID NO: 287 or comprising an amino acid sequence having at least 80%, 85%, 90%, 95%, 96%, 96%, 97%, 98%, or 99% identity to the amino acid set forth in SEQ ID NO: 287;
(r) a heavy chain variable region comprising the amino acid sequence set forth in SEQ ID NO: 331 or comprising an amino acid sequence having at least 80%, 85%, 90%, 95%, 96%, 96%, 97%, 98%, or 99% identity to the amino acid set forth in SEQ ID NO: 331; and a light chain variable region comprising the amino acid sequence set forth in SEQ ID NO: 288 or comprising an amino acid sequence having at least 80%, 85%, 90%, 95%, 96%, 96%, 97%, 98%, or 99% identity to the amino acid set forth in SEQ ID NO: 288;
(s) a heavy chain variable region comprising the amino acid sequence set forth in SEQ ID NO: 332 or comprising an amino acid sequence having at least 80%, 85%, 90%, 95%, 96%, 96%, 97%, 98%, or 99% identity to the amino acid set forth in SEQ ID NO: 332; and a light chain variable region comprising the amino acid sequence set forth in SEQ ID NO: 289 or comprising an amino acid sequence having at least 80%, 85%, 90%, 95%, 96%, 96%, 97%, 98%, or 99% identity to the amino acid set forth in SEQ ID NO: 289;
(t) a heavy chain variable region comprising the amino acid sequence set forth in SEQ ID NO: 333 or comprising an amino acid sequence having at least 80%, 85%, 90%, 95%, 96%, 96%, 97%, 98%, or 99% identity to the amino acid set forth in SEQ ID NO: 333; and a light chain variable region comprising the amino acid sequence set forth in SEQ ID NO: 290 or comprising an amino acid sequence having at least 80%, 85%, 90%, 95%, 96%, 96%, 97%, 98%, or 99% identity to the amino acid set forth in SEQ ID NO: 290;
(u) a heavy chain variable region comprising the amino acid sequence set forth in SEQ ID NO: 334 or comprising an amino acid sequence having at least 80%, 85%, 90%, 95%, 96%, 96%, 97%, 98%, or 99% identity to the amino acid set forth in SEQ ID NO: 334; and a light chain variable region comprising the amino acid sequence set forth in SEQ ID NO: 291 or comprising an amino acid sequence having at least 80%, 85%, 90%, 95%, 96%, 96%, 97%, 98%, or 99% identity to the amino acid set forth in SEQ ID NO: 291;
(v) a heavy chain variable region comprising the amino acid sequence set forth in SEQ ID NO: 335 or comprising an amino acid sequence having at least 80%, 85%, 90%, 95%, 96%, 96%, 97%, 98%, or 99% identity to the amino acid set forth in SEQ ID NO: 335; and a light chain variable region comprising the amino acid sequence set forth in SEQ ID NO: 292 or comprising an amino acid sequence having at least 80%, 85%, 90%, 95%, 96%, 96%, 97%, 98%, or 99% identity to the amino acid set forth in SEQ ID NO: 292;
(w) a heavy chain variable region comprising the amino acid sequence set forth in SEQ ID NO: 336 or comprising an amino acid sequence having at least 80%, 85%, 90%, 95%, 96%, 96%, 97%, 98%, or 99% identity to the amino acid set forth in SEQ ID NO: 336; and a light chain variable region comprising the amino acid sequence set forth in SEQ ID NO: 293 or comprising an amino acid sequence having at least 80%, 85%, 90%, 95%, 96%, 96%, 97%, 98%, or 99% identity to the amino acid set forth in SEQ ID NO: 293;
(x) a heavy chain variable region comprising the amino acid sequence set forth in SEQ ID NO: 337 or comprising an amino acid sequence having at least 80%, 85%, 90%, 95%, 96%, 96%, 97%, 98%, or 99% identity to the amino acid set forth in SEQ ID NO: 337; and a light chain variable region comprising the amino acid sequence set forth in SEQ ID NO: 294 or comprising an amino acid sequence having at least 80%, 85%, 90%, 95%, 96%, 96%, 97%, 98%, or 99% identity to the amino acid set forth in SEQ ID NO: 294;
(y) a heavy chain variable region comprising the amino acid sequence set forth in SEQ ID NO: 338 or comprising an amino acid sequence having at least 80%, 85%, 90%, 95%, 96%, 96%, 97%, 98%, or 99% identity to the amino acid set forth in SEQ ID NO: 338; and a light chain variable region comprising the amino acid sequence set forth in SEQ ID NO: 295 or comprising an amino acid sequence having at least 80%, 85%, 90%, 95%, 96%, 96%, 97%, 98%, or 99% identity to the amino acid set forth in SEQ ID NO: 295;
(z) a heavy chain variable region comprising the amino acid sequence set forth in SEQ ID NO: 339 or comprising an amino acid sequence having at least 80%, 85%, 90%, 95%, 96%, 96%, 97%, 98%, or 99% identity to the amino acid set forth in SEQ ID NO: 339; and a light chain variable region comprising the amino acid sequence set forth in SEQ ID NO: 296 or comprising an amino acid sequence having at least 80%, 85%, 90%, 95%, 96%, 96%, 97%, 98%, or 99% identity to the amino acid set forth in SEQ ID NO: 296;
(aa) a heavy chain variable region comprising the amino acid sequence set forth in SEQ ID NO: 340 or comprising an amino acid sequence having at least 80%, 85%, 90%, 95%, 96%, 96%, 97%, 98%, or 99% identity to the amino acid set forth in SEQ ID NO: 340; and a light chain variable region comprising the amino acid sequence set forth in SEQ ID NO: 297 or comprising an amino acid sequence having at least 80%, 85%, 90%, 95%, 96%, 96%, 97%, 98%, or 99% identity to the amino acid set forth in SEQ ID NO: 297;
(bb) a heavy chain variable region comprising the amino acid sequence set forth in SEQ ID NO: 341 or comprising an amino acid sequence having at least 80%, 85%, 90%, 95%, 96%, 96%, 97%, 98%, or 99% identity to the amino acid set forth in SEQ ID NO: 341; and a light chain variable region comprising the amino acid sequence set forth in SEQ ID NO: 298 or comprising an amino acid sequence having at least 80%, 85%, 90%, 95%, 96%, 96%, 97%, 98%, or 99% identity to the amino acid set forth in SEQ ID NO: 298;
(cc) a heavy chain variable region comprising the amino acid sequence set forth in SEQ ID NO: 342 or comprising an amino acid sequence having at least 80%, 85%, 90%, 95%, 96%, 96%, 97%, 98%, or 99% identity to the amino acid set forth in SEQ ID NO: 342; and a light chain variable region comprising the amino acid sequence set forth in SEQ ID NO: 299 or comprising an amino acid sequence having at least 80%, 85%, 90%, 95%, 96%, 96%, 97%, 98%, or 99% identity to the amino acid set forth in SEQ ID NO: 299;
(dd) a heavy chain variable region comprising the amino acid sequence set forth in SEQ ID NO: 343 or comprising an amino acid sequence having at least 80%, 85%, 90%, 95%, 96%, 96%, 97%, 98%, or 99% identity to the amino acid set forth in SEQ ID NO: 343; and a light chain variable region comprising the amino acid sequence set forth in SEQ ID NO: 300 or comprising an amino acid sequence having at least 80%, 85%, 90%, 95%, 96%, 96%, 97%, 98%, or 99% identity to the amino acid set forth in SEQ ID NO: 300;
(ee) a heavy chain variable region comprising the amino acid sequence set forth in SEQ ID NO: 344 or comprising an amino acid sequence having at least 80%, 85%, 90%, 95%, 96%, 96%, 97%, 98%, or 99% identity to the amino acid set forth in SEQ ID NO: 344; and a light chain variable region comprising the amino acid sequence set forth in SEQ ID NO: 301 or comprising an amino acid sequence having at least 80%, 85%, 90%, 95%, 96%, 96%, 97%, 98%, or 99% identity to the amino acid set forth in SEQ ID NO: 301;
(ff) a heavy chain variable region comprising the amino acid sequence set forth in SEQ ID NO: 345 or comprising an amino acid sequence having at least 80%, 85%, 90%, 95%, 96%, 96%, 97%, 98%, or 99% identity to the amino acid set forth in SEQ ID NO: 345; and a light chain variable region comprising the amino acid sequence set forth in SEQ ID NO: 302 or comprising an amino acid sequence having at least 80%, 85%, 90%, 95%, 96%, 96%, 97%, 98%, or 99% identity to the amino acid set forth in SEQ ID NO: 302;
(gg) a heavy chain variable region comprising the amino acid sequence set forth in SEQ ID NO: 346 or comprising an amino acid sequence having at least 80%, 85%, 90%, 95%, 96%, 96%, 97%, 98%, or 99% identity to the amino acid set forth in SEQ ID NO: 346; and a light chain variable region comprising the amino acid sequence set forth in SEQ ID NO: 303 or comprising an amino acid sequence having at least 80%, 85%, 90%, 95%, 96%, 96%, 97%, 98%, or 99% identity to the amino acid set forth in SEQ ID NO: 303;
(hh) a heavy chain variable region comprising the amino acid sequence set forth in SEQ ID NO: 347 or comprising an amino acid sequence having at least 80%, 85%, 90%, 95%, 96%, 96%, 97%, 98%, or 99% identity to the amino acid set forth in SEQ ID NO: 347; and a light chain variable region comprising the amino acid sequence set forth in SEQ ID NO: 304 or comprising an amino acid sequence having at least 80%, 85%, 90%, 95%, 96%, 96%, 97%, 98%, or 99% identity to the amino acid set forth in SEQ ID NO: 304;
(ii) a heavy chain variable region comprising the amino acid sequence set forth in SEQ ID NO: 348 or comprising an amino acid sequence having at least 80%, 85%, 90%, 95%, 96%, 96%, 97%, 98%, or 99% identity to the amino acid set forth in SEQ ID NO: 348; and a light chain variable region comprising the amino acid sequence set forth in SEQ ID NO: 305 or comprising an amino acid sequence having at least 80%, 85%, 90%, 95%, 96%, 96%, 97%, 98%, or 99% identity to the amino acid set forth in SEQ ID NO: 305;
(jj) a heavy chain variable region comprising the amino acid sequence set forth in SEQ ID NO: 349 or comprising an amino acid sequence having at least 80%, 85%, 90%, 95%, 96%, 96%, 97%, 98%, or 99% identity to the amino acid set forth in SEQ ID NO: 349; and a light chain variable region comprising the amino acid sequence set forth in SEQ ID NO: 306 or comprising an amino acid sequence having at least 80%, 85%, 90%, 95%, 96%, 96%, 97%, 98%, or 99% identity to the amino acid set forth in SEQ ID NO: 306;
(kk) a heavy chain variable region comprising the amino acid sequence set forth in SEQ ID NO: 350 or comprising an amino acid sequence having at least 80%, 85%, 90%, 95%, 96%, 96%, 97%, 98%, or 99% identity to the amino acid set forth in SEQ ID NO: 350; and a light chain variable region comprising the amino acid sequence set forth in SEQ ID NO: 307 or comprising an amino acid sequence having at least 80%, 85%, 90%, 95%, 96%, 96%, 97%, 98%, or 99% identity to the amino acid set forth in SEQ ID NO: 307;
(ll) a heavy chain variable region comprising the amino acid sequence set forth in SEQ ID NO: 351 or comprising an amino acid sequence having at least 80%, 85%, 90%, 95%, 96%, 96%, 97%, 98%, or 99% identity to the amino acid set forth in SEQ ID NO: 351; and a light chain variable region comprising the amino acid sequence set forth in SEQ ID NO: 308 or comprising an amino acid sequence having at least 80%, 85%, 90%, 95%, 96%, 96%, 97%, 98%, or 99% identity to the amino acid set forth in SEQ ID NO: 308;
(mm) a heavy chain variable region comprising the amino acid sequence set forth in SEQ ID NO: 352 or comprising an amino acid sequence having at least 80%, 85%, 90%, 95%, 96%, 96%, 97%, 98%, or 99% identity to the amino acid set forth in SEQ ID NO: 352; and a light chain variable region comprising the amino acid sequence set forth in SEQ ID NO: 309 or comprising an amino acid sequence having at least 80%, 85%, 90%, 95%, 96%, 96%, 97%, 98%, or 99% identity to the amino acid set forth in SEQ ID NO: 309;
(nn) a heavy chain variable region comprising the amino acid sequence set forth in SEQ ID NO: 353 or comprising an amino acid sequence having at least 80%, 85%, 90%, 95%, 96%, 96%, 97%, 98%, or 99% identity to the amino acid set forth in SEQ ID NO: 353; and a light chain variable region comprising the amino acid sequence set forth in SEQ ID NO: 310 or comprising an amino acid sequence having at least 80%, 85%, 90%, 95%, 96%, 96%, 97%, 98%, or 99% identity to the amino acid set forth in SEQ ID NO: 310;
(oo) a heavy chain variable region comprising the amino acid sequence set forth in SEQ ID NO: 354 or comprising an amino acid sequence having at least 80%, 85%, 90%, 95%, 96%, 96%, 97%, 98%, or 99% identity to the amino acid set forth in SEQ ID NO: 354; and a light chain variable region comprising the amino acid sequence set forth in SEQ ID NO: 311 or comprising an amino acid sequence having at least 80%, 85%, 90%, 95%, 96%, 96%, 97%, 98%, or 99% identity to the amino acid set forth in SEQ ID NO: 311;
(pp) a heavy chain variable region comprising the amino acid sequence set forth in SEQ ID NO: 355 or comprising an amino acid sequence having at least 80%, 85%, 90%, 95%, 96%, 96%, 97%, 98%, or 99% identity to the amino acid set forth in SEQ ID NO: 355; and a light chain variable region comprising the amino acid sequence set forth in SEQ ID NO: 312 or comprising an amino acid sequence having at least 80%, 85%, 90%, 95%, 96%, 96%, 97%, 98%, or 99% identity to the amino acid set forth in SEQ ID NO: 312; or
(qq) a heavy chain variable region comprising the amino acid sequence set forth in SEQ ID NO: 356 or comprising an amino acid sequence having at least 80%, 85%, 90%, 95%, 96%, 96%, 97%, 98%, or 99% identity to the amino acid set forth in SEQ ID NO: 356; and a light chain variable region comprising the amino acid sequence set forth in SEQ ID NO: 313 or comprising an amino acid sequence having at least 80%, 85%, 90%, 95%, 96%, 96%, 97%, 98%, or 99% identity to the amino acid set forth in SEQ ID NO: 313.

10. The CAR of claim 8, wherein the antigen binding domain comprises: a heavy chain variable region comprising an amino acid sequence of any of the heavy chain variable regions set forth in SEQ ID NOs: 314-356 or comprises a heavy chain variable region comprising an amino acid sequence having at least 95%-99% identity to an amino acid sequence of any of the heavy chain variable regions set forth in SEQ ID NOs: 314-356; and/or a light chain variable region comprising an amino acid sequence of any of the light chain variable regions set forth in SEQ ID NOs: 271-313 or comprises a light chain variable region comprising an amino acid sequence having at least 95-99% identity to an amino acid sequence of any of the light chain variable regions set forth in SEQ ID NOs: 271-313.

11. The CAR of claim 8, wherein the antigen binding domain comprises any one of the amino acid sequences set forth in SEQ ID NOs: 44-86.

12. The CAR of claim 8, wherein the intracellular signaling domain is derived from a human NK cell receptor (NKR), and wherein the CAR is an NKR-CAR.

13. The CAR of claim 12, wherein the human NK cell receptor (NKR) comprises a NKR domain selected from the group consisting of an actKIR domain, an NCR domain, a SLAMF domain, an FcR domain, a CD16 domain, a CD64 domain, an actLy49 domain, and a an inhLy49 domain.

14. The CAR of claim 8, wherein the intracellular signaling domain comprises a KIR signaling domain selected from the group consisting of a KIR2DS2 signaling domain, a KIR2DS2 signaling domain, a KIR2DL3 signaling domain, a KIR2DL1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain, and a KIR3DP1 signaling domain.

15. The CAR of claim 8, wherein the transmembrane domain comprises a KIR transmembrane domain selected from the group consisting of a KIR2DS2 transmembrane domain, a KIR2DS2 transmembrane domain, a KIR2DL3 transmembrane domain, a KIR2DL 1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL1 transmembrane domain, a KIR3DS1 transmembrane domain, a KIR3DL2 transmembrane domain, a KIR3DL3 transmembrane domain, a KIR2DP1 transmembrane domain, and a KIR3DP1 transmembrane domain.

16. The CAR of claim 8, wherein transmembrane domain can bind the transmembrane domain of DAP12.

17. The CAR of claim 14, wherein the KIR signaling domain comprises the amino acid sequence set forth in SEQ ID NO: 266.

18. The CAR of claim 8, wherein the CAR comprises an amino acid sequence set forth in SEQ ID NOs: 370-412.

19. The CAR of claim 8, comprising:
(a) the CD8 leader amino acid sequence set forth in SEQ ID NO: 357;
(b) an scFv amino acid sequence selected from the group consisting of SEQ ID NOs: 44-86;
(c) the myc tag amino acid sequence set forth in SEQ ID NO: 264; and
(d) the transmembrane/KIR2DS2 signaling domain amino acid sequence set forth in SEQ ID NO: 266.

20. An NK cell receptor (NKR)-CAR complex, comprising an NKR-CAR that comprises the antigen-binding fragment of claim 1 and an adaptor molecule.

21. The NKR-CAR complex of claim 20, wherein the NKR-CAR comprises the amino acid sequence set forth in any one of SEQ ID NOs: 370-412.

22. The NKR-CAR complex of claim 20, wherein the adaptor molecule comprises DAP12 or FcεRγ.

23. The NKR-CAR complex of claim 20, wherein the NKR-CAR interacts with the adaptor molecule upon binding of the antigen binding domain of the NKR-CAR to human CD19.

24. The NKR-CAR complex of claim 20, wherein the NKR-CAR further comprises
(a) an NKR transmembrane domain; and/or
(b) an NKR intracellular signaling domain.

25. The NKR-CAR complex of claim 24, wherein the NKR intracellular signaling domain is selected from the group consisting of an actKIR domain, an NCR domain, a SLAMF domain, an FcR domain, a CD16 domain, a CD64 domain, an actLy49 domain, and an inhLy49 domain.

26. The NKR-CAR complex of claim 20, wherein the NKR-CAR is a KIR-CAR.

27. The NKR-CAR complex of claim 24, wherein the intracellular signaling domain comprises a KIR signaling domain selected from the group consisting of a KIR2DS2 signaling domain, a KIR2DS2 signaling domain, a KIR2DL3 signaling domain, a KIR2DL1 signaling domain, a KIR2DL2 signaling domain, a KIR2DL4 signaling domain, a KIR2DL5A signaling domain, a KIR2DL5B signaling domain, a KIR2DS1 signaling domain, a KIR2DS3 signaling domain, a KIR2DS4 signaling domain, a KIR2DS5 signaling domain, a KIR3DL1 signaling domain, a KIR3DS1 signaling domain, a KIR3DL2 signaling domain, a KIR3DL3 signaling domain, a KIR2DP1 signaling domain and a KIR3DP1 signaling domain.

28. The NKR-CAR complex of claim 24, wherein the transmembrane domain comprises a KIR transmembrane domain selected from the group consisting of a KIR2DS2 transmembrane domain, a KIR2DS2 transmembrane domain, a KIR2DL3 transmembrane domain, a KIR2DL1 transmembrane domain, a KIR2DL2 transmembrane domain, a KIR2DL4 transmembrane domain, a KIR2DL5A transmembrane domain, a KIR2DL5B transmembrane domain, a KIR2DS1 transmembrane domain, a KIR2DS3 transmembrane domain, a KIR2DS4 transmembrane domain, a KIR2DS5 transmembrane domain, a KIR3DL1 transmembrane domain, a KIR3DS1 transmembrane domain, a KIR3DL2 transmembrane domain, a KIR3DL3 transmembrane domain, a KIR2DP1 transmembrane domain, and a KIR3DP1 transmembrane domain.

29. The NKR-CAR complex of claim 24, wherein transmembrane domain is capable of binding and/or activating DAP12 through the transmembrane domain of DAP12.

30. The NKR-CAR complex of claim 27, wherein the KIR signaling domain comprises the amino acid sequence set forth in SEQ ID NO: 266.

31. The NKR-CAR complex of claim 20, wherein the CAR comprising an amino acid sequence set forth in SEQ ID NOs: 370-412.

32. An isolated nucleic acid encoding the antibody or antigen-binding fragment thereof of claim 1.

33. An isolated nucleic acid encoding the CAR of claim 8.

34. The isolated nucleic acid of claim 32, comprising a polynucleotide sequence set forth in SEQ ID NOs: 1-43.

35. A vector comprising the isolated nucleic acid of claim 32.

36. A modified immune cell comprising the isolated nucleic acid of claim 32.

37. The modified immune cell of claim 36, wherein the modified immune cell is mammalian in origin.

38. The modified immune cell of claim 37, wherein the modified immune cell is a T cell.

39. A pharmaceutical composition comprising: (a) the modified immune cell of claim 36, and (b) a pharmaceutically acceptable excipient, carrier, or diluent.

40. A method of treating cancer or an autoimmune disorder or disease in a subject in need thereof, comprising administering to the subject an effective amount of the modified immune cell of claim 36.

41. The method of claim 40, wherein the cancer is a hematologic cancer.

42. The method of claim 40, wherein the cancer is associated with the expression of CD19.

43. The method of claim 42, wherein the CD19 is expressed on tumor cells.

44. The method of claim 40, wherein the cancer is selected from the group consisting of Burkitt lymphoma, chronic lymphocytic leukemia (CLL), acute lymphoblastic leukemia (ALL), B-cell lymphoma, and B-cell leukemia.

45. The method of claim 40, wherein administration of the modified immune cell depletes autoimmune B cells.

46. The method of claim 40, wherein the autoimmune disorder is selected from the group consisting of rheumatoid arthritis, systemic lupus erythematosus (SLE), lupus nephritis (LN), idiopathic/autoimmune thrombocytopenia purpura (ITP), idiopathic thrombotic thrombocytopeniaurpura (TTP), pemphigus-related disorders, diabetes, scleroderma, myasthenia gravis, multiple sclerosis, vasculitis, and autoimmune hemolytic anemia.

47. The method of claim 40, wherein the autoimmune disorder includes the production of autoantibodies and the administration of the modified immune cell depletes B cells that generate autoantibodies.
